(19) United States
(12) Patent Application Publication
(10) Pub. No.: US 2024/0150471 A1
(43) Pub. Date: May 9, 2024

(54) NATURAL KILLER CELL ANTIBODIES WITH INCREASED ADCC ACTIVITY

(71) Applicant: Xencor, Inc., Pasadena, CA (US)

(72) Inventor: Matthew S. Faber, Glendora, CA (US)

(21) Appl. No.: 18/502,668

(22) Filed: Nov. 6, 2023

Related U.S. Application Data
(60) Provisional application No. 63/382,630, filed on Nov. 7, 2022.
(60) Provisional application No. 63/382,602, filed on Nov. 7, 2022.

(51) Int. Cl. C07K 16/28; C07K 16/30; C07K 16/32

(52) U.S. Cl. CPC C07K 16/2851; C07K 16/28; C07K 16/2803; C07K 16/2818; C07K 16/2827; C07K 16/283; C07K 16/2863; C07K 16/2866; C07K 16/2878; C07K 16/2887; C07K 16/2896; C07K 16/30; C07K 16/3007; C07K 16/32; C07K 2317/31; C07K 2317/52; C07K 2317/732

(57) ABSTRACT

Described herein are antibodies, including multispecific antibodies, that bind to NK cells, optionally a tumor target antigen (TTA) and have increased binding to the CD16A receptor. The antibodies can include an NK cell antigen binding domain (ABD) that binds to the extracellular domain of an NK cell antigen such as NKG2D, NKp30, and NKp46.

### CROSS REFERENCE TO RELATED APPLICATIONS

This application claims the benefit of U.S. Provisional Patent Application Nos. 63/382,630 and 63/382,602, both filed on Nov. 7, 2022, which are hereby incorporated by reference in their entirety.

### SEQUENCE LISTING

The instant application contains a Sequence Listing which has been submitted electronically in XML format and is hereby incorporated by reference in its entirety. The XML copy, created on Oct. 24, 2023, is named 51096_4007_WO.xml and is 2,285,568 bytes in size.

### BACKGROUND

Natural killer cell engagers (NKEs), a new class of immune-oncology therapeutics, contain fragments of antibodies such as antibody binding domains and are designed to exploit the immune functions of NK cells in cancer.

NK cells are part of the innate immune system and represent 5-20% of circulating lymphocytes in humans. NK cells infiltrate virtually all tissues and were originally characterized by their ability to kill tumor cells effectively without the need for prior sensitization. This ability to infiltrate also occurs in the tumor microenvironment and this infiltration is associated with better overall survival in patients.

Activated NK cells kill target cells by means similar to cytotoxic T cells, using cytolytic granules as well as through death receptor pathways. When NK cells encounter foreign or cancer cells, they are activated via there activating receptors, including NKG2D, NKp30 and NKp46. Thus, similar to “T Cell Engagers” (TCEs), that bind to CD3 on T Cells and a tumor antigen on a tumor cell to drive cytotoxic killing of the tumor cell, Natural Killer Engagers (NKEs) can be made that bind to the NK cells and a tumor cell and similarly facilitate cytotoxic killing.

Thus, there remains a need for novel NKEs for the treatment of cancers.

### SUMMARY

In one aspect, provided herein is an antibody comprising: (a) means for binding the extracellular domain of human NKG2D; and (b) a variant IgG1 Fc dimeric domain comprising an amino acid substitution as compared to human IgG1, wherein the variant that has increased in vitro binding to the human FcγRIIIa receptor as compared to human IgG1.

In some embodiments, the variant IgG1 Fc domain comprises an amino acid substitution selected from the group including: S239D, S239E, I332D, I332E, S239D/I332E, S239D/I332D, S239E/I332D and S239E/I332E.

In some embodiments, the variant IgG1 Fc domain comprises an amino acid substitution selected from the group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

In some embodiments, the antibody further comprises means for binding a human tumor antigen selected from the group including: EGFR, Trop2, CD20, B7H3, FLT3, DLL3, CD19, CEA, MSLN, BCMA, CAIX, ANO1, CD19, CD123, CD22, CD38, HER2 and CLDN18.2.

In another aspect, provided herein is an antibody comprising: (a) means for binding the extracellular domain of human NKp46; and (b) a variant IgG1 Fc domain comprising an amino acid substitution as compared to human IgG1, wherein the variant that has increased in vitro binding to the human FcγRIIIa receptor as compared to human IgG1.

In some embodiments, the variant IgG1 Fc domain comprises an amino acid substitution selected from the group including: S239D, S239E, I332D, I332E, S239D/I332E, S239D/I332D, S239E/I332D and S239E/I332E.

In some embodiments, the variant IgG1 Fc domain comprises an amino acid substitution selected from the group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

In some embodiments, the antibody further comprises means for binding a human tumor antigen selected from the group including: EGFR, Trop2, CD20, B7H3, FLT3, DLL3, CD19, CEA, MSLN, BCMA, CAIX, ANO1, CD19, CD123, CD22, CD38, HER2 and CLDN18.2.

In a further aspect, provided herein is an antibody comprising: (a) means for binding the extracellular domain of human NKp30; and (b) a variant IgG1 Fc domain comprising an amino acid substitution as compared to human IgG1, wherein the variant that has increased in vitro binding to the human FcγRIIIa receptor as compared to human IgG1.

In some embodiments, the variant IgG1 Fc domain comprises an amino acid substitution selected from the group including: S239D, S239E, I332D, I332E, S239D/I332E, S239D/I332D, S239E/I332D and S239E/I332E.

In some embodiments, the variant IgG1 Fc domain comprises an amino acid substitution selected from the group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

In some embodiments, the antibody further comprises means for binding a human tumor antigen selected from the group including: EGFR, Trop2, CD20, B7H3, FLT3, DLL3, CD19, CEA, MSLN, BCMA, CAIX, ANO1, CD19, CD123, CD22, CD38, HER2 and CLDN18.2.

In one aspect, a heterodimeric antibody comprising: (a) means for binding the extracellular domain of human NKG2D; (b) means for binding the extracellular domain of a tumor antigen selected from the group including: EGFR, Trop2, CD20, B7H3, FLT3, DLL3, CD19, CEA, MSLN, BCMA, CAIX, ANO1, CD19, CD22, CD38, CD123, HER2 and CLDN18.2; (c) a variant IgG1 dimeric Fc domain comprising an amino acid substitution as compared to human IgG1 wherein the variant that has increased in vitro binding to the human FcγRIIIa receptor as compared to human IgG1.

In some embodiments, the variant IgG1 dimeric Fc domain comprises two monomeric Fc domains, wherein one of the monomeric Fc domains comprises an amino acid substitution(s) selected from the group including: S239D, S239E, I332D, I332E, S239D/I332E, S239E/I332E, S239D/I332D, S239E/I332D, S239D/330L/I332E, 239D/330L, 330L/332E, 243L, F243L/R292P/Y300L/V305I/P396L, 332I/247I/339Q, S298A/E333A/K334A, V264I/I332E, S298A, S298A/I332E, S298A/E333A.K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, 3293R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E AND S239D/K274E/A330L/I332E.

In some embodiments, both of the monomeric Fc domains comprises the amino acid substitution(s).

In some embodiments, one of the monomeric Fc domains comprises an S239D amino acid substitution. In various embodiments, one of the monomeric Fc domains comprises an S239E amino acid substitution. In various embodiments, one of the monomeric Fc domains comprises an S239D/I332E amino acid substitution. In many embodiments, one of the monomeric Fc domains comprises an I332E amino acid substitution. In further embodiments, wherein one of the monomeric Fc domains comprises an I332D amino acid substitution.

In one aspect, a heterodimeric antibody comprising: (a) means for binding the extracellular domain of human NKp46; (b) means for binding the extracellular domain of a tumor antigen selected from the group including: EGFR, Trop2, CD20, B7H3, FLT3, DLL3, CD19, CEA, MSLN, BCMA, CAIX, ANO1, CD19, CD22, CD38, CD123, HER2 and CLDN18.2; (c) a variant IgG1 dimeric Fc domain comprising an amino acid substitution as compared to human IgG1 wherein the variant that has increased in vitro binding to the human FcγRIIIa receptor as compared to human IgG1.

In some embodiments, the variant IgG1 dimeric Fc domain comprises two monomeric Fe domains, wherein one of the monomeric Fc domains comprises an amino acid substitution(s) selected from the group including: S239D, S239E, I332D, I332E, S239D/I332E, S239E/I332E, S239D/I332D, S239E/I332D, S239D/330L/I332E, 239D/330L, 330L/332E, 243L, F243L/R292P/Y300L/V305I/P396L, 332I/247I/339Q, S298A/E333A/K334A, V264I/I332E, S298A, S298A/I332E, S298A/E333A.K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, 3293R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E AND S239D/K274E/A330L/I332E.

In some embodiments, both of the monomeric Fc domains comprises the amino acid substitution(s).

In some embodiments, the one of the monomeric Fc domains comprises an S239D amino acid substitution. In various embodiments, the one of the monomeric Fc domains comprises an S239E amino acid substitution. In various embodiments, one of the monomeric Fc domains comprises an S239D/I332E amino acid substitution. In many embodiments, one of the monomeric Fc domains comprises an I332E amino acid substitution. In further embodiments, one of the monomeric Fc domains comprises an I332D amino acid substitution.

In one aspect, a heterodimeric antibody comprising: (a) means for binding the extracellular domain of human NKp30; (b) means for binding the extracellular domain of a tumor antigen selected from the group including: EGFR, Trop2, CD20, B7H3, FLT3, DLL3, CD19, CEA, MSLN, BCMA, CAIX, ANO1, CD19, CD22, CD38, CD123, HER2 and CLDN18.2; (c) a variant IgG1 dimeric Fc domain comprising an amino acid substitution as compared to human IgG1 wherein the variant that has increased in vitro binding to the human FcγRIIIa receptor as compared to human IgG1.

In some embodiments, the variant IgG1 dimeric Fc domain comprises two monomeric Fe domains, wherein one of the monomeric Fc domains comprises an amino acid substitution(s) selected from the group including: S239D, S239E, I332D, I332E, S239D/I332E, S239E/I332E, S239D/I332D, S239E/I332D, S239D/330L/I332E, 239D/330L, 330L/332E, 243L, F243L/R292P/Y300L/V305I/P396L, 332I/247I/339Q, S298A/E333A/K334A, V264I/I332E, S298A, S298A/I332E, S298A/E333A.K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, 3293R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E AND S239D/K274E/A330L/I332E.

In some embodiments, both of the monomeric Fc domains comprises the amino acid substitution(s).

In some embodiments, the one of the monomeric Fc domains comprises an S239D amino acid substitution. In various embodiments, the one of the monomeric Fc domains comprises an S239E amino acid substitution. In various embodiments, one of the monomeric Fc domains comprises an S239D/I332E amino acid substitution. In many embodiments, one of the monomeric Fc domains comprises an I332E amino acid substitution. In further embodiments, wherein one of the monomeric Fc domains comprises an I332D amino acid substitution.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Fig.,” “FIG.,” “Figure,” “Figures,” “Figs.,” and “FIGs.” herein) of which:

FIGS. 1A-1F depict useful pairs of heterodimerization variant sets (including skew and pI variants). In FIG. 1F, there are variants for which there are no corresponding “monomer 2” variants. Such variants are pI variants that can be used alone on either monomer of a NK cell antigen×TTA bsAb, or included, for example, on the non-scFv side of a format that utilizes an scFv as a component and an appropriate charged scFv linker can be used on the second monomer that utilizes an scFv as a binding domain. Suitable charged linkers are shown in FIG. 5.

FIG. 2 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 3 depicts useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 4 depicts useful ablation variants that enhance FcγR binding (also referred to as ADCC-enhanced variants). In some embodiments, such ADCC-enhanced variants are included in the Fc domain of both monomers of the subject antibody described herein. In other embodiments, the variants are only included on only one variant Fc domain.

FIG. 5 depicts a number of charged scFv linkers that find use in increasing or decreasing the pI of the subject heterodimeric αNK cell antigen×αTTA bsAbs that utilize one or more scFv as a component, as described herein. The (+H) positive linker finds particular use herein, particularly with anti-NK cell antigen V_{L} and V_{H} sequences shown herein. A single prior art scFv linker with a single charge is referenced as “Whitlow,” from Whitlow et al., Protein Engineering, 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc, 2+1 Fab₂-scFv-Fc, 2+1 mAb-scFv, etc.).

FIG. 6 depicts a number of exemplary domain linkers. In some embodiments, these linkers find use linking a single-chain Fv to an Fc chain. In some embodiments, these linkers may be combined in any orientation. For example, a (G)₄S linker (SEQ ID NO: 94) may be combined with a “lower half hinge” linker at the N-terminus or at the C-terminus.

FIG. 7 depicts a number of additional exemplary domain linkers, specifically, glycine-serine polymers, including for example (GS)n (SEQ ID NO: 120), (GSGGS)n (SEQ ID NO: 117), (GGGGS)n (SEQ ID NO: 118), and (GGGS)n (SEQ ID NO: 119), where n is an integer of at least one up to 10 (where n is generally about 3 to about 4 for some exemplary embodiments).

FIGS. 8A-8N depict several formats of the present invention. FIG. 8A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 8B depicts the “2+1 Fab₂-scFv-Fc” format, with a first Fab arm and a second Fab-scFv arm, wherein the Fab binds a first antigen and the scFv binds second antigen. The 2+1 Fab₂-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 8C depicts the “1+1 common light chain” (or “1+1 CLC”) format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising V_{L}-C_{L}. The V_{L} pairs with the VH1 to form a binding domain with a first antigen binding specificity; and the V_{L} pairs with the VH2 to form a binding domain with a second antigen binding specificity. FIG. 8D depicts the “2+1 common light chain” (or “2+1 CLC”) format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising V_{L}—C_{L}. The V_{L} pairs with the first and second VH1 to form binding domains with a first antigen binding specificity; and the V_{L} pairs with the VH2 to form a binding domain with a second antigen binding specificity. FIG. 8E depicts the “2+1 mAb-scFv” (or “mAb-scFv”) format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising V_{L}—C_{L}. The V_{L} pairs with the first and second VH1 to form binding domains with binding specificity for the first antigen. FIGS. 8F-8N depict several additional formats (e.g., the “dual scFv” format, the “one-armed scFv-mAb” format, the “scFv-mAb” format, the “bispecific mAb” format, the “one-arm central-scFv” format, the “mAb-Fv” format, the “central-Fv” format, the “trident” format, and the “stack Fab₂-scFv-Fc” format, as are described in further detail herein).

FIG. 9 depicts the sequences for human and cynomolgus NKG2D antigens, including the extracellular domains. Such NKG2D are useful for the development of cross-reactive NKG2D antigen binding domains for ease of clinical development.

FIGS. 10A-10B depict the sequences for human, mouse, and cynomolgus B7H3, including the extracellular domains. Such B7H3 are useful for the development of cross-reactive B7H3 antigen binding domains for ease of clinical development.

FIGS. 11A-11C depict anti-human Nkp46 antigen binding domains of use in the present invention.

FIG. 12 depicts anti-human Nkp30 antigen binding domains of use in the present invention.

FIGS. 13A-13C depict anti-human EGFR antigen binding domains of use in the present invention.

FIG. 14 depicts anti-human CD19 antigen binding domains of use in the present invention.

FIGS. 15A-15C depict anti-human CD20 antigen binding domains of use in the present invention.

FIG. 16 depicts anti-human CLDN18.2 antigen binding domains of use in the present invention.

FIGS. 17A-17C depict anti-human BCMA antigen binding domains of use in the present invention.

FIGS. 18A-18C depict anti-human CEA antigen binding domains of use in the present invention.

FIG. 19 depicts anti-human FLT3 antigen binding domains of use in the present invention.

FIGS. 20A-20B depict anti-human HER2 antigen binding domains of use in the present invention.

FIGS. 21A-21Z and 21AA-21FF depict anti-human MSLN antigen binding domains of use in the present invention.

FIGS. 22A-22R depict anti-human Trop-2 antigen binding domains of use in the present invention.

FIG. 23 depicts anti-CAIX and CD123 antigen binding domains of use in the present invention.

FIGS. 24A-24C depict anti-human DLL3 antigen binding domains of use in the present invention.

FIGS. 25A-25G depict anti-human PD-1 antigen binding domains of use in the present invention. Note that slashes (“/”) are placed between variable (VH and VL) and constant domains of the ABDs.

FIGS. 26A-26Z depict variable heavy and variable light domains as well as CDRS for NKG2D binding clones 1D7B4, 1D2B4, mAb-C, and mAb-D, as well as several additional NKG2D variable heavy and variable light chain sequences, and affinity detuned variable heavy domains of the anti-NKG2D 1D7B4 clone and their CDRs (as in Kabat). As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIG. 27 depicts the variable heavy and variable light chain sequences for 6A1 and 3C4, exemplary humanized rat hybridoma-derived B7H3 binding domains.

FIG. 28 depicts the variable heavy and variable light chain sequences for 4F12 and 38E2, exemplary humanized rabbit hybridoma-derived B7H3 binding domains.

FIGS. 29A-29H depict the variable heavy and variable light chain sequences for 2E4A3.189, an exemplary phage-derived B7H3 binding domain, and additional sequences for affinity-optimized variable heavy domains from anti-B7H3 clone 2E4A3.189. It should be noted that the variable heavy domains can be paired with either 2E4A3.189_L1, as depicted in FIG. 29A.

FIGS. 30A-30R depict the variable heavy and variable light chain sequences for additional B7H3 binding domains which find use in the NK cell antigen×TTA bsAb of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the V_{H} and V_{L} domains using other numbering systems. Furthermore, as for all the sequences in the Figures, these V_{H} and V_{L} sequences can be used either in a scFv format or in a Fab format.

FIGS. 31A-31C show the sequences of several useful heterodimeric NK cell antigen×TTA bsAb backbones based on human IgG1 and having WT effector function. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and N297S variant that removes glycosylation on both chains. Heterodimeric Fc backbone 8 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434S Xtend variants on both chains. Heterodimeric Fc backbone 9 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 10 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains. Heterodimeric Fc backbone 11 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

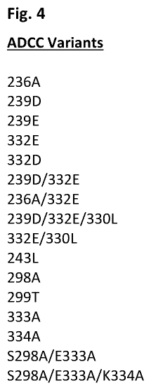

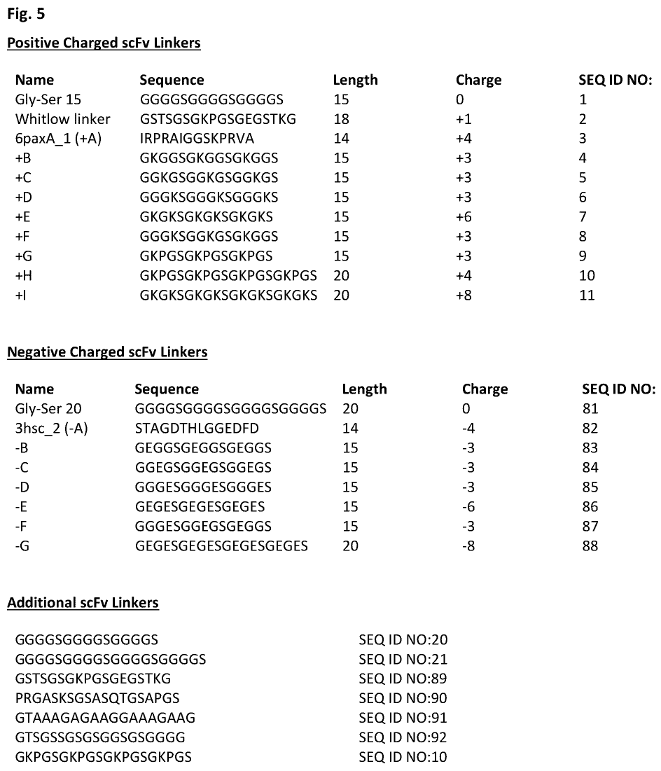

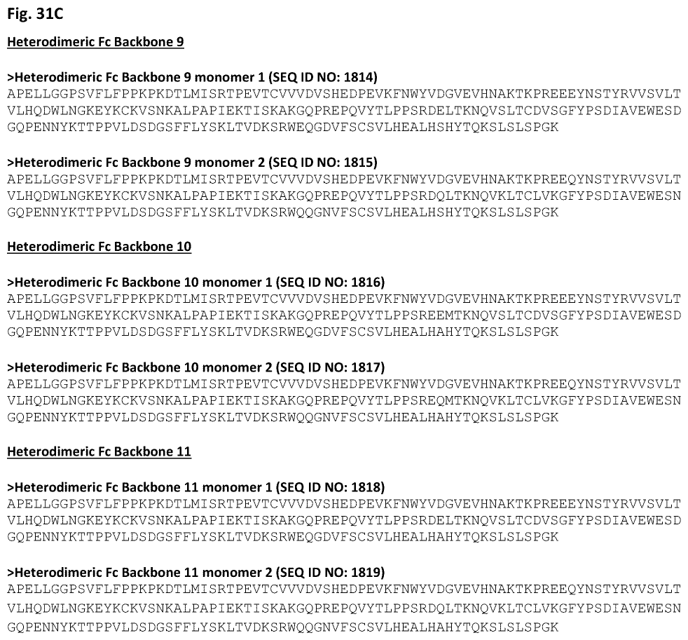

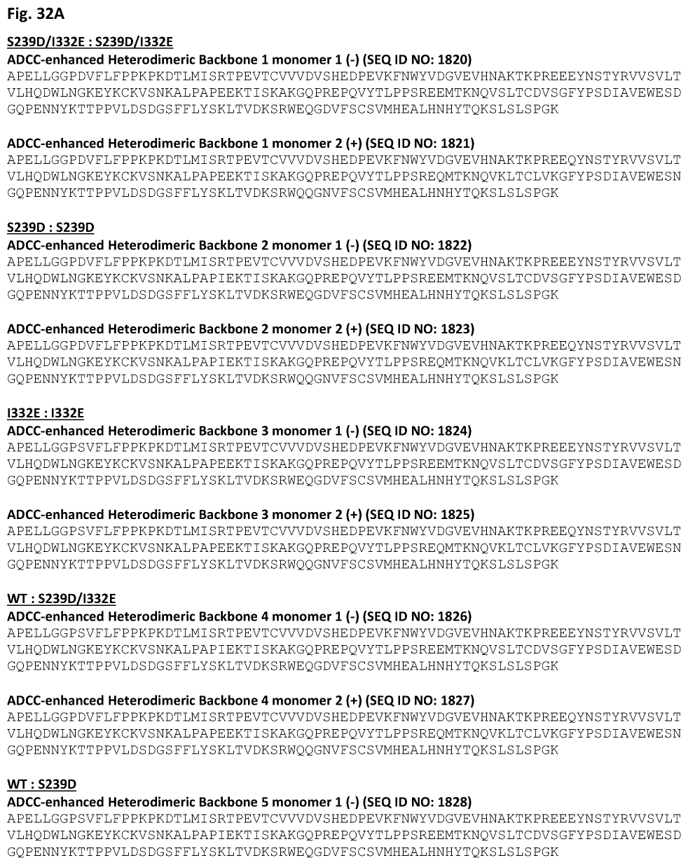

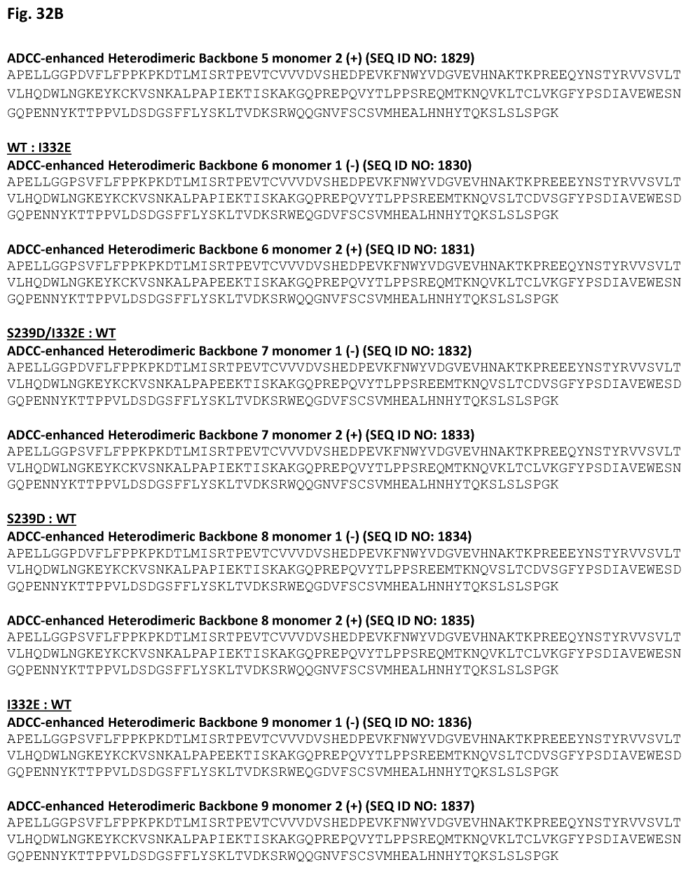

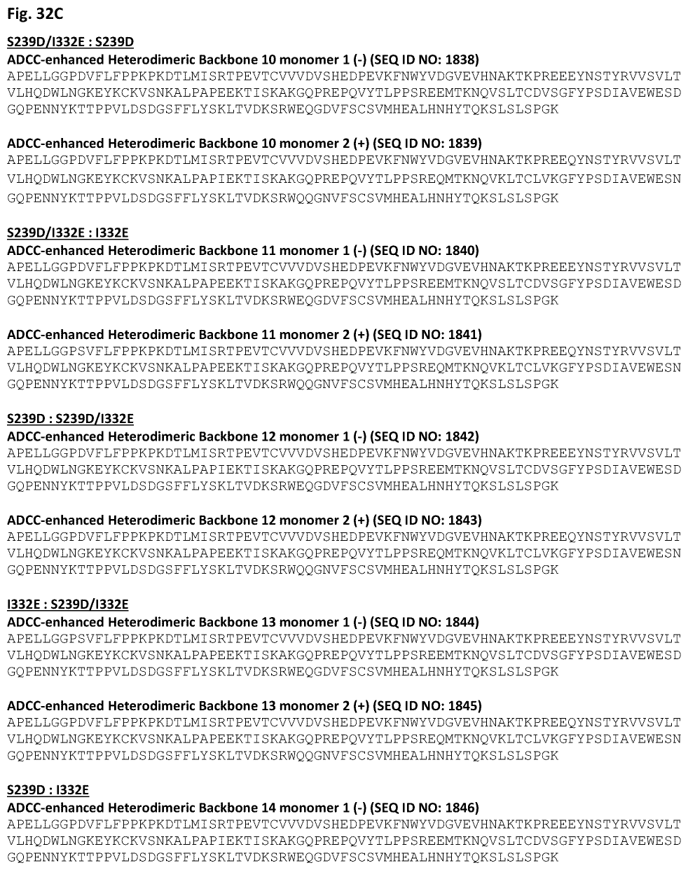

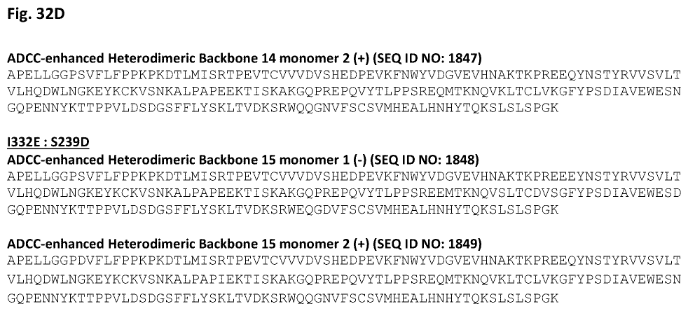

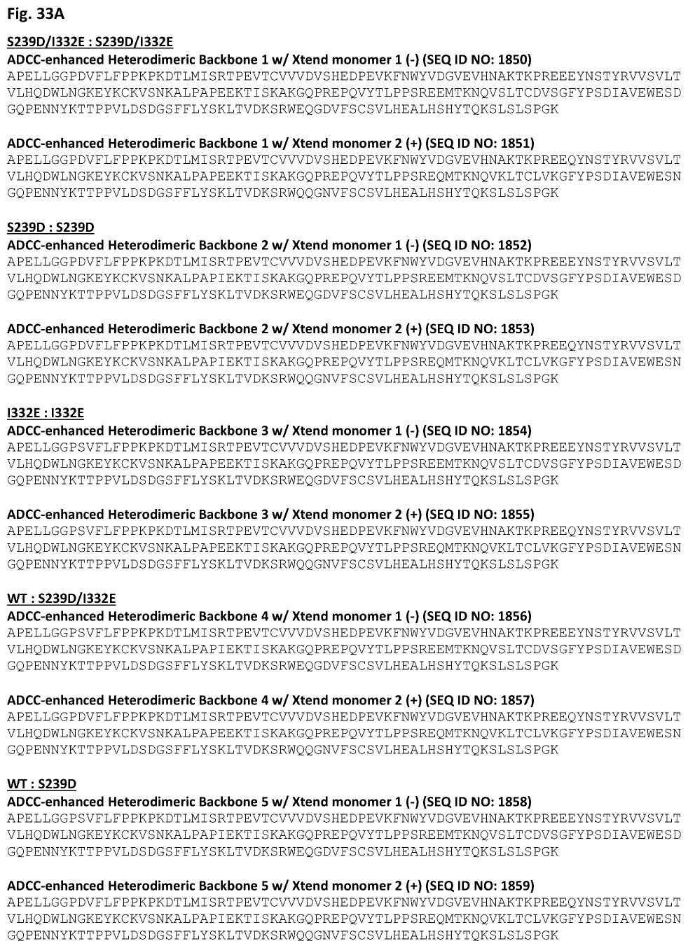

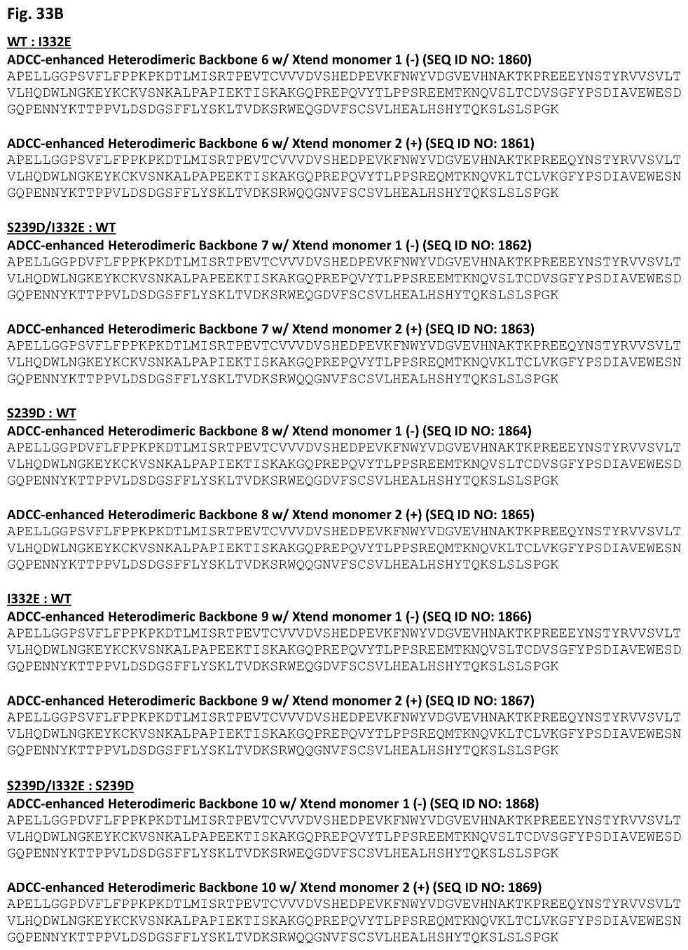

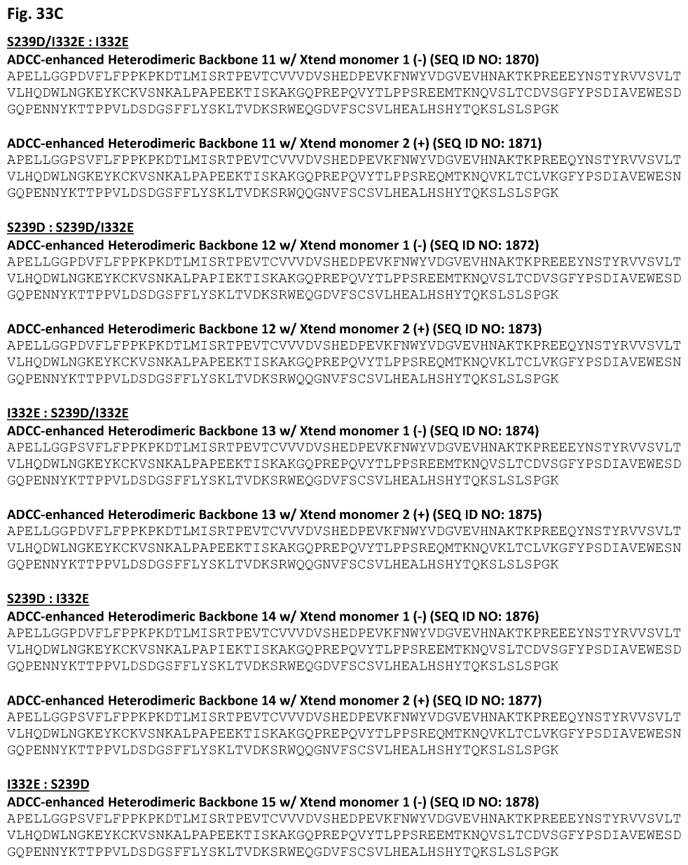

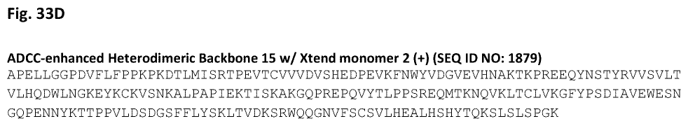

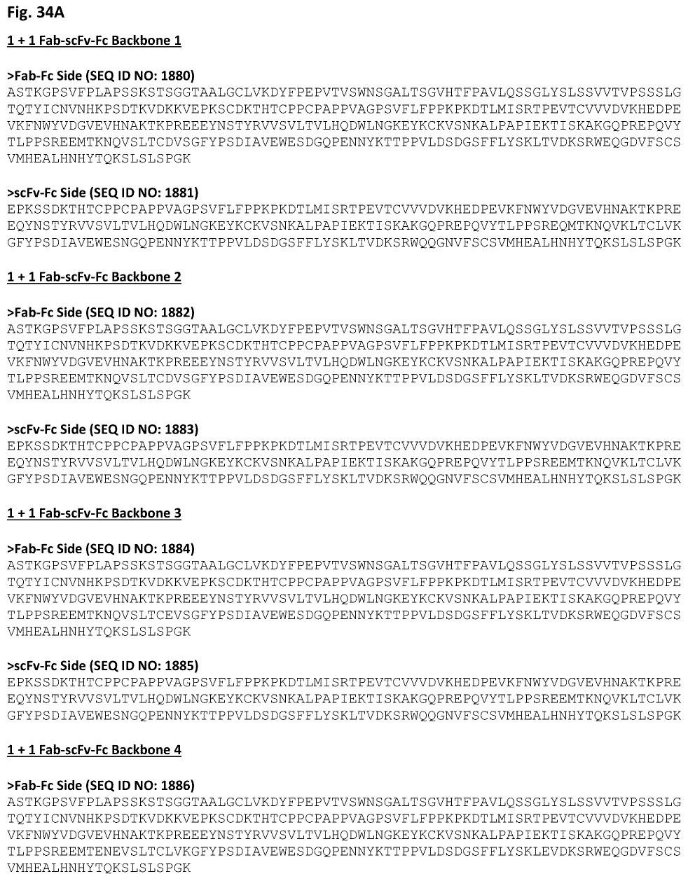

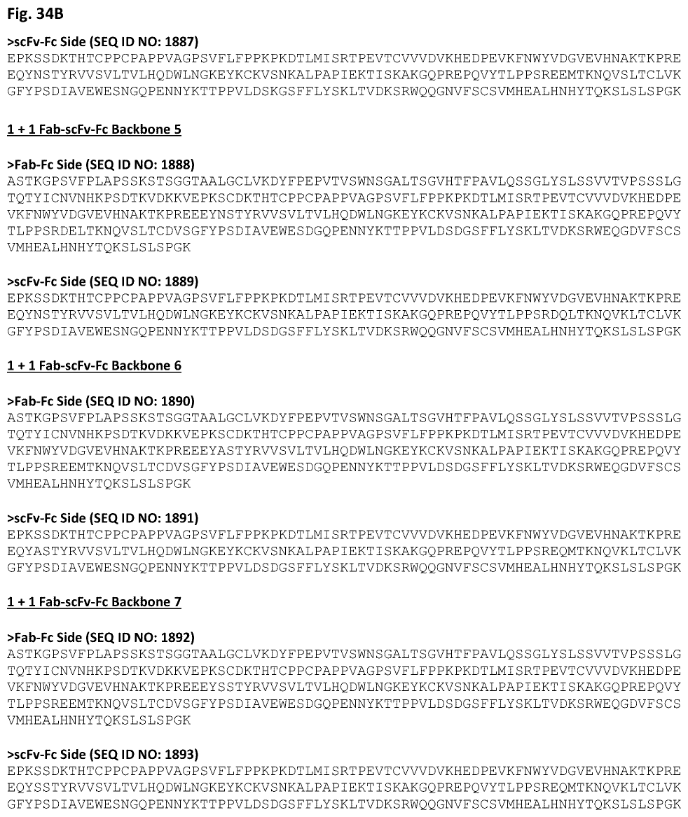

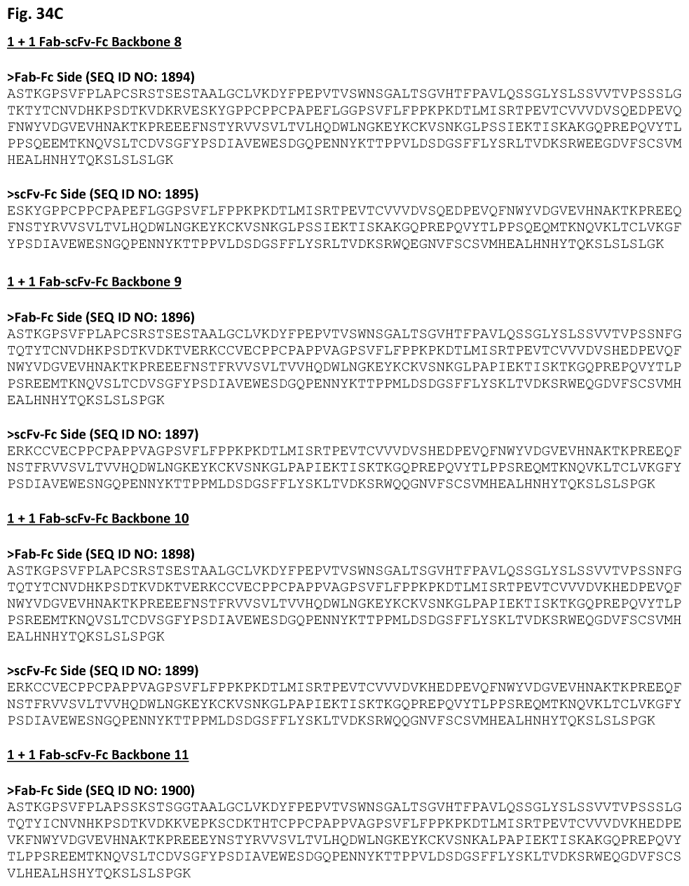

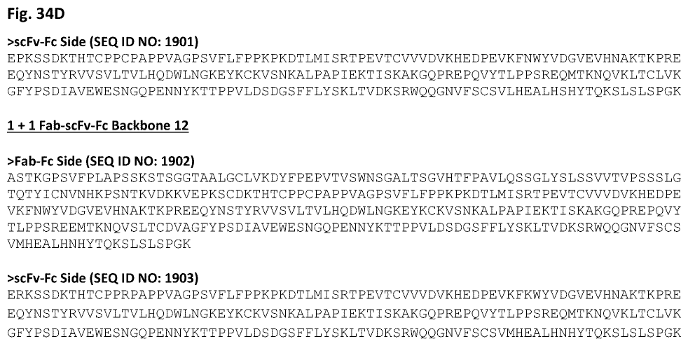

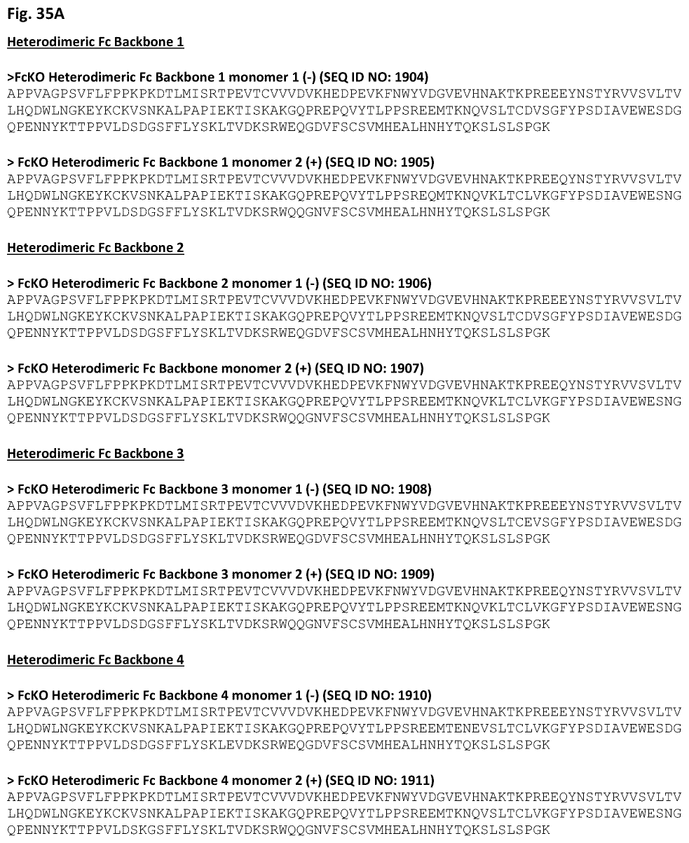

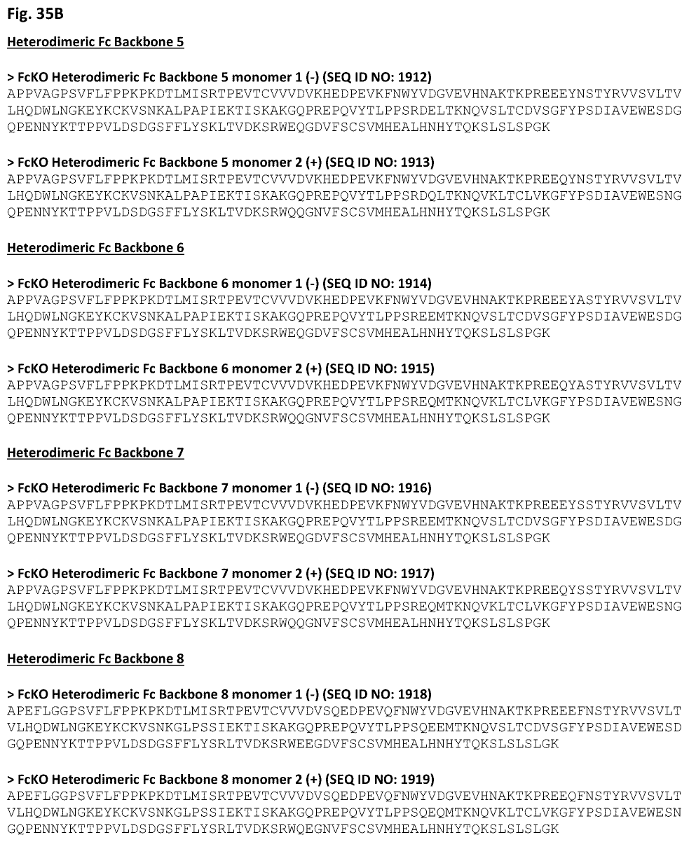

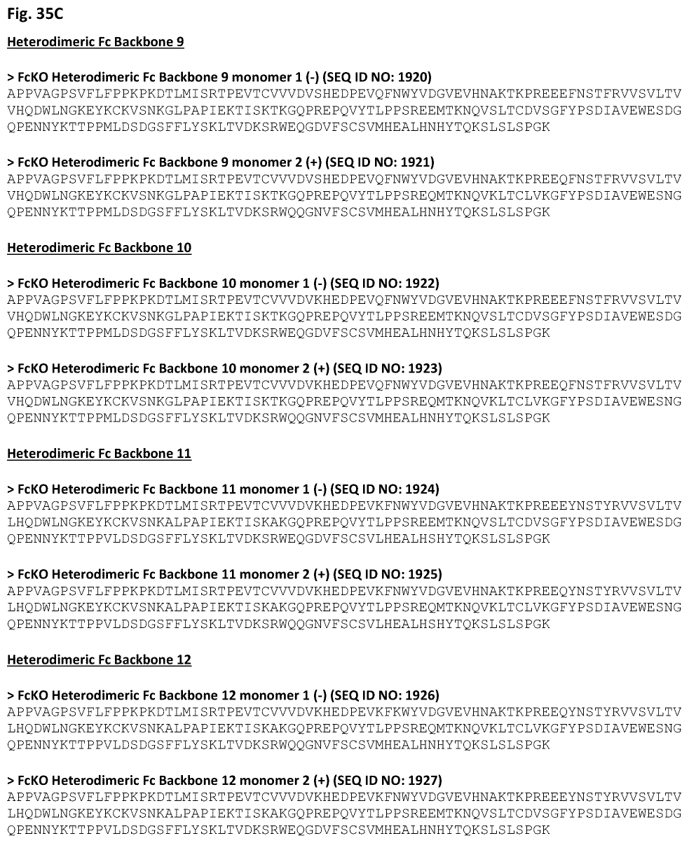

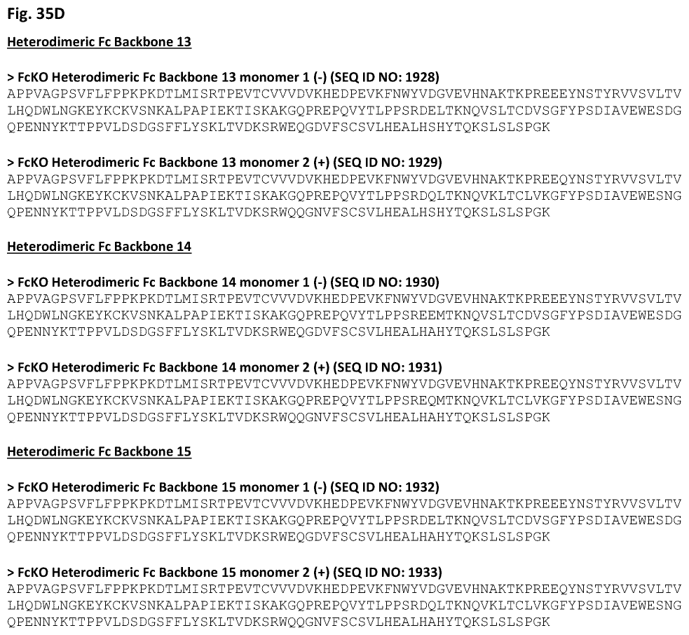

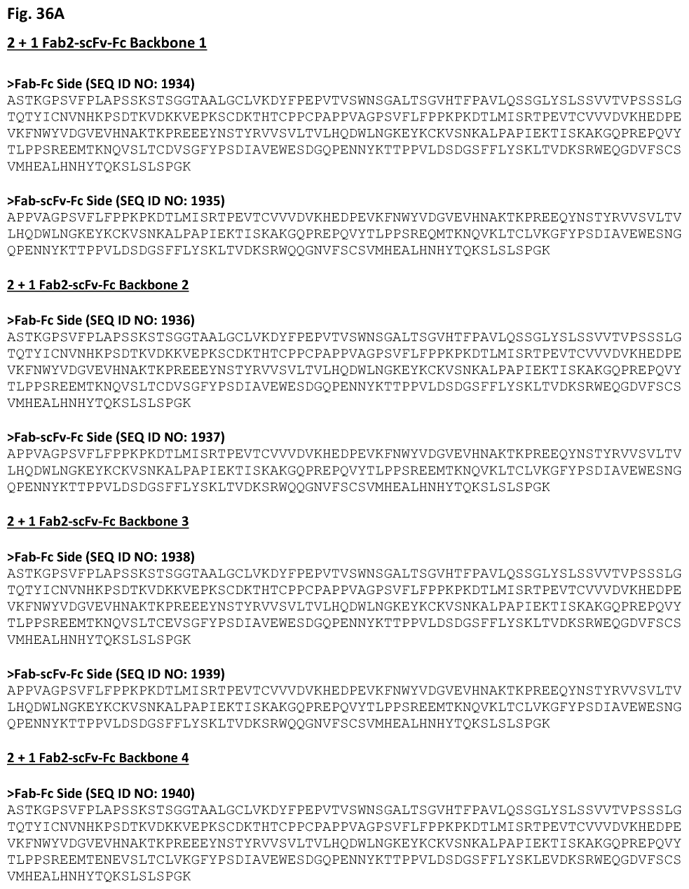

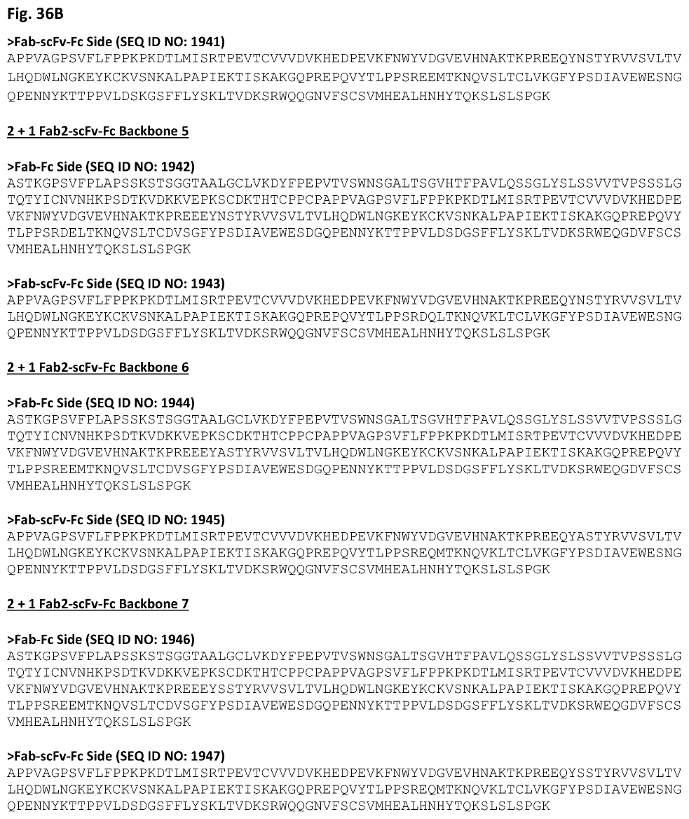

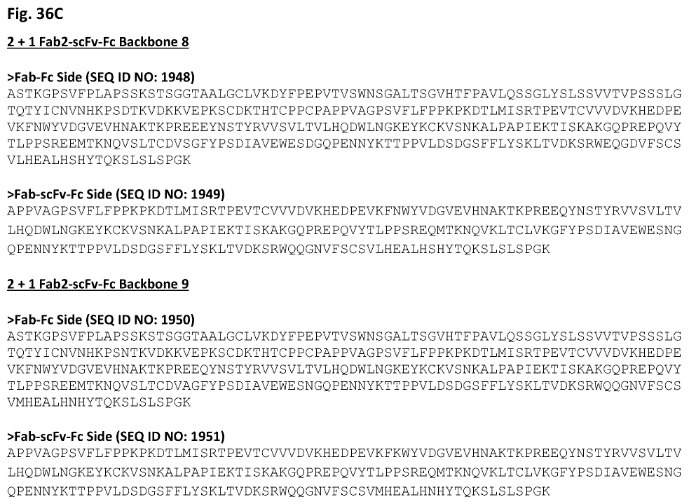

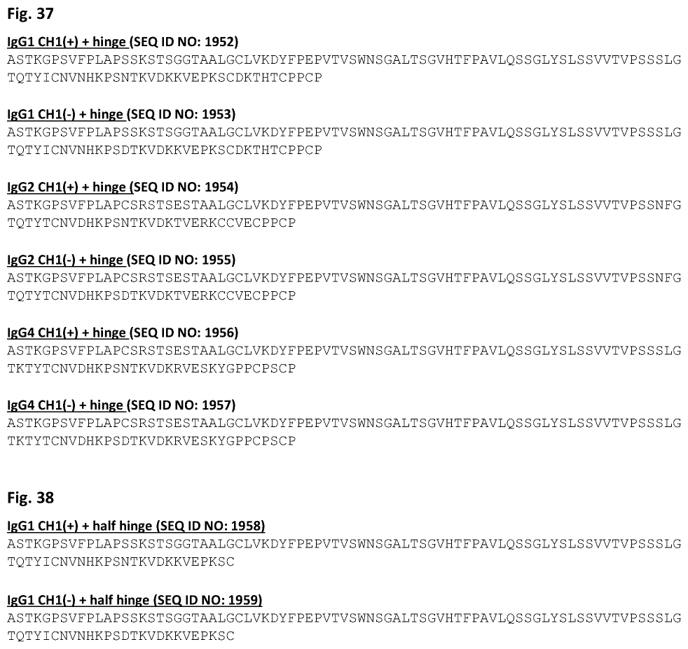

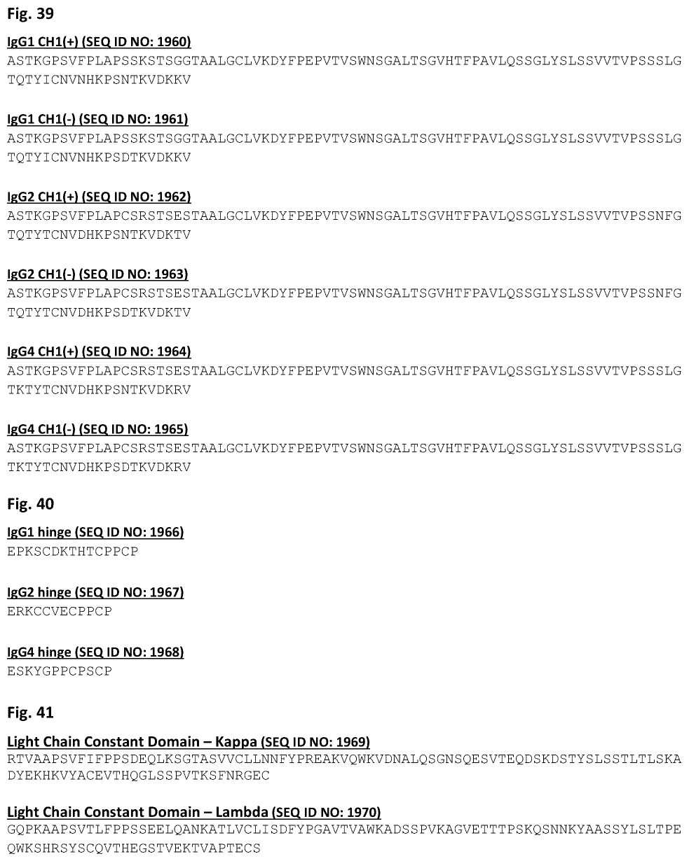

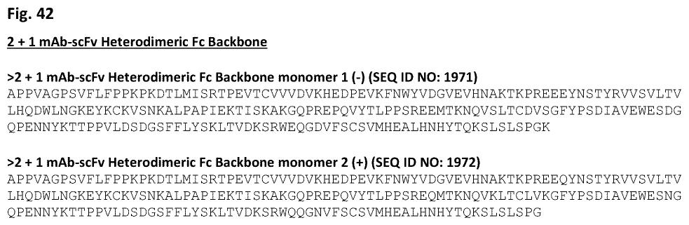

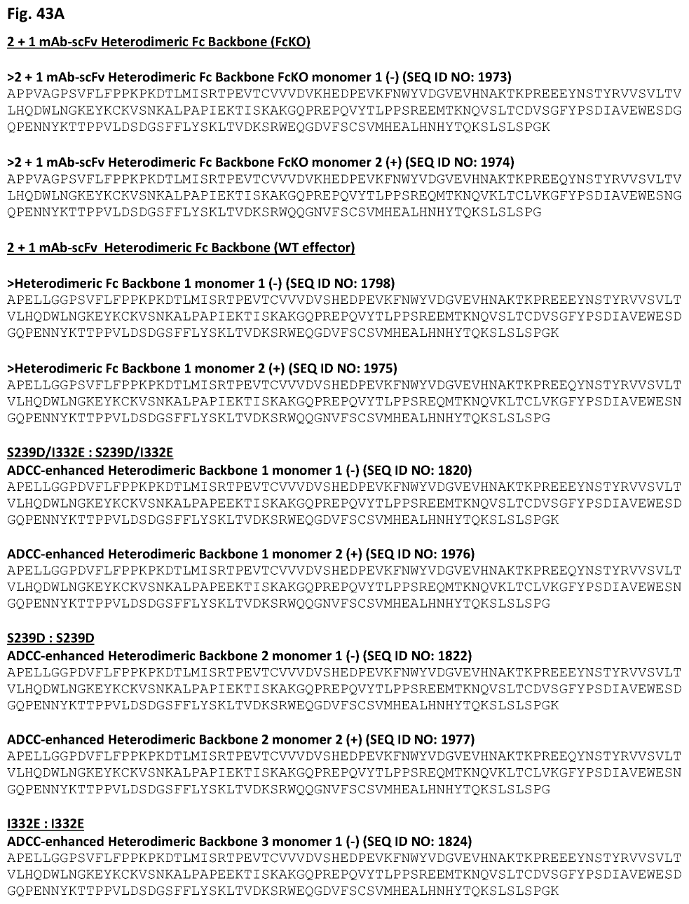

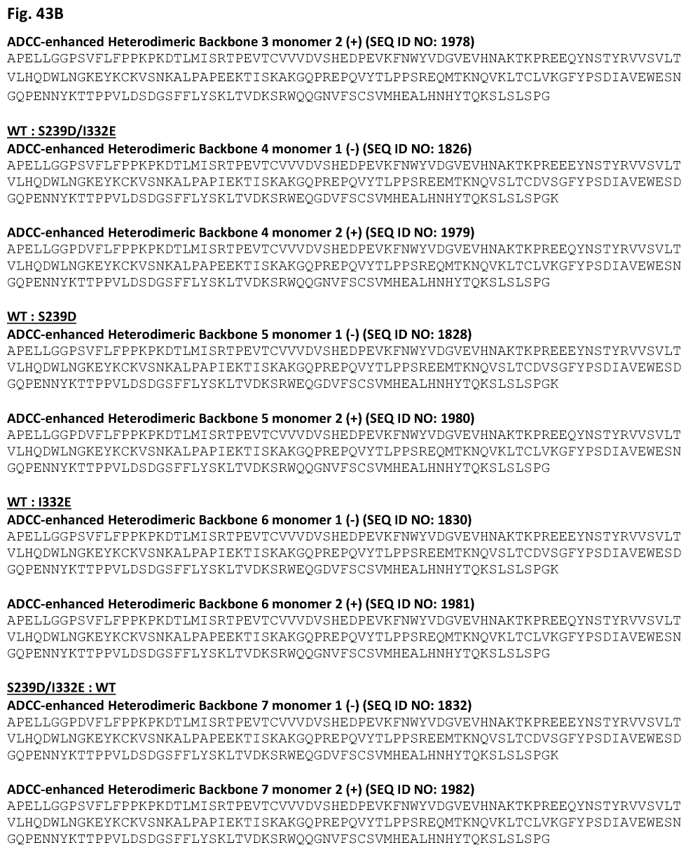

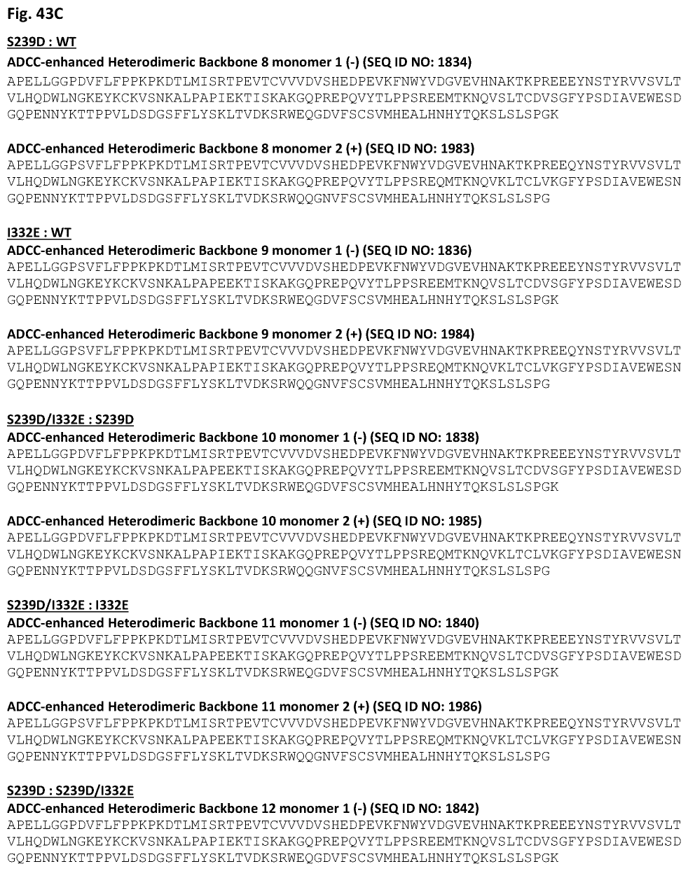

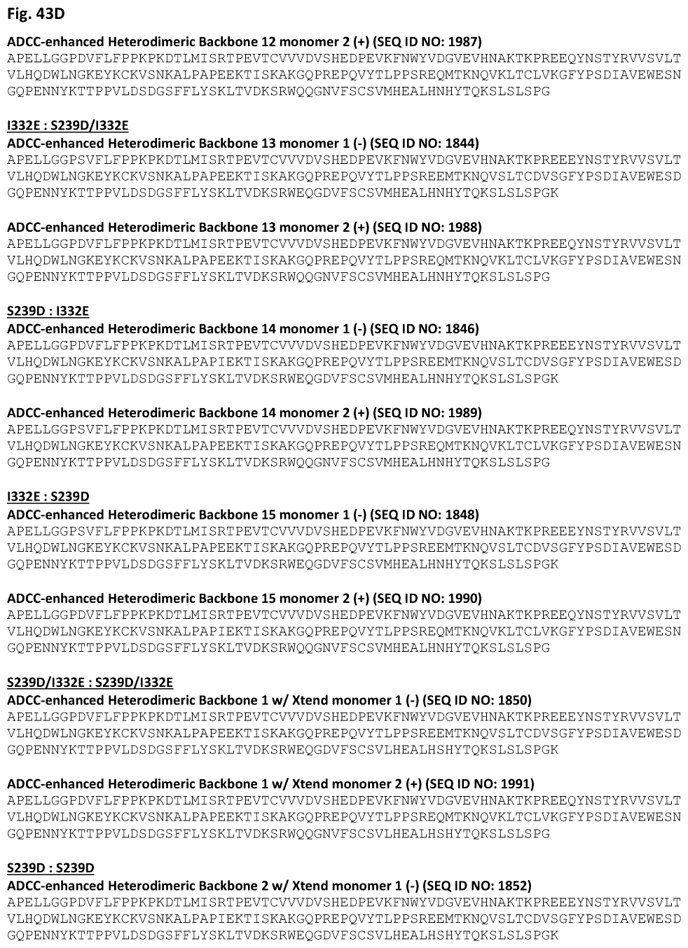

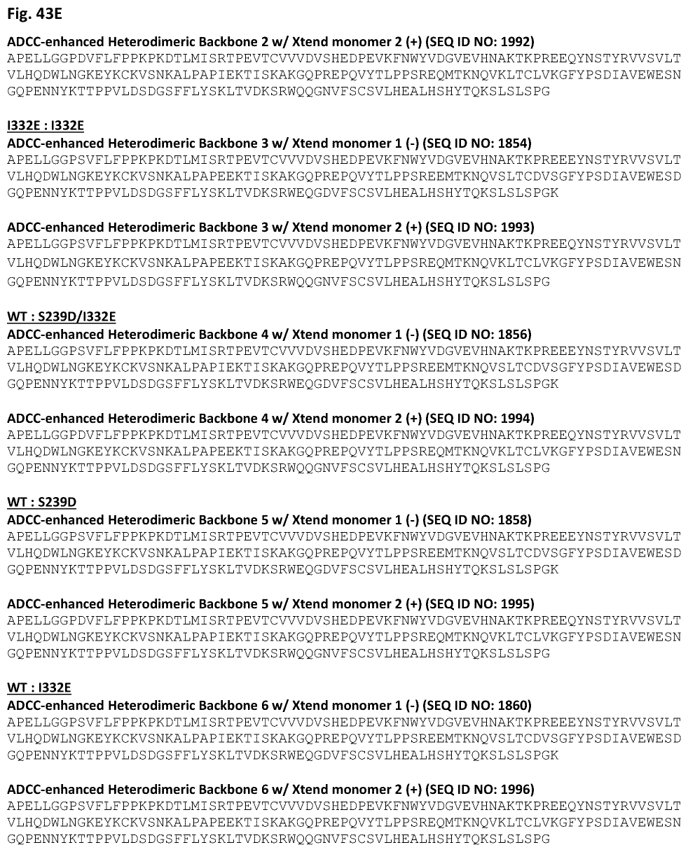

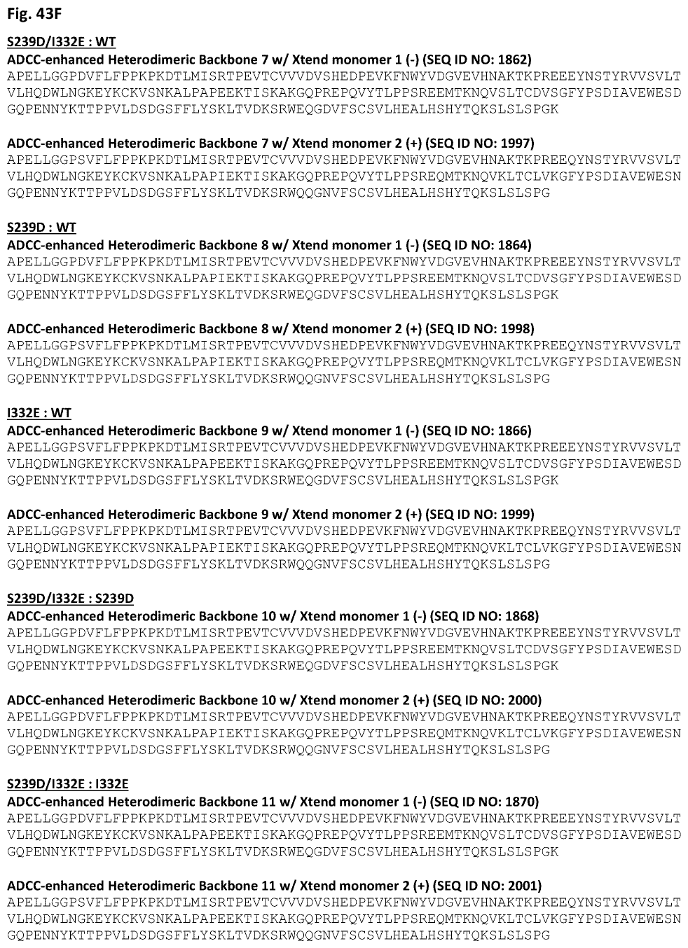

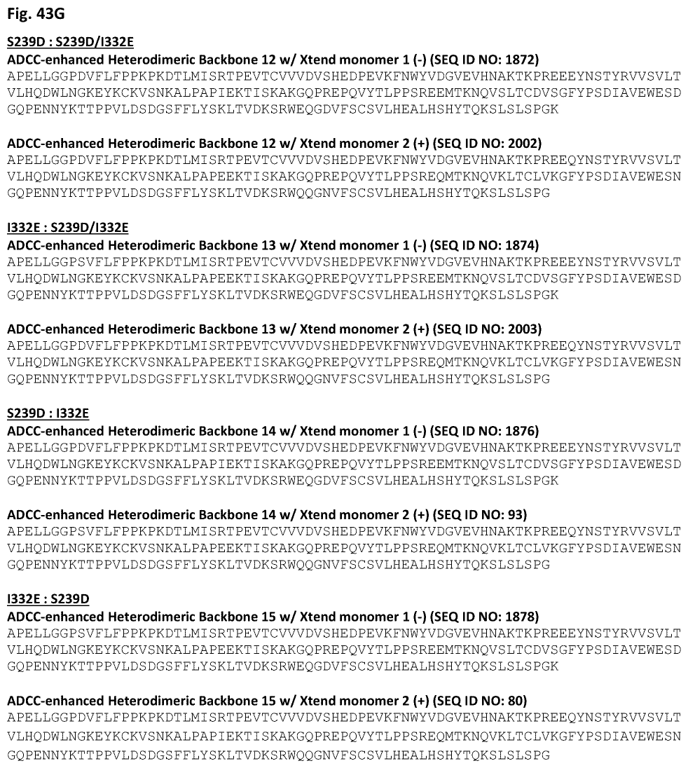

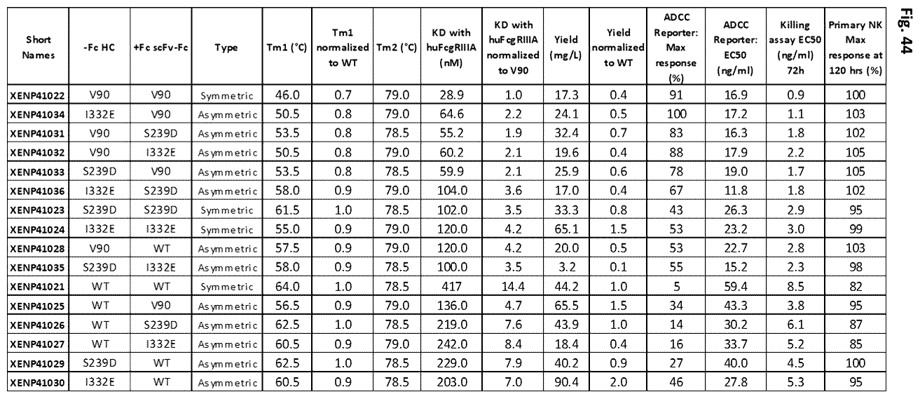

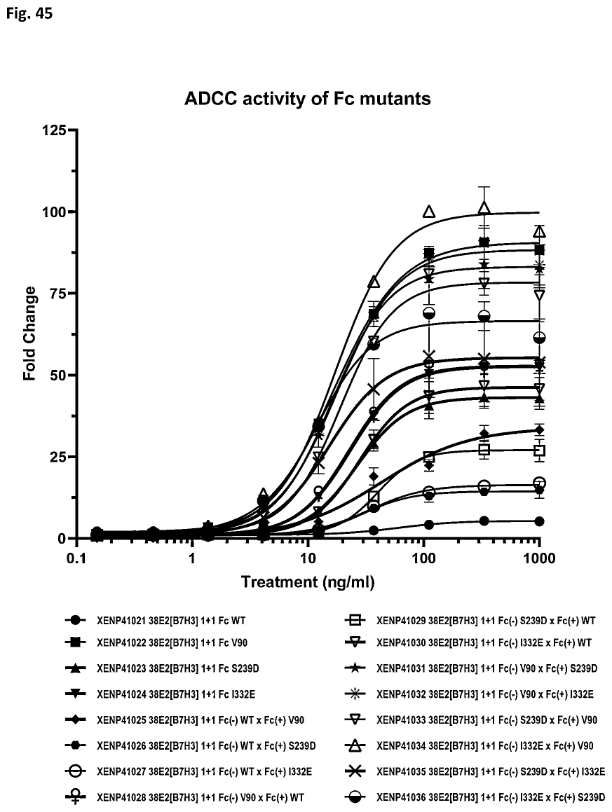

FIGS. 32A-32D show the sequences of several useful heterodimeric NK cell antigen×TTA bsAb backbones based on human IgG1 and having enhanced ADCC function. The sequences here are based on heterodimeric Fc backbone 1 in FIG. 31, although the ADCC variants in FIG. 4 may also be included in any of the other heterodimeric Fc backbones in FIG. 31 ADCC-enhanced Heterodimeric Backbone 1 includes S239D/I332E on both the first and the second heterodimeric Fc chain. ADCC-enhanced Heterodimeric Backbone 2 includes S239D on both the first and the second heterodimeric Fc chain. ADCC-enhanced Heterodimeric Backbone 3 includes I332E on both the first and the second heterodimeric Fc chain. ADCC-enhanced Heterodimeric Backbone 4 includes S239D/I332E on the second heterodimeric Fc chain. ADCC-enhanced Heterodimeric Backbone 5 includes S239D on the second heterodimeric Fc chain. ADCC-enhanced Heterodimeric Backbone 6 includes I332E on the second heterodimeric Fc chain. ADCC-enhanced Heterodimeric Backbone 7 includes S239D/I332E on the first heterodimeric Fc chain. ADCC-enhanced Heterodimeric Backbone 8 includes S239D on the first heterodimeric Fc chain. ADCC-enhanced Heterodimeric Backbone 9 includes I332E on the first heterodimeric Fc chain. ADCC-enhanced Heterodimeric Backbone 10 includes S239D/I332E on the first heterodimeric Fc chain and S239D on the second heterodimeric Fc chain. ADCC-enhanced Heterodimeric Backbone 11 includes S239D/I332E on the first heterodimeric Fc chain and I332E on the second heterodimeric Fc chain. ADCC-enhanced Heterodimeric Backbone 12 includes S239D on the first heterodimeric Fc chain and S239D/I332E on the second heterodimeric Fc chain. ADCC-enhanced Heterodimeric Backbone 13 includes I332E on the first heterodimeric Fc chain and S239D/I332E on the second heterodimeric Fc chain. ADCC-enhanced Heterodimeric Backbone 14 includes S239D on the first heterodimeric Fc chain and I332E on the second heterodimeric Fc chain. ADCC-enhanced Heterodimeric Backbone 15 includes I332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 33A-33D show the sequences of several useful heterodimeric NK cell antigen×TTA bsAb backbones based on human IgG1 and having enhanced ADCC function and enhanced serum half-life. The sequences here are based on heterodimeric Fc backbone 8 in FIG. 31 although the ADCC variants in FIG. 4 may also be included in any of the other heterodimeric Fc backbones in FIG. 31 ADCC-enhanced Heterodimeric Backbone 1 with Xtend includes S239D/I332E on both the first and the second heterodimeric Fc chain. ADCC-enhanced Heterodimeric Backbone 2 with Xtend includes S239D on both the first and the second heterodimeric Fc chain. ADCC-enhanced Heterodimeric Backbone 3 with Xtend includes I332E on both the first and the second heterodimeric Fc chain. ADCC-enhanced Heterodimeric Backbone 4 with Xtend includes S239D/I332E on the second heterodimeric Fc chain. ADCC-enhanced Heterodimeric Backbone 5 with Xtend includes S239D on the second heterodimeric Fc chain. ADCC-enhanced Heterodimeric Backbone 6 with Xtend includes I332E on the second heterodimeric Fc chain. ADCC-enhanced Heterodimeric Backbone 7 with Xtend includes S239D/I332E on the first heterodimeric Fc chain. ADCC-enhanced Heterodimeric Backbone 8 with Xtend includes S239D on the first heterodimeric Fc chain. ADCC-enhanced Heterodimeric Backbone 9 with Xtend includes I332E on the first heterodimeric Fc chain. ADCC-enhanced Heterodimeric Backbone 10 with Xtend includes S239D/I332E on the first heterodimeric Fc chain and S239D on the second heterodimeric Fc chain. ADCC-enhanced Heterodimeric Backbone 11 with Xtend includes S239D/I332E on the first heterodimeric Fc chain and I332E on the second heterodimeric Fc chain. ADCC-enhanced Heterodimeric Backbone 12 with Xtend includes S239D on the first heterodimeric Fc chain and S239D/I332E on the second heterodimeric Fc chain. ADCC-enhanced Heterodimeric Backbone 13 with Xtend includes I332E on the first heterodimeric Fc chain and S239D/I332E on the second heterodimeric Fc chain. ADCC-enhanced Heterodimeric Backbone 14 with Xtend includes S239D on the first heterodimeric Fc chain and I332E on the second heterodimeric Fc chain. ADCC-enhanced Heterodimeric Backbone 15 with Xtend includes I332E on the first heterodimeric Fc chain and S239D on the second heterodimeric Fc chain.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1. That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 34A-34D depict the sequences of heterodimeric antibody format heavy chain backbones with ablated effector function, also referred to as the FcKO variants. Backbone 1 is based on human IgG1 (356E/358M allotype), and includes the S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K: L368D/K370S skew variants, C220S on the chain with the S364K skew variant, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K: L368E/K370S skew variants, C220S on the chain with the S364K skew variant, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, C220S on the chain with the D401K skew variant, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q: L368D/K370S skew variants, C220S on the chain with the S364K/E357Q skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is based on human IgG4, and includes the S364K/E357Q: L368D/K370S skew variants, the N208D/Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q: L368D/K370S skew variants, the N208D/Q295E/N384D/Q418E/N421D pI variants on the chain with L368D/K370S skew variants. Backbone 10 is based on human IgG2, and includes the S364K/E357Q: L368D/K370S skew variants, the N208D/Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes S364K/E357Q: L368D/K370S skew variants, C220S and the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIGS. 35A-35D show the sequences of multimeric NK cell antigen x TTA bsAb backbones with ablated effector function, also referred to as the FcKO variants. Backbone 1 is based on human IgG1 (356E/358M allotype), and includes the S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K: L368D/K370S skew variants, C220S on the chain with the S364K skew variant, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K: L368E/K370S skew variants, C220S on the chain with the S364K skew variant, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, C220S on the chain with the D401K skew variant, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q: L368D/K370S skew variants, C220S on the chain with the S364K/E357Q skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q: L368D/K370S skew variants, C220S on the chain with the S364K/E357Q skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is based on human IgG4, and includes the S364K/E357Q: L368D/K370S skew variants, the N208D/Q295E/N384D/Q418E/N421D pI variants on the chain with L368D/K370S skew variants, as well as a S228P (EU numbering, this is S241P in Kabat) variant on both chains that ablates Fab arm exchange as is known in the art. Backbone 9 is based on human IgG2, and includes the S364K/E357Q: L368D/K370S skew variants, the N208D/Q295E/N384D/Q418E/N421D pI variants on the chain with L368D/K370S skew variants. Backbone 10 is based on human IgG2, and includes the S364K/E357Q: L368D/K370S skew variants, the N208D/Q295E/N384D/Q418E/N421D pI variants on the chain with L368D/K370S skew variants as well as a S267K variant on both chains. Backbone 11 is identical to backbone 1, except it includes M428L/N434S Xtend mutations. Backbone 12 is based on human IgG1 (356E/358M allotype), and includes S364K/E357Q: L368D/K370S skew variants, C220S and the P217R/P229R/N276K pI variants on the chain with S364K/E357Q skew variants and the E233P/L234V/L235A/G236del/S267K ablation variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

FIGS. 36A-36C show the sequences of several useful 2+1 Fab₂-scFv-Fc bispecific antibody format heavy chain backbones based on human IgG1, without the Fv sequences (e.g., the scFv and the VH for the Fab side). Backbone 1 is based on human IgG1 (356E/358M allotype), and includes the S364K/E357Q: L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 2 is based on human IgG1 (356E/358M allotype), and includes S364K: L368D/K370S skew variants, the N208D/Q295E/N384D/Q418E/N421D pI variants on the chain with L368D/K370S skew variants, and the E233P/L234V/L235A/G236del/S267K ablation variants on both chains. Backbone 3 is based on human IgG1 (356E/358M allotype), and includes S364K: L368E/K370S skew variants, the N208D/Q295E/N384D/Q418E/N421D pI variants on the chain with L368E/K370S skew variants and the E233P/L234V/L235A/G236del/S267K ablation variants on both chains. Backbone 4 is based on human IgG1 (356E/358M allotype), and includes D401K: K360E/Q362E/T411E skew variants, the N208D/Q295E/N384D/Q418E/N421D pI variants on the chain with K360E/Q362E/T411E skew variants and the E233P/L234V/L235A/G236del/S267K ablation variants on both chains. Backbone 5 is based on human IgG1 (356D/358L allotype), and includes S364K/E357Q: L368D/K370S skew variants, the N208D/Q295E/N384D/Q418E/N421D pI variants on the chain with L368D/K370S skew variants and the E233P/L234V/L235A/G236del/S267K ablation variants on both chains. Backbone 6 is based on human IgG1 (356E/358M allotype), and includes S364K/E357Q: L368D/K370S skew variants, N208D/Q295E/N384D/Q418E/N421D pI variants on the chain with L368D/K370S skew variants and the E233P/L234V/L235A/G236del/S267K ablation variants on both chains, as well as an N297A variant on both chains. Backbone 7 is identical to 6 except the mutation is N297S. Backbone 8 is identical to backbone 1, except it includes M428L/N434S Xtend mutations. Backbone 9 is based on human IgG1 (356E/358M allotype), and includes S364K/E357Q: L368D/K370S skew variants, the P217R/P229R/N276K pI variants on the chain with S364K/E357Q skew variants and the E233P/L234V/L235A/G236del/S267K ablation variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition to the skew, pI and ablation variants contained within the backbones of this figure.

FIG. 37 depicts sequences for “CH1+hinge” that find use in embodiments of NK cell antigen x TTA bsAbs. For particular embodiments wherein the Fab is on the (+) side, the “CH1(+)+hinge” sequences may find use. For particular embodiments wherein the Fab is on the (−) side, the “CH1(−)+hinge” sequences may find use.

FIG. 38 depicts sequences for “CH1+half hinge” domain linker that find use in embodiments of NK cell antigen x TTA bsAbs in the 2+1 Fab₂-scFv-Fc format. In the 2+1 Fab₂-scFv-Fc format, the “CH1+half hinge” sequences find use linking the variable heavy domain (VH) to the scFv domain on the Fab-scFv-Fc side of the bispecific antibody. It should be noted that other linkers may be used in place of the “CH1+half hinge”. It should also be noted that although the sequences here are based on the IgG1 sequence, equivalents can be constructed based on the IgG2 or IgG4 sequences.

FIG. 39 depicts sequences for “CH1” that find use in embodiments of FIG. 38 depicts sequences for “CH1” that find use in embodiments of αNKG2D×αB7H3 bsAbs.

FIG. 40 depicts sequences for “hinge” that find use in embodiments of NK cell antigen×TTA bsAbs.

FIG. 41 depicts the constant domain of the cognate light chains which find use in the subject NK cell antigen x TTA bsAbs that utilize a Fab binding domain.

FIG. 42 shows illustrative sequences of heterodimeric NK cell antigen x TTA bsAb backbones for use in the 2+1 mAb-scFv format. The format depicted here is based on heterodimeric Fc backbone 1 as depicted in FIG. 36, except further including G446_on monomer 1 (−) and G446_/K447_on monomer 2 (+). It should be noted that any of the additional backbones depicted in FIG. 36 may be adapted for use in the 2+1 mAb-scFv format with or without including K447_on one or both chains. It should be noted that these sequences may further include the M428L/N434S or M428L/N434A variants.

FIGS. 43A-43G depict illustrative sequences of heterodimeric NK cell antigen x TTA bsAb backbones for use in the 2+1 mAb-scFv format. The format depicted here is based on heterodimeric Fc backbone 1 as depicted in FIG. 36, except further including K447_on monomer 2 (+). It should be noted that any of the additional backbones depicted in FIGS. 31-33, 35, and 36 may be adapted for use in the 2+1 mAb-scFv format with or without including K447_on one or both chains.

FIG. 44 depicts a matrix of symmetric and asymmetric ADCC-enhanced Fc variants that have been engineered, as well as the corresponding Tm data, affinity data, production yield, ADCC activity and target cell killing activity. As shown, each Fc monomer (−Fc HC or +Fc-scfv-Fc) has either the S239D and I332E (V90) variants, the S239D variant alone, the I332E variant alone, or is wild-type at the 239 and 332 positions; and each test article has a different combination of these Fc monomers.

FIG. 45 depicts the range of ADCC activity of the various symmetric and asymmetric V90 variants outlined in FIG. 44. The results show a large range in levels of fold change in ADCC activity of each construct compared to wild type, with V90 having one of the highest fold changes in ADCC activity compared to WT, and the various S239D and I332E combinations showing a broad range of intermediate levels fold changes.

### DETAILED DESCRIPTION

### I. Overview

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, and sequences from GenBank, and other databases referred to herein are incorporated by reference in their entirety with respect to the related technology.

### II. Nomenclature

The antibodies provided herein are listed in several different formats. In some instances, each monomer of a particular antibody is given a unique “XENP” number, although as will be appreciated in the art, a longer sequence might contain a shorter one.

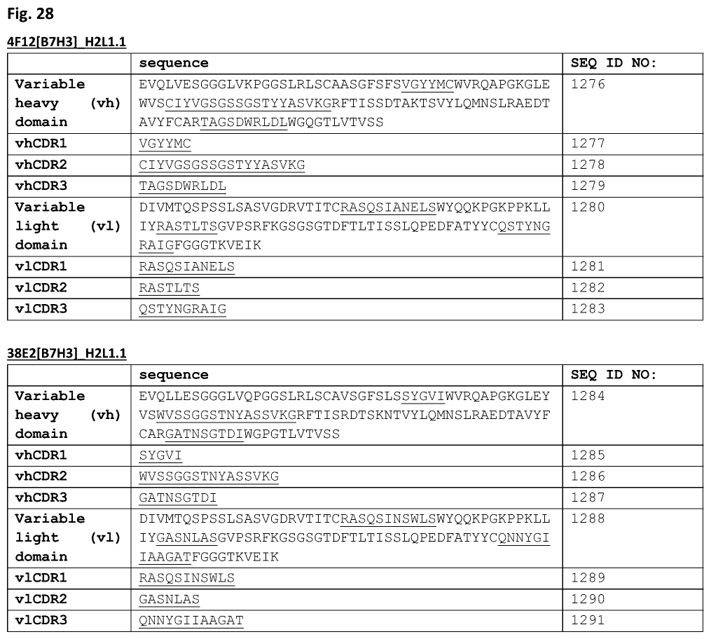

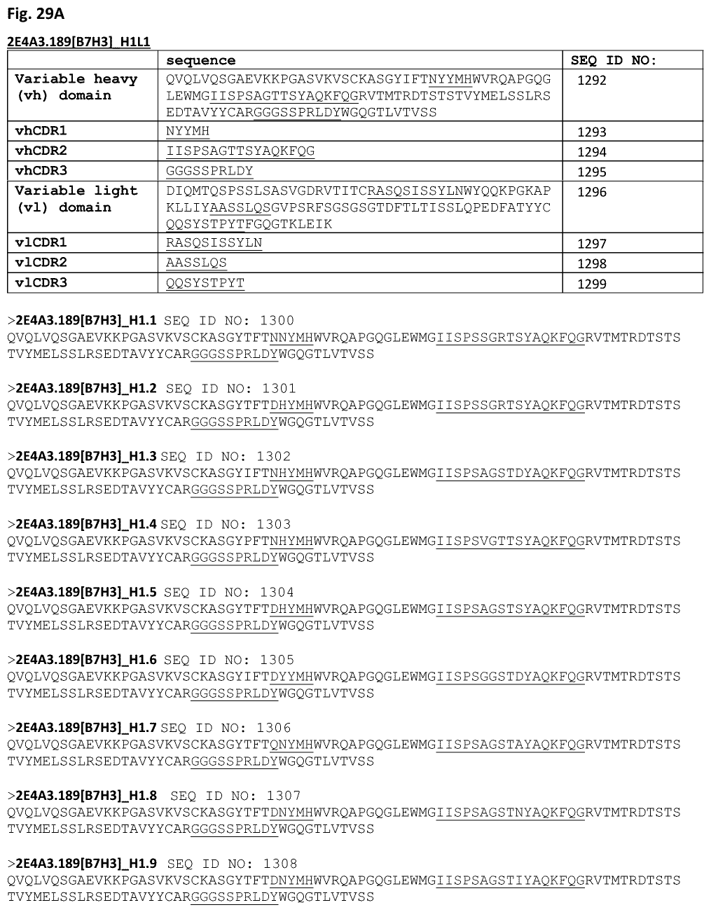

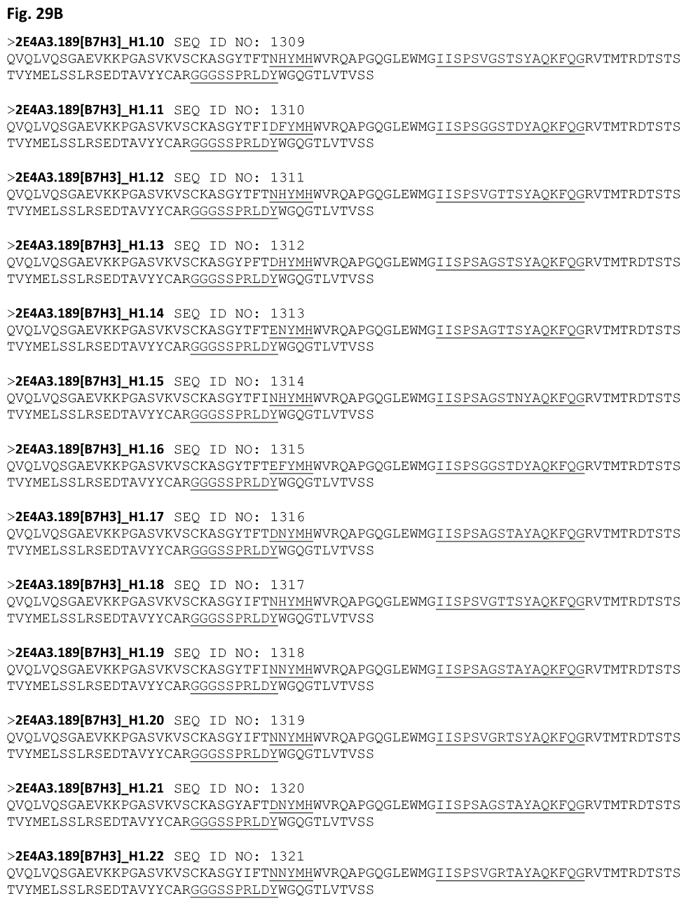

In addition, the naming nomenclature of particular antigen binding domains (e.g., NK cell and TTA binding domains) use a “Hx.xx_Ly.yy” or “Hx.xxLy.yy” type of format, with the numbers being unique identifiers to particular variable chain sequences. Thus, the variable domain of the Fab side of B7H3 binding domain 38E2[B7H3] (e.g., FIG. 28) is “H2L1.1”, which indicates that the variable heavy domain, H2, was combined with the light domain L1.1. In the case that these sequences are used as scFvs, the designation “H2_L1.1” or “H2L1.1”, indicates that the variable heavy domain, H2 is combined with the light domain, L1.1, and is in VH-linker-VL orientation, from N- to C-terminus. This molecule with the identical sequences of the heavy and light variable domains but in the reverse order (VL-linker-VH orientation, from N- to C-terminus) would be designated “L1.1_H2”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments, and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

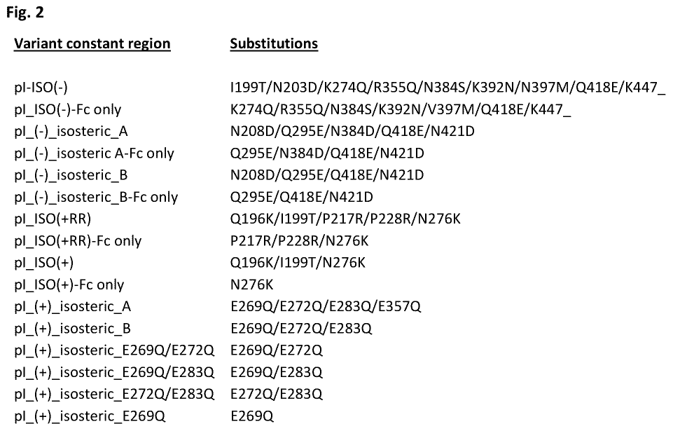

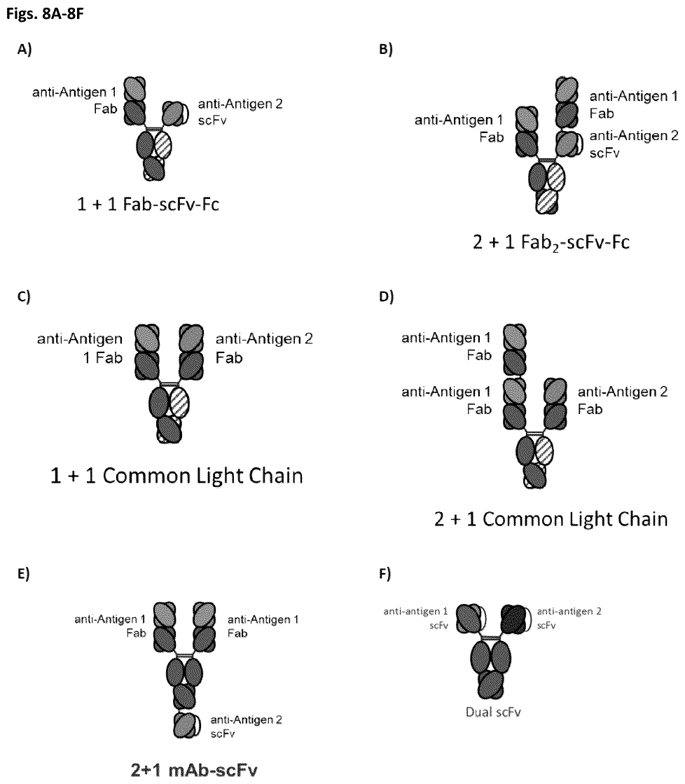

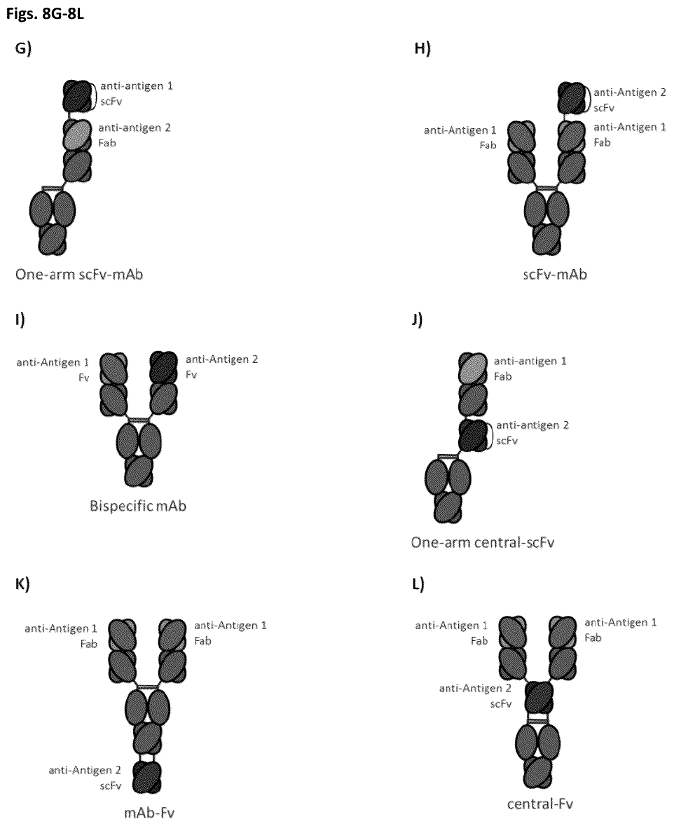

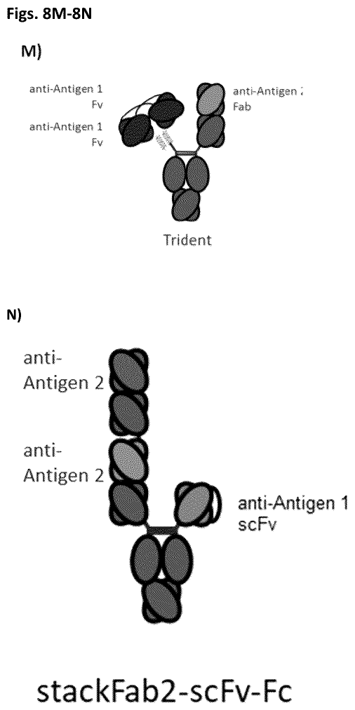

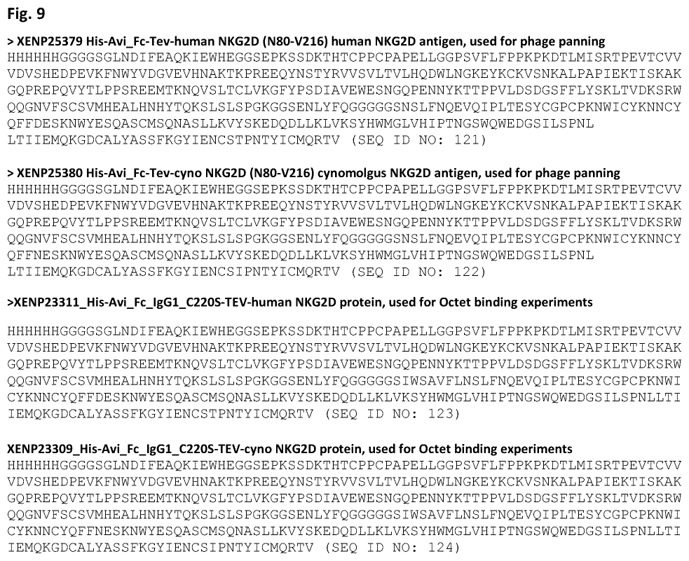

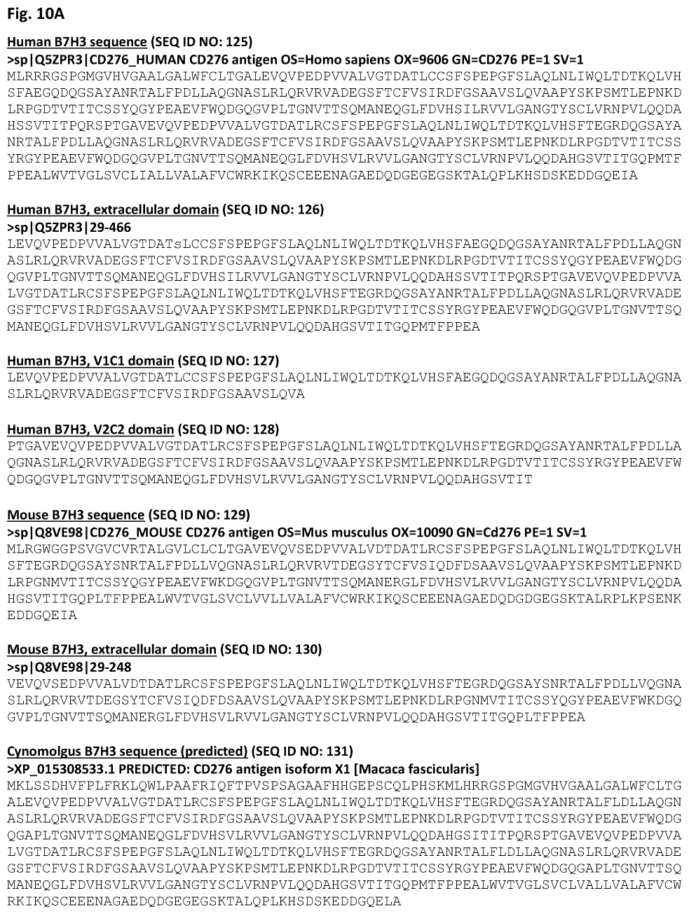

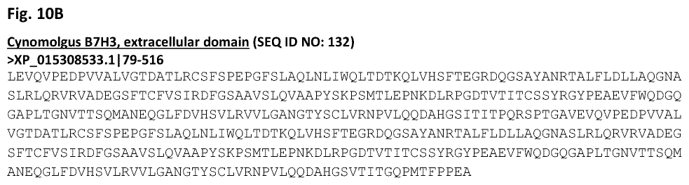

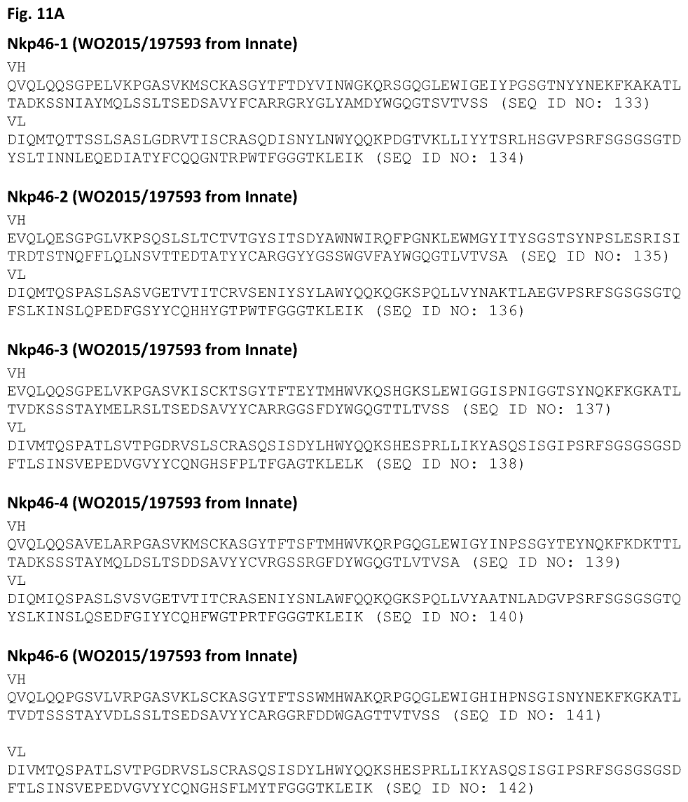

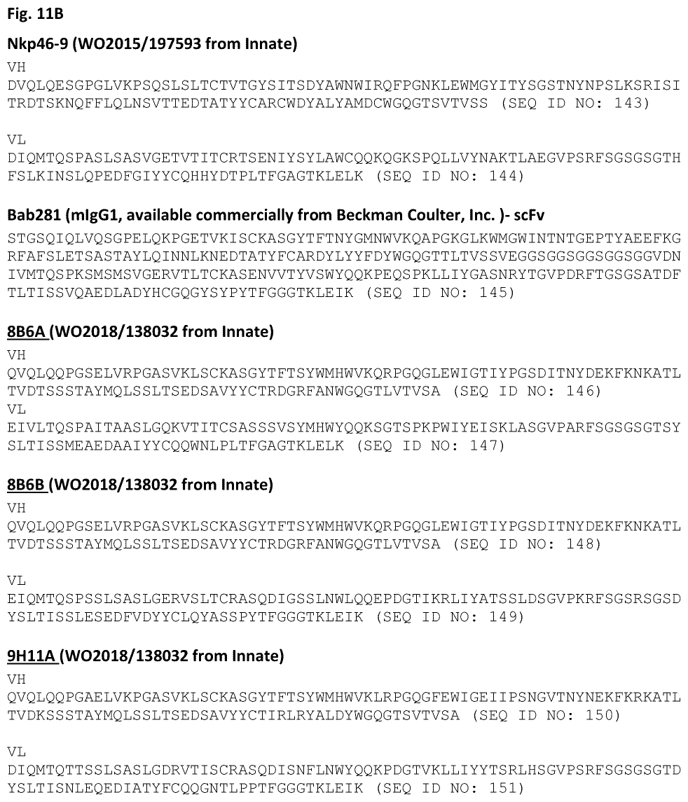

Other useful antibody formats include, but are not limited to, the “1+1 Fab-scFv-Fc,” “2+1 Fab₂-scFv-Fc,” and “2+1 mAb-scFv” formats provided herein (see, e.g., FIGS. 8A, 8B, and 8E). Additional useful antibody formats include, but are not limited to, “mAb-Fv,” “central-Fv,” “one armed scFv-mAb,” “scFv-mAb,” “dual scFv,” “1+1 CLC,” “2+1 CLC,” “one-armed central scFv,” “2+1 stack Fab₂-scFv-Fc,” “bispecific mAb,” and “trident” format antibodies, as disclosed in US20180127501A1, which is incorporated by reference herein, particularly in pertinent part relating to antibody formats (see, e.g., FIG. 2 of US20180127501A1), and as are shown in FIG. 8.

Antibody heavy chains typically include a variable heavy (V_{H}) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: V_{H}-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (C_{H}) domains and the hinge domains. In the context of IgG antibodies, the IgG isotypes each have three CH regions. Accordingly, “C_{H}” (or “CH”) domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn. As will be appreciated by those in the art, an Fc domain is a self-assembling dimer composed of two Fc regions, Fc monomer 1 and Fc monomer 2. Thus there are monomeric Fc domains (meaning just one monomer) and dimeric Fc domains. As outlined herein, the NK engagers generally rely on dimeric Fc domains that have different amino acid substitutions in each monomer, e.g. they are heterodimeric and not homodimeric.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as C_{L} or C_{K}). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., B7H3 or MICA/B) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1): 55-77 (2003).

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 23-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH or V_{H}; containing the vhCDR1, vhCDR2 and vhCDR3) and the variable light domain (vl or VL or V_{L}; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the V_{κ}, V_{λ}, and/or V_{H} genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the V_{H}, CH1, V_{L}, and C_{L} immunoglobulin domains, generally on two different polypeptide chains (e.g., V_{H}-CH1 on one chain and V_{L}—C_{L} on the other). Fab may refer to this region in isolation, or this region in the context of a bispecific antibody of the invention. In the context of a Fab, the Fab comprises an Fv region in addition to the CH1 and C_{L} domains.

By “Fv” or “Fv fragment” or “Fv region” as used herein is meant the antibody region that comprises the V_{L} and V_{H} domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the Figures, the order of the vh and v domain is indicated in the name, e.g., H.X L.Y means N- to C-terminal is vh-linker-vl, and L.Y H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as V_{H}-scFv linker-V_{L}, this can be reversed for any of the scFv domains (or those constructed using vh and vl sequences from Fabs), to V_{L}-scFv linker-V_{H}, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233− or E233 #, E233( ), E233_, or E233del designates a deletion of glutamic acid at position 233. Additionally, EDA233− or EDA233 #designates a deletion of the sequence GluAspAla that begins at position 233.

By “variant protein” or “protein variant,” or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, or IgG4, although human sequences with variants can also serve as “parent polypeptides,” for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 96, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides, and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190: 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor,” “FcγR,” “FcγR,” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcγRIIIa” or “CD16A” as used herein is meant a receptor for the Fc region of immunoglobulin IgG. Additional details about human CD16A such as an amino acid sequence, nucleic acid sequence and genomic information can be found in UniProtKB/Swiss-Prot No. P08637, NCBI GenPept Nos. NP_000560.6 and NP_001316049.1, NCBI GenBank No. NM_000569.8, and the like.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide.

Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild-type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M.S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W.R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻² M, or greater, where K_{D} refers to a dissociation rate of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a K_{A} or Kₐ for an antigen or epitope of at least 20-, 50-, 100-, 500-, 1000-, 5,000-, 10,000- or more times greater for the epitope relative to a control, where K_{A} or Kₐ refers to an association rate of a particular antibody-antigen interaction. Binding affinity is generally measured using a Biacore, SPR or BLI assay.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone)”.

As used herein, the term “about” a value (or parameter) refers to, for example, ±1%, ±2%, ±3%, ±4%, ±5%, 6%, ±7%, ±8%, ±9%, ±10% and the like of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

### IV. Introduction

The present invention provides natural killer cell engagers (NKEs), a growing class of immune-oncology biological medicines.

One aspect of the mechanism of NKEs is the ability to bind to the human receptor CD16A, also referred to as FcγRIIIa. The engagement of CD16A is, by itself, sufficient to produce cytokines and trigger cytotoxic activity on NK cells. Thus, while many TCE multispecific antibodies utilize variant Fc domains that ablate FcγR receptor binding to minimize off target interactions, NKEs require binding to CD16A. In some cases, NKEs do this by utilizing a separate CD16A antigen binding domain (see, for example, WO2022/074206). In others, such as those described herein, the NKE constructs utilize human IgG1 Fc domain amino acid sequences that retain the ability to bind CD16A by utilizing Fc domains derived from human IgG1 Fc domains. In the present invention, additional amino acid substitutions in the Fc domains of the NKE antibodies are utilized to increase the binding of the NKE to CD16A. Alternatively, it is also possible to produce the NKE antibodies in cell lines that lead to less fucosylation of the antibody that is produced, which leads to increased ADCC.

Accordingly, the present invention provides antibodies, including multispecific antibodies, that bind to NK cells, optionally a tumor target antigen (TTA) and have increased binding to the CD16A receptor. That is, the antibodies of the invention comprise one or two NK cell antigen binding domains (ABDs), an Fc domain that has increased binding to CD16A as compared to a human IgG1 Fc domain, and one or two ABDs to a TTA. These are generally described as heterodimeric bispecific antibodies, where the Fc regions of each heavy chain are different, generally including amino acid substitutions that allow for heterodimeric Fc domain formation, increased binding to CD16A and other functionalities. As is more fully described below, the amino acid substitutions that allow for increased binding to CD16A can be incorporated into either or both of the monomeric Fc domains. Additionally, as is more fully discussed below, the multispecific antibodies of the invention can have a variety of different formats, with some formats having one or two identical binding domains.

### V. Antigen Binding Domains

A. Antigen Binding Domains that Bind Human NK Cells

The present invention provides antibodies, including multispecific antibodies, with antigen binding domains (ABDs) that bind to the extracellular domain (ECD) of human NK cell surface receptors, including, but not limited to, human NKG2D, human NKp46, and human NKp30. Binding affinities of a specific NK cell ABD can be evaluated using an assay recognized by those skilled in the art including, but not limited to, a Surface Plasmon Resonance (SPR) and/or a BLI binding assay (such as Biacore, Octet, or Caterra LSA).

1. NKG2D Antigen Binding Domains

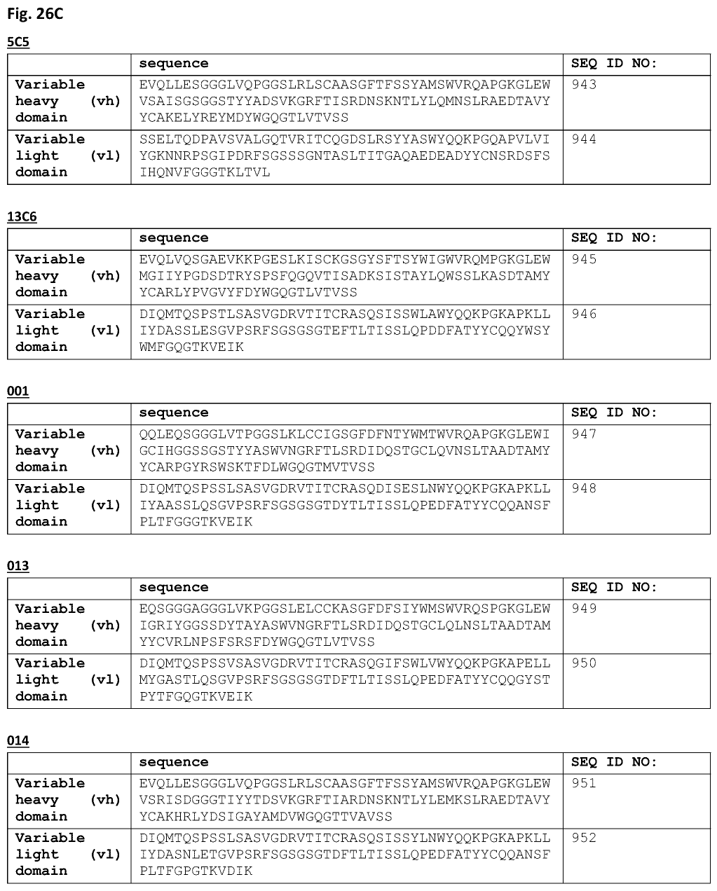

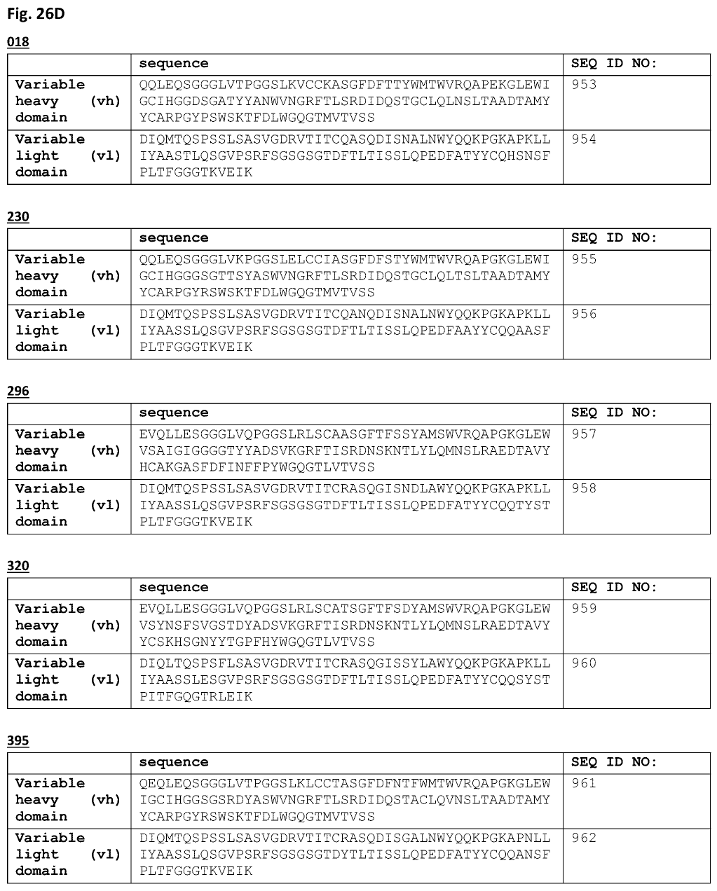

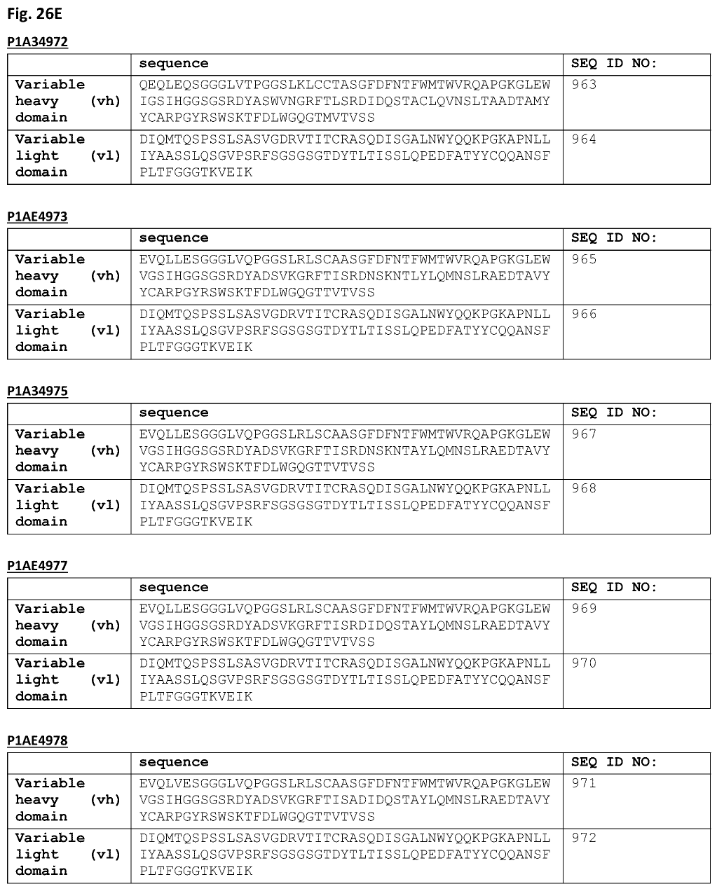

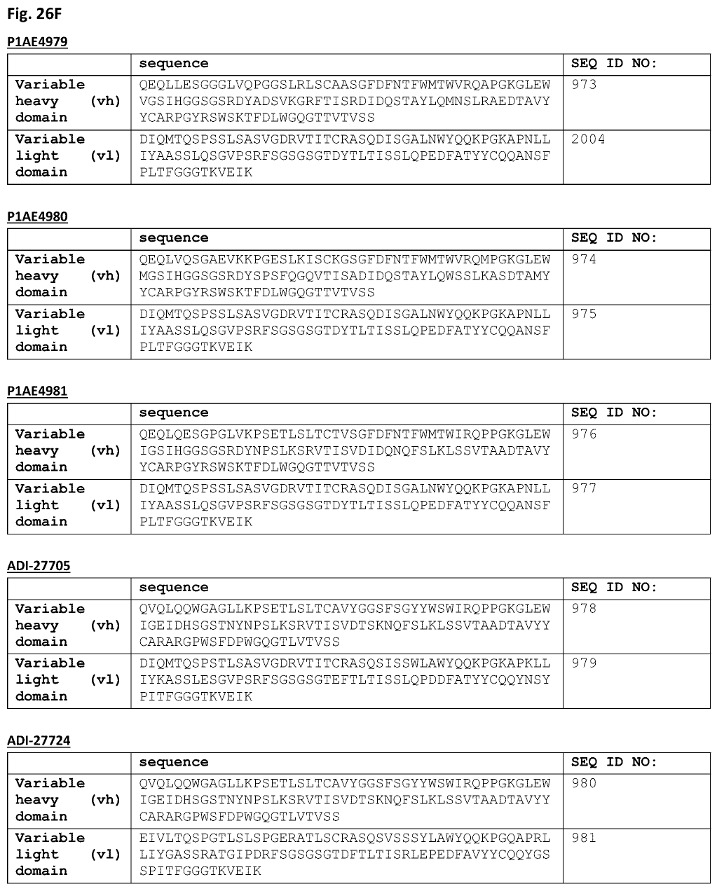

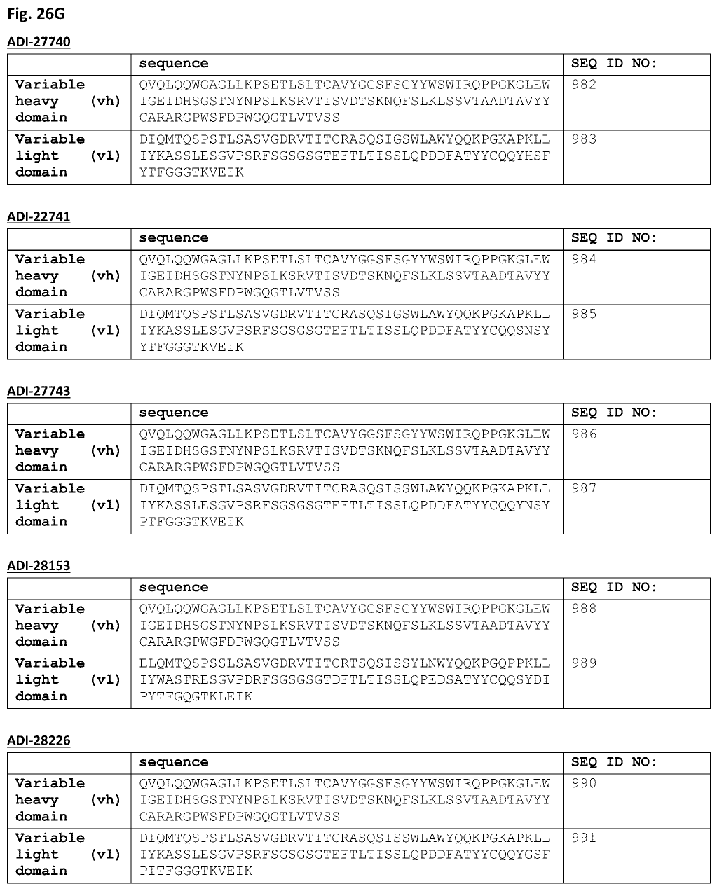

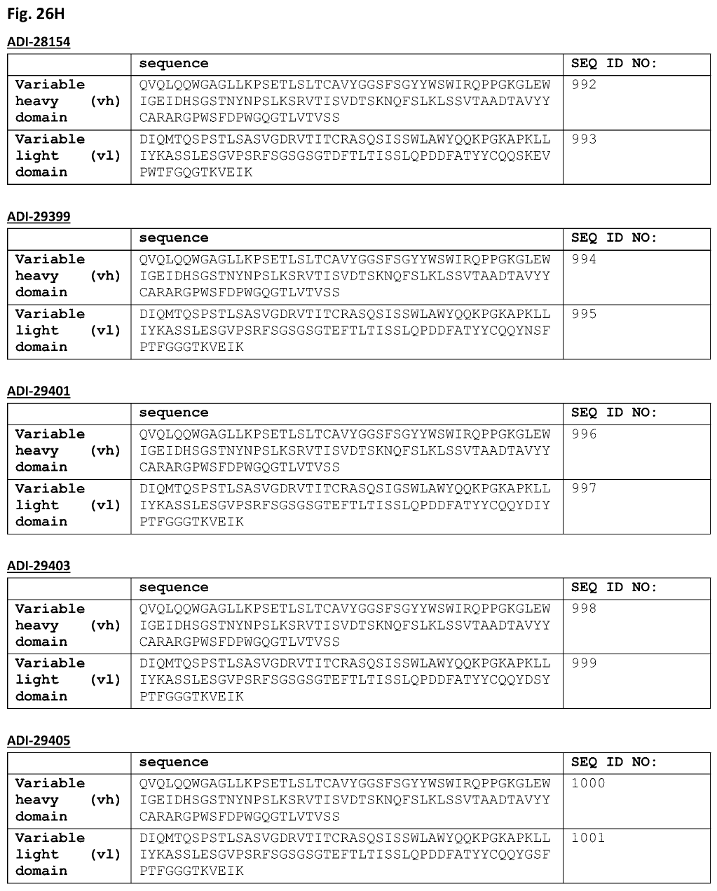

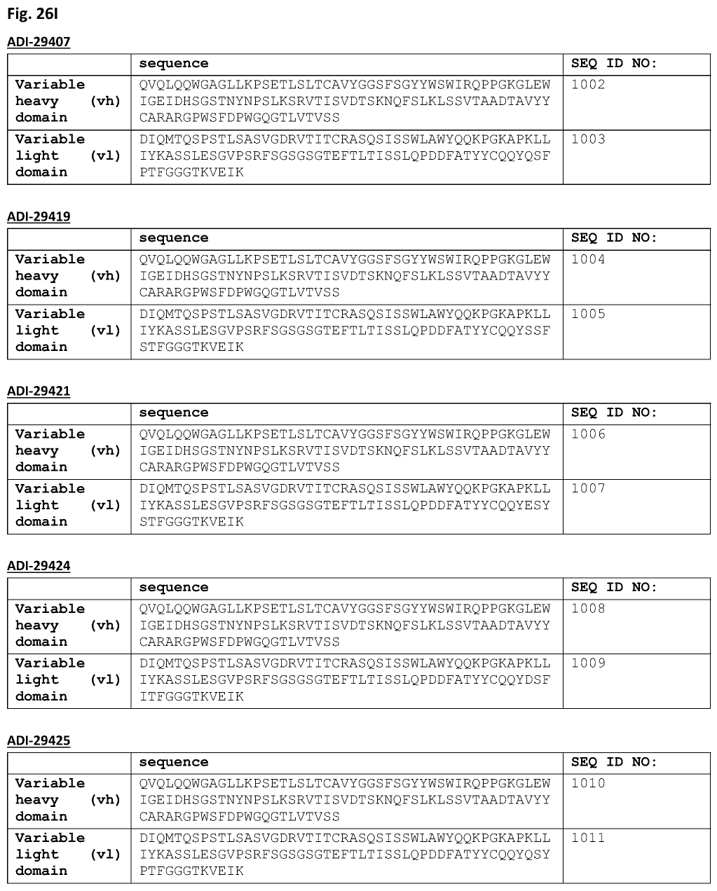

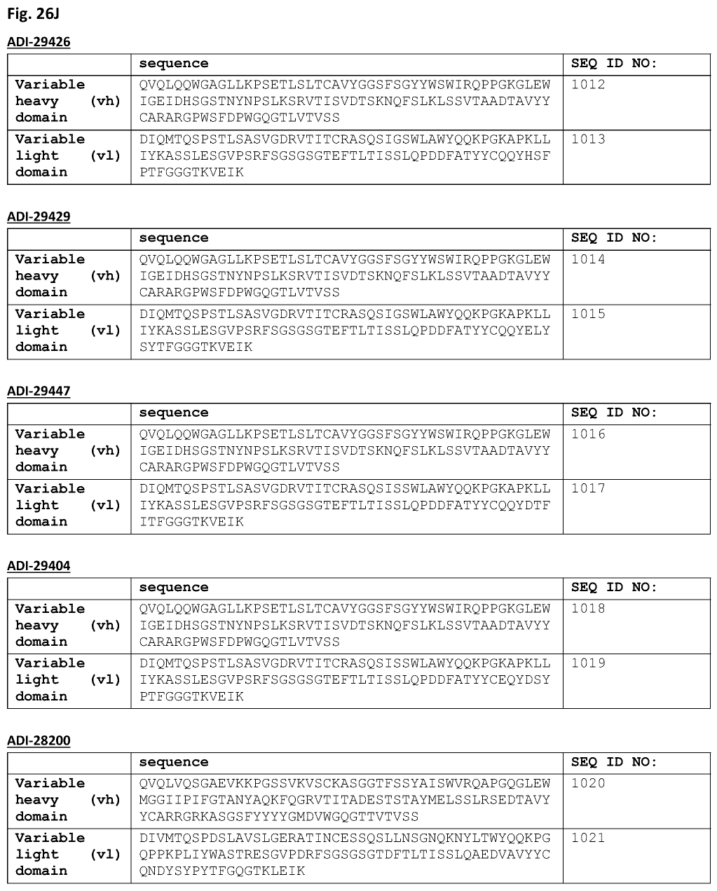

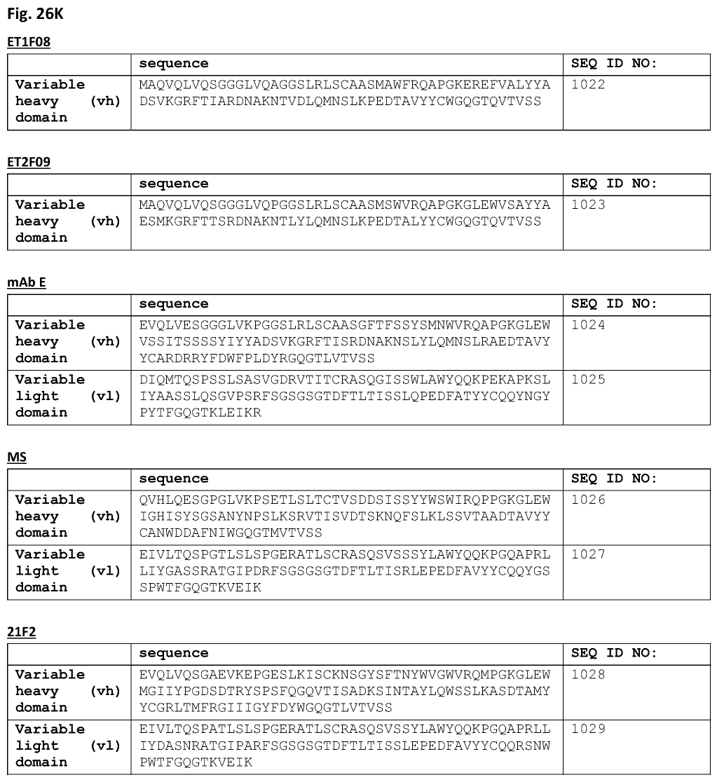

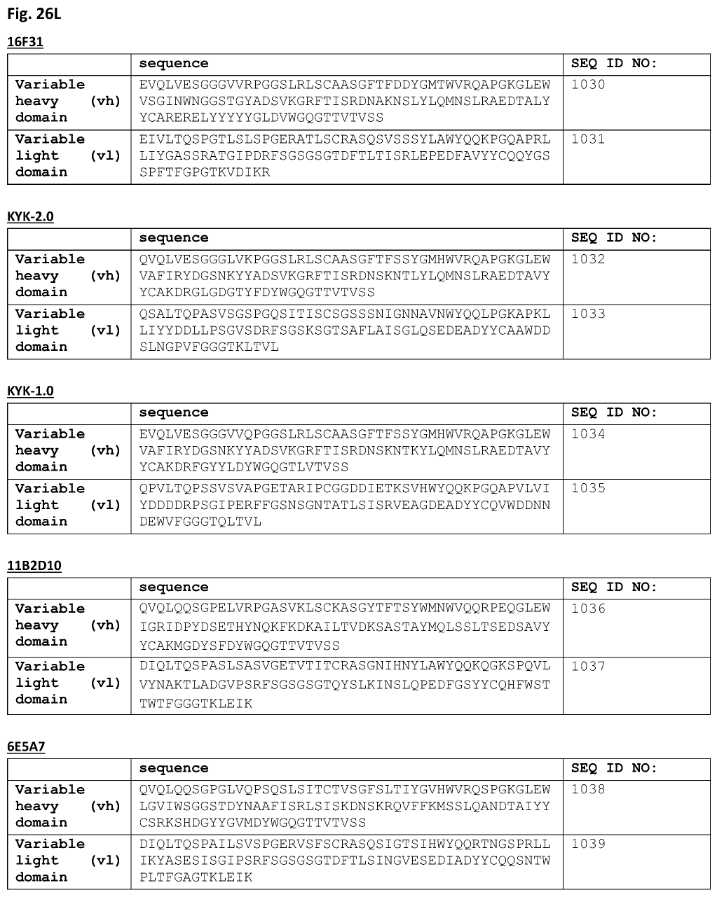

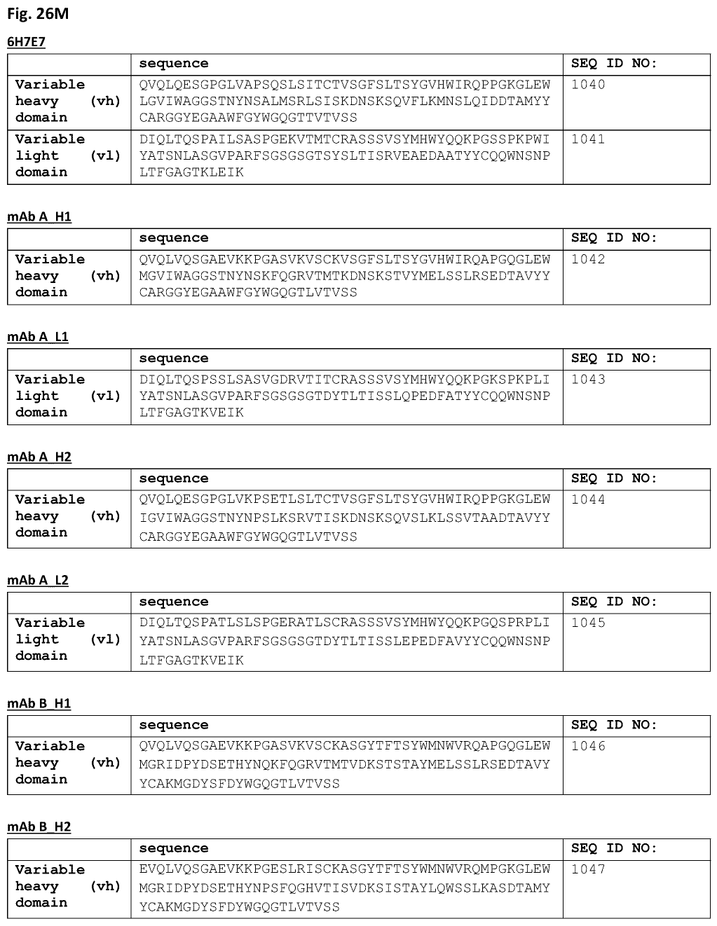

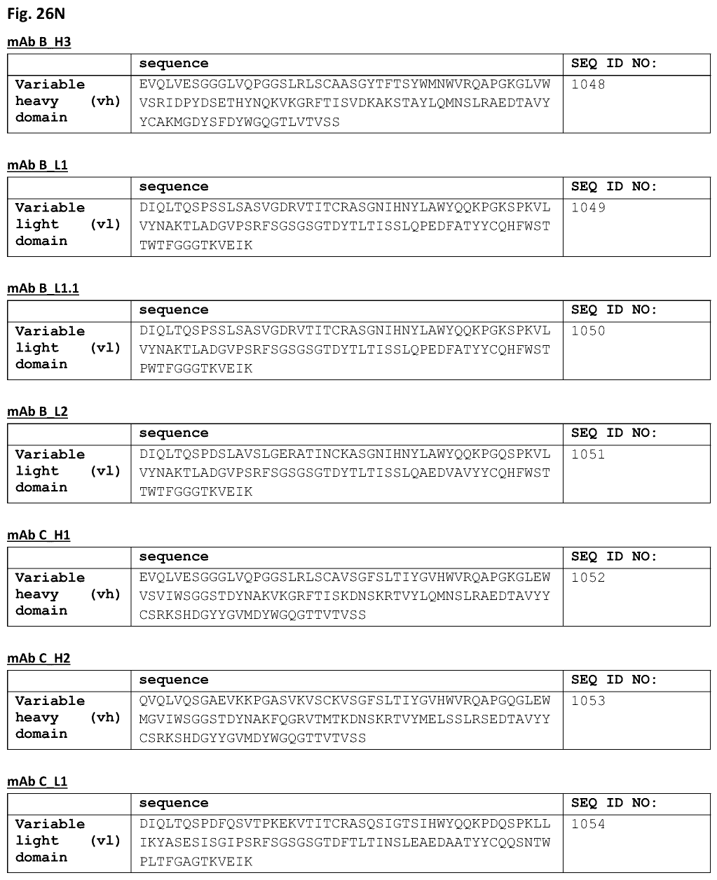

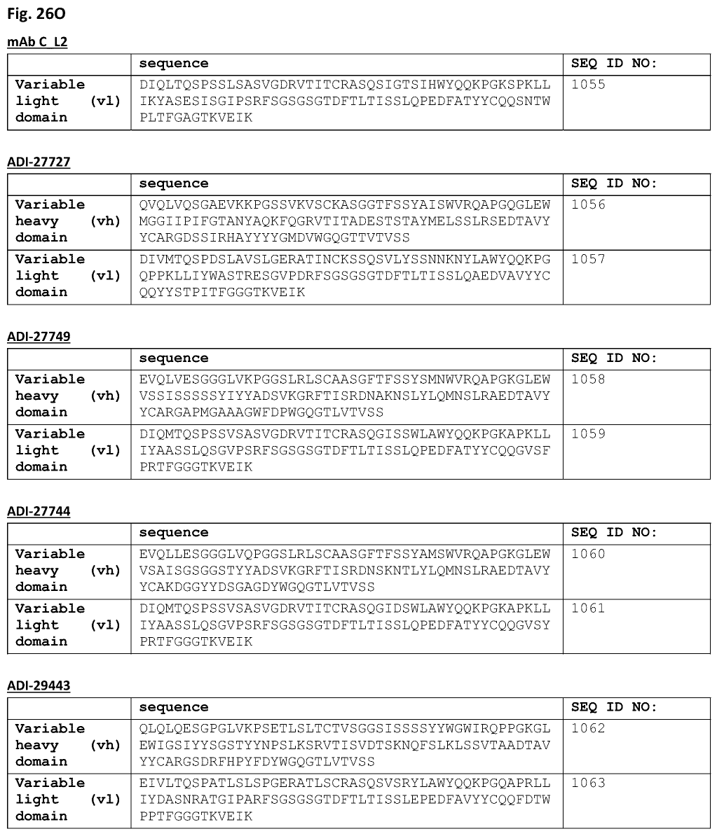

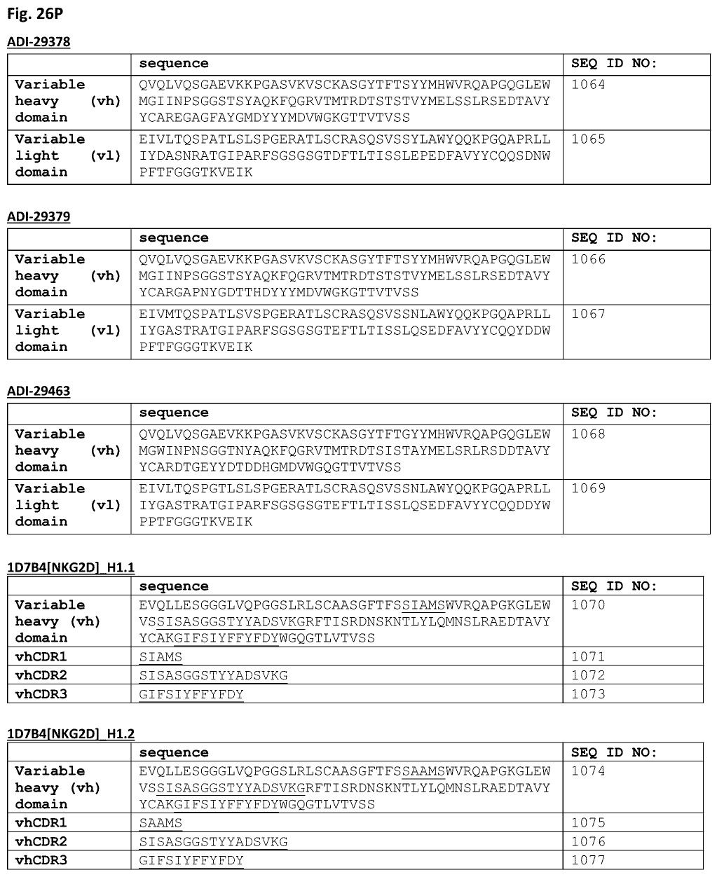

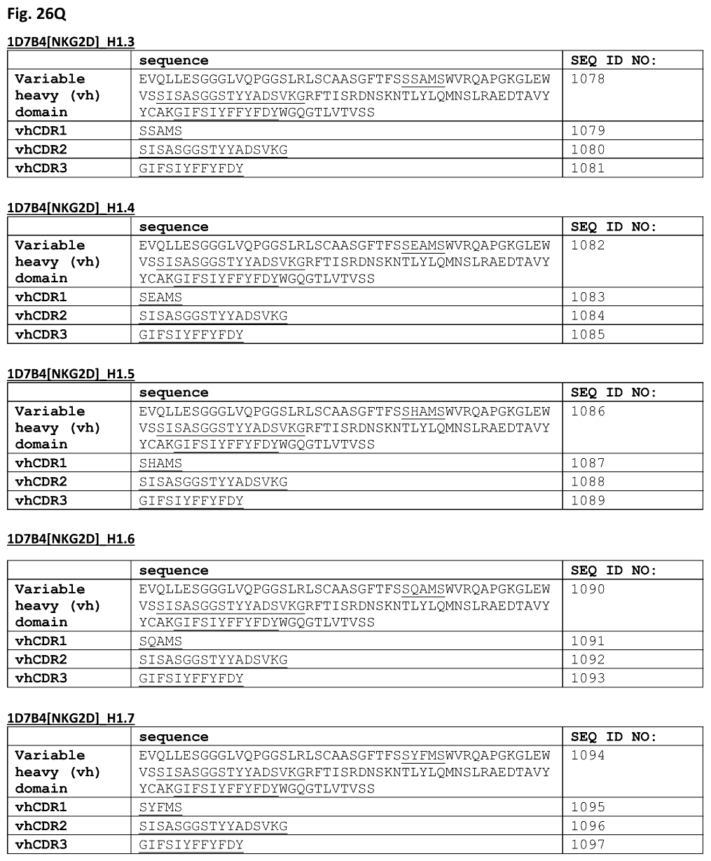

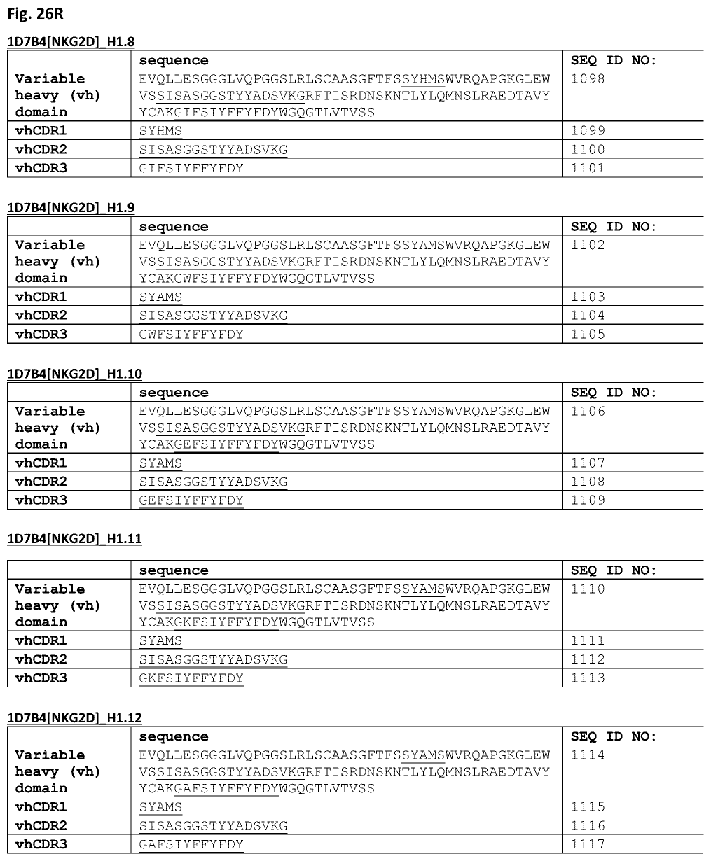

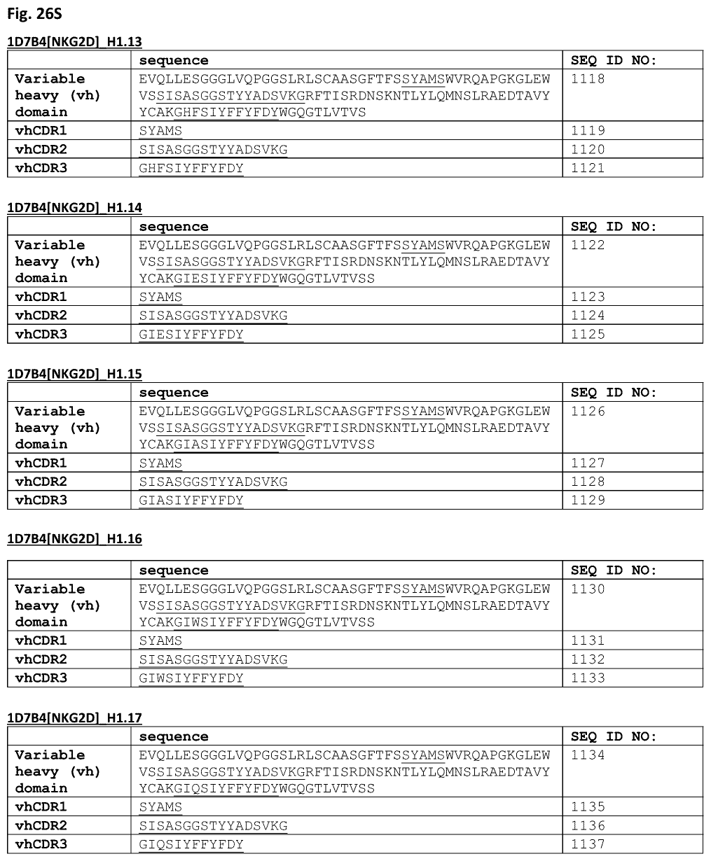

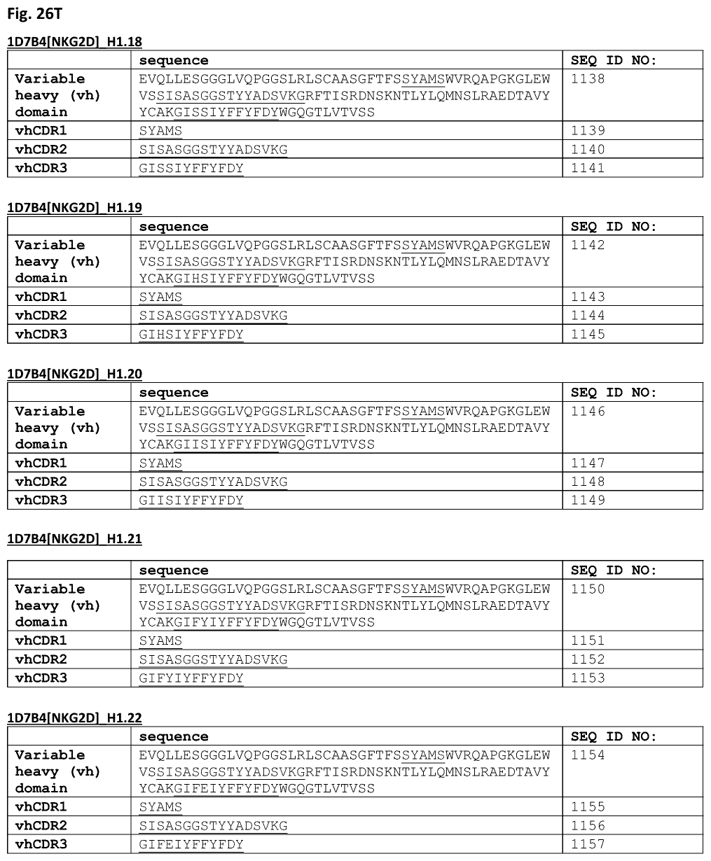

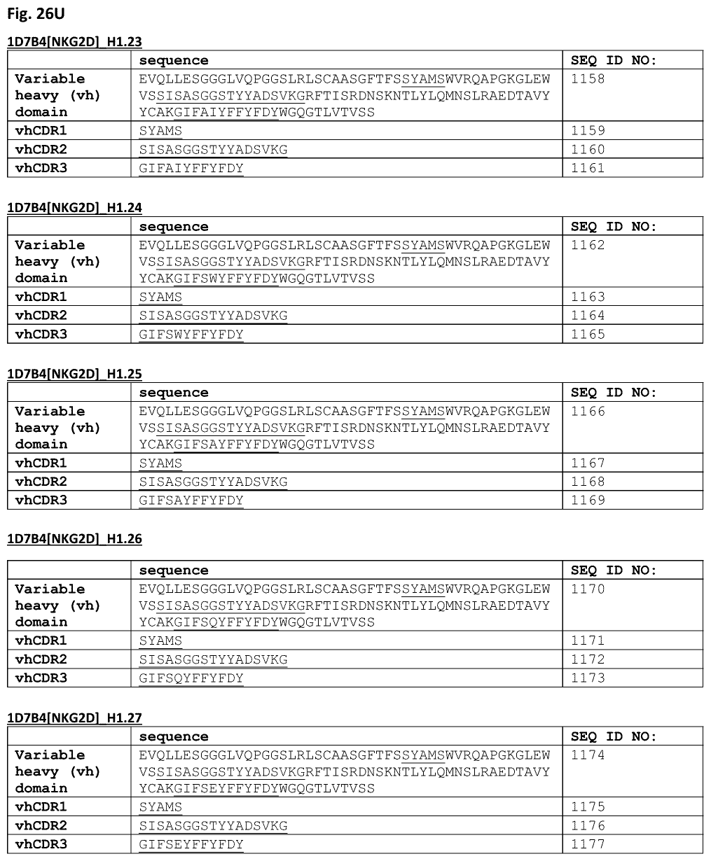

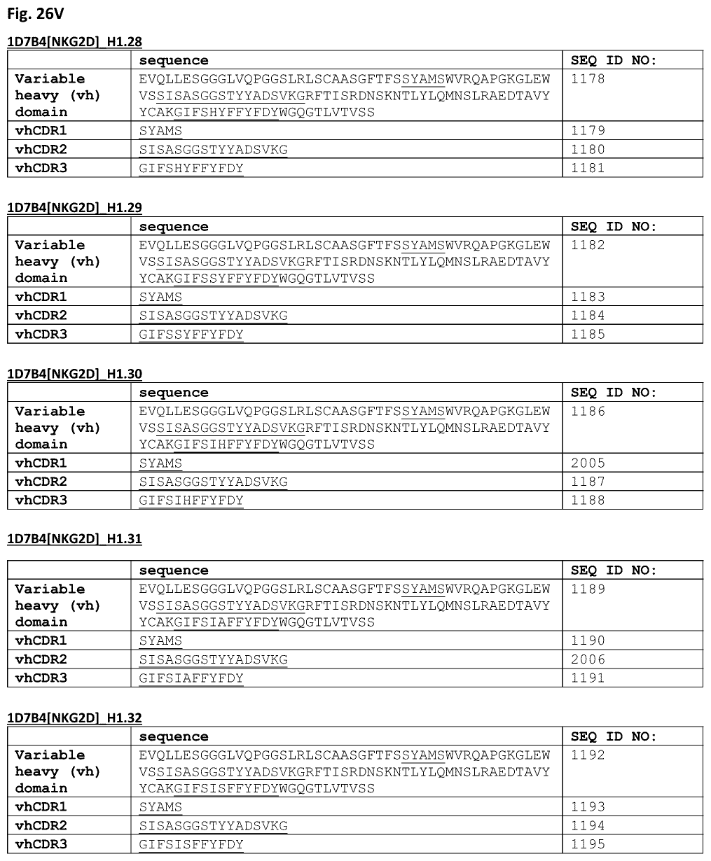

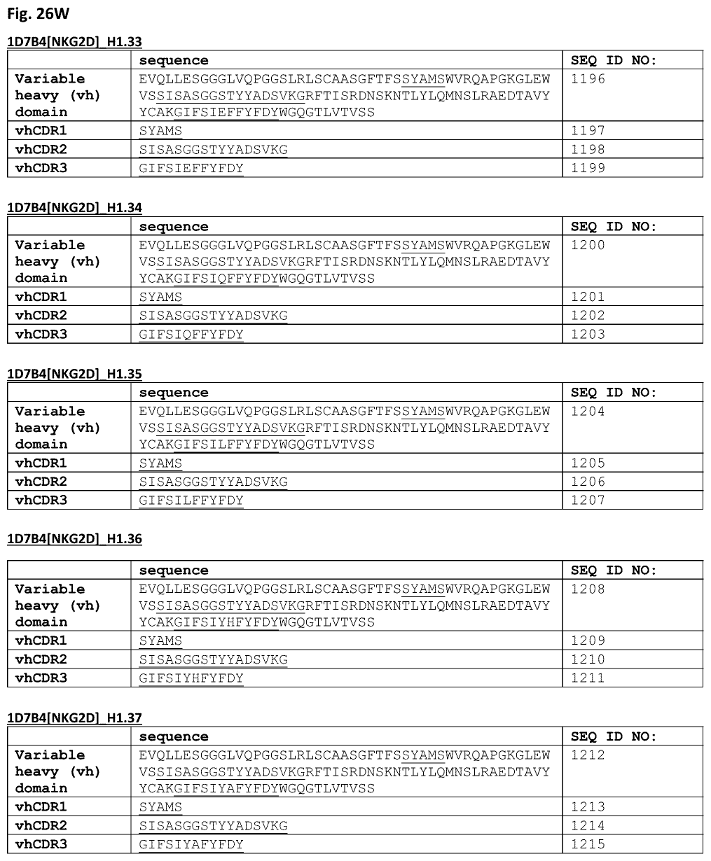

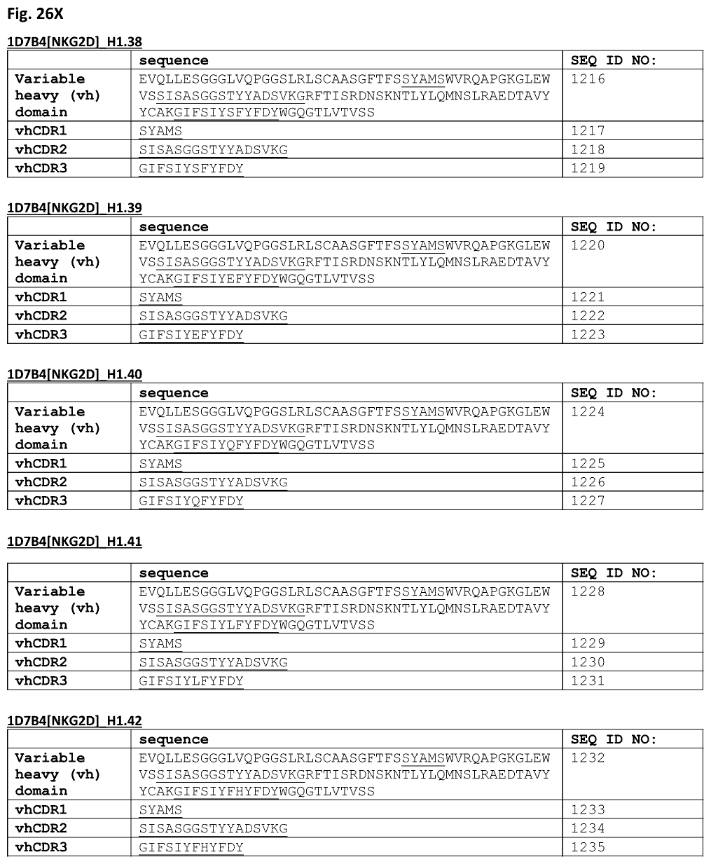

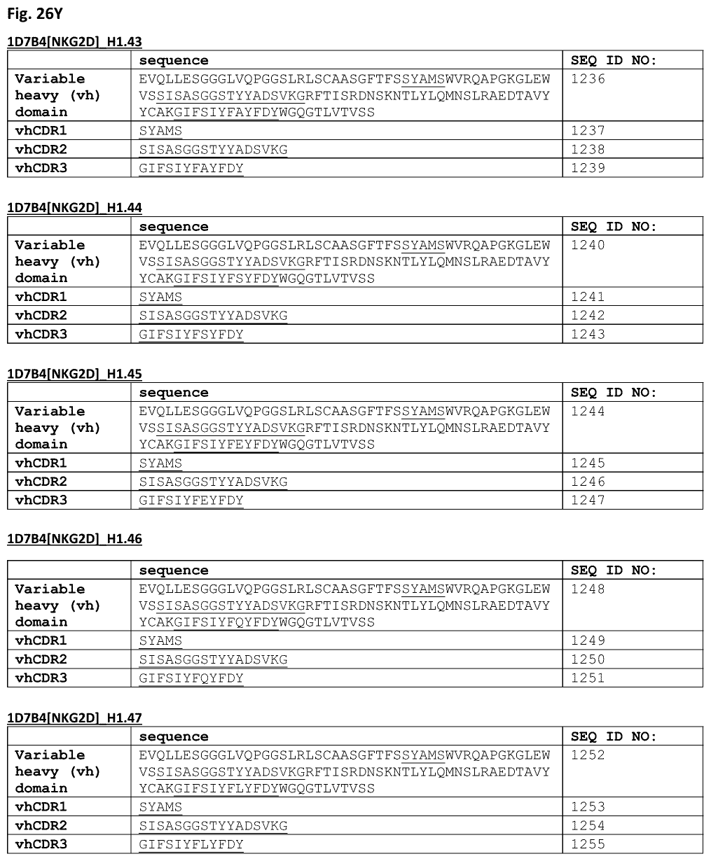

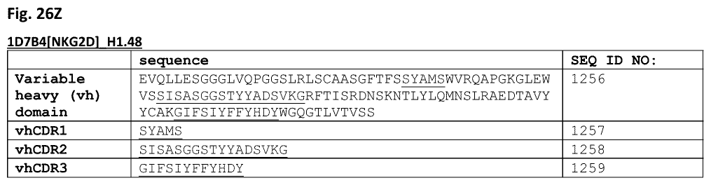

In some embodiments, the NKEs include an ABD that binds to the ECD of human NKG2D. Suitable variable heavy VH and variable light VL domains that bind to the ECD of human NKG2D include, but are not limited to, VH/VL pairs selected from the group including: (i) SEQ ID NOs: 911 and 915, (ii) SEQ ID NOs: 919 and 923, (iii) SEQ ID NOs: 927 and 931, (iv) SEQ ID NOs: 935 and 939, (v) SEQ ID NOs: 943 and 944, (vi) SEQ ID NOs: 945 and 946, (vii) SEQ ID NOs: 947 and 948, (viii) SEQ ID NOs: 949 and 950, (ix) SEQ ID NOs: 951 and 952, (x) SEQ ID NOs: 953 and 954, (xi) SEQ ID NOs: 955 and 956, (xii) SEQ ID NOs: 957 and 958, (xiii) SEQ ID NOs: 959 and 960, (xiv) SEQ ID NOs: 961 and 962, (xv) SEQ ID NOs: 963 and 964, (xvi) SEQ ID NOs: 965 and 966, (xvii) SEQ ID NOs: 967 and 968, (xviii) SEQ ID NOs: 969 and 970, (xix) SEQ ID NOs: 971 and 972, (xx) SEQ ID NOs: 973 and 2004, (xxi) SEQ ID NOs: 974 and 975, (xxii) SEQ ID NOs: 976 and 977, (xxiii) SEQ ID NOs: 978 and 979, (xxiv) SEQ ID NOs: 980 and 981, (xxv) SEQ ID NOs: 982 and 983, (xxvi) SEQ ID NOs: 984 and 985, (xxvii) SEQ ID NOs: 986 and 987, (xxviii) SEQ ID NOs: 988 and 989, (xxix) SEQ ID NOs: 990 and 991, (xxx) SEQ ID NOs: 992 and 993, (xxxi) SEQ ID NOs: 994 and 995, (xxxii) SEQ ID NOs: 996 and 997, (xxxiii) SEQ ID NOs: 998 and 999, (xxxiv) SEQ ID NOs: 1000 and 1001, (xxxv) SEQ ID NOs: 1002 and 1003, (xxxvi) SEQ ID NOs: 1004 and 1005, (xxxvii) SEQ ID NOs: 1006 and 1007, (xxxviii) SEQ ID NOs: 1008 and 1009, (xxxix) SEQ ID NOs: 1010 and 1011, (xl) SEQ ID NOs: 1012 and 1013, (xli) SEQ ID NOs: 1014 and 1015, (xlii) SEQ ID NOs: 1016 and 1017, (xliii) SEQ ID NOs: 1018 and 1019, (xliv) SEQ ID NOs: 1020 and 1021, (xlv) SEQ ID NOs: 1024 and 1025, (xlvi) SEQ ID NOs: 1026 and 1027, (xlvii) SEQ ID NOs: 1028 and 1029, (xlviii) SEQ ID NOs: 1030 and 1031, (xlix) SEQ ID NOs: 1032 and 1033, (1) SEQ ID NOs: 1034 and 1035, (li) SEQ ID NOs: 1036 and 1037, (lii) SEQ ID NOs: 1038 and 1039, (liii) SEQ ID NOs: 1040 and 1041, (liv) SEQ ID NOs: 1042 and 1043, (lv) SEQ ID NOs: 1042 and 1045, (lvi) SEQ ID NOs: 1044 and 1043, (lvii) SEQ ID NOs: 1044 and 1045, (lviii) SEQ ID NOs: 1046 and 1049, (lix) SEQ ID NOs: 1046 and 1050, (lx) SEQ ID NOs: 1046 and 1051, (lxi) SEQ ID NOs: 1047 and 1049, (lxii) SEQ ID NOs: 1047 and 1050, (lxiii) SEQ ID NOs: 1047 and 1051, (lxiv) SEQ ID NOs: 1048 and 1049, (lxv) SEQ ID NOs: 1048 and 1050, (lxvi) SEQ ID NOs: 1048 and 1051, (lxvii) SEQ ID NOs: 1052 and 1054, (lxviii) SEQ ID NOs: 1052 and 1055, (lxix) SEQ ID NOs: 1053 and 1054, (lxx) SEQ ID NOs: 1053 and 1055, (lxxi) SEQ ID NOs: 1056 and 1057, (lxxii) SEQ ID NOs: 1058 and 1059, (lxxiii) SEQ ID NOs: 1060 and 1061, (lxxiv) SEQ ID NOs: 1062 and 1063, (lxxv) SEQ ID NOs: 1064 and 1065, (lxxvi) SEQ ID NOs: 1066 and 1067, (lxxvii) SEQ ID NOs: 1068 and 1069, (lxxviii) SEQ ID NOs: 1070 and 915, (lxxix) SEQ ID NOs: 1074 and 915, (lxxx) SEQ ID NOs: 1078 and 915, (lxxxi) SEQ ID NOs: 1082 and 915, (lxxxii) SEQ ID NOs: 1086 and 915, (lxxxiii) SEQ ID NOs: 1090 and 915, (lxxxix) SEQ ID NOs: 1094 and 915, (lxxxv) SEQ ID NOs: 1098 and 915, (lxxxvi) SEQ ID NOs: 1102 and 915, (lxxxvii) SEQ ID NOs: 1106 and 915, (lxxxviii) SEQ ID NOs: 1110 and 915, (lxxxix) SEQ ID NOs: 1114 and 915, (xc) SEQ ID NOs: 1118 and 915, (xci) SEQ ID NOs: 1122 and 915, (xcii) SEQ ID NOs: 1126 and 915, (xciii) SEQ ID NOs: 1130 and 915, (xciv) SEQ ID NOs: 1134 and 915, (xcv) SEQ ID NOs: 1138 and 915, (xcvi) SEQ ID NOs: 1142 and 915, (xcvii) SEQ ID NOs: 1146 and 915, (xcviii) SEQ ID NOs: 1150 and 915, (xcix) SEQ ID NOs: 1154 and 915, (c) SEQ ID NOs: 1158 and 915, (ci) SEQ ID NOs: 1162 and 915, (cii) SEQ ID NOs: 1166 and 915, (ciii) SEQ ID NOs: 1170 and 915, (civ) SEQ ID NOs: 1174 and 915, (cv) SEQ ID NOs: 1178 and 915, (cvi) SEQ ID NOs: 1182 and 915, (cvii) SEQ ID NOs: 1186 and 915, (cviii) SEQ ID NOs: 1189 and 915, (cix) SEQ ID NOs: 1192 and 915, (cx) SEQ ID NOs: 1196 and 915, (cxi) SEQ ID NOs: 1200 and 915, (cxii) SEQ ID NOs: 1204 and 915, (cxiii) SEQ ID NOs: 1208 and 915, (cxiv) SEQ ID NOs: 1212 and 915, (cxv) SEQ ID NOs: 1216 and 915, (cxvi) SEQ ID NOs: 1220 and 915, (cxvii) SEQ ID NOs: 1224 and 915, (cxviii) SEQ ID NOs: 1228 and 915, (cxix) SEQ ID NOs: 1232 and 915, (cxx) SEQ ID NOs: 1236 and 915, (cxxi) SEQ ID NOs: 1240 and 915, (cxxii) SEQ ID NOs: 1244 and 915, (cxxiii) SEQ ID NOs: 1248 and 915, (cxxiv) SEQ ID NOs: 1252 and 915, and (cxxv) SEQ ID NOs: 1256 and 915, as shown in FIGS. 26A-26Z. Additionally, any sequence included in FIG. 26 can also be individually included or excluded. It should be noted that these ABDs can be included in any of the heterodimeric formats described herein. In some embodiments, the NKEs of the invention include at least one anti-NKG2D antigen binding domain, as described herein and in the Figures. Described herein are a plurality of means for binding the ECD of human NKG2D.

2. NKp46 Antigen Binding Domains

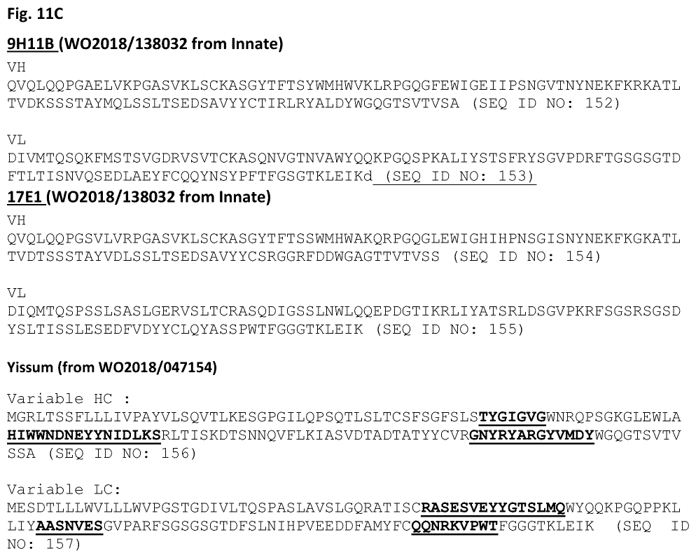

In some embodiments, the NKEs include an ABD that binds to the ECD of human NKp46. Suitable variable heavy VH and variable light VL domains that bind to the ECD of human NKp46 include, but are not limited to, VH/VL pairs selected from the group including: (i) SEQ ID NOs: 133 and 134, (ii) SEQ ID NOs: 135 and 136, (iii) SEQ ID NOs: 137 and 138, (iv) SEQ ID NOs: 139 and 140, (v) SEQ ID NOs: 141 and 142, (vi) SEQ ID NOs: 143 and 144, (vii) SEQ ID NOs: 146 and 147, (viii) SEQ ID NOs: 148 and 149, (ix) SEQ ID NOs: 150 and 151, (x) SEQ ID NOs: 152 and 153, (xi) SEQ ID NOs: 154 and 155, and (xii) SEQ ID NOs: 156 and 157, as shown in FIGS. 11A-11C. Additionally, any sequence included in FIG. 11 can also be individually included or excluded. It should be noted that these ABDs can be included in any of the formats described herein. In some embodiments, the NKEs of the invention include at least one anti-NKp46 antigen binding domain, as described herein and in the Figures. Described herein are a plurality of means for binding the ECD of human NKp46.

3. NKp30 Antigen Binding Domains

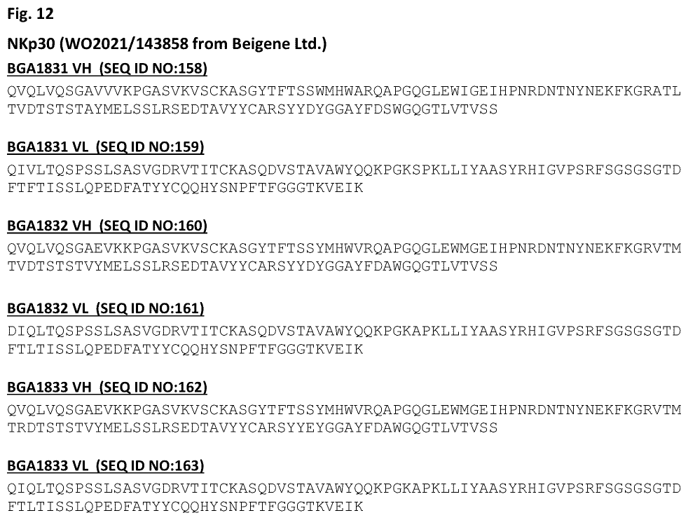

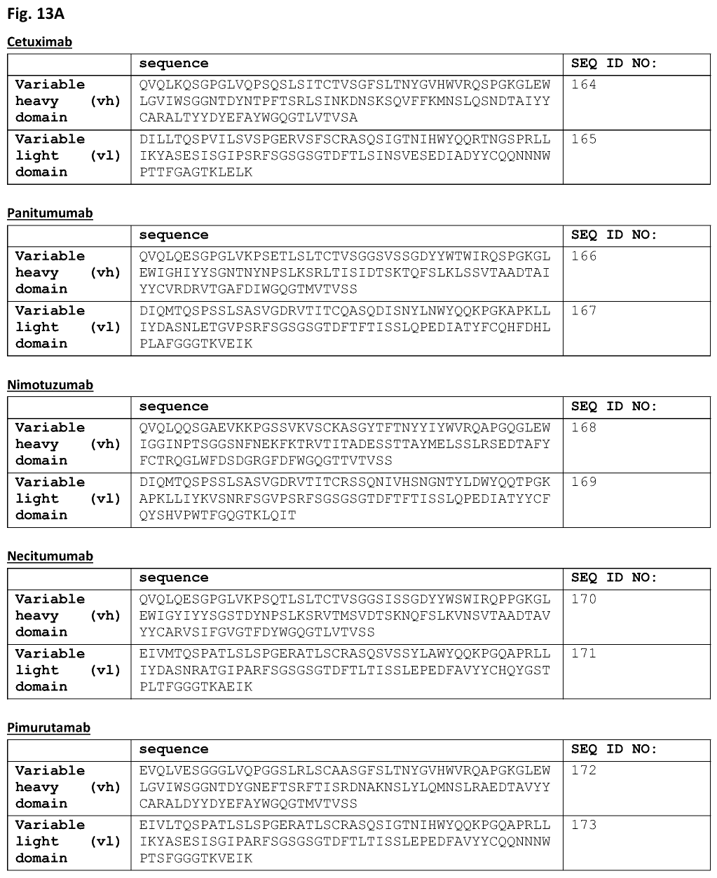

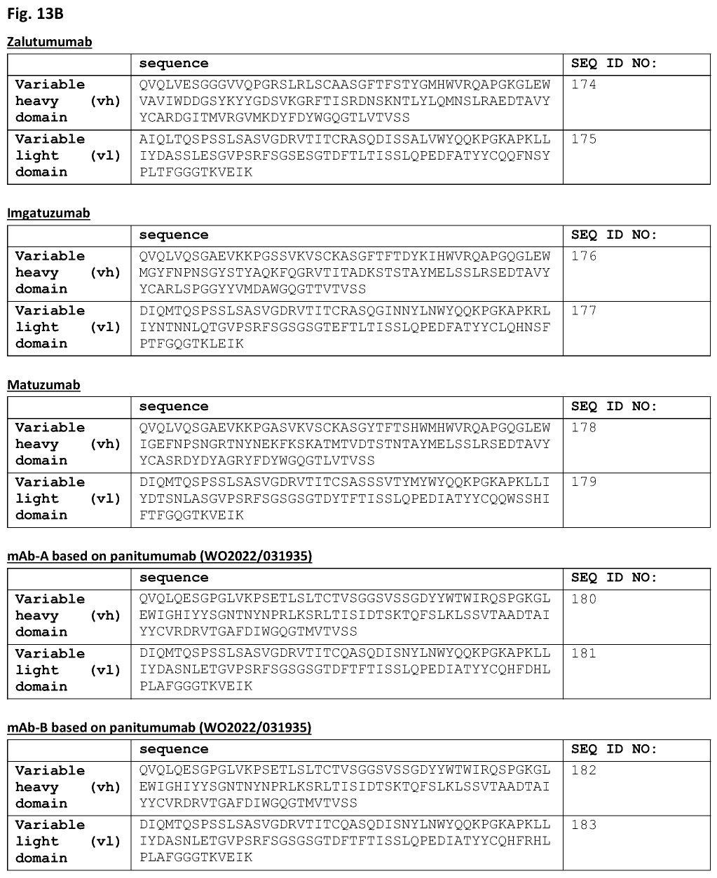

In some embodiments, the NKEs include an ABD that binds to the ECD of human NKp30. Suitable variable heavy VH and variable light VL domains that bind to the ECD of human NKp30 include, but are not limited to, VH/VL pairs selected from the group including: (i) SEQ ID NOs: 158 and 159, (ii) SEQ ID NOs: 160 and 161, and (iii) SEQ ID NOs: 162 and 163, as shown in FIGS. 12, as well as those disclosed herein. Additionally, any sequence included in FIG. 12 can also be individually included or excluded. It should be noted that these ABDs can be included in any of the formats described herein. In some embodiments, the NKEs of the invention include at least one anti-NKp30 antigen binding domain, as described herein and in the Figures. Described herein are a plurality of means for binding the ECD of human NKp30.

In some embodiments, provided herein is an anti-human NKp30 antigen binding domain with a variable heavy domain and a variable light domain set selected from the group including: (a) a VH with the amino acid sequence depicted in SEQ ID NO:7302 and a VL with the amino acid sequence depicted in SEQ ID NO:7305; (b) a VH with the amino acid sequence depicted in SEQ ID NO:7302 and a VL with the amino acid sequence depicted in SEQ ID NO:7309; (c) a VH with the amino acid sequence depicted in SEQ ID NO:6121 and a VL with the amino acid sequence depicted in SEQ ID NO:7294; and (d) a VH with an amino acid sequence selected from the group including SEQ ID NO:7298 or 7300-7304 and a VL with an amino acid sequence selected from the group including SEQ ID NO:7299 or 7305-7309, wherein the sequences are as shown in US2021/0371523 hereby expressly incorporated by reference for the sequences. Additionally, any sequence included above from US2021/0371523 can also be individually included or excluded.

In some embodiments, provided herein is an anti-human NKp30 antigen binding domain with a variable heavy domain and a variable light domain set selected from the group including: (a) a VH with the amino acid sequence depicted in SEQ ID NO:30 and a VL with the amino acid sequence depicted in SEQ ID NO:32; (b) a VH with the amino acid sequence depicted in SEQ ID NO21: and a VL with the amino acid sequence depicted in SEQ ID NO:23; and (c) a VH with the amino acid sequence depicted in SEQ ID NO:11 and a VL with the amino acid sequence depicted in SEQ ID NO:13; wherein the sequences are as shown in WO2021/143858 hereby expressly incorporated by reference for the sequences. Additionally, any sequence included above from WO2021/143858 can also be individually included or excluded.

An anti-human NKp30 antigen binding domain with a variable heavy domain and a variable light domain set selected from the group including: (a) a VH with the amino acid sequence depicted in SEQ ID NO:14 and a VL with the amino acid sequence depicted in SEQ ID NO:18; (b) a VH with the amino acid sequence depicted in SEQ ID NO:25 and a VL with the amino acid sequence depicted in SEQ ID NO:18; (c) a VH with the amino acid sequence depicted in SEQ ID NO:72 and a VL with the amino acid sequence depicted in SEQ ID NO:18; (d) a VH with the amino acid sequence depicted in SEQ ID NO:78 and a VL with the amino acid sequence depicted in SEQ ID NO:80; and E a VH with the amino acid sequence depicted in SEQ ID NO:79 and a VL with the amino acid sequence depicted in SEQ ID NO:81; wherein the sequences are as shown in WO2019/226617 hereby expressly incorporated by reference for the sequences. For instance, the antibodies mAb8, mAb9, mAb10 and mAb11 of WO2019/226617 are useful NKp30 ABDs. Additionally, any sequence included above from WO2019/226617 can also be individually included or excluded.

B. Tumor Target Antigen Binding Domains

The present invention provides NKEs that bind to human NK cells and additionally bind to the extracellular domains of human tumor target antigens (TTAs), including, but not limited to, EGFR, Trop2 (or “Trop-2”), CD20, B7H3, FLT3, DLL3, CD19, CD123, CEA, MSLN, BCMA, CAIX, CLDN18.2, HER2, PD-1, CD22, CD38, and ANOL. Binding affinities of a specific TTA ABD can be evaluated using an assay recognized by those skilled in the art including, but not limited to, a Surface Plasmon Resonance (SPR) and/or a BLI binding assay (such as Biacore, Octet, or Caterra LSA).

1. B7H3 Antigen Binding Domains

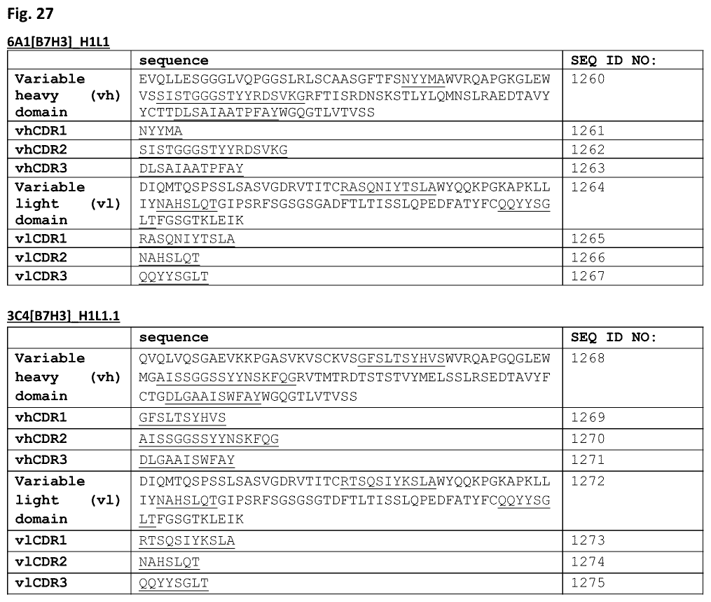

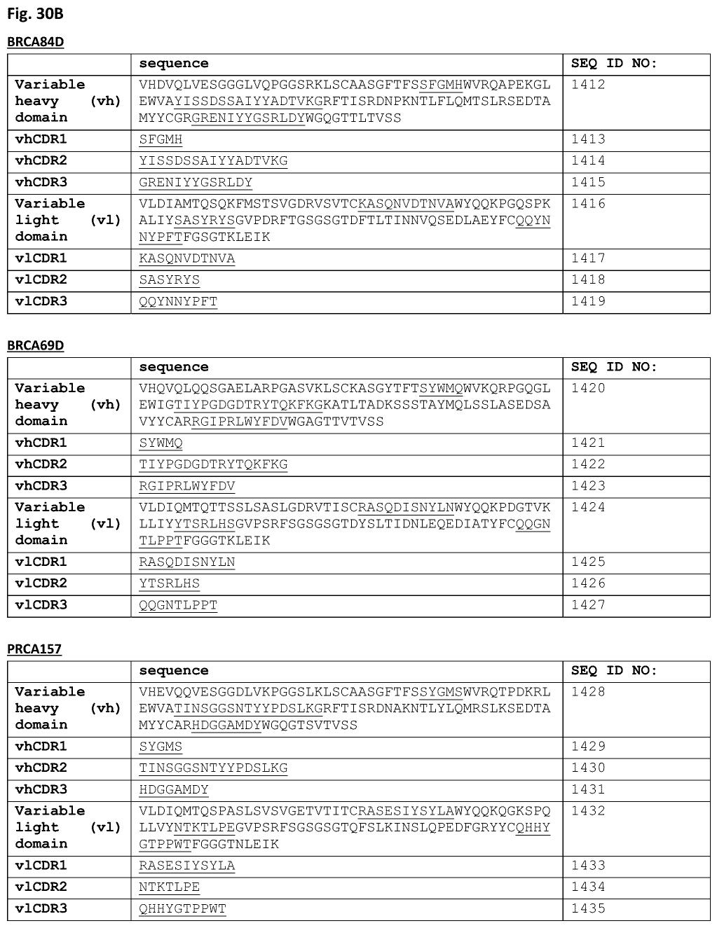

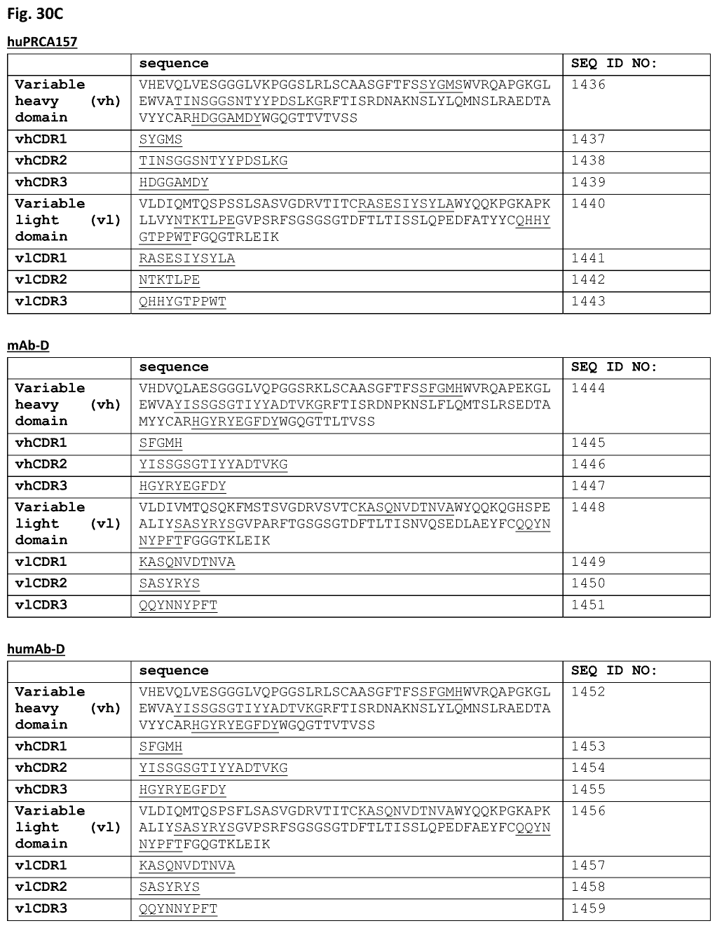

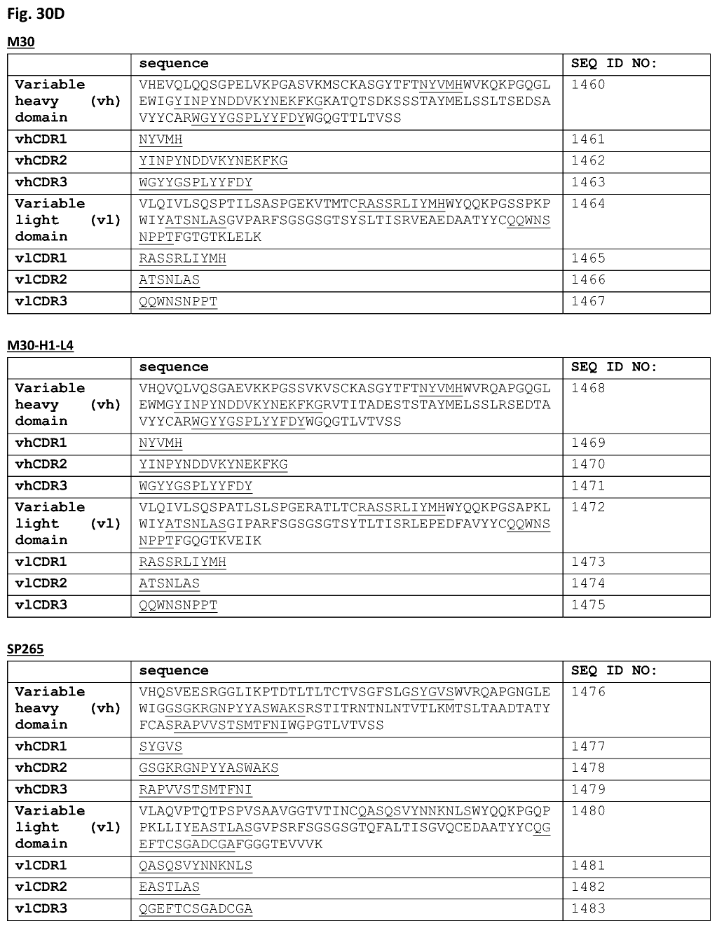

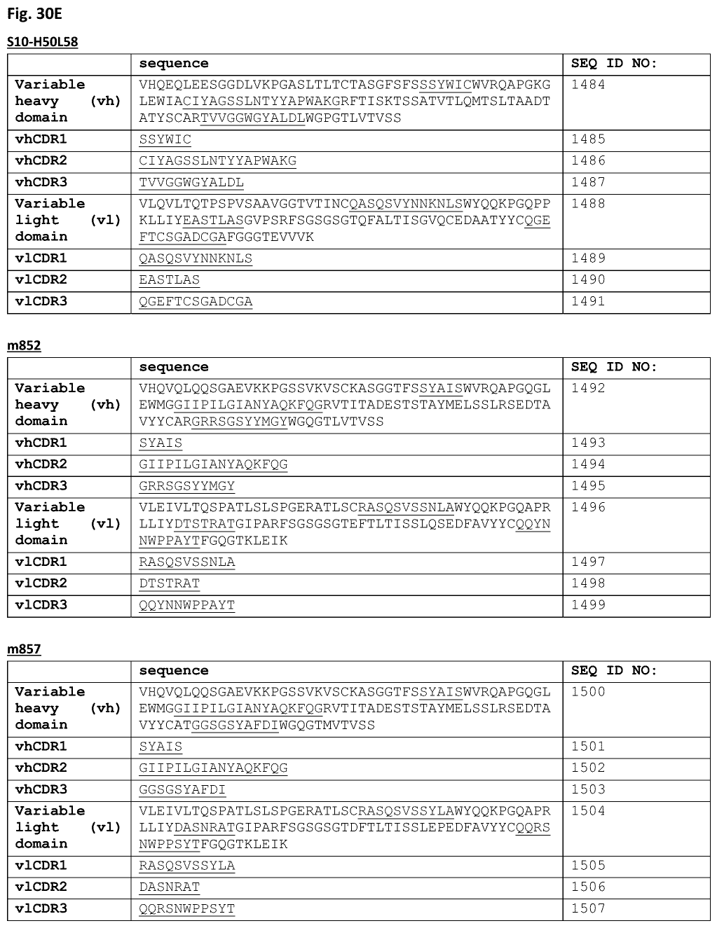

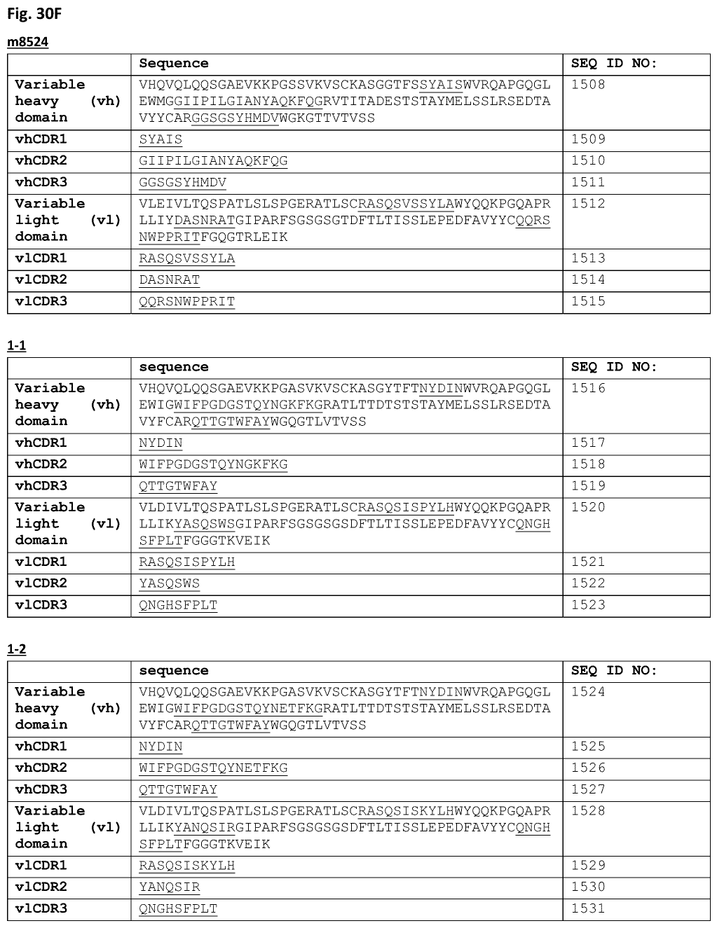

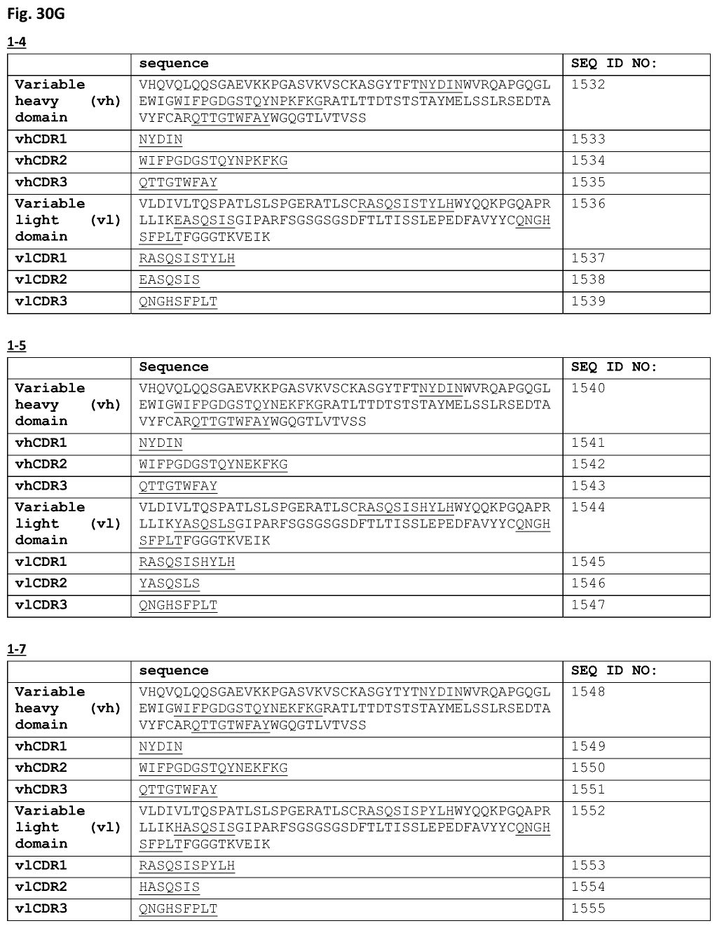

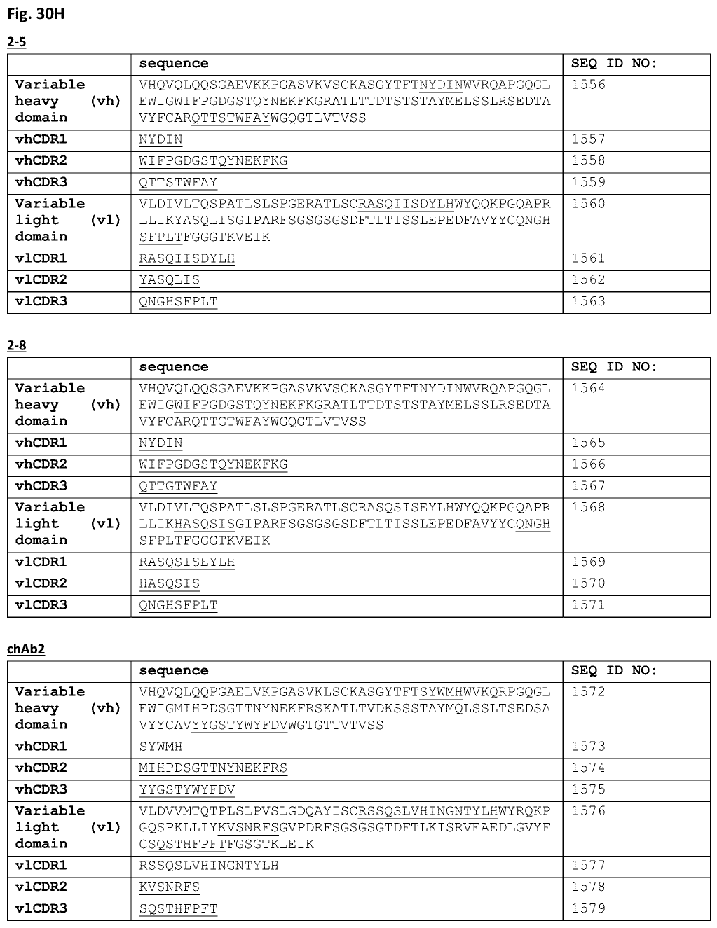

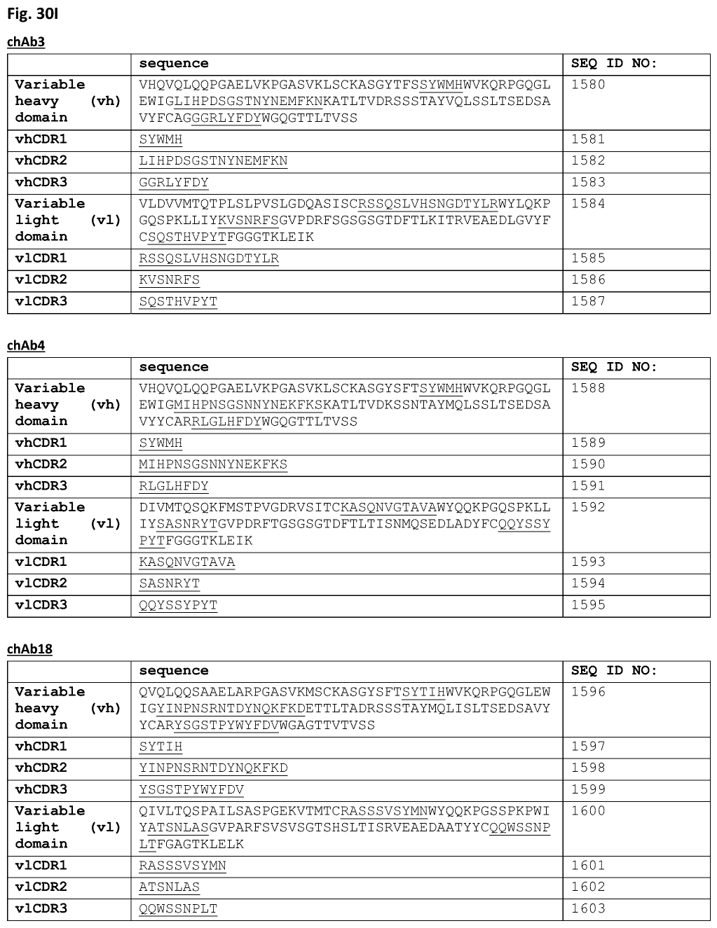

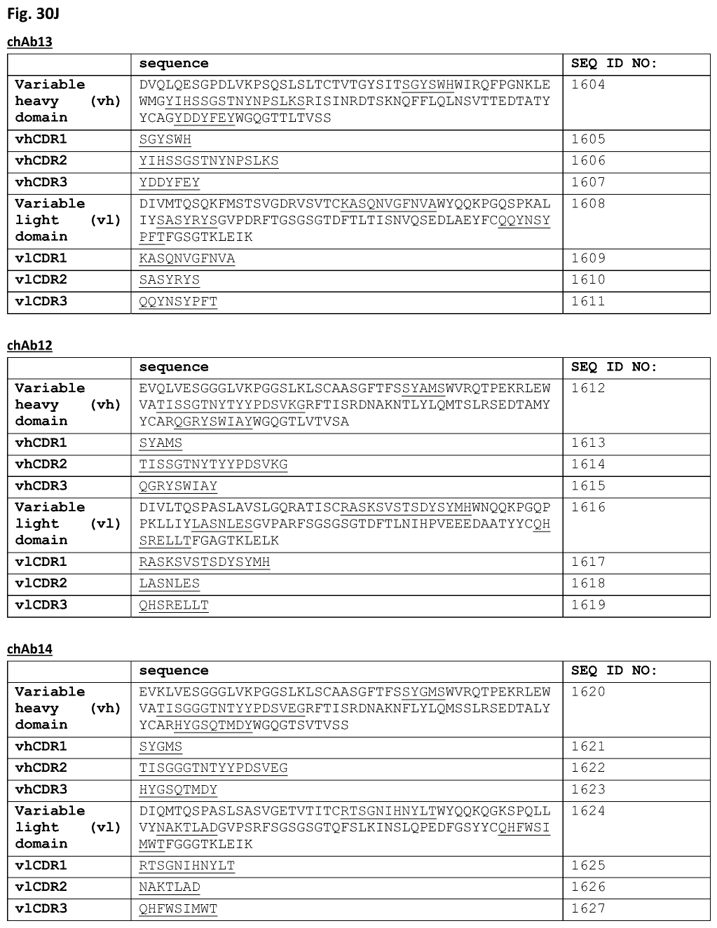

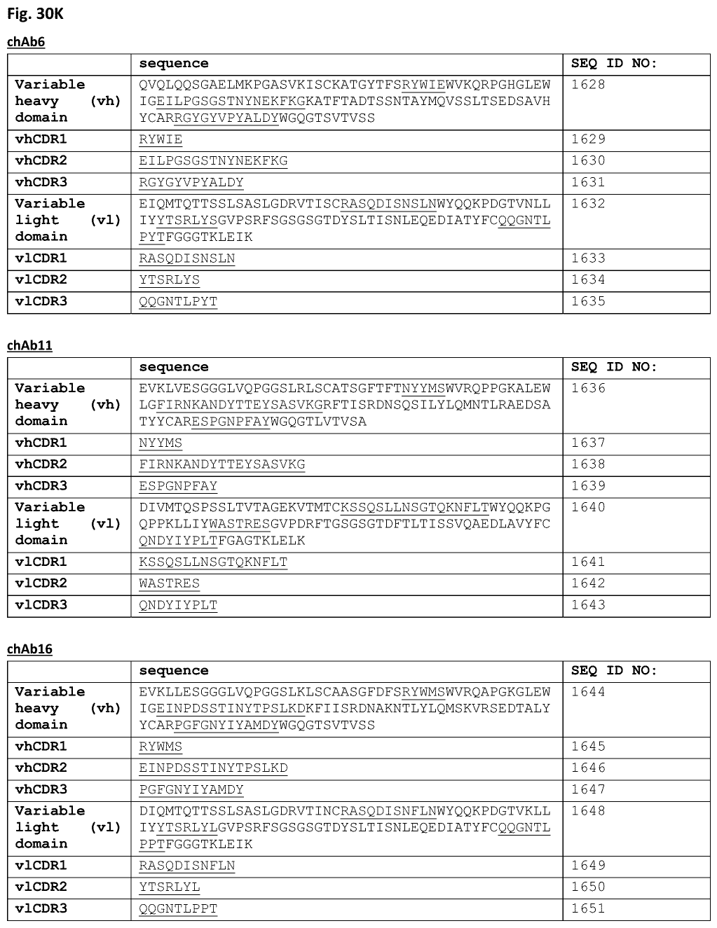

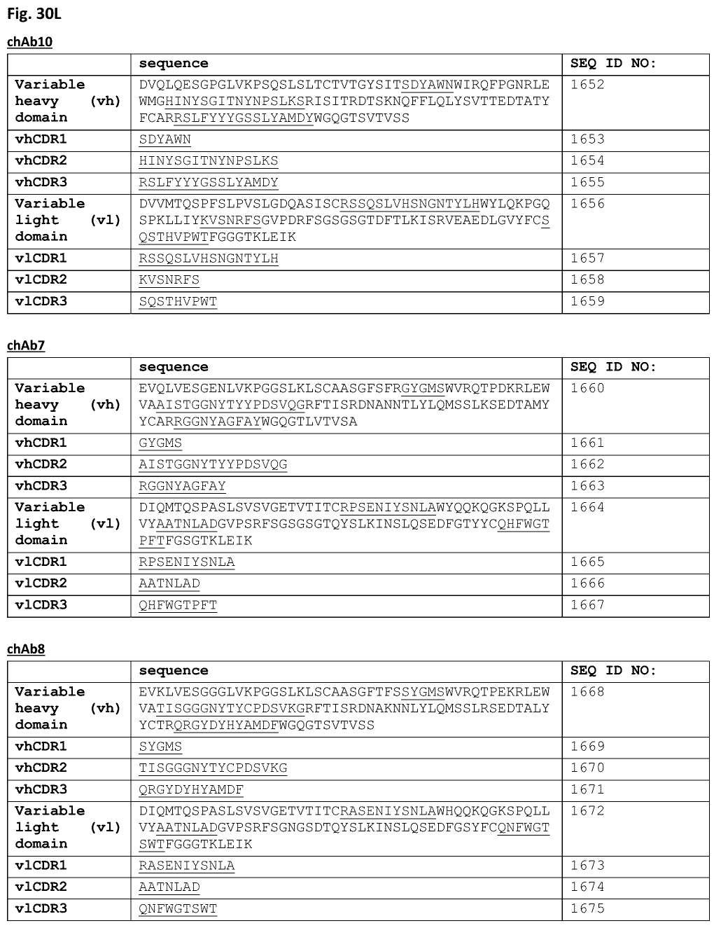

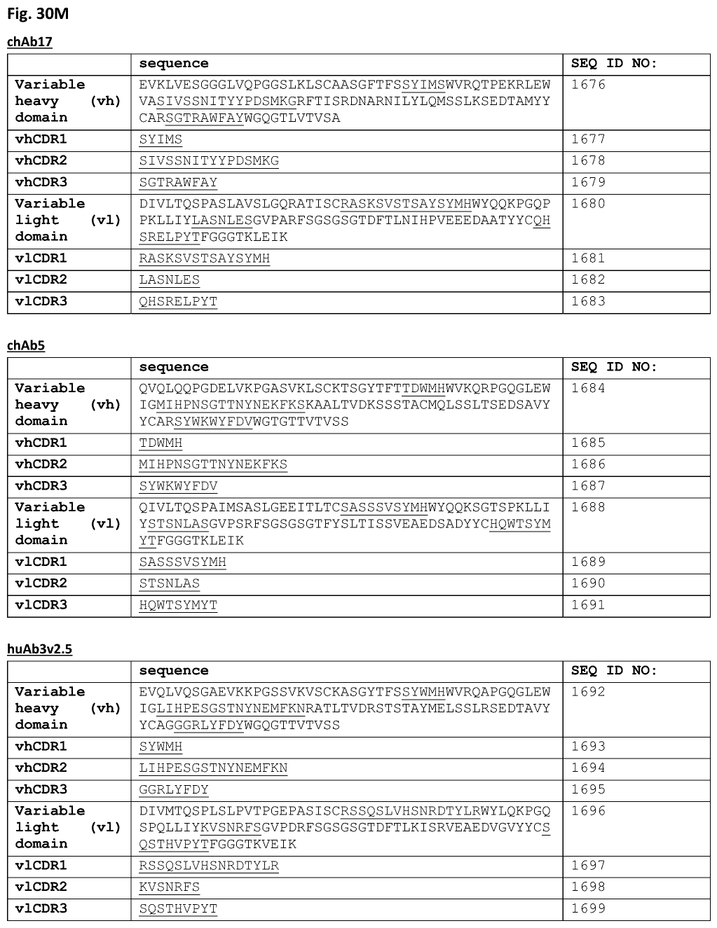

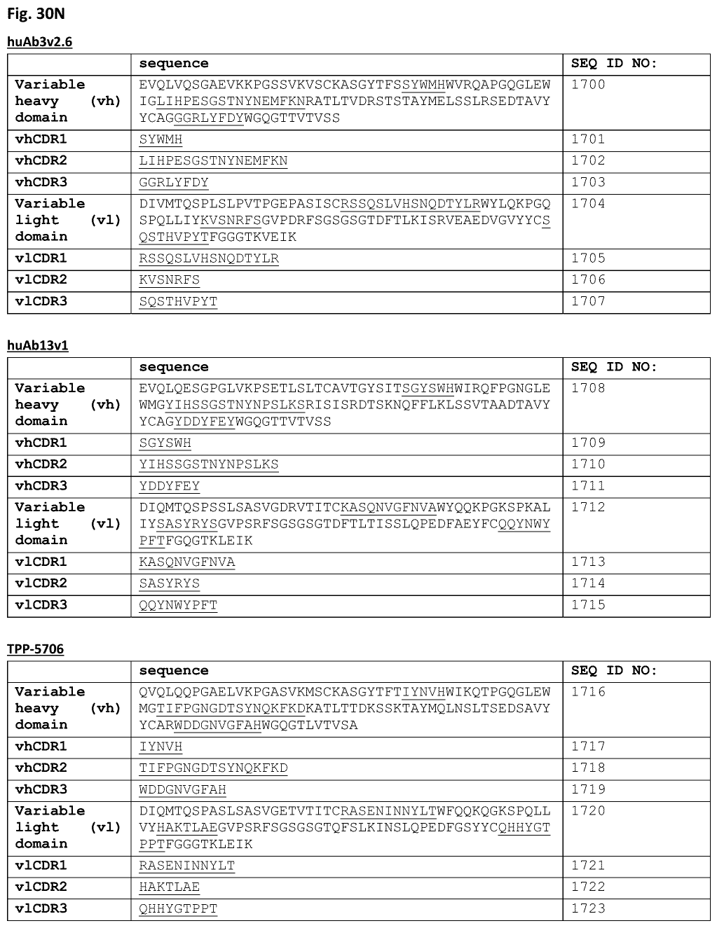

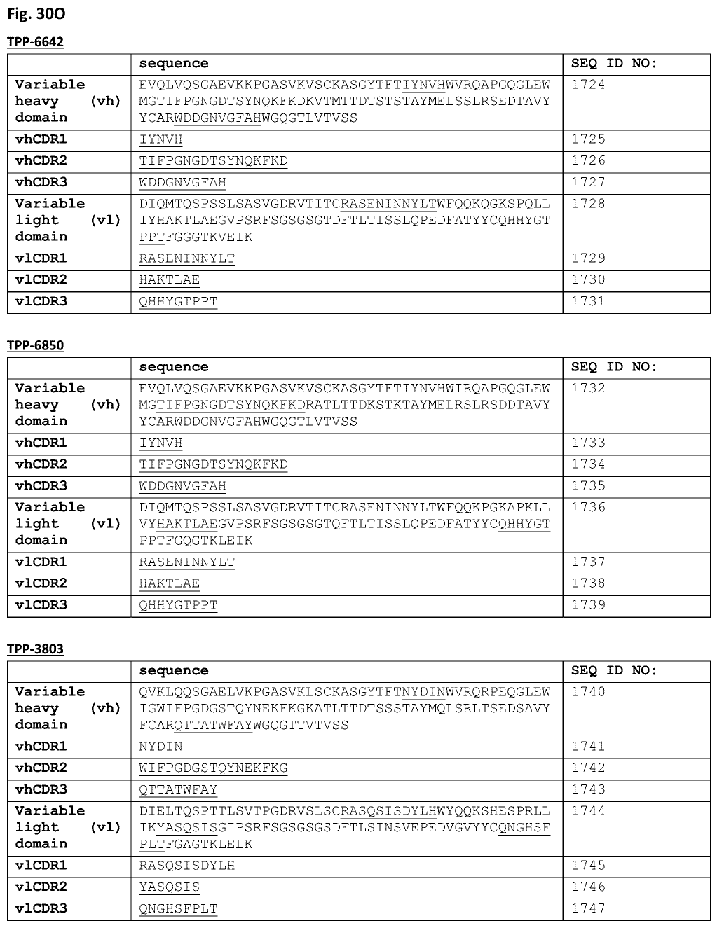

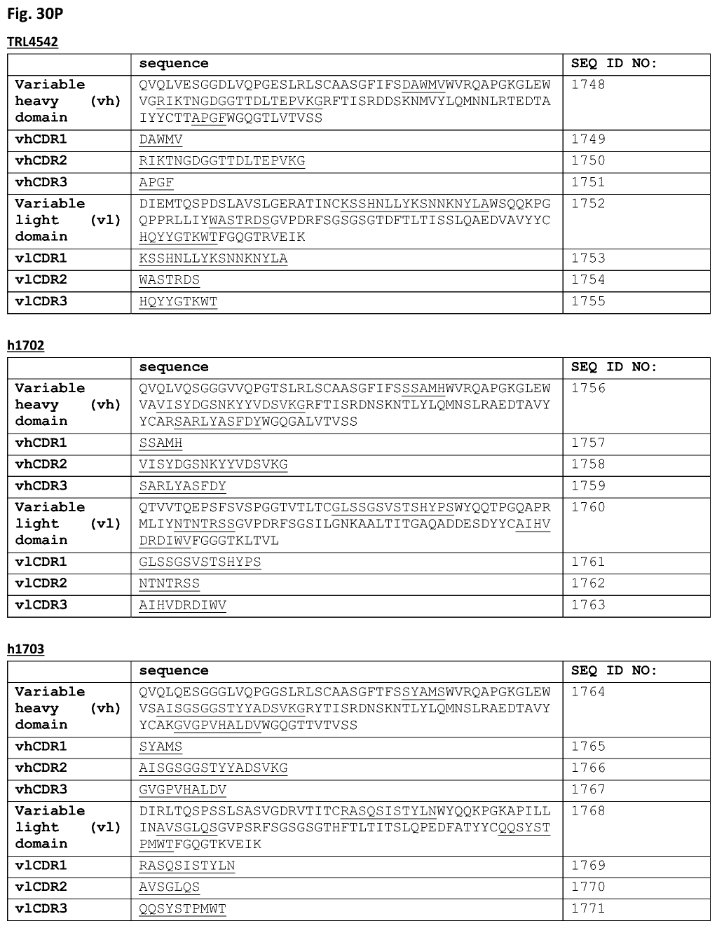

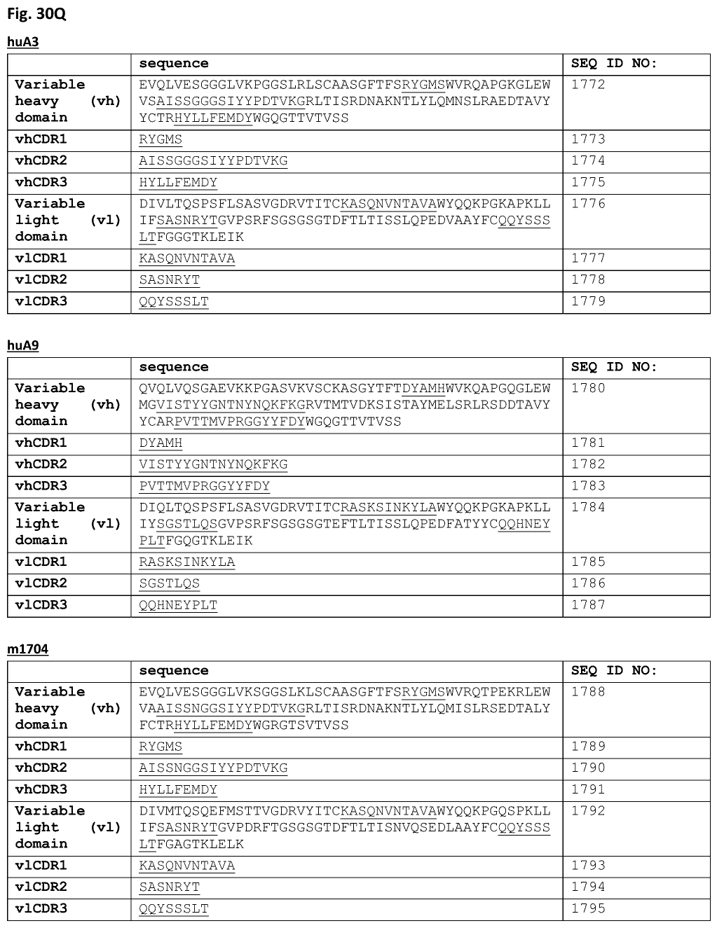

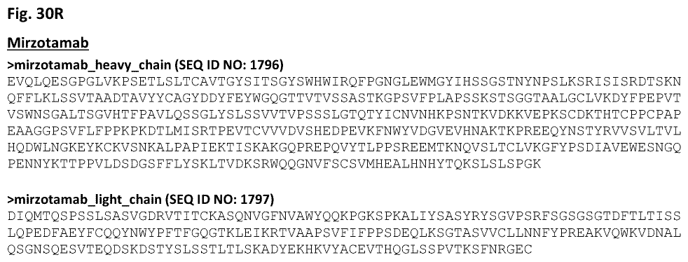

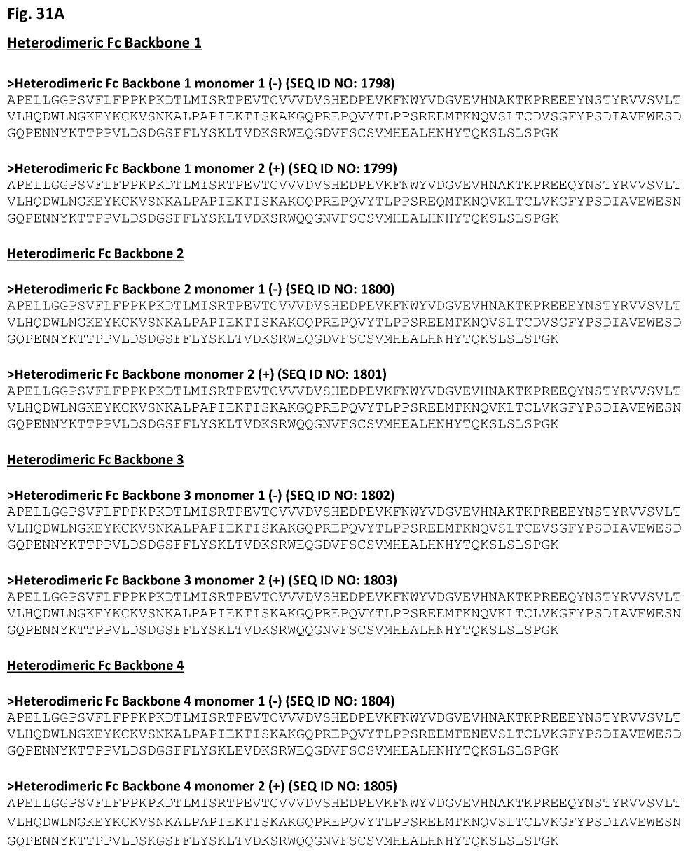

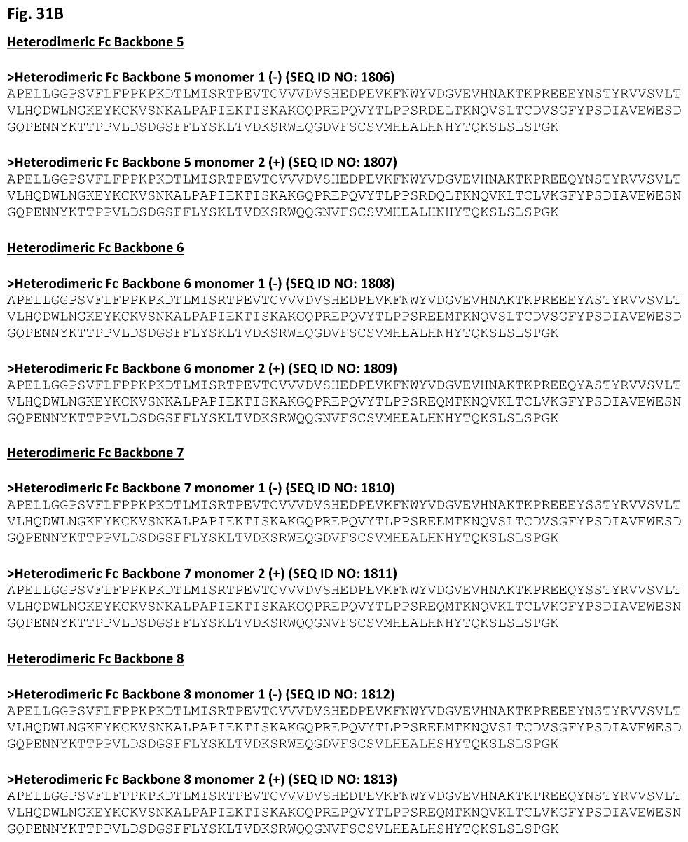

In some embodiments, the NKEs include an ABD that binds to the ECD of human B7H3. Suitable variable heavy VH and variable light VL domains that bind to the ECD of human B7H3 include, but are not limited to, VH/VL pairs selected from the group including: (i) SEQ ID NOs: 1260 and 1264, (ii) SEQ ID NOs: 1268 and 1272, (iii) SEQ ID NOs: 1276 and 1280, (iv) SEQ ID NOs: 1284 and 1288, (v) SEQ ID NOs: 1292 and 1296, (vi) SEQ ID NOs: 1300 and 1296, (vii) SEQ ID NOs: 1301 and 1296, (viii) SEQ ID NOs: 1302 and 1296, (ix) SEQ ID NOs: 1303 and 1296, (x) SEQ ID NOs: 1304 and 1296, (xi) SEQ ID NOs: 1305 and 1296, (xii) SEQ ID NOs: 1306 and 1296, (xiii) SEQ ID NOs: 1307 and 1296, (xiv) SEQ ID NOs: 1308 and 1296, (xv) SEQ ID NOs: 1309 and 1296, (xvi) SEQ ID NOs: 1310 and 1296, (xvii) SEQ ID NOs: 1311 and 1296, (xviii) SEQ ID NOs: 1312 and 1296, (xix) SEQ ID NOs: 1313 and 1296, (xx) SEQ ID NOs: 1314 and 1296, (xxi) SEQ ID NOs: 1315 and 1296, (xxii) SEQ ID NOs: 1316 and 1296, (xxiii) SEQ ID NOs: 1317 and 1296, (xxiv) SEQ ID NOs: 1318 and 1296, (xxv) SEQ ID NOs: 1319 and 1296, (xxvi) SEQ ID NOs: 1320 and 1296, (xxvii) SEQ ID NOs: 1321 and 1296, (xxviii) SEQ ID NOs: 1322 and 1296, (xxix) SEQ ID NOs: 1323 and 1296, (xxx) SEQ ID NOs: 1324 and 1296, (xxxi) SEQ ID NOs: 1325 and 1296, (xxxii) SEQ ID NOs: 1326 and 1296, (xxxiii) SEQ ID NOs: 1327 and 1296, (xxxiv) SEQ ID NOs: 1328 and 1296, (xxxv) SEQ ID NOs: 1329 and 1296, (xxxvi) SEQ ID NOs: 1330 and 1296, (xxxvii) SEQ ID NOs: 1331 and 1296, (xxxviii) SEQ ID NOs: 1332 and 1296, (xxxix) SEQ ID NOs: 1333 and 1296, (xl) SEQ ID NOs: 1334 and 1296, (xli) SEQ ID NOs: 1335 and 1296, (xlii) SEQ ID NOs: 1336 and 1296, (xliii) SEQ ID NOs: 1337 and 1296, (xliv) SEQ ID NOs: 1338 and 1296, (xlv) SEQ ID NOs: 1339 and 1296, (xlvi) SEQ ID NOs: 1340 and 1296, (xlvii) SEQ ID NOs: 1341 and 1296, (xlviii) SEQ ID NOs: 1342 and 1296, (xlix) SEQ ID NOs: 1343 and 1296, (1) SEQ ID NOs: 1344 and 1296, (li) SEQ ID NOs: 1345 and 1296, (lii) SEQ ID NOs: 1346 and 1296, (liii) SEQ ID NOs: 1347 and 1296, (liv) SEQ ID NOs: 1348 and 1296, (lv) SEQ ID NOs: 1349 and 1296, (lvi) SEQ ID NOs: 1350 and 1296, (lvii) SEQ ID NOs: 1351 and 1296, (lviii) SEQ ID NOs: 1352 and 1296, (lix) SEQ ID NOs: 1353 and 1296, (lx) SEQ ID NOs: 1354 and 1296, (lxi) SEQ ID NOs: 1355 and 1296, (lxii) SEQ ID NOs: 1356 and 1296, (lxiii) SEQ ID NOs: 1357 and 1296, (lxiv) SEQ ID NOs: 1358 and 1296, (lxv) SEQ ID NOs: 1359 and 1296, (lxvi) SEQ ID NOs: 1360 and 1296, (lxvii) SEQ ID NOs: 1361 and 1296, (lxviii) SEQ ID NOs: 1362 and 1296, (lxix) SEQ ID NOs: 1363 and 1296, (lxx) SEQ ID NOs: 1364 and 1296, (lxxi) SEQ ID NOs: 1365 and 1296, (lxxii) SEQ ID NOs: 1366 and 1296, (lxxiii) SEQ ID NOs: 1367 and 1296, (lxxiv) SEQ ID NOs: 1368 and 1296, (lxxv) SEQ ID NOs: 1369 and 1296, (lxxvi) SEQ ID NOs: 1370 and 1296, (lxxvii) SEQ ID NOs: 1371 and 1296, (lxxviii) SEQ ID NOs: 1372 and 1296, (lxxix) SEQ ID NOs: 1373 and 1296, (lxxx) SEQ ID NOs: 1374 and 1296, (lxxxi) SEQ ID NOs: 1375 and 1296, (lxxxii) SEQ ID NOs: 1376 and 1296, (lxxxiii) SEQ ID NOs: 1377 and 1296, (lxxxix) SEQ ID NOs: 1378 and 1296, (lxxxv) SEQ ID NOs: 1379 and 1296, (lxxxvi) SEQ ID NOs: 1380 and 1296, (lxxxvii) SEQ ID NOs: 1381 and 1296, (lxxxviii) SEQ ID NOs: 1382 and 1296, (lxxxix) SEQ ID NOs: 1383 and 1296, (xc) SEQ ID NOs: 1384 and 1296, (xci) SEQ ID NOs: 1385 and 1296, (xcii) SEQ ID NOs: 1386 and 1296, and (xciii) SEQ ID NOs: 1387 and 1296, as shown in FIGS. 27, 28, 29A-29H, and 30A-30R, as well as those disclosed herein. Additionally, any sequence included in FIGS. 27, 28, 29 and 30 can also be individually included or excluded. It should be noted that these ABDs can be included in any of the heterodimeric formats described herein.

Additionally, included herein are B7H3 ABDs that have the variable heavy chain depicted in SEQ ID NO: 11 or SEQ ID NO: 13, and variable light chain depicted in SEQ ID NO: 12 or SEQ ID NO: 14 from U.S. Pat. No. 10,501,544, incorporated by reference in its entirety, with particularity for relevant disclosure pertaining to B7H3 ABDs and the accompanying sequences described therein. Additionally, any sequence included above from U.S. Pat. No. 10,501,544 can also be individually included or excluded. Further, included herein are B7H3 antigen binding domain with: a) the VH CDRs depicted in SEQ ID NO: 1, SEQ ID NO: 25, and SEQ ID NO: 33 in combination with the VL CDRs depicted in SEQ ID NO: 34, SEQ ID NO: 36, and SEQ ID NO: 6; or b) the VH CDRs depicted in SEQ ID NO: 1, SEQ ID NO: 25, and SEQ ID NO: 10 in combination with the VL CDRs depicted in SEQ ID NO: 38, SEQ ID NO: 30, and SEQ ID NO: 6; both from U.S. Pat. No. 9,963,509, incorporated by reference in its entirety, with particularity for relevant disclosure pertaining to B7H3 ABDs and the accompanying sequences described therein. Additionally, any sequence included above from U.S. Pat. No. 9,963,509 can also be individually included or excluded. Further, included herein are anti-B7H3 antigen binding domains with the VH CDRs depicted in SEQ ID NO: 1, SEQ ID NO: 9, and SEQ ID NO: 10 in combination with the VL CDRs depicted in SEQ ID NO: 11, SEQ ID NO: 312, and SEQ ID NO: 6; from U.S. Pat. No. 10,865,245, incorporated by reference in its entirety, with particularity for relevant disclosure pertaining to B7H3 ABDs and the accompanying sequences described therein. Additionally, any sequence included above from U.S. Pat. No. 10,865,245 can also be individually included or excluded. Still further, included herein are anti-B7H3 antigen binding domain with: a) vhCDR1 with the sequence depicted in SEQ ID NO: 110; b) vhCDR2 with the sequence depicted in SEQ ID NO: 111; c) vhCDR3 with the sequence depicted in SEQ ID NO: 113; d) vlCDR1 with the sequence depicted in SEQ ID NO: 114; e) vlCDR2 with the sequence depicted in SEQ ID NO: 115; and f) vlCDR3 with the sequence depicted in SEQ ID NO: 116, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Yet further, included herein is an anti-B7H3 antigen binding domain with: a) vhCDR1 with the sequence depicted in SEQ ID NO: 118; b) vhCDR2 with the sequence depicted in SEQ ID NO: 119; c) vhCDR3 with the sequence depicted in SEQ ID NO: 120; d) vlCDR1 with the sequence depicted in SEQ ID NO: 121; e) vlCDR2 with the sequence depicted in SEQ ID NO: 122; and f) vlCDR3 with the sequence depicted in SEQ ID NO: 123, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Still even further, included herein is an anti-B7H3 antigen binding domain with: a) vhCDR1 with the sequence depicted in SEQ ID NO: 371; b) vhCDR2 with the sequence depicted in SEQ ID NO: 372; c) vhCDR3 with the sequence depicted in SEQ ID NO: 373; d) vlCDR1 with the sequence depicted in SEQ ID NO: 374; e) vlCDR2 with the sequence depicted in SEQ ID NO: 375; and f) vlCDR3 with the sequence depicted in SEQ ID NO: 376, from WO2020/033702, incorporated by reference in its entirety, with particularity for relevant disclosure pertaining to B7H3 ABDs and the accompanying sequences described therein. Additionally, any sequence included above from WO2020/033702 can also be individually included or excluded. Yet still even further, included herein is an anti-B7H3 antigen binding domain comprising: a) a heavy chain variable region comprising a vhCDR1 having SEQ ID NO: 6, a vhCDR2 having SEQ ID NO: 7, and a vhCDR3 having SEQ ID NO: 8; and b) a light chain variable region comprising a vlCDR1 having SEQ ID NO: 9, a vlCDR2 with the sequence WAS (tryptophan-alanine-serine) and a vlCDR3 having SEQ ID NO: 10, the sequences are depicted in WO2022/105879, hereby incorporated by reference for these sequences. Further, included herein is an anti-B7H3 antigen binding domain comprising a variable heavy chain and a variable light chain pair selected from the group including: a) a VH having SEQ ID NO:1 and a VL having SEQ ID NO:5; b) a VH having SEQ ID NO:11 and a VL having SEQ ID NO:15; c) a VH having SEQ ID NO:21 and a VL having SEQ ID NO:25; d) a VH having SEQ ID NO:31 and a VL having SEQ ID NO:35; e) a VH having SEQ ID NO:41 and a VL having SEQ ID NO:45; f) a VH having SEQ ID NO:55 and a VL having SEQ ID NO:57; g) a VH having SEQ ID NO:63 and a VL having SEQ ID NO:35; and h) a VH having SEQ ID NO:63 and a VL having SEQ ID NO:68, wherein the sequences are as depicted in U.S. Pat. No. 11,071,788, hereby incorporated by reference for these sequences. Additionally, any sequence included above from U.S. Pat. No. 11,071,788 can also be individually included or excluded.

In some embodiments, the NKEs of the invention include at least one anti-B7H3 antigen binding domain, as described herein and in the Figures. Described herein is a plurality of means for binding the ECD of human B7H3.

2. EGFR Antigen Binding Domains

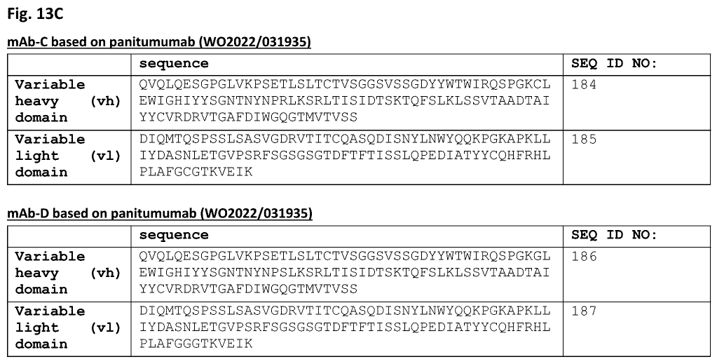

In some embodiments, the NKEs include an ABD that binds to the ECD of human EGFR. Suitable variable heavy VH and variable light VL domains that bind to the ECD of human EGFR include, but are not limited to, VH/VL pairs selected from the group including: (i) SEQ ID NOs: 164 and 165, (ii) SEQ ID NOs: 166 and 167, (iii) SEQ ID NOs: 168 and 169, (iv) SEQ ID NOs: 170 and 171, (v) SEQ ID NOs: 172 and 173, (vi) SEQ ID NOs: 174 and 175, (vii) SEQ ID NOs: 176 and 177, (viii) SEQ ID NOs: 178 and 179, (ix) SEQ ID NOs: 180 and 181, (x) SEQ ID NOs: 182 and 183, (xi) SEQ ID NOs: 184 and 185, and (xii) SEQ ID NOs: 186 and 187, as shown in FIGS. 13A-13C, as well as those disclosed herein. Additionally, any sequence included in FIG. 13 can also be individually included or excluded. It should be noted that these ABDs can be included in any of the formats described herein.

Further, included herein is an anti-EGFR antigen binding domain with sequences selected from the group including: a) a VH shown in SEQ ID NO:11 and VL shown in SEQ ID NO:13; b) VH shown in SEQ ID NO:11 and VL shown in SEQ ID NO:16; c) a VH shown in SEQ ID NO:32 and VL shown in SEQ ID NO:33; and d) VH shown in SEQ ID NO:1 and VL shown in SEQ ID NO:16, wherein the sequences are as shown in WO2022/031935, hereby expressly incorporated by reference for the sequences. Additionally, any sequence included above from WO2022/031935 can also be individually included or excluded.

In some embodiments, the NKEs of the invention include at least one anti-EGFR antigen binding domain, as described herein and in the Figures. Described herein is a plurality of means for binding the ECD of human EGFR.

3. HER2 Antigen Binding Domains

In some embodiments, the TTA binds to the ECD of human HER2. Suitable variable heavy VH and variable light VL domains that bind to the ECD of human HER2 include, but are not limited to, VH/VL pairs selected from the group including: (i) SEQ ID NOs: 275 and 276, (ii) SEQ ID NOs: 277 and 278, (iii) SEQ ID NOs: 279 and 280, (iv) SEQ ID NOs: 281 and 282, (v) SEQ ID NOs: 283 and 284, (vi) SEQ ID NOs: 285 and 286, and (vii) SEQ ID NOs: 287 and 288, as shown in FIGS. 20A-20B. Additionally, any sequence included in FIG. 20 can also be individually included or excluded. It should be noted that these ABDs can be included in any of the formats described herein.

In some embodiments, the NKEs of the invention include at least one anti-HER2 antigen binding domain, as described herein and in the Figures. Described herein is a plurality of means for binding the ECD of human HER2.

4. CD19 Antigen 1Binding Domains

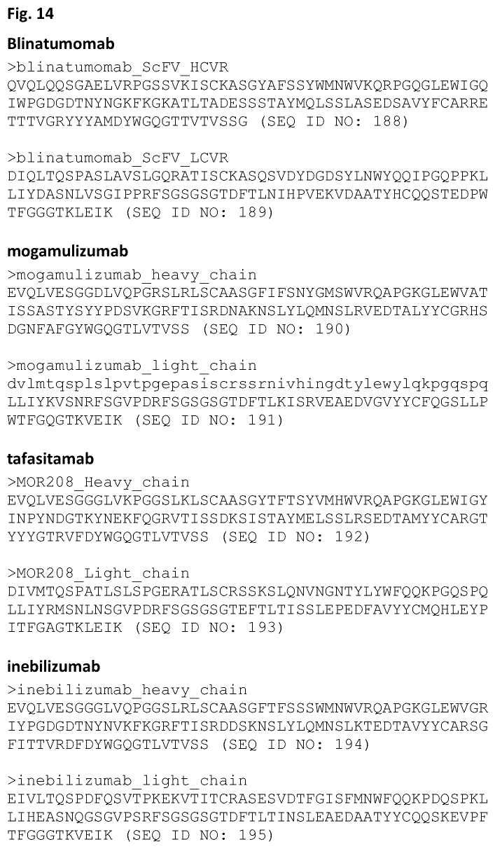

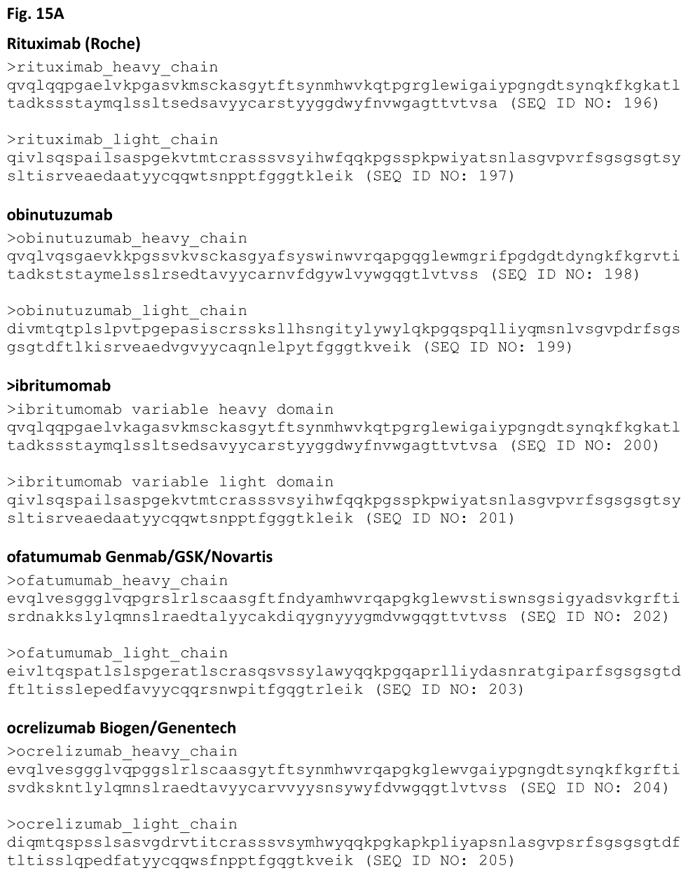

In some embodiments, the TTA binds to the ECD of human CD19. Suitable variable heavy VH and variable light VL domains that bind to the ECD of human CD19 include, but are not limited to, VH/VL pairs selected from the group including: (i) SEQ ID NOs: 188 and 189, (ii) SEQ ID NOs: 190 and 191, (iii) SEQ ID NOs: 192 and 193, and (iv) SEQ ID NOs: 194 and 195, as shown in FIG. 14. Additionally, any sequence included in FIG. 14 can also be individually included or excluded. It should be noted that these ABDs can be included in any of the formats described herein.

In some embodiments, the NKEs of the invention include at least one anti-CD19 antigen binding domain, as described herein and in the Figures. Described herein is a plurality of means for binding the ECD of human CD19.

5. CD20 Antigen Binding Domains

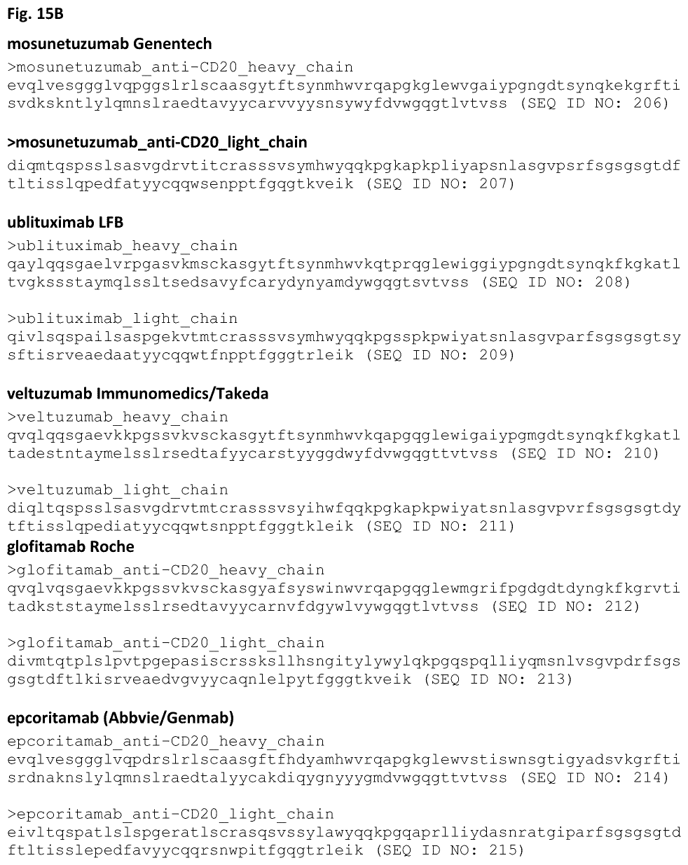

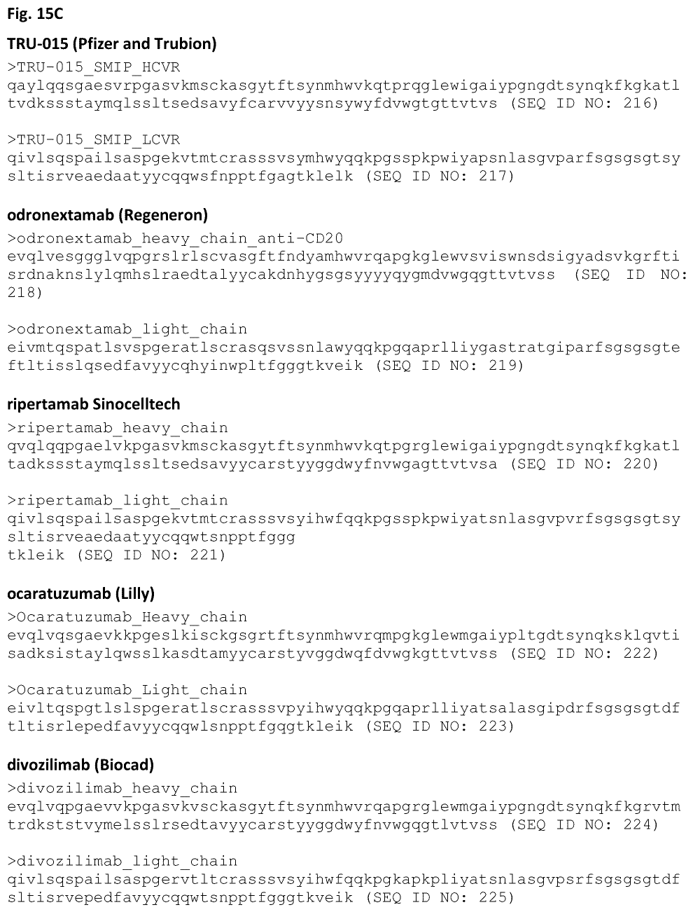

In some embodiments, the NKEs include an ABD that binds to the ECD of human CD20. Suitable variable heavy VH and variable light VL domains that bind to the ECD of human CD20 include, but are not limited to, VH/VL pairs selected from the group including: (i) SEQ ID NOs: 196 and 197, (ii) SEQ ID NOs: 198 and 199, (iii) SEQ ID NOs: 200 and 201, (iv) SEQ ID NOs: 202 and 203, (v) SEQ ID NOs: 204 and 205, (vi) SEQ ID NOs: 206 and 207, (vii) SEQ ID NOs: 208 and 209, (viii) SEQ ID NOs: 210 and 211, (ix) SEQ ID NOs: 212 and 213, (x) SEQ ID NOs: 214 and 215, (xi) SEQ ID NOs: 216 and 217, (xii) SEQ ID NOs: 218 and 219, (xiii) SEQ ID NOs: 220 and 221, (xiv) SEQ ID NOs: 222 and 223, and (xv) SEQ ID NOs: 224 and 225, as shown in FIGS. 15A-15C. Additionally, any sequence included in FIG. 15 can also be individually included or excluded. It should be noted that these ABDs can be included in any of the formats described herein.

In some embodiments, the NKEs of the invention include at least one anti-CD20 antigen binding domain, as described herein and in the Figures. Described herein is a plurality of means for binding the ECD of human CD20.

6. CD123 Antigen Binding Domains

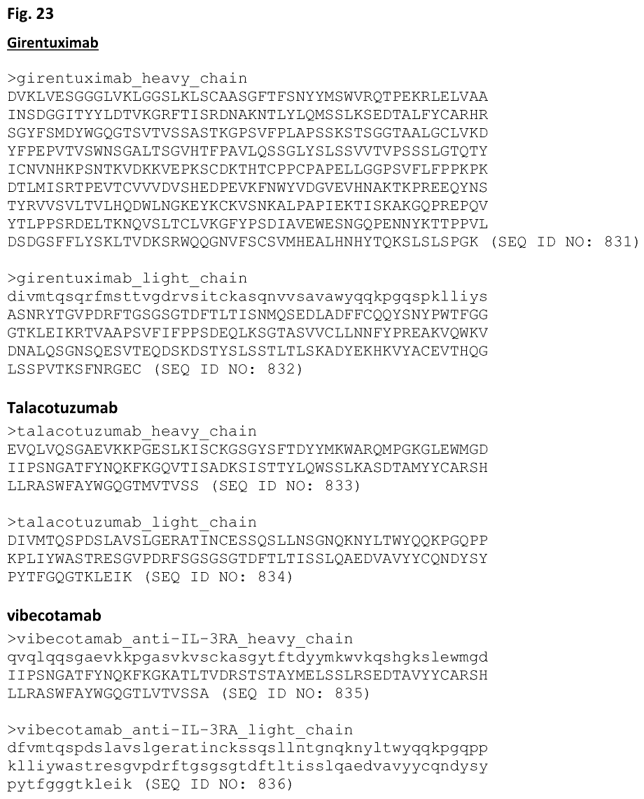

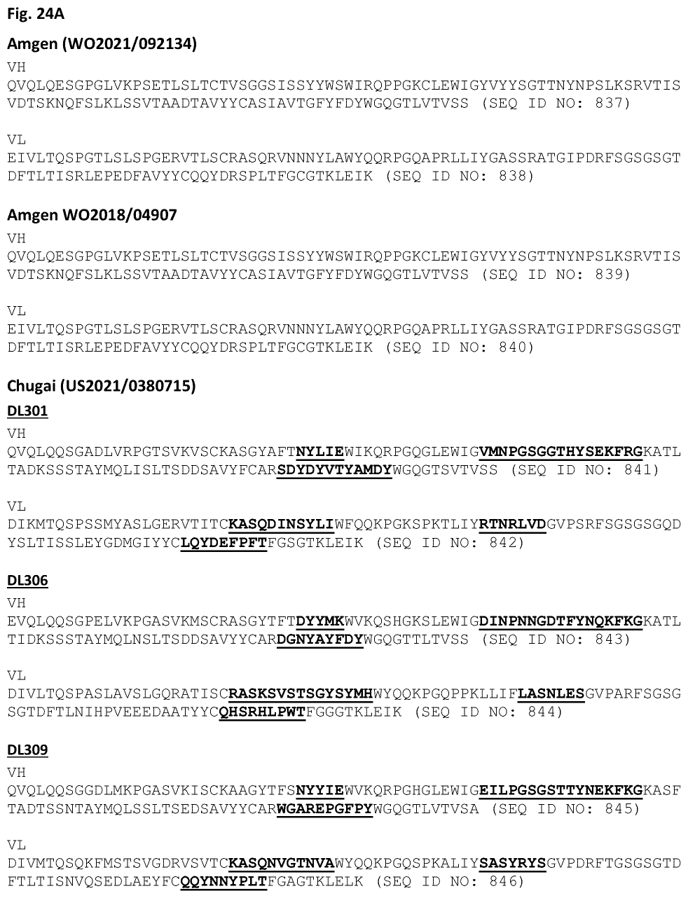

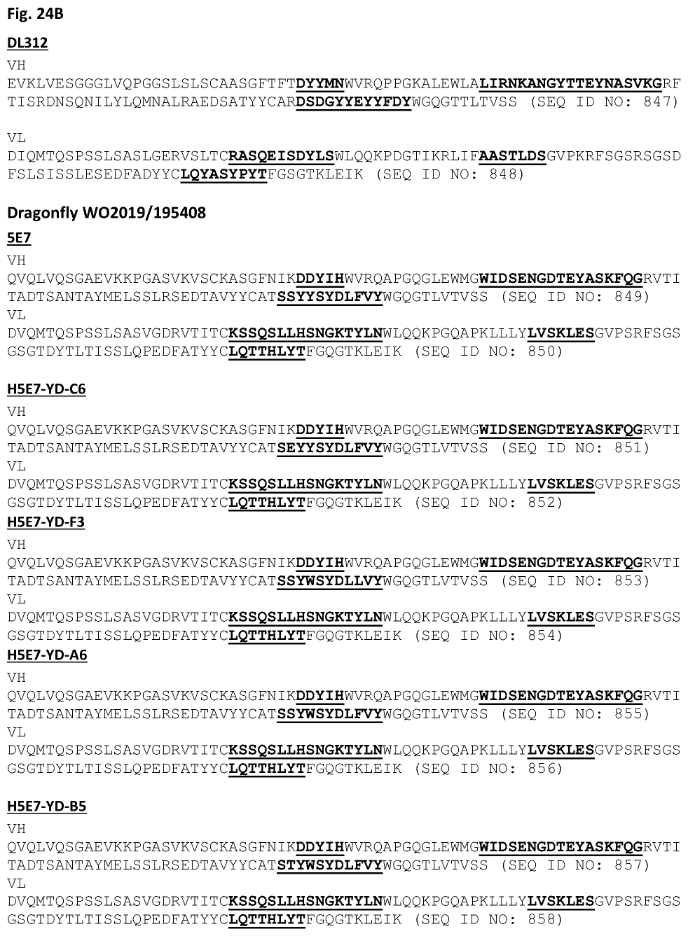

In some embodiments, the TTA binds to the ECD of human CD123. Suitable variable heavy VH and variable light VL domains that bind to the ECD of human CD123 include, but are not limited to, VH/VL pairs selected from the group including: (i) SEQ ID NOs: 833 and 834, and (ii) SEQ ID NOs: 835 and 836, as shown in FIG. 23. Additionally, any sequence included in FIG. 23 can also be individually included or excluded. It should be noted that these ABDs can be included in any of the formats described herein.

In some embodiments, the NKEs of the invention include at least one anti-CD123 antigen binding domain, as described herein and in the Figures. Described herein is a means for binding the ECD of human CD123 antigen.

7. CAIX Antigen Binding Domains

In some embodiments, the tumor target antigen (TTA) for use herein bind to the ECD of human carbonic anhydrase 9 (CAIX or CA9). In this case, the amino acid sequences of the variable heavy and variable light chain can be selected from the group including the amino acid sequences depicted in SEQ ID NO:72 and SEQ ID NO:73 of WO2018/157147; SEQ ID NO:80 and SEQ ID NO:81 as depicted in WO2018/157147, and SEQ ID NO:88 and SEQ ID NO:89 as depicted in WO2018/157147; the heavy and light variable domains of girentuximab, shown in FIG. 23; SEQ ID NO:9 and SEQ ID NO:10 as depicted in US2009/0162382; and SEQ ID NO:17 and SEQ ID NO:19 as depicted in US2009/0162382. Additionally, any sequence described in this paragraph can also be individually included or excluded.

In some embodiments, the NKEs of the invention include at least one anti-CAIX antigen binding domain, as described herein and in the Figures (see, e.g., FIG. 23 and SEQ ID NOs: 831 and 832). Described herein is a plurality of means for binding the ECD of human CAIX.

8. FLT3 Antigen Binding Domains

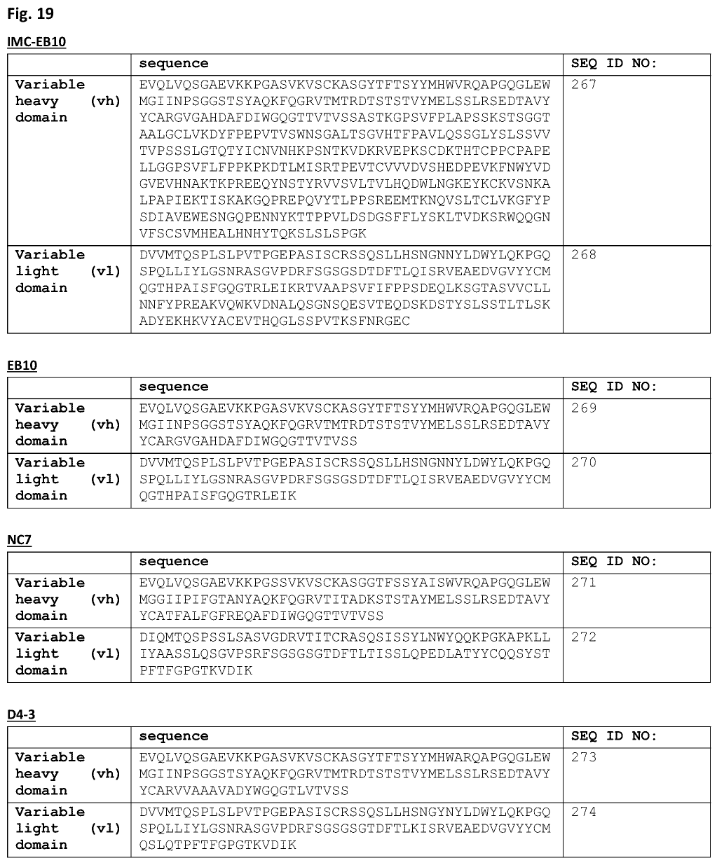

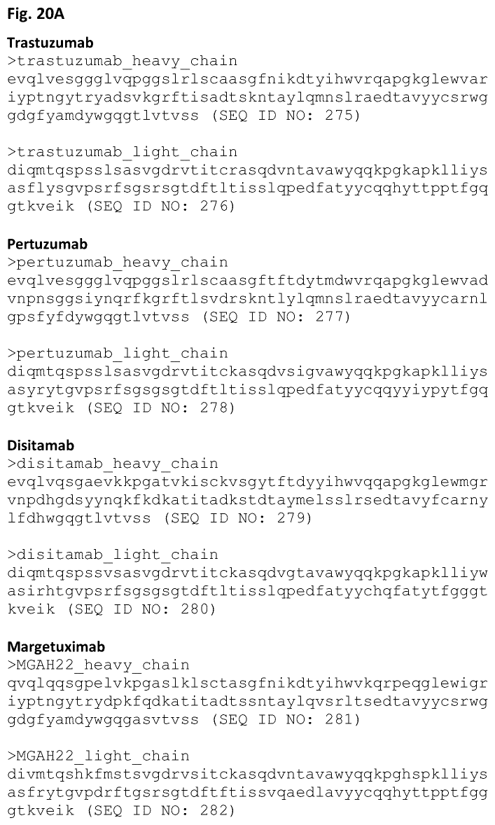

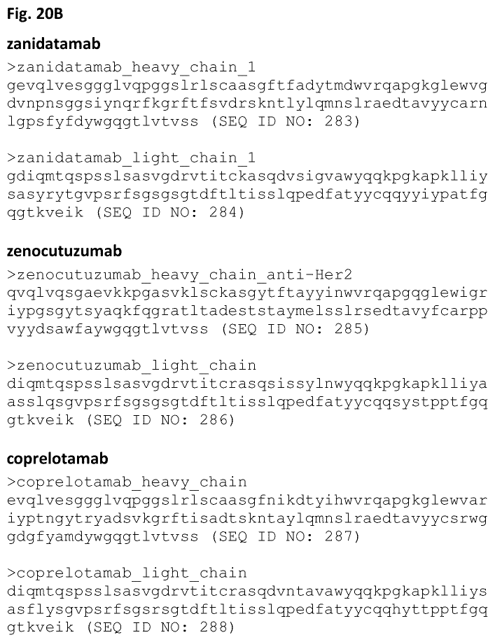

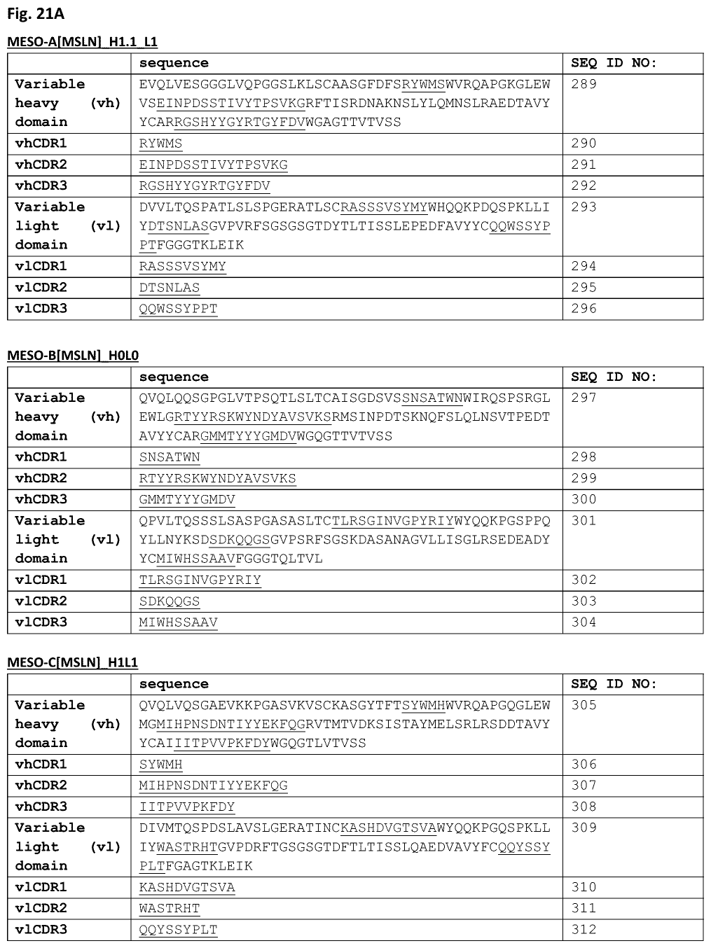

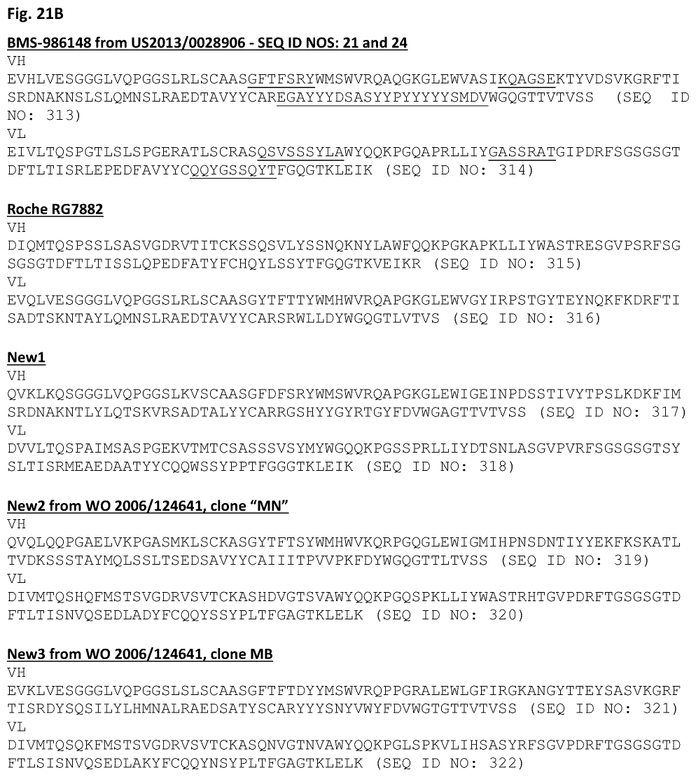

In some embodiments, the NKEs include an ABD that binds to the ECD of human FLT3. Suitable variable heavy VH and variable light VL domains that bind to the ECD of human FLT3 include, but are not limited to, VH/VL pairs selected from the group including: (i) SEQ ID NOs: 267 and 268, (ii) SEQ ID NOs: 269 and 270, (iii) SEQ ID NOs: 271 and 272, (iv) SEQ ID NOs: 273 and 274, (v) SEQ ID NOs: 275 and 276, (vi) SEQ ID NOs: 277 and 278, (vii) SEQ ID NOs: 279 and 280, and (viii) SEQ ID NOs: 281 and 282, as shown in FIG. 19, as well as those disclosed herein. Additionally, any sequence included in FIG. 19 can also be individually included or excluded. It should be noted that these ABDs can be included in any of the formats described herein.

Further, included herein is an anti-FLT3 antigen binding domain with a VH/VL pair selected from the group including: a) a variable heavy domain with SEQ ID NO: 187 and a variable light domain with SEQ ID NO: 188; b) a variable heavy domain with SEQ ID NO: 207 and a variable light domain with SEQ ID NO: 208; c) a variable heavy domain with SEQ ID NO: 217 and a variable light domain with SEQ ID NO: 218; d) a variable heavy domain with SEQ ID NO: 227 and a variable light domain with SEQ ID NO: 228; e) a variable heavy domain with SEQ ID NO: 257 and a variable light domain with SEQ ID NO: 258; f) a variable heavy domain with SEQ ID NO: 267 and a variable light domain with SEQ ID NO: 268; g) a variable heavy domain with SEQ ID NO: 277 and a variable light domain with SEQ ID NO: 278; h) a variable heavy domain with SEQ ID NO: 287 and a variable light domain with SEQ ID NO: 288; i) a variable heavy domain with SEQ ID NO: 297 and a variable light domain with SEQ ID NO: 298; j) a variable heavy domain with SEQ ID NO: 307 and a variable light domain with SEQ ID NO: 308; k) a variable heavy domain with SEQ ID NO: 317 and a variable light domain with SEQ ID NO: 318; l) a variable heavy domain with SEQ ID NO: 337 and a variable light domain with SEQ ID NO: 338; m) a variable heavy domain with SEQ ID NO: 347 and a variable light domain with SEQ ID NO: 348; n) a variable heavy domain with SEQ ID NO: 437 and a variable light domain with SEQ ID NO: 438; o) a variable heavy domain with SEQ ID NO: 457 and a variable light domain with SEQ ID NO: 458; p) a variable heavy domain with SEQ ID NO: 467 and a variable light domain with SEQ ID NO: 468; q) a variable heavy domain with SEQ ID NO: 477 and a variable light domain with SEQ ID NO: 478; r) a variable heavy domain with SEQ ID NO: 497 and a variable light domain with SEQ ID NO: 498; s) a variable heavy domain with SEQ ID NO: 537 and a variable light domain with SEQ ID NO: 538; t) a variable heavy domain with SEQ ID NO: 547 and a variable light domain with SEQ ID NO: 548; u) a variable heavy domain with SEQ ID NO: 567 and a variable light domain with SEQ ID NO: 568; v) a variable heavy domain with SEQ ID NO: 597 and a variable light domain with SEQ ID NO: 598; w) a variable heavy domain with SEQ ID NO: 617 and a variable light domain with SEQ ID NO: 618; x) a variable heavy domain with SEQ ID NO: 637 and a variable light domain with SEQ ID NO: 638; y) a variable heavy domain with SEQ ID NO: 707 and a variable light domain with SEQ ID NO: 708; all as depicted in U.S. Pat. No. 11,447,567 and incorporated by reference herein. Additionally, any sequence included above from U.S. Pat. No. 11,447,567 can also be individually included or excluded.

Further, included herein is an anti-FLT3 antigen binding domain with a VH/VL pair selected from the group including: a) a variable heavy domain with SEQ ID NO: 20 and a variable light domain with SEQ ID NO: 19; b) a variable heavy domain with SEQ ID NO: 6 and a variable light domain with SEQ ID NO: 5; c) a variable heavy domain with SEQ ID NO: 4 and a variable light domain with SEQ ID NO: 3; d) a variable heavy domain with SEQ ID NO: 8 and a variable light domain with SEQ ID NO: 7; e) a variable heavy domain with SEQ ID NO: 10 and a variable light domain with SEQ ID NO: 9; f) a variable heavy domain with SEQ ID NO: 12 and a variable light domain with SEQ ID NO: 11; g) a variable heavy domain with SEQ ID NO: 14 and a variable light domain with SEQ ID NO: 13; h) a variable heavy domain with SEQ ID NO: 16 and a variable light domain with SEQ ID NO: 15; i) a variable heavy domain with SEQ ID NO: 18 and a variable light domain with SEQ ID NO: 17; j) a variable heavy domain with SEQ ID NO: 2 and a variable light domain with SEQ ID NO: 1; k) a variable heavy domain with SEQ ID NO: 22 and a variable light domain with SEQ ID NO: 21; l) a variable heavy domain with SEQ ID NO: 24 and a variable light domain with SEQ ID NO: 23; m) a variable heavy domain with SEQ ID NO: 26 and a variable light domain with SEQ ID NO: 25; n) a variable heavy domain with SEQ ID NO: 28 and a variable light domain with SEQ ID NO: 27; o) a variable heavy domain with SEQ ID NO: 30 and a variable light domain with SEQ ID NO: 29; p) a variable heavy domain with SEQ ID NO: 32 and a variable light domain with SEQ ID NO: 31; q) a variable heavy domain with SEQ ID NO: 34 and a variable light domain with SEQ ID NO: 33; r) a variable heavy domain with SEQ ID NO: 36 and a variable light domain with SEQ ID NO: 35; s) a variable heavy domain with SEQ ID NO: 205 and a variable light domain with SEQ ID NO: 204; t) a variable heavy domain with SEQ ID NO: 207 and a variable light domain with SEQ ID NO: 206; u) a variable heavy domain with SEQ ID NO: 209 and a variable light domain with SEQ ID NO: 208; v) a variable heavy domain with SEQ ID NO: 211 and a variable light domain with SEQ ID NO: 210; w) a variable heavy domain with SEQ ID NO: 213 and a variable light domain with SEQ ID NO: 212; x) a variable heavy domain with SEQ ID NO: 215 and a variable light domain with SEQ ID NO: 214; y) a variable heavy domain with SEQ ID NO: 217 and a variable light domain with SEQ ID NO: 216; z) a variable heavy domain with SEQ ID NO: 219 and a variable light domain with SEQ ID NO: 218; aa) a variable heavy domain with SEQ ID NO: 221 and a variable light domain with SEQ ID NO: 220; bb) a variable heavy domain with SEQ ID NO: 223 and a variable light domain with SEQ ID NO: 222; cc) a variable heavy domain with SEQ ID NO: 225 and a variable light domain with SEQ ID NO: 224; dd) a variable heavy domain with SEQ ID NO: 227 and a variable light domain with SEQ ID NO: 226; ee) a variable heavy domain with SEQ ID NO: 229 and a variable light domain with SEQ ID NO: 228; ff) a variable heavy domain with SEQ ID NO: 231 and a variable light domain with SEQ ID NO: 230 and gg) a variable heavy domain with SEQ ID NO: 233 and a variable light domain with SEQ ID NO: 232, all as shown in U.S. Pat. No. 11,421,040, hereby incorporated by reference for the sequences. Additionally, any sequence included above from U.S. Pat. No. 11,421,040 can also be individually included or excluded.

Further, included herein is an anti-FLT3 antigen binding domain (“CHv62.21”) with, the variable heavy domain depicted as part of SEQ ID NO: 11, and the variable light domain depicted as part of SEQ ID NO: 10 from U.S. Pat. No. 10,751,422, hereby incorporated by reference for the sequences. Additionally, any sequence included above from U.S. Pat. No. 10,751,422 can also be individually included or excluded.

Further, included herein is an anti-FLT3 antigen binding domain with a VH/VL pair selected from the group including: a) a variable heavy domain with SEQ ID NO: 9 and a variable light domain with SEQ ID NO: 10; b) a variable heavy domain with SEQ ID NO: 13 and a variable light domain with SEQ ID NO: 10; c) a variable heavy domain with SEQ ID NO: 17 and a variable light domain with SEQ ID NO: 10; d) a variable heavy domain with SEQ ID NO: 9 and a variable light domain with SEQ ID NO: 22; e) a variable heavy domain with SEQ ID NO: 37 and a variable light domain with SEQ ID NO: 38; f) a variable heavy domain with SEQ ID NO: 41 and a variable light domain with SEQ ID NO: 42; g) a variable heavy domain with SEQ ID NO: 45 and a variable light domain with SEQ ID NO: 42; h) a variable heavy domain with SEQ ID NO: 49 and a variable light domain with SEQ ID NO: 42; and i) a variable heavy domain with SEQ ID NO: 53 and a variable light domain with SEQ ID NO: 42; all as shown in WO2021/076564, hereby incorporated by reference for the sequences. Additionally, any sequence included above from WO2021/076564 can also be individually included or excluded.

Further, included herein is an anti-FLT3 antigen binding domain with a VH/VL pair selected from the group including: a) a variable heavy domain with SEQ ID NO: 187 and a variable light domain with SEQ ID NO: 288; b) a variable heavy domain with SEQ ID NO: 207 and a variable light domain with SEQ ID NO: 208; c) a variable heavy domain with SEQ ID NO: 217 and a variable light domain with SEQ ID NO: 218; d) a variable heavy domain with SEQ ID NO: 227 and a variable light domain with SEQ ID NO: 228; e) a variable heavy domain with SEQ ID NO: 257 and a variable light domain with SEQ ID NO: 258; f) a variable heavy domain with SEQ ID NO: 267 and a variable light domain with SEQ ID NO: 268; g) a variable heavy domain with SEQ ID NO: 277 and a variable light domain with SEQ ID NO: 278; h) a variable heavy domain with SEQ ID NO: 287 and a variable light domain with SEQ ID NO: 288; i) a variable heavy domain with SEQ ID NO: 297 and a variable light domain with SEQ ID NO: 298; j) a variable heavy domain with SEQ ID NO: 307 and a variable light domain with SEQ ID NO: 308; k) a variable heavy domain with SEQ ID NO: 317 and a variable light domain with SEQ ID NO: 318; l) a variable heavy domain with SEQ ID NO: 337 and a variable light domain with SEQ ID NO: 338; m) a variable heavy domain with SEQ ID NO: 347 and a variable light domain with SEQ ID NO: 348; n) a variable heavy domain with SEQ ID NO: 437 and a variable light domain with SEQ ID NO: 438; o) a variable heavy domain with SEQ ID NO: 457 and a variable light domain with SEQ ID NO: 458; p) a variable heavy domain with SEQ ID NO: 467 and a variable light domain with SEQ ID NO: 468; q) a variable heavy domain with SEQ ID NO: 477 and a variable light domain with SEQ ID NO: 478; r) a variable heavy domain with SEQ ID NO: 497 and a variable light domain with SEQ ID NO: 498; s) a variable heavy domain with SEQ ID NO: 537 and a variable light domain with SEQ ID NO: 538; t) a variable heavy domain with SEQ ID NO: 547 and a variable light domain with SEQ ID NO: 548; u) a variable heavy domain with SEQ ID NO: 567 and a variable light domain with SEQ ID NO: 568; v) a variable heavy domain with SEQ ID NO: 597 and a variable light domain with SEQ ID NO: 598; w) a variable heavy domain with SEQ ID NO: 617 and a variable light domain with SEQ ID NO: 618; x) a variable heavy domain with SEQ ID NO: 637 and a variable light domain with SEQ ID NO: 638; y) a variable heavy domain with SEQ ID NO: 707 and a variable light domain with SEQ ID NO: 708; all as shown in U.S. Pat. No. 11,447,567, hereby incorporated by reference for the sequences. Additionally, any sequence included above from U.S. Pat. No. 11,447,567 can also be individually included or excluded.

In some embodiments, the NKEs of the invention include at least one anti-FLT3 antigen binding domain, as described herein and in the Figures. Described herein is a plurality of means for binding the ECD of human FLT3.

9. Mesothelin (MSLN) Antigen Binding Domains

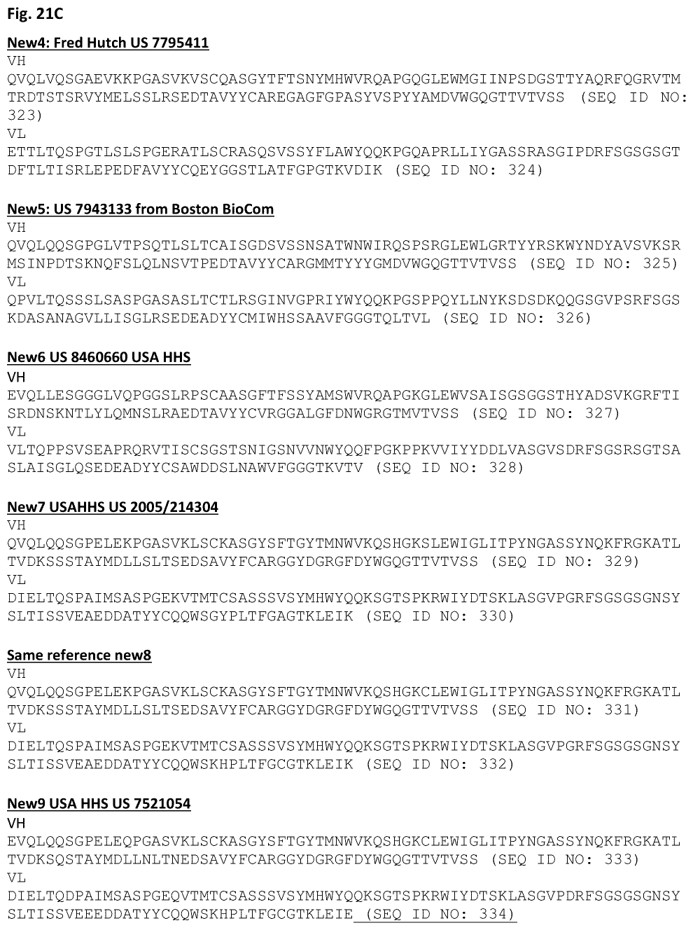

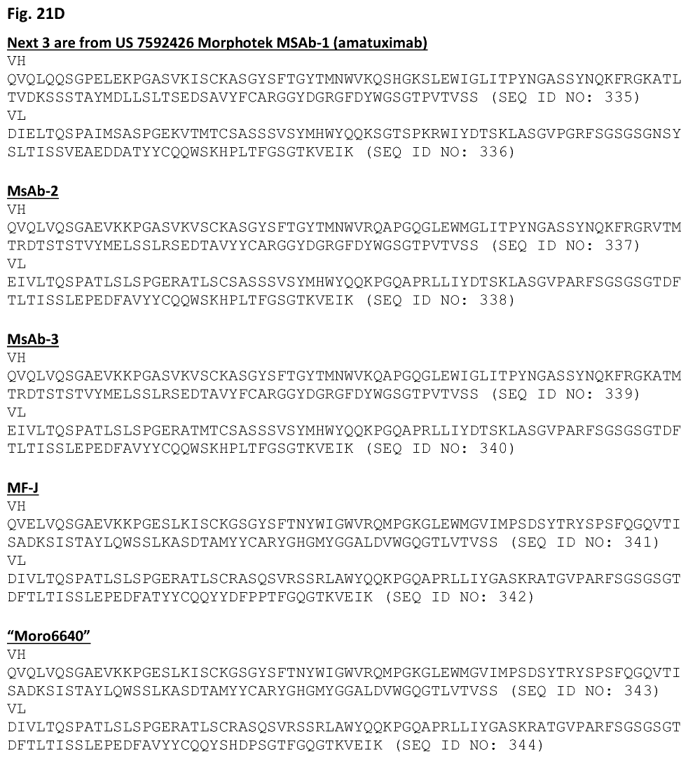

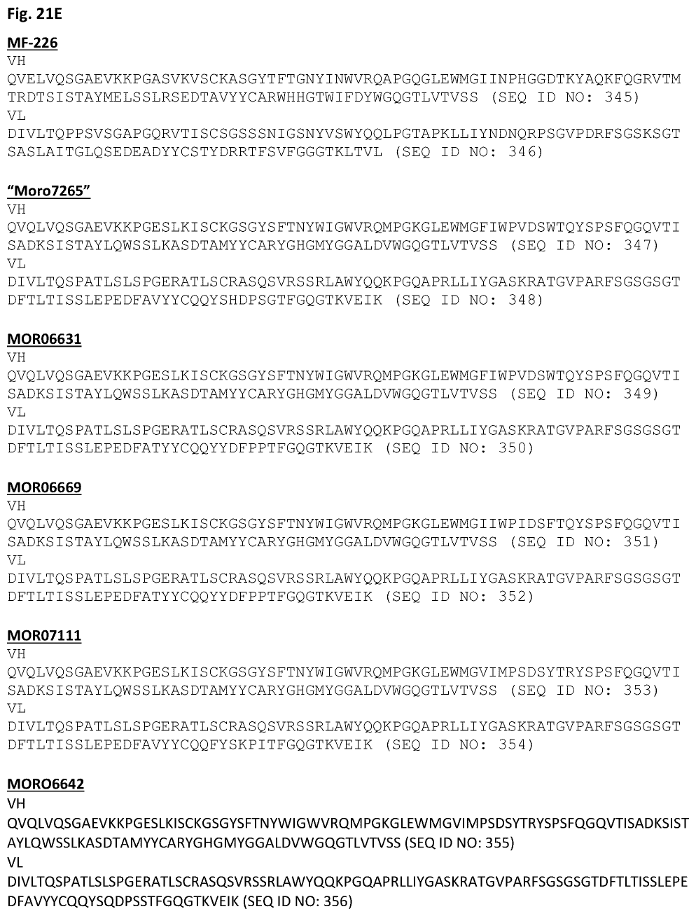

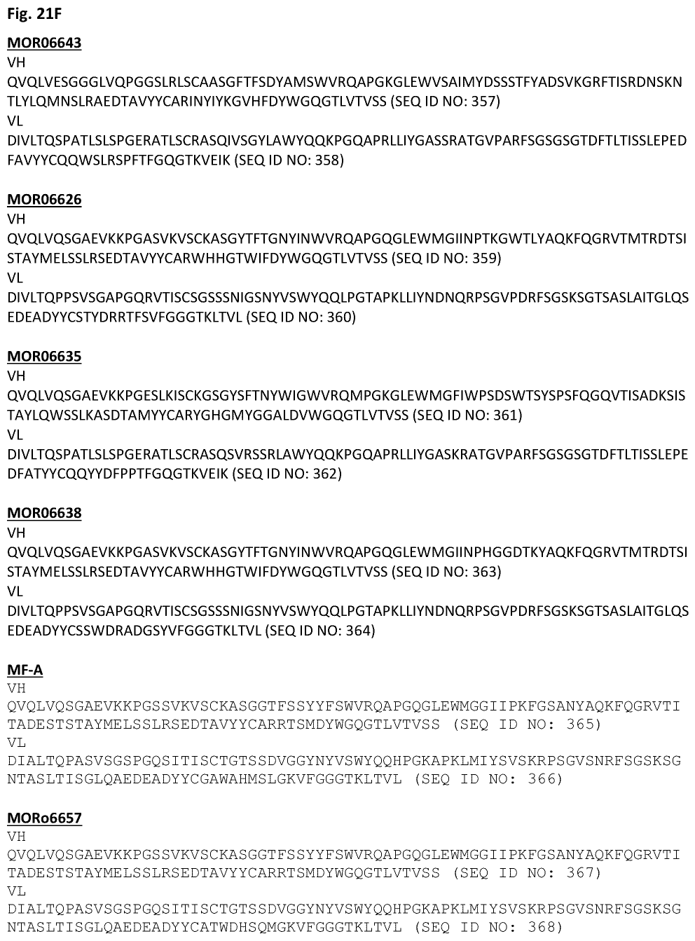

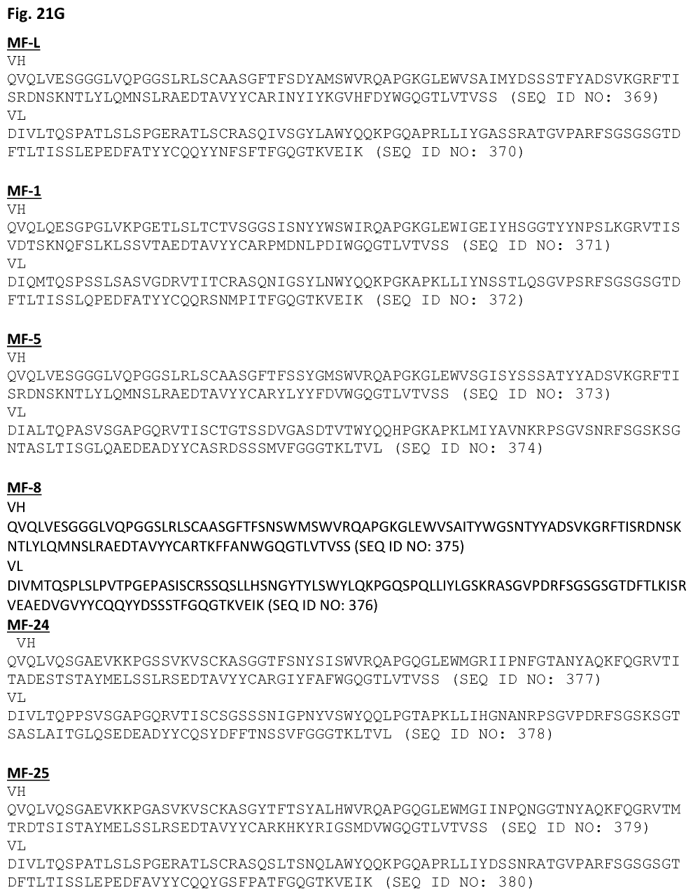

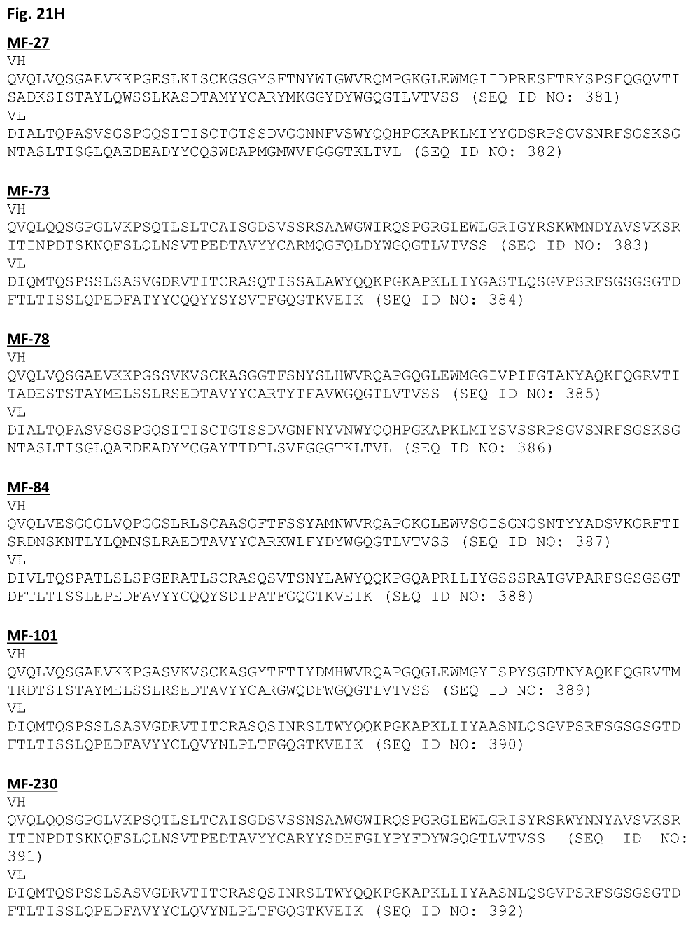

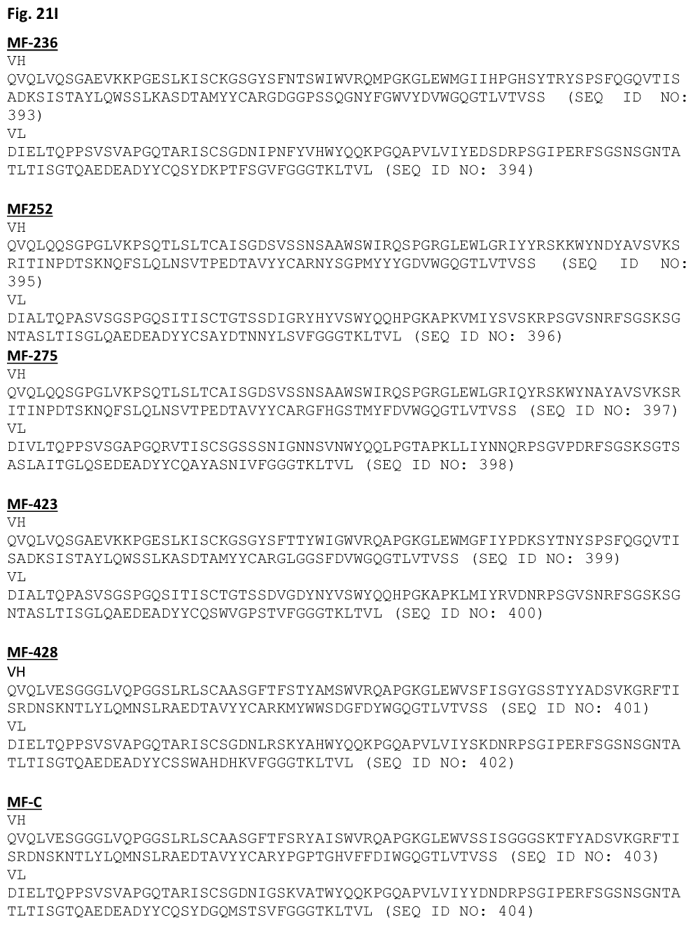

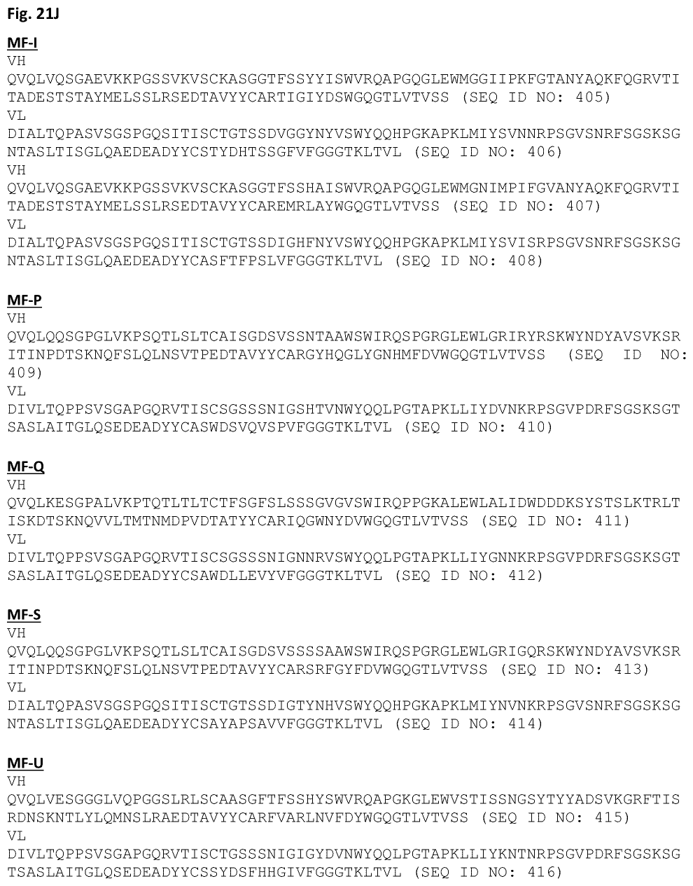

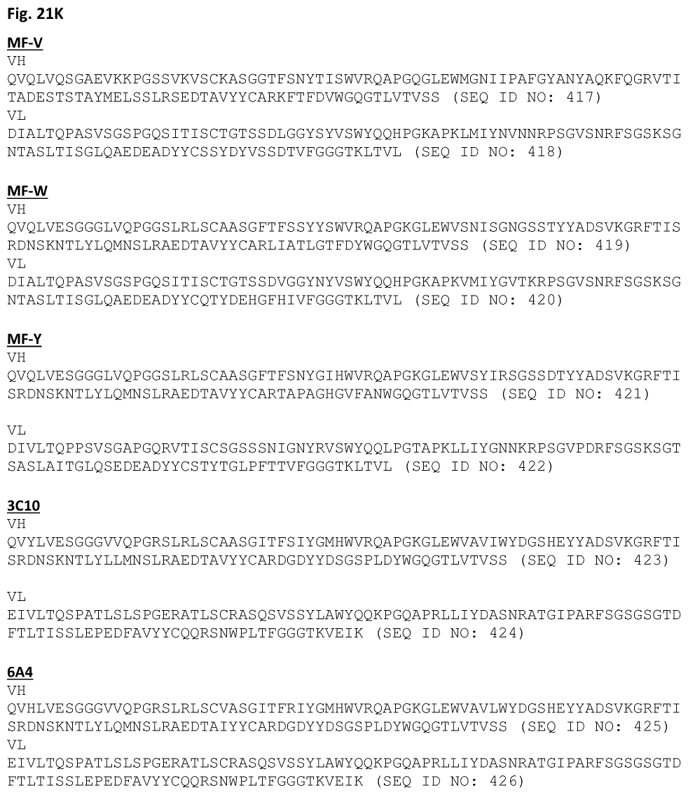

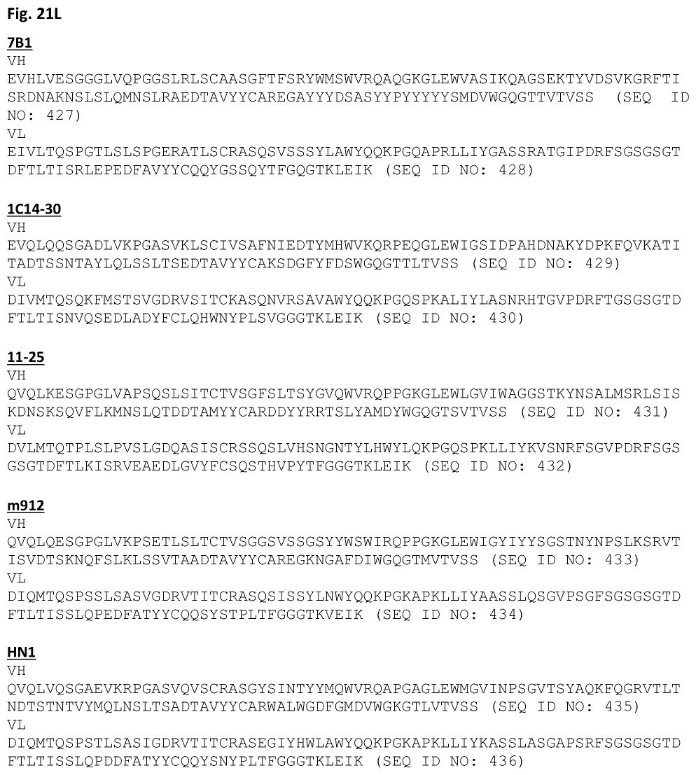

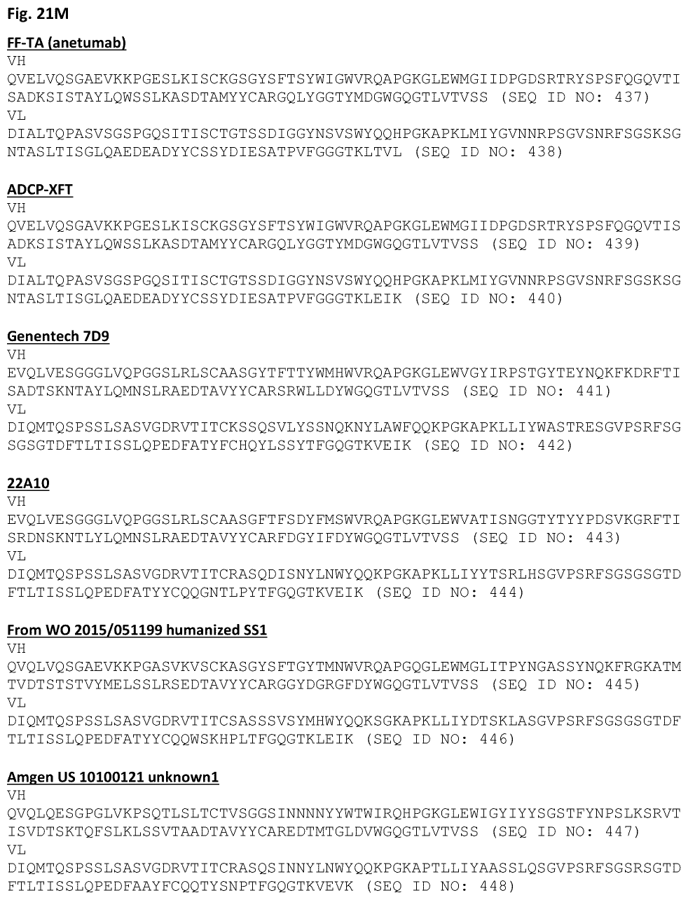

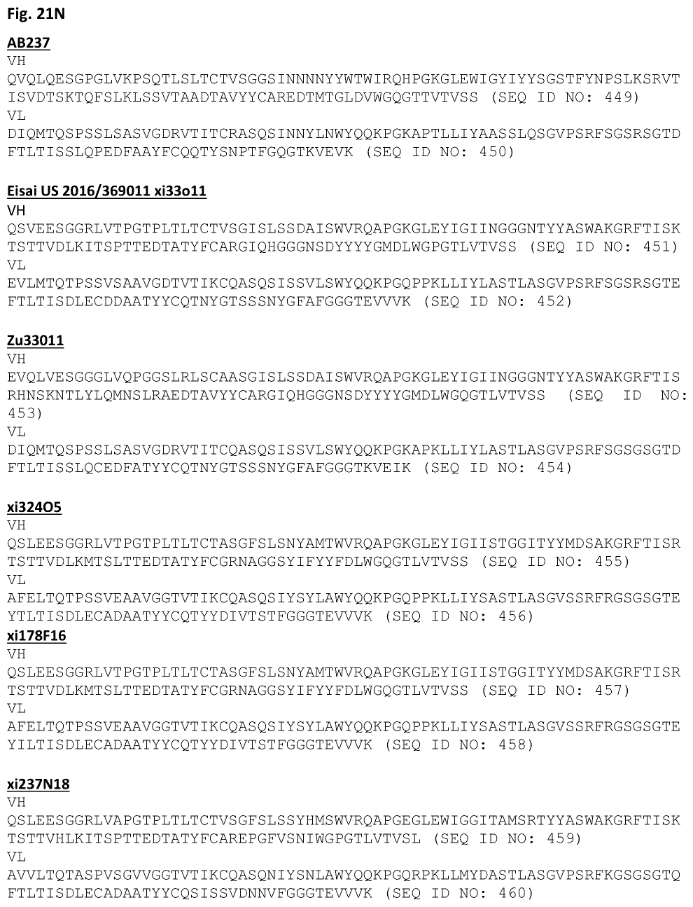

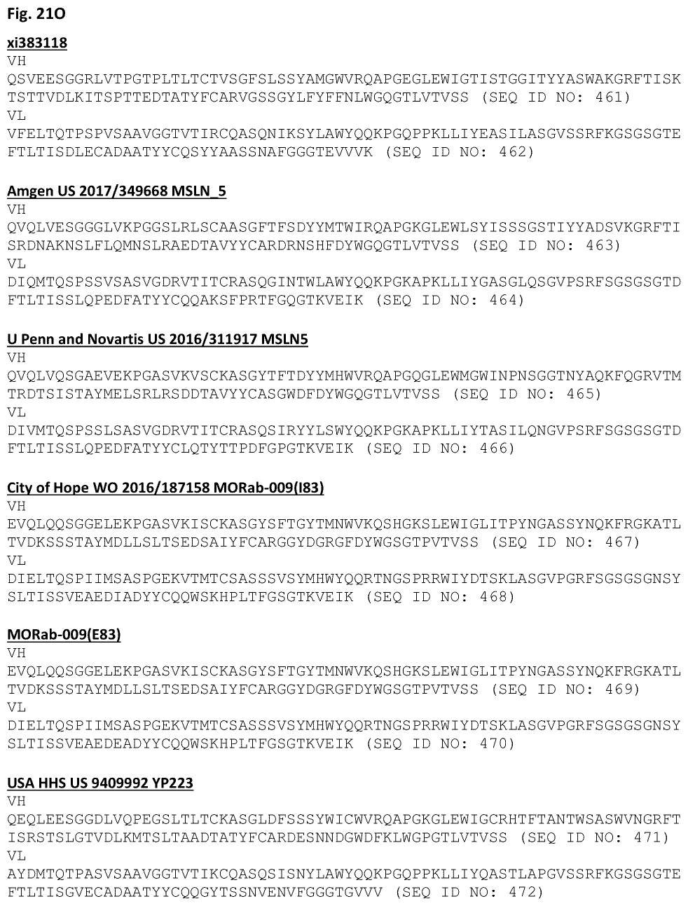

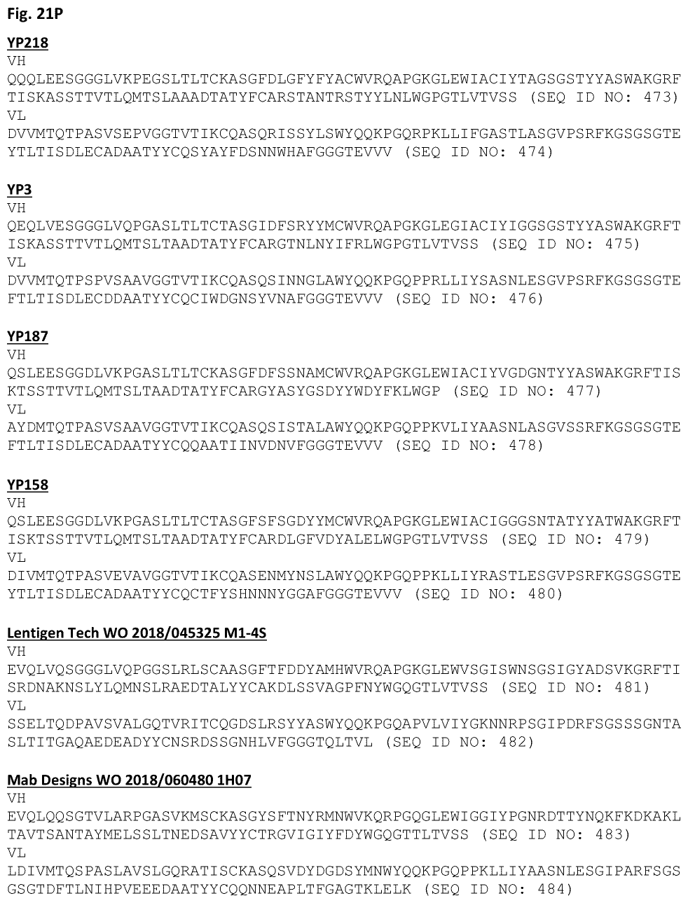

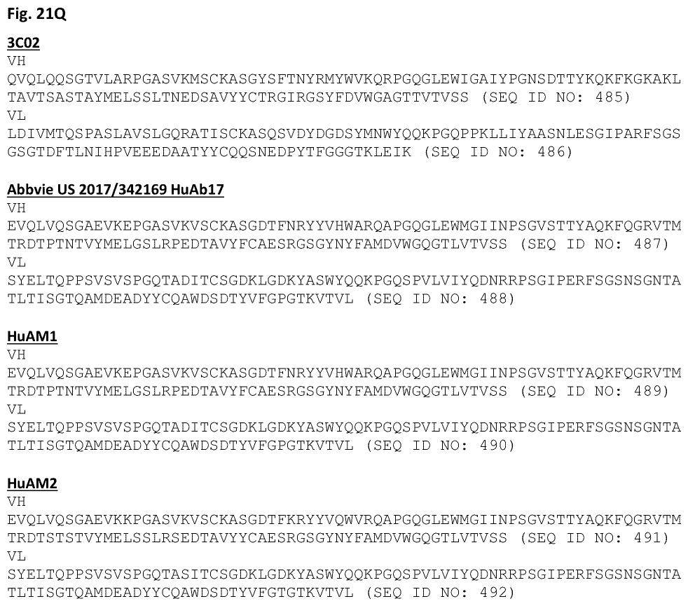

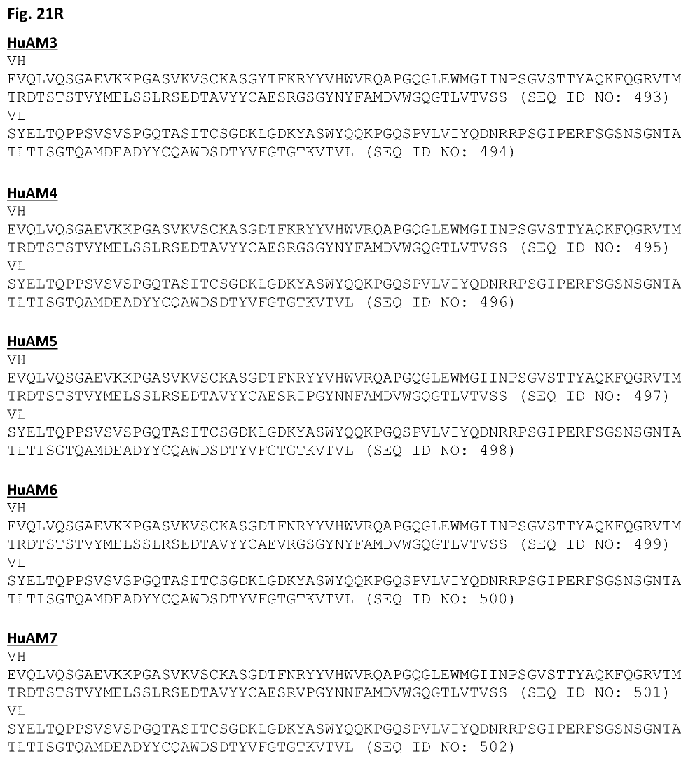

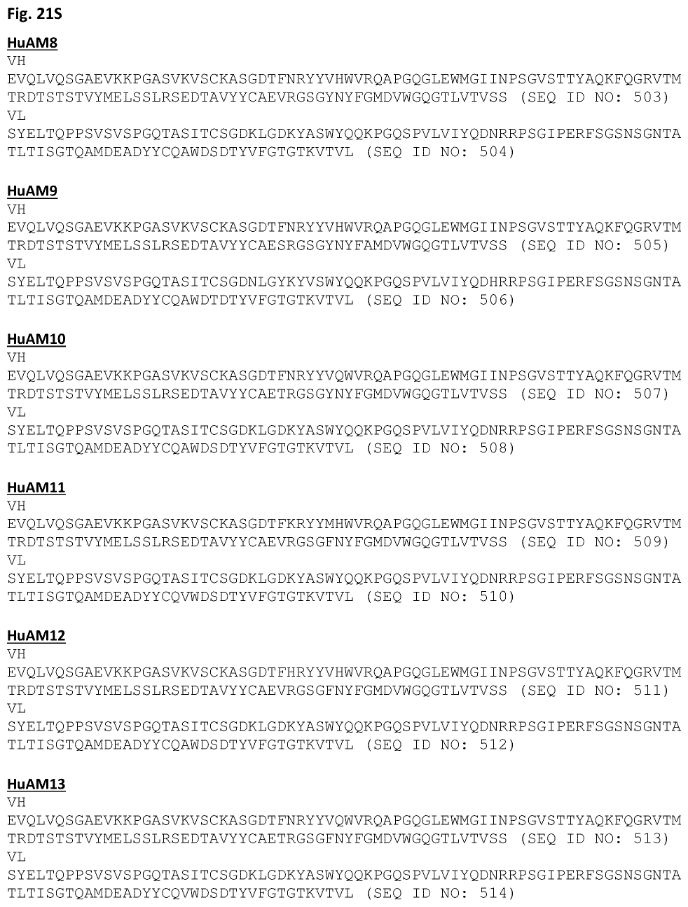

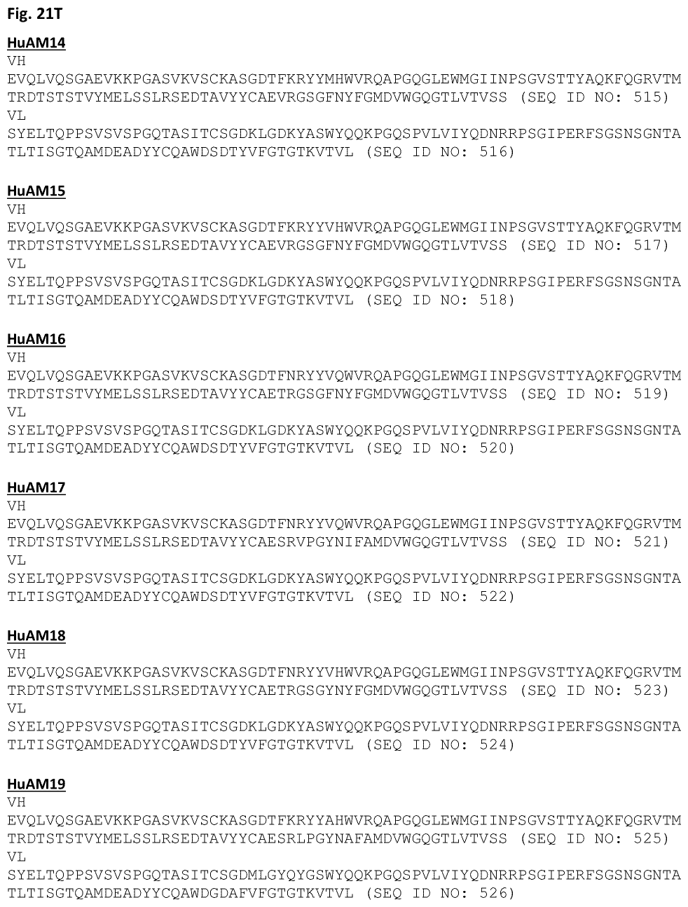

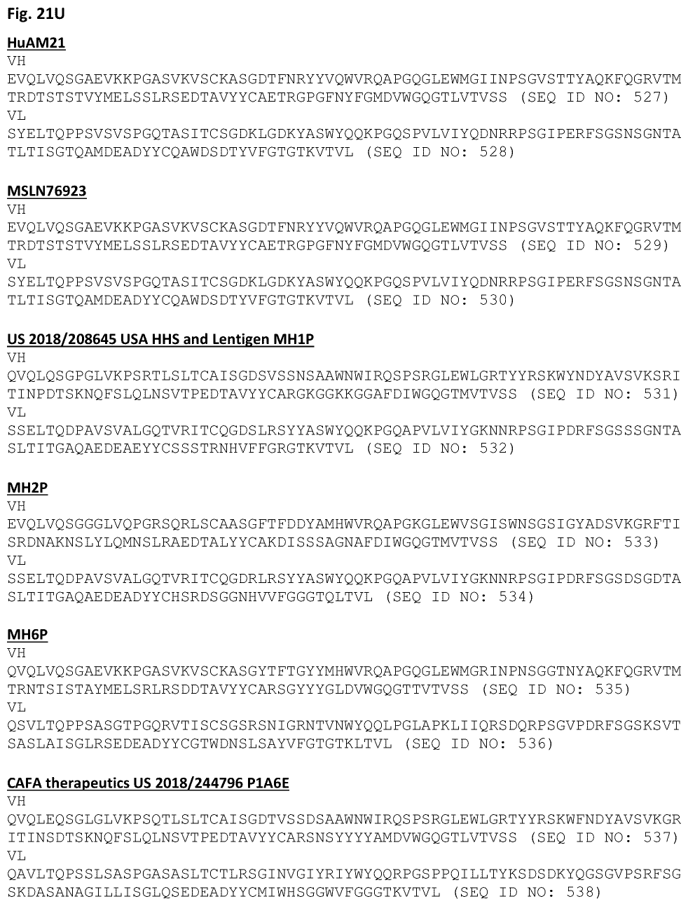

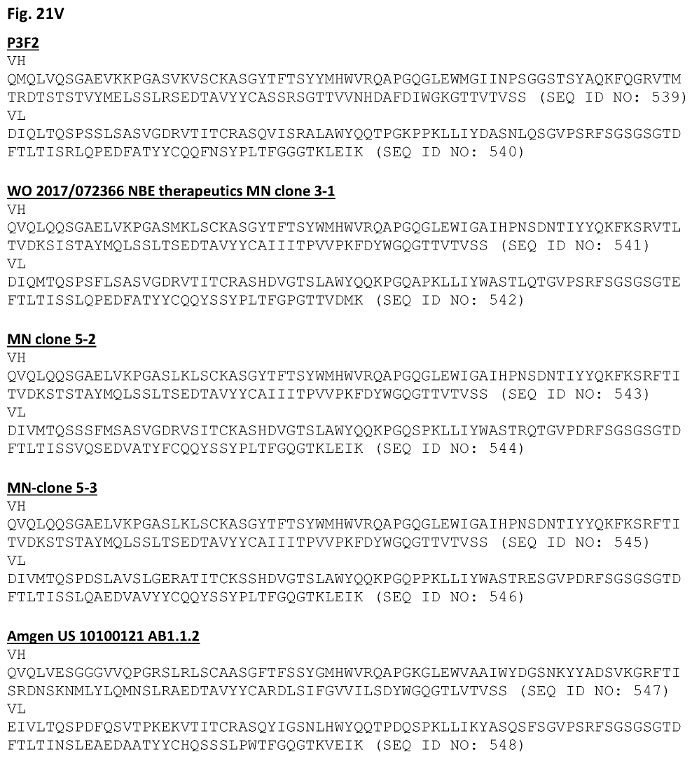

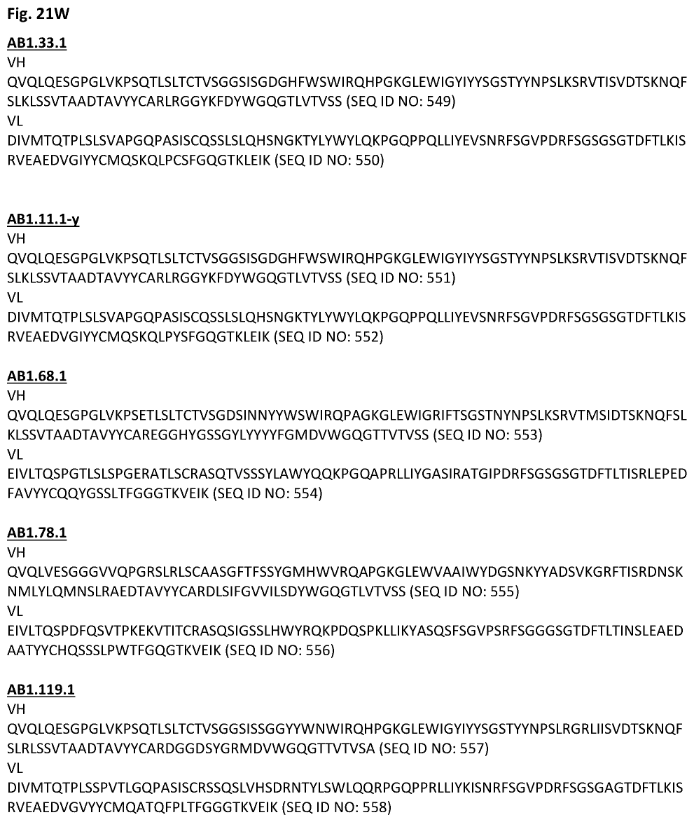

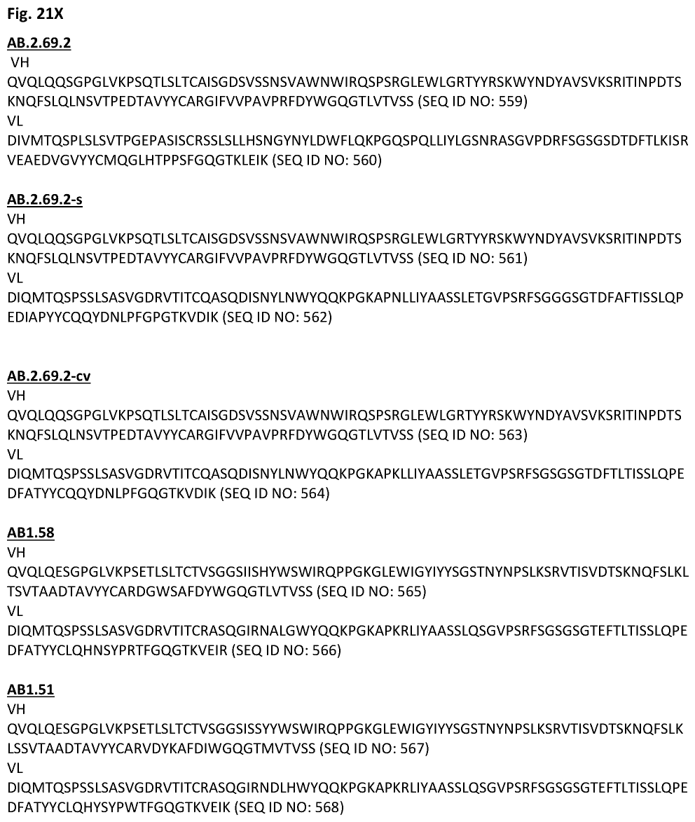

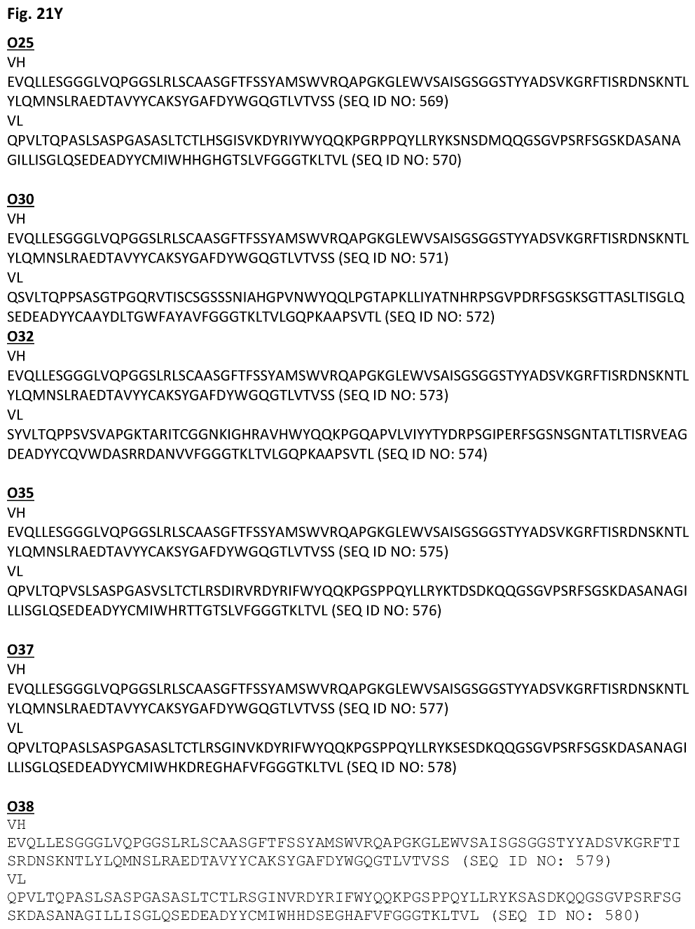

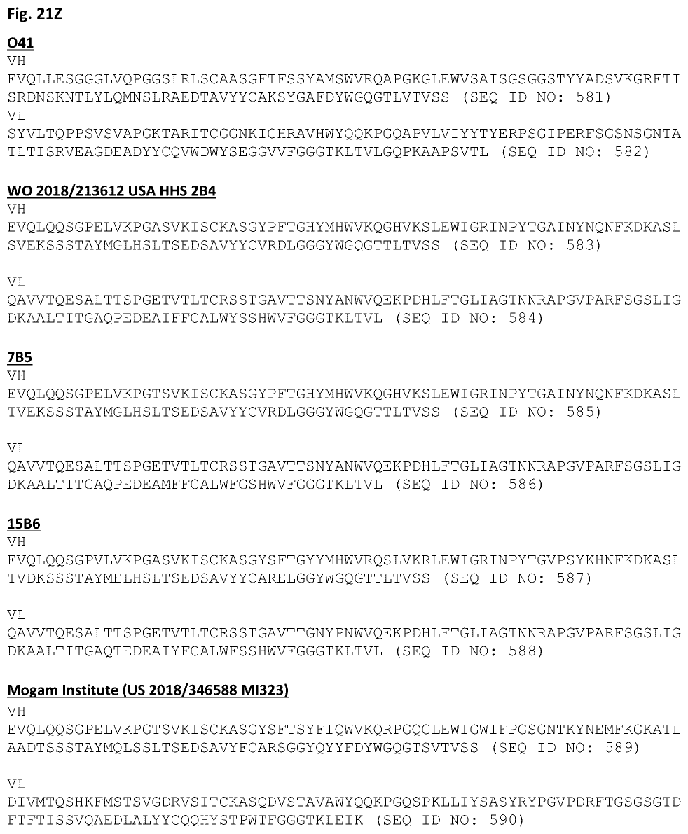

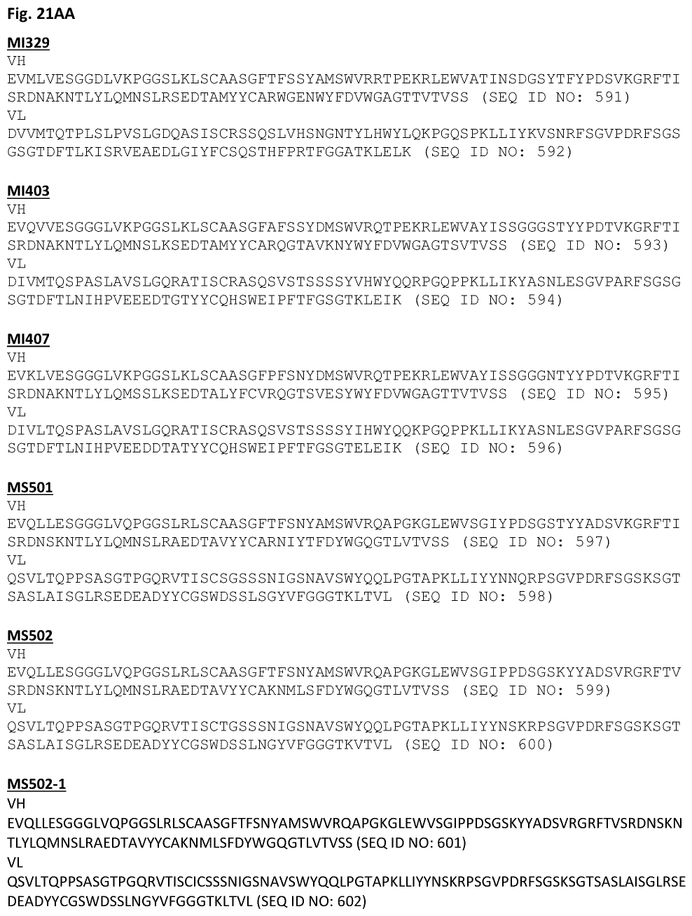

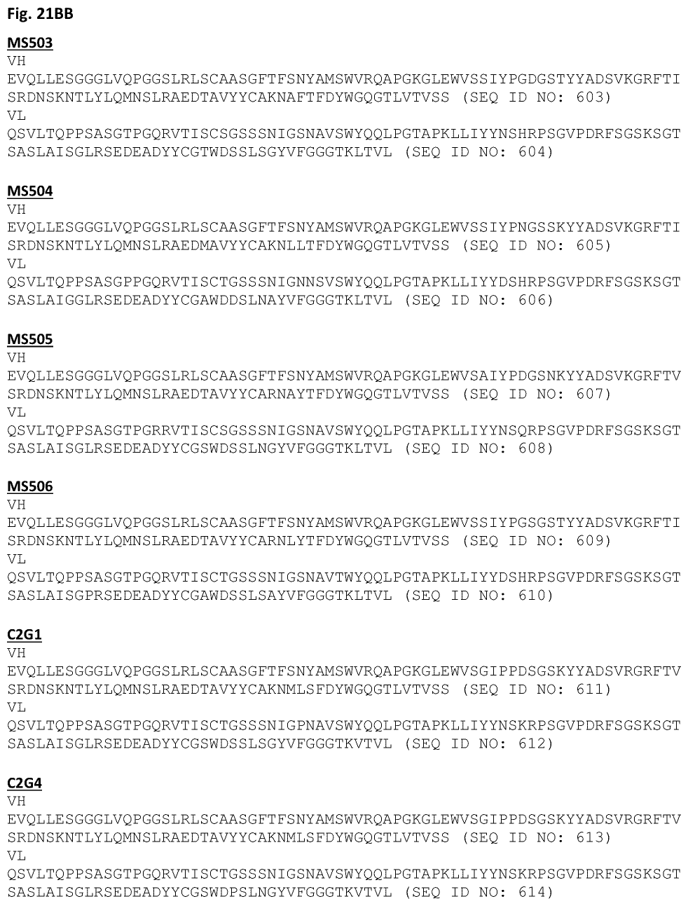

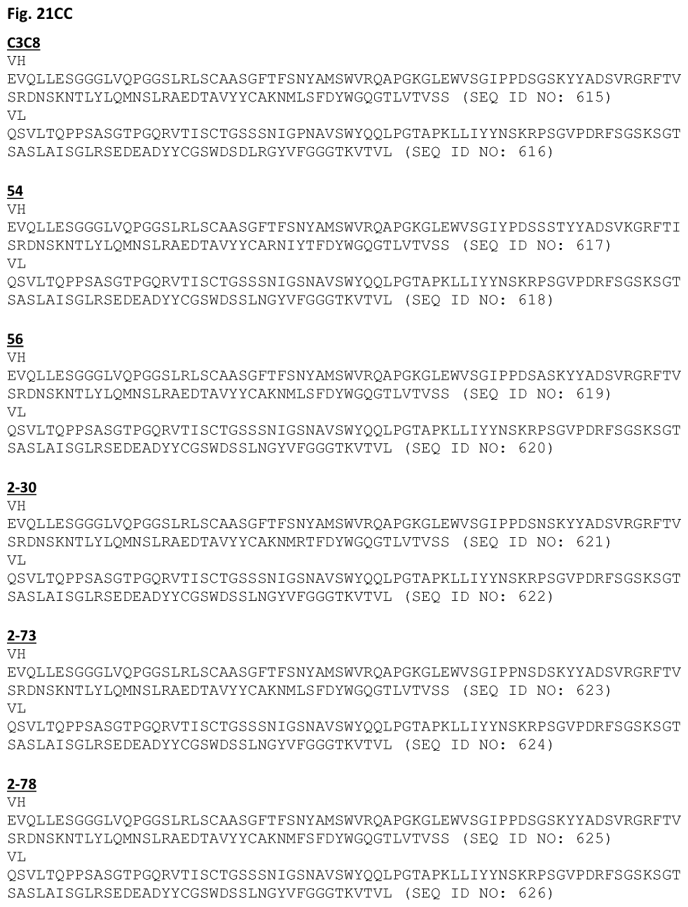

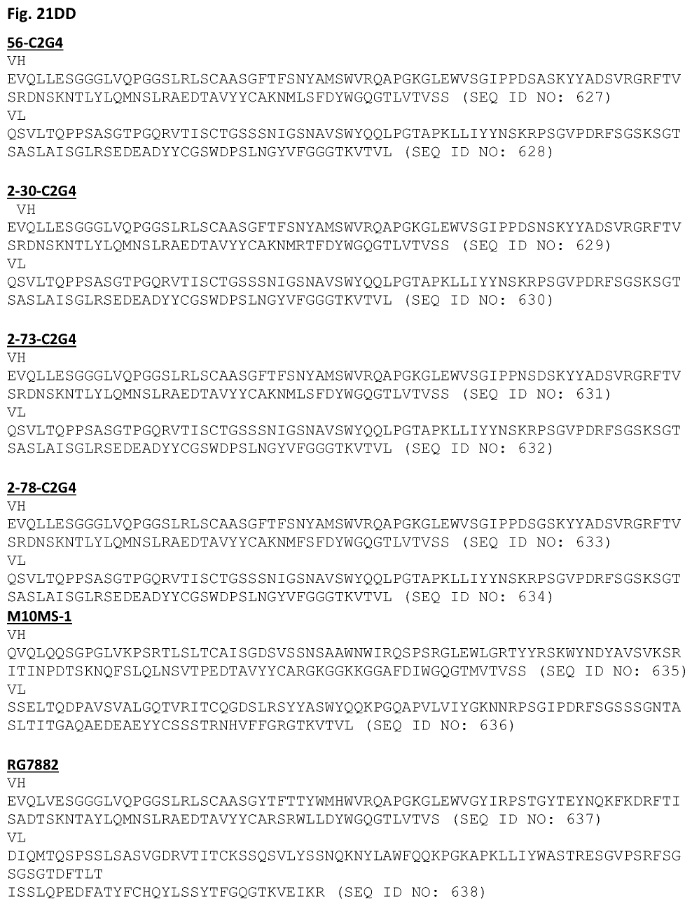

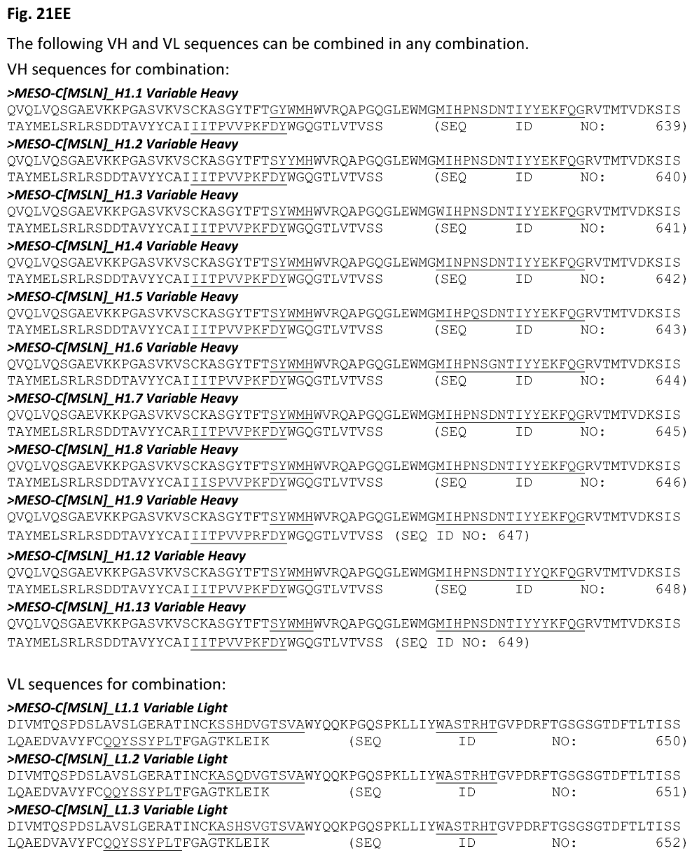

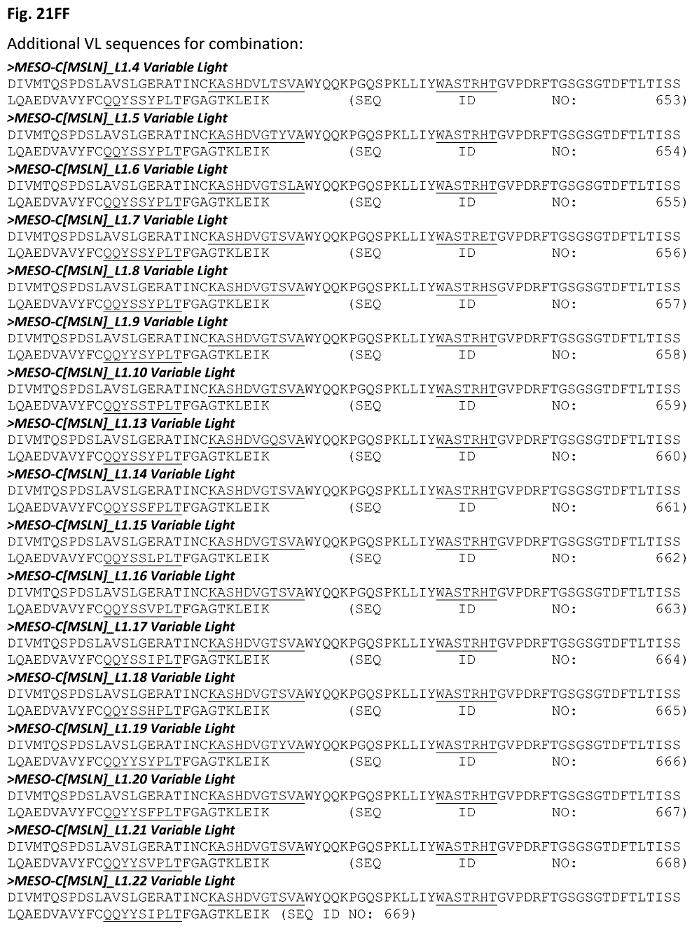

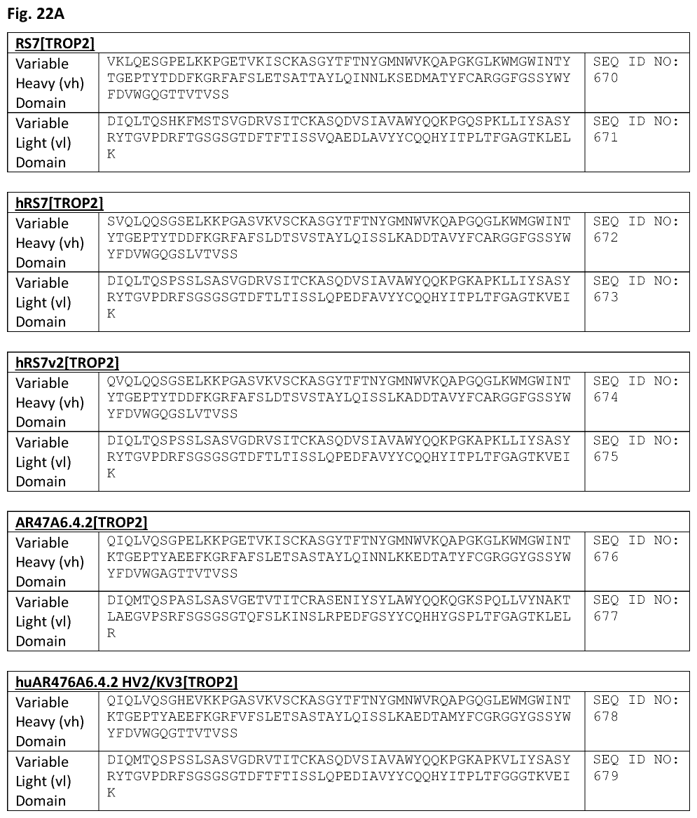

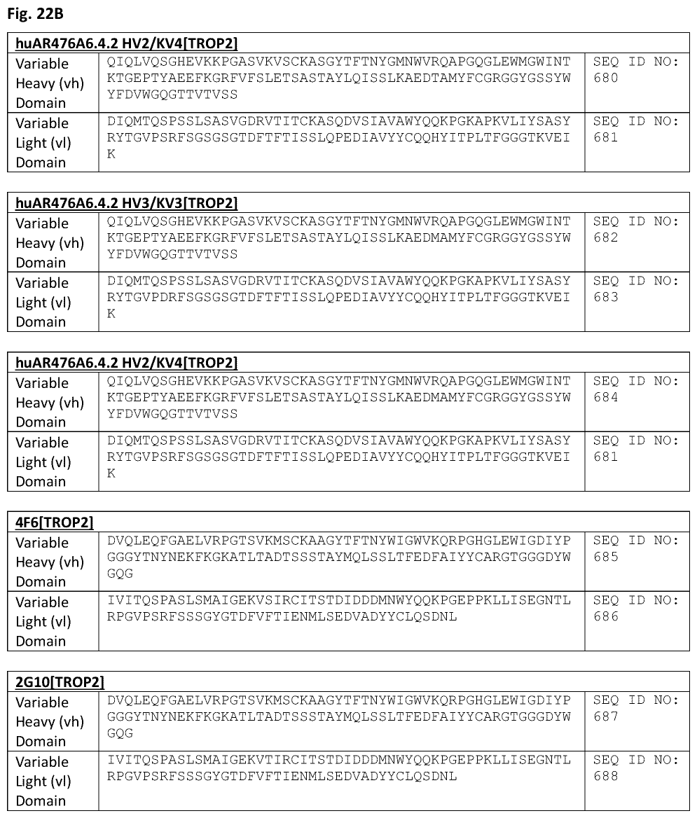

In some embodiments, the NKEs include an ABD that binds to the ECD of human MSLN. Suitable variable heavy VH and variable light VL domains that bind to the ECD of human MSLN include, but are not limited to, VH/VL pairs selected from the group including: (i) SEQ ID NOs: 289 and 293, (ii) SEQ ID NOs: 297 and 301, (iii) SEQ ID NOs: 305 and 309, (iv) SEQ ID NOs: 313 and 314, (v) SEQ ID NOs: 315 and 316, (vi) SEQ ID NOs: 317 and 318, (vii) SEQ ID NOs: 319 and 320, (viii) SEQ ID NOs: 321 and 322, (ix) SEQ ID NOs: 323 and 324, (x) SEQ ID NOs: 325 and 326, (xi) SEQ ID NOs: 327 and 328, (xii) SEQ ID NOs: 329 and 330, (xiii) SEQ ID NOs: 331 and 332, (xiv) SEQ ID NOs: 333 and 334, (xv) SEQ ID NOs: 335 and 336, (xvi) SEQ ID NOs: 337 and 338, (xvii) SEQ ID NOs: 339 and 340, (xviii) SEQ ID NOs: 341 and 342, (xix) SEQ ID NOs: 343 and 344, (xx) SEQ ID NOs: 345 and 346, (xxi) SEQ ID NOs: 347 and 348, (xxii) SEQ ID NOs: 349 and 350, (xxiii) SEQ ID NOs: 351 and 352, (xxiv) SEQ ID NOs: 353 and 354, (xxv) SEQ ID NOs: 355 and 356, (xxvi) SEQ ID NOs: 357 and 358, (xxvii) SEQ ID NOs: 359 and 360, (xxviii) SEQ ID NOs: 361 and 362, (xxix) SEQ ID NOs: 363 and 364, (xxx) SEQ ID NOs: 365 and 366, (xxxi) SEQ ID NOs: 367 and 368, (xxxii) SEQ ID NOs: 369 and 370, (xxxiii) SEQ ID NOs: 371 and 372, (xxxiv) SEQ ID NOs: 373 and 374, (xxxv) SEQ ID NOs: 375 and 376, (xxxvi) SEQ ID NOs: 377 and 378, (xxxvii) SEQ ID NOs: 379 and 380, (xxxviii) SEQ ID NOs: 381 and 382, (xxxix) SEQ ID NOs: 383 and 384, (xl) SEQ ID NOs: 385 and 386, (xli) SEQ ID NOs: 387 and 388, (xlii) SEQ ID NOs: 389 and 390, (xliii) SEQ ID NOs: 391 and 392, (xliv) SEQ ID NOs: 393 and 394, (xlv) SEQ ID NOs: 395 and 396, (xlvi) SEQ ID NOs: 397 and 398, (xlvii) SEQ ID NOs: 399 and 400, (xlviii) SEQ ID NOs: 401 and 402, (xlix) SEQ ID NOs: 403 and 404, (1) SEQ ID NOs: 405 and 406, (li) SEQ ID NOs: 405 and 408, (lii) SEQ ID NOs: 407 and 406, (liii) SEQ ID NOs: 407 and 408, (liv) SEQ ID NOs: 409 and 410, (lv) SEQ ID NOs: 411 and 412, (lvi) SEQ ID NOs: 413 and 414, (lvii) SEQ ID NOs: 415 and 416, (lviii) SEQ ID NOs: 417 and 418, (lix) SEQ ID NOs: 419 and 420, (lx) SEQ ID NOs: 421 and 422, (lxi) SEQ ID NOs: 423 and 424, (lxii) SEQ ID NOs: 425 and 426, (lxiii) SEQ ID NOs: 427 and 428, (lxiv) SEQ ID NOs: 429 and 430, (lxv) SEQ ID NOs: 431 and 432, (lxvi) SEQ ID NOs: 433 and 434, (lxvii) SEQ ID NOs: 435 and 436, (lxviii) SEQ ID NOs: 437 and 438, (lxix) SEQ ID NOs: 439 and 440, (lxx) SEQ ID NOs: 441 and 442, (lxxi) SEQ ID NOs: 443 and 444, (lxxii) SEQ ID NOs: 445 and 446, (lxxiii) SEQ ID NOs: 447 and 448, (lxxiv) SEQ ID NOs: 449 and 450, (lxxv) SEQ ID NOs: 451 and 452, (lxxvi) SEQ ID NOs: 453 and 454, (lxxvii) SEQ ID NOs: 455 and 456, (lxxviii) SEQ ID NOs: 457 and 458, (lxxix) SEQ ID NOs: 459 and 460, (lxxx) SEQ ID NOs: 461 and 462, (lxxxi) SEQ ID NOs: 463 and 464, (lxxxii) SEQ ID NOs: 465 and 466, (lxxxiii) SEQ ID NOs: 467 and 468, (lxxxix) SEQ ID NOs: 469 and 470, (lxxxv) SEQ ID NOs: 471 and 472, (lxxxvi) SEQ ID NOs: 473 and 474, (lxxxvii) SEQ ID NOs: 475 and 476, (lxxxviii) SEQ ID NOs: 477 and 478, (lxxxix) SEQ ID NOs: 479 and 480, (xc) SEQ ID NOs: 481 and 482, (xci) SEQ ID NOs: 483 and 484, (xcii) SEQ ID NOs: 485 and 486, (xciii) SEQ ID NOs: 487 and 488, (xciv) SEQ ID NOs: 489 and 490, (xcv) SEQ ID NOs: 491 and 492, (xcvi) SEQ ID NOs: 493 and 494, (xcvii) SEQ ID NOs: 495 and 496, (xcviii) SEQ ID NOs: 497 and 498, (xcix) SEQ ID NOs: 499 and 500, (c) SEQ ID NOs: 501 and 502, (ci) SEQ ID NOs: 503 and 504, (cii) SEQ ID NOs: 505 and 506, (ciii) SEQ ID NOs: 507 and 508, (civ) SEQ ID NOs: 509 and 510, (cv) SEQ ID NOs: 511 and 512, (cvi) SEQ ID NOs: 513 and 514, (cvii) SEQ ID NOs: 515 and 516, (cviii) SEQ ID NOs: 517 and 518, (cix) SEQ ID NOs: 519 and 520, (cx) SEQ ID NOs: 521 and 522, (cxi) SEQ ID NOs: 523 and 524, (cxii) SEQ ID NOs: 525 and 526, (cxiii) SEQ ID NOs: 527 and 528, (cxiv) SEQ ID NOs: 529 and 530, (cxv) SEQ ID NOs: 531 and 532, (cxvi) SEQ ID NOs: 533 and 534, (cxvii) SEQ ID NOs: 535 and 536, (cxviii) SEQ ID NOs: 537 and 538, (cxix) SEQ ID NOs: 539 and 540, (cxx) SEQ ID NOs: 541 and 542, (cxxi) SEQ ID NOs: 543 and 544, (cxxii) SEQ ID NOs: 545 and 546, (cxxiii) SEQ ID NOs: 547 and 548, (cxxiv) SEQ ID NOs: 549 and 550, (cxxv) SEQ ID NOs: 551 and 552, (cxxvi) SEQ ID NOs: 553 and 554, (cxxvii) SEQ ID NOs: 555 and 556, (cxxviii) SEQ ID NOs: 557 and 558, (cxxix) SEQ ID NOs: 559 and 560, (cxxx) SEQ ID NOs: 561 and 562, (cxxxi) SEQ ID NOs: 563 and 564, (cxxxii) SEQ ID NOs: 565 and 566, (cxxxiii) SEQ ID NOs: 567 and 568, (cxxxiv) SEQ ID NOs: 569 and 570, (cxxxv) SEQ ID NOs: 571 and 572, (cxxxvi) SEQ ID NOs: 573 and 574, (cxxxvii) SEQ ID NOs: 575 and 576, (cxxxviii) SEQ ID NOs: 577 and 578, (cxl) SEQ ID NOs: 579 and 580, (cxli) SEQ ID NOs: 581 and 582, (cxlii) SEQ ID NOs: 583 and 584, (cxliii) SEQ ID NOs: 585 and 586, (cxliv) SEQ ID NOs: 587 and 588, (cxlv) SEQ ID NOs: 589 and 590, (cxlvi) SEQ ID NOs: 591 and 592, (cxlvii) SEQ ID NOs: 593 and 594, (cxlviii) SEQ ID NOs: 595 and 596, (cxlix) SEQ ID NOs: 597 and 598, (cl) SEQ ID NOs: 599 and 600, (cli) SEQ ID NOs: 601 and 602, (clii) SEQ ID NOs: 603 and 604, (cliii) SEQ ID NOs: 605 and 606, (cliv) SEQ ID NOs: 607 and 608, (clv) SEQ ID NOs: 609 and 610, (clvi) SEQ ID NOs: 611 and 612, (clvii) SEQ ID NOs: 613 and 614, (clviii) SEQ ID NOs: 615 and 616, (clviii) SEQ ID NOs: 617 and 618, (clix) SEQ ID NOs: 619 and 620, (clx) SEQ ID NOs: 621 and 622, (clxi) SEQ ID NOs: 623 and 624, (clxii) SEQ ID NOs: 625 and 626, (clxiii) SEQ ID NOs: 627 and 628, (clxiv) SEQ ID NOs: 629 and 630, (clxv) SEQ ID NOs: 631 and 632, (clxvi) SEQ ID NOs: 633 and 634, (clxvii) SEQ ID NOs: 635 and 636, (clxviii) SEQ ID NOs: 637 and 638, and (clxix) any one of SEQ ID NOs: 639-649 and any one of SEQ ID NOs: 650-669, as shown in FIGS. 21A-21FF, as well as those disclosed herein. It should be noted that these ABDs can be included in any of the formats described herein. Additionally, any sequence included above from FIG. 21 can also be individually included or excluded.

In some embodiments, an anti-human MSLN antigen binding domain includes a combination of VH and VL sequences, such as those shown in FIGS. 21EE and 21FF (see, e.g., SEQ ID NOs: 639-649 (VH sequences) and SEQ ID NOs: 650-669 (VL sequences)).

Additionally, any sequence included above from FIG. 21 can also be individually included or excluded.

Included herein is an anti-human MSLN antigen binding domain with sequences selected from the group including: a) a VH with the amino acid sequence shown in SEQ ID NO:106 and a VL with the amino acid sequence shown in SEQ ID NO:106; b) a VH with the amino acid sequence shown in SEQ ID NO:114 and a VL with the amino acid sequence shown in SEQ ID NO:15; and c) a VH with the amino acid sequence shown in SEQ ID NO:122 and a VL with the amino acid sequence shown in SEQ ID NO:123; wherein the sequences are as shown in Table 4 of WO2019/234220, hereby expressly incorporated by reference for the sequences. Additionally, any sequence included above from Table 4 of WO2019/234220 can also be individually included or excluded. In some cases, other anti-human MSLN antigen binding domains are described in WO2018/157147. Additionally, any sequence included above from WO2018/157147 can also be individually included or excluded.

In some embodiments, the NKEs of the invention include at least one anti-MSLN antigen binding domain, as described herein and in the Figures. Described herein is a plurality of means for binding the ECD of human MSLN.

10. Trop-2 Antigen Binding Domains

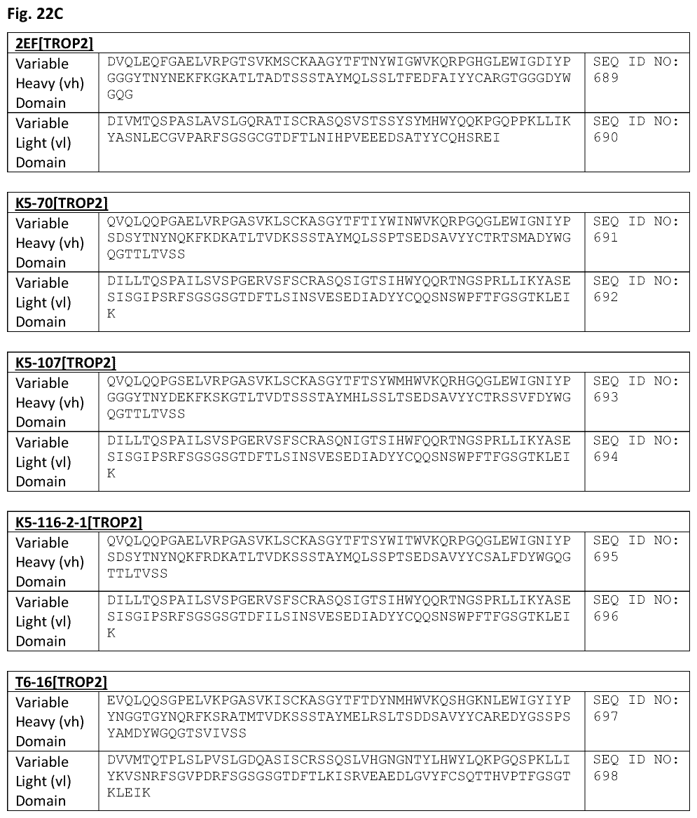

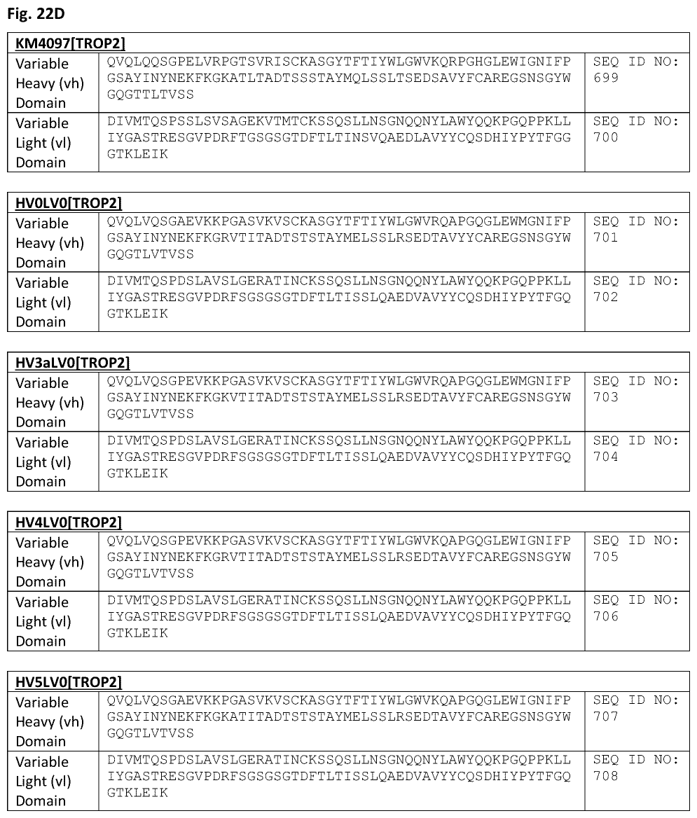

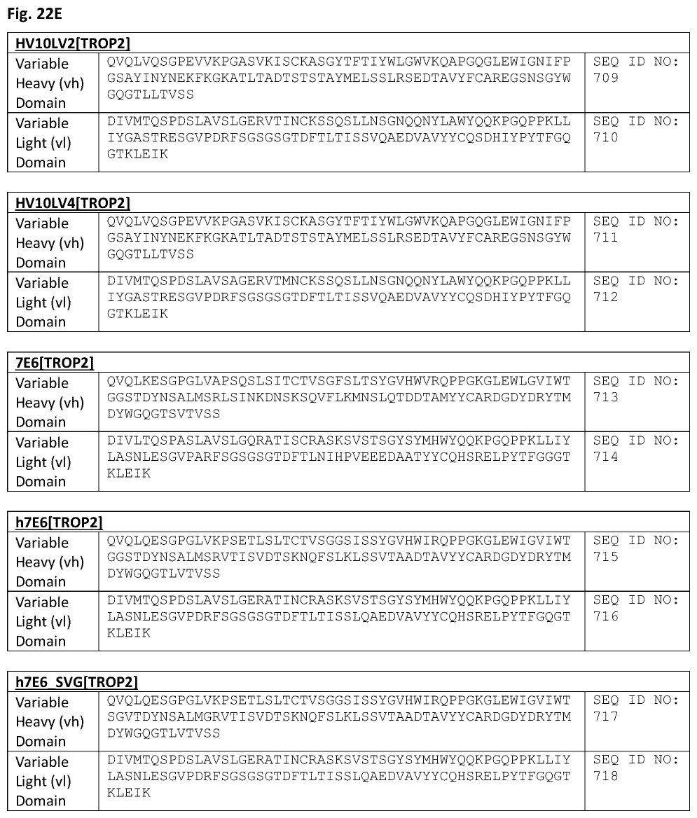

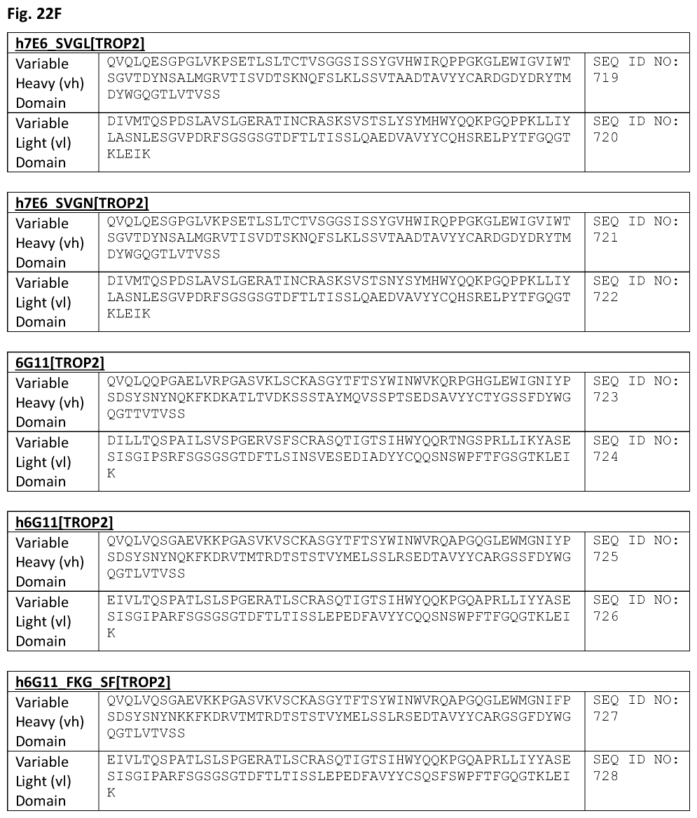

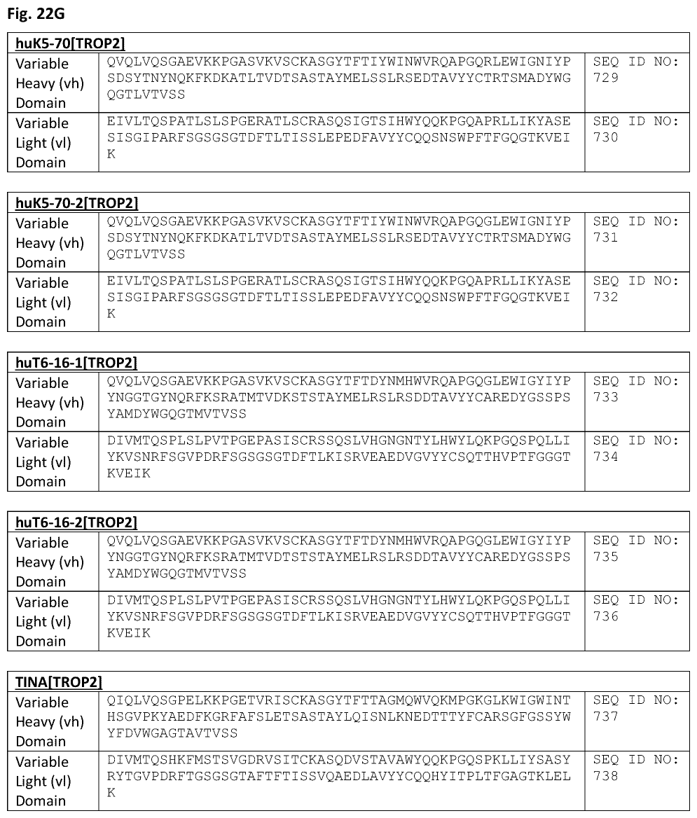

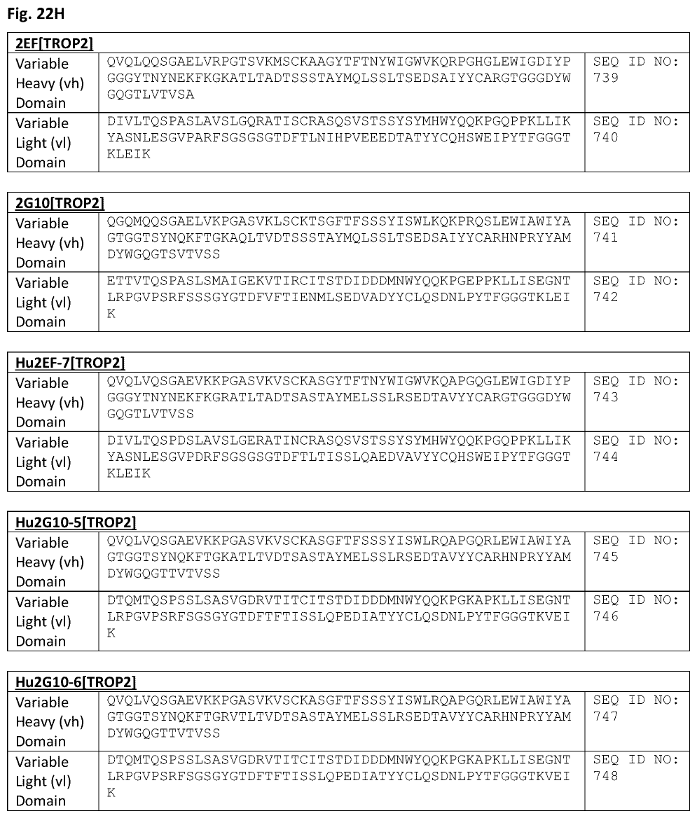

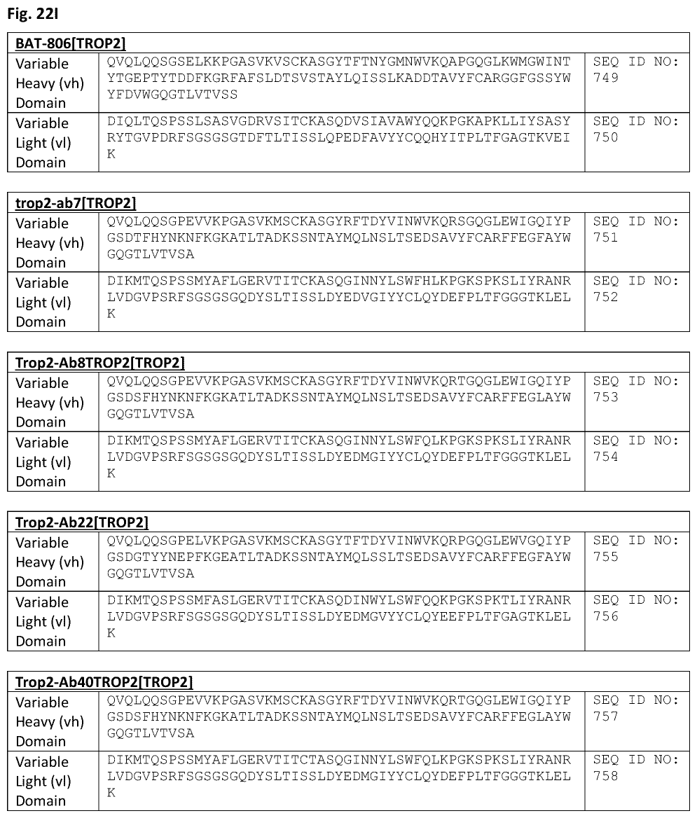

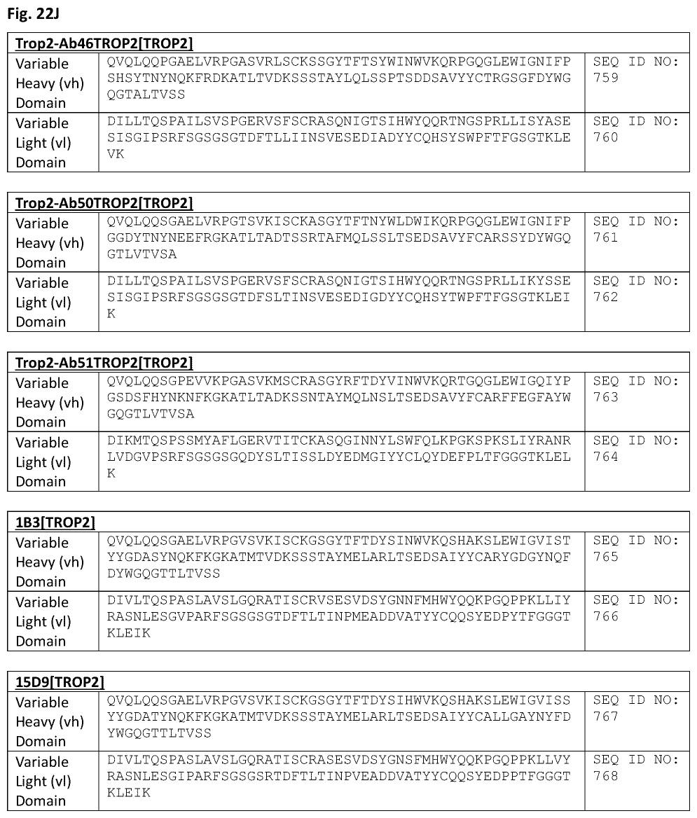

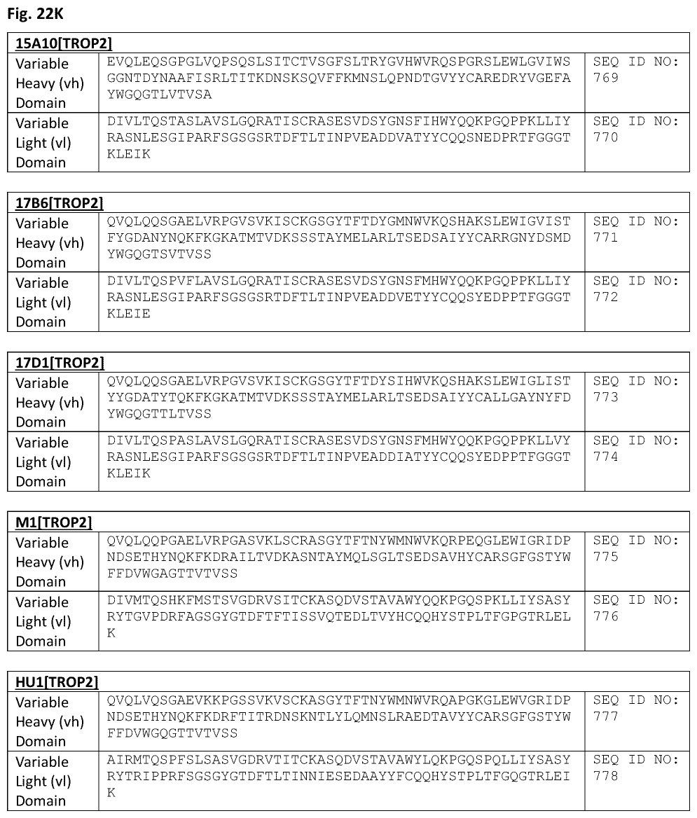

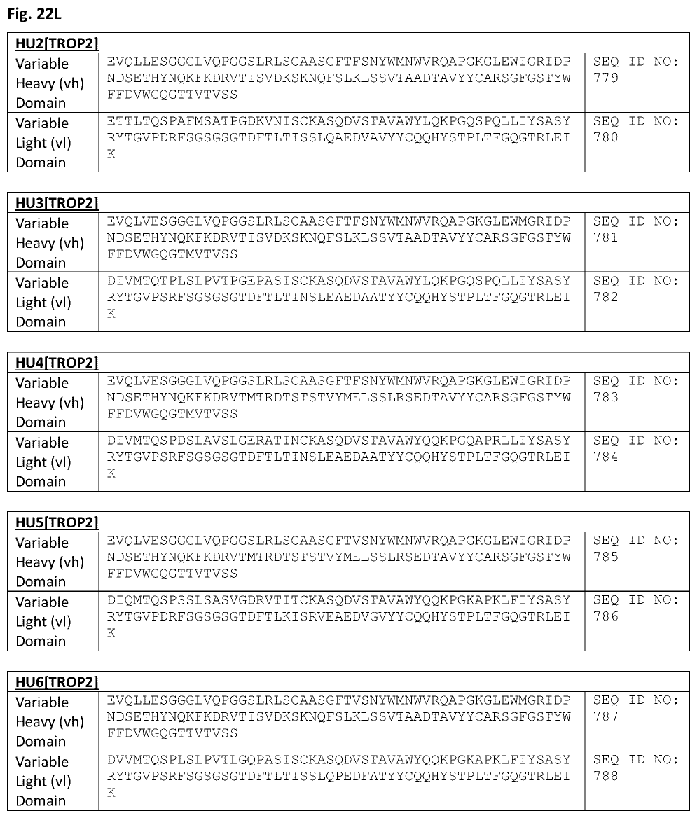

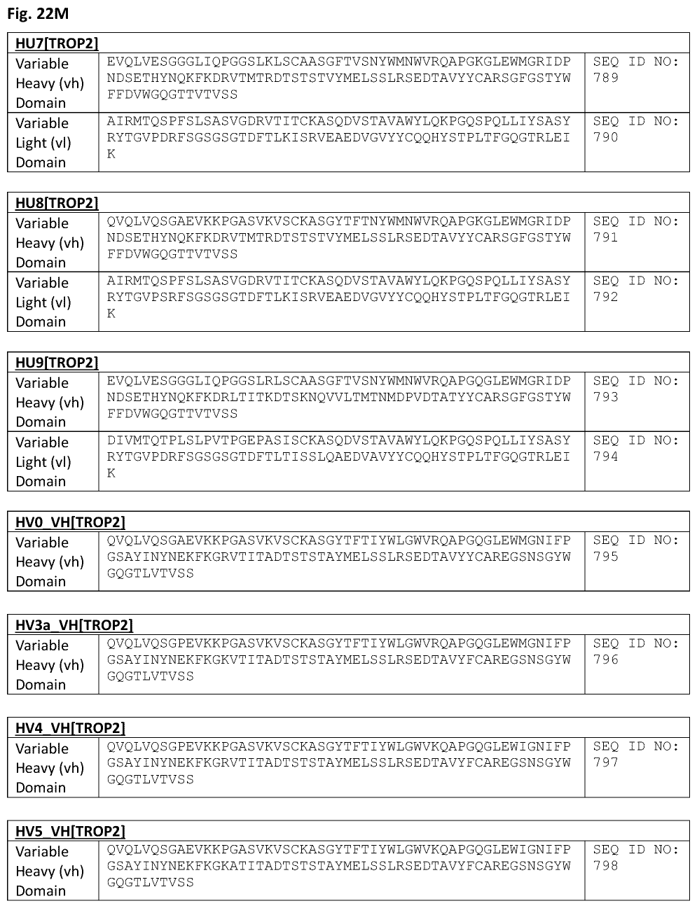

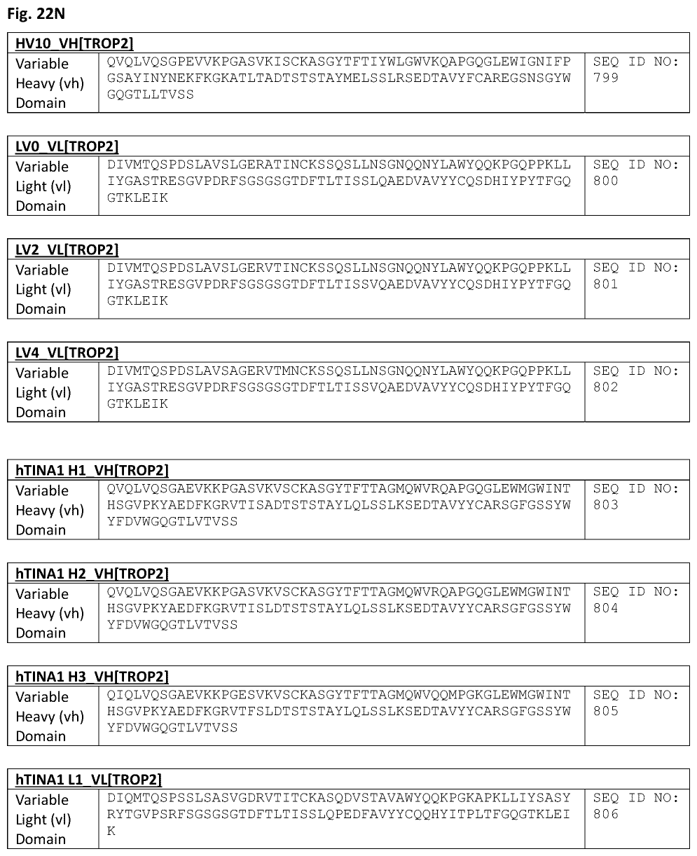

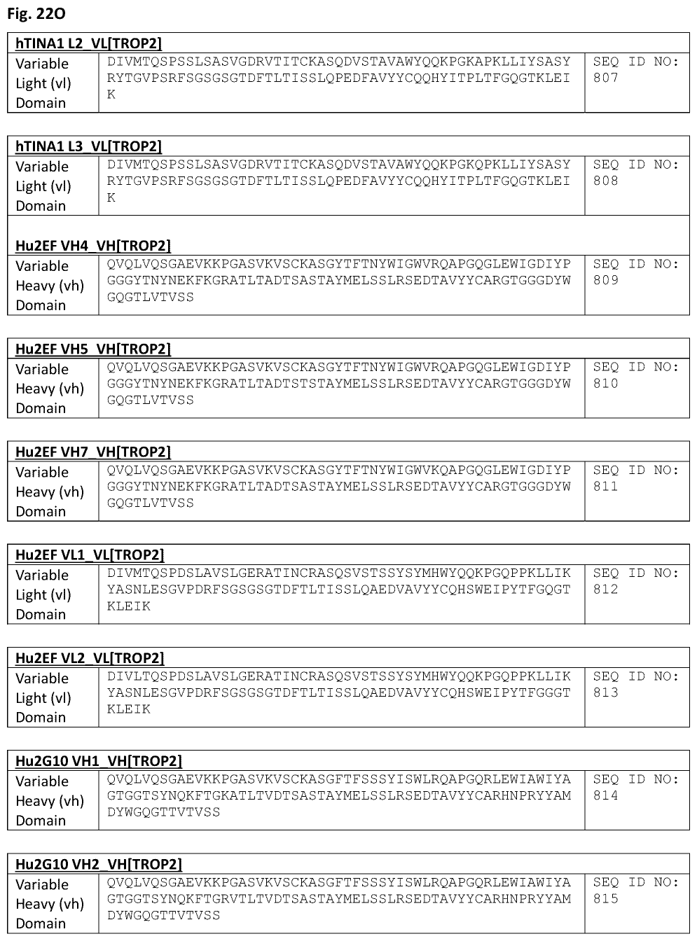

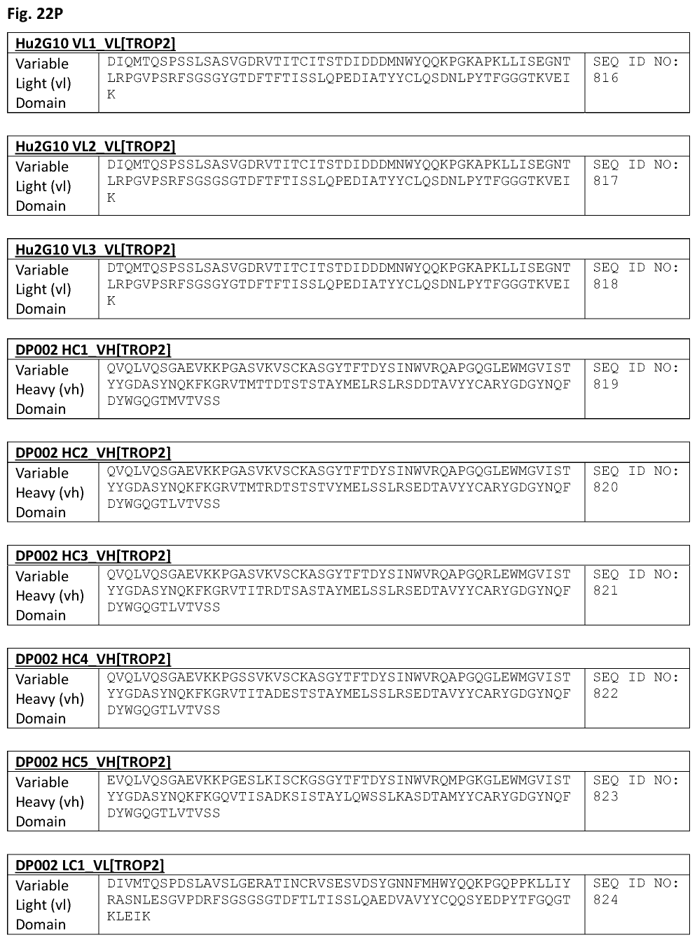

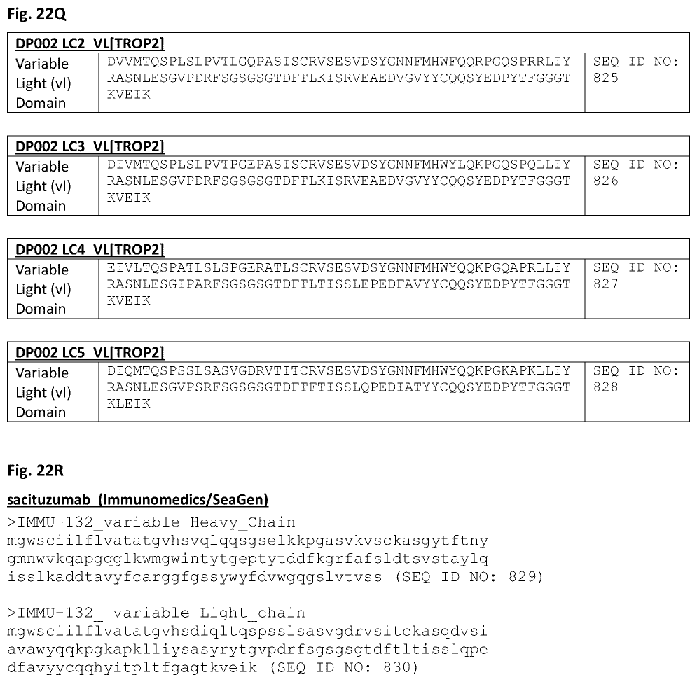

In some embodiments, the NKEs include an ABD that binds to the ECD of human Trop2. Suitable variable heavy VH and variable light VL domains that bind to the ECD of human Trop2 include, but are not limited to, VH/VL pairs selected from the group including: (i) SEQ ID NOs: 670 and 671, (ii) SEQ ID NOs: 672 and 673, (iii) SEQ ID NOs: 674 and 675, (iv) SEQ ID NOs: 676 and 677, (v) SEQ ID NOs: 678 and 679, (vi) SEQ ID NOs: 680 and 681, (vii) SEQ ID NOs: 682 and 683, (viii) SEQ ID NOs: 684 and 681, (ix) SEQ ID NOs: 685 and 686, (x) SEQ ID NOs: 687 and 688, (xi) SEQ ID NOs: 689 and 690, (xii) SEQ ID NOs: 691 and 692, (xiii) SEQ ID NOs: 693 and 694, (xiv) SEQ ID NOs: 695 and 696, (xv) SEQ ID NOs: 697 and 698, (xvi) SEQ ID NOs: 699 and 700, (xvii) SEQ ID NOs: 701 and 702, (xviii) SEQ ID NOs: 703 and 704, (xix) SEQ ID NOs: 705 and 706, (xx) SEQ ID NOs: 707 and 708, (xxi) SEQ ID NOs: 709 and 710, (xxii) SEQ ID NOs: 711 and 712, (xxiii) SEQ ID NOs: 713 and 714, (xxiv) SEQ ID NOs: 715 and 716, (xxv) SEQ ID NOs: 717 and 718, (xxvi) SEQ ID NOs: 719 and 720, (xxvii) SEQ ID NOs: 721 and 722, (xxviii) SEQ ID NOs: 723 and 724, (xxix) SEQ ID NOs: 725 and 726, (xxx) SEQ ID NOs: 727 and 728, (xxxi) SEQ ID NOs: 729 and 730, (xxxii) SEQ ID NOs: 731 and 732, (xxxiii) SEQ ID NOs: 733 and 734, (xxxiv) SEQ ID NOs: 735 and 736, (xxxv) SEQ ID NOs: 737 and 738, (xxxvi) SEQ ID NOs: 739 and 740, (xxxvii) SEQ ID NOs: 741 and 742, (xxxviii) SEQ ID NOs: 743 and 744, (xxxix) SEQ ID NOs: 745 and 746, (xl) SEQ ID NOs: 747 and 748, (xli) SEQ ID NOs: 749 and 750, (xlii) SEQ ID NOs: 751 and 752, (xliii) SEQ ID NOs: 753 and 754, (xliv) SEQ ID NOs: 755 and 756, (xlv) SEQ ID NOs: 757 and 758, (xlvi) SEQ ID NOs: 759 and 760, (xlvii) SEQ ID NOs: 761 and 762, (xlviii) SEQ ID NOs: 763 and 764, (xlix) SEQ ID NOs: 765 and 766, (1) SEQ ID NOs: 767 and 768, (li) SEQ ID NOs: 769 and 770, (lii) SEQ ID NOs: 771 and 772, (liii) SEQ ID NOs: 773 and 774, (liv) SEQ ID NOs: 775 and 776, (lv) SEQ ID NOs: 777 and 778, (lvi) SEQ ID NOs: 779 and 780, (lvii) SEQ ID NOs: 781 and 782, (lviii) SEQ ID NOs: 783 and 784, (lix) SEQ ID NOs: 785 and 786, (lx) SEQ ID NOs: 787 and 788, (lxi) SEQ ID NOs: 789 and 790, (lxii) SEQ ID NOs: 791 and 792, (lxiii) SEQ ID NOs: 793 and 794, (lxiv) SEQ ID NOs: 795 and 800, (lxv) SEQ ID NOs: 795 and 801, (lxvi) SEQ ID NOs: 795 and 802, (lxvii) SEQ ID NOs: 796 and 800, (lxviii) SEQ ID NOs: 796 and 801, (lxix) SEQ ID NOs: 796 and 802, (lxx) SEQ ID NOs: 797 and 800, (lxxi) SEQ ID NOs: 797 and 801, (lxxii) SEQ ID NOs: 797 and 802, (lxxiii) SEQ ID NOs: 798 and 800, (lxxiv) SEQ ID NOs: 798 and 801, (lxxv) SEQ ID NOs: 798 and 802, (lxxvi) SEQ ID NOs: 799 and 800, (lxxvii) SEQ ID NOs: 799 and 801, (lxxviii) SEQ ID NOs: 799 and 802, (lxxix) SEQ ID NOs: 803 and 806, (lxxx) SEQ ID NOs: 803 and 807, (lxxxi) SEQ ID NOs: 803 and 808, (lxxxii) SEQ ID NOs: 804 and 806, (lxxxiii) SEQ ID NOs: 804 and 807, (lxxxix) SEQ ID NOs: 804 and 808, (lxxxv) SEQ ID NOs: 805 and 806, (lxxxvi) SEQ ID NOs: 805 and 807, (lxxxvii) SEQ ID NOs: 805 and 808, (lxxxviii) SEQ ID NOs: 809 and 812, (lxxxix) SEQ ID NOs: 809 and 813, (xc) SEQ ID NOs: 810 and 812, (xci) SEQ ID NOs: 810 and 813, (xcii) SEQ ID NOs: 811 and 812, (xciii) SEQ ID NOs: 811 and 813, (xciv) SEQ ID NOs: 814 and 816, (xcv) SEQ ID NOs: 814 and 817, (xcvi) SEQ ID NOs: 814 and 818, (xcvii) SEQ ID NOs: 815 and 816, (xcviii) SEQ ID NOs: 815 and 817, (xcix) SEQ ID NOs: 815 and 818, (c) SEQ ID NOs: 819 and 824, (ci) SEQ ID NOs: 819 and 825, (cii) SEQ ID NOs: 819 and 826, (ciii) SEQ ID NOs: 819 and 827, (civ) SEQ ID NOs: 819 and 828, (cv) SEQ ID NOs: 820 and 824, (cvi) SEQ ID NOs: 820 and 825, (cvii) SEQ ID NOs: 820 and 826, (cviii) SEQ ID NOs: 820 and 827, (cix) SEQ ID NOs: 820 and 828, (cx) SEQ ID NOs: 821 and 824, (cxi) SEQ ID NOs: 821 and 825, (cxii) SEQ ID NOs: 821 and 826, (cxiii) SEQ ID NOs: 821 and 827, (cxiv) SEQ ID NOs: 821 and 828, (cxv) SEQ ID NOs: 822 and 824, (cxvi) SEQ ID NOs: 822 and 825, (cxvii) SEQ ID NOs: 822 and 826, (cxviii) SEQ ID NOs: 822 and 827, (cxix) SEQ ID NOs: 822 and 828, (cxx) SEQ ID NOs: 823 and 824, (cxxi) SEQ ID NOs: 823 and 825, (cxxii) SEQ ID NOs: 823 and 826, (cxxiii) SEQ ID NOs: 823 and 827, (cxxiv) SEQ ID NOs: 823 and 828, and (cxxv) SEQ ID NOs: 829 and 830, as shown in FIGS. 22A-22R, as well as those disclosed herein. Additionally, any sequence included above from FIG. 22 can also be individually included or excluded. It should be noted that these ABDs can be included in any of the formats described herein.

An anti-human Trop-2 antigen binding domain with VH and VL sequences selected from the group including: a) a VH having the amino acid sequence of positions 20 to 140 of SEQ ID NO:12 and a VL having the amino acid sequence of positions 21 to 129 of SEQ ID NO:18; b) a VH having the amino acid sequence of positions 20 to 140 of SEQ ID NO:14 and a VL having the amino acid sequence of positions 21 to 129 of SEQ ID NO:18; c) a VH having the amino acid sequence of positions 20 to 140 of SEQ ID NO: 14 and a VL having the amino acid sequence of positions 21 to 129 of SEQ ID NO:20; d) a VH having the amino acid sequence of positions 20 to 140 of SEQ ID NO:16 and a VL having the amino acid sequence of positions 21 to 129 of SEQ ID NO:22; wherein the sequences are as shown in U.S. Pat. No. 9,850,312, hereby expressly incorporated by reference for the sequences. Additionally, any sequence included above from U.S. Pat. No. 9,850,312 can also be individually included or excluded.

An anti-human Trop-2 antigen binding domain with VH and VL sequences selected from the group including: a) a VH having the amino acid sequence of SEQ ID NO: 172 and a VL having the amino acid sequence of SEQ ID NO:173; b) a VH having the amino acid sequence of SEQ ID NO:180 and a VL having the amino acid sequence of SEQ ID NO: 181; c) a VH having the amino acid sequence of SEQ ID NO:188 and a VL having the amino acid sequence of SEQ ID NO: 189; d) a VH having the amino acid sequence of SEQ ID NO:190 and a VL having the amino acid sequence of SEQ ID NO:191; and e) a VH having the amino acid sequence of SEQ ID NO:192 and a VL having the amino acid sequence of SEQ ID NO:193; wherein the sequences are as shown in Table 5 of WO2018/157147 hereby expressly incorporated by reference for the sequences. Additionally, any sequence included above from Table 5 of WO2018/157147 can also be individually included or excluded.

In some embodiments, the NKEs of the invention include at least one anti-Trop2 antigen binding domain, as described herein and in the Figures. Described herein is plurality of a means for binding the ECD of human Trop2.

11. CEA Antigen Binding Domains

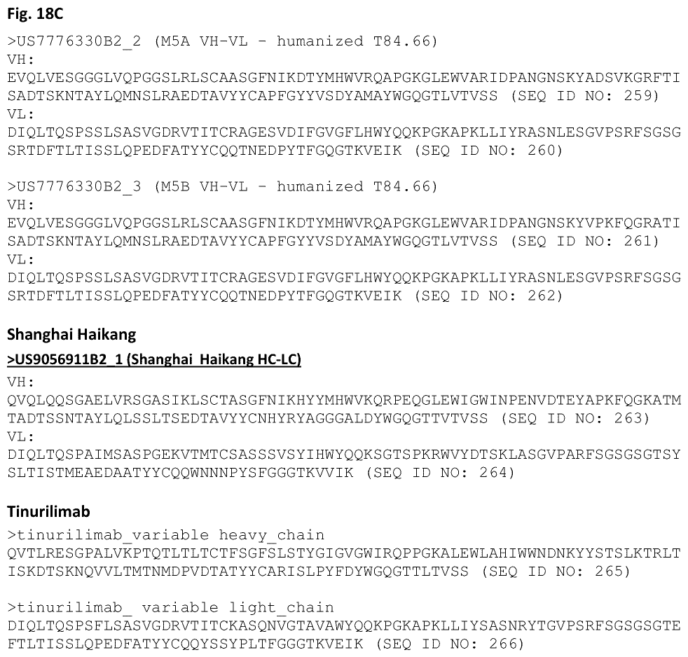

In some embodiments, the NKEs include an ABD that binds to the ECD of human CEA. Suitable variable heavy VH and variable light VL domains that bind to the ECD of human CEA include, but are not limited to, VH/VL pairs selected from the group including: (i) SEQ ID NOs: 239 and 240, (ii) SEQ ID NOs: 241 and 242, (iii) SEQ ID NOs: 243 and 244, (iv) SEQ ID NOs: 245 and 246, (v) SEQ ID NOs: 247 and 248, (vi) SEQ ID NOs: 249 and 250, (vii) SEQ ID NOs: 251 and 252, (viii) SEQ ID NOs: 253 and 254, (ix) SEQ ID NOs: 255 and 256, (x) SEQ ID NOs: 257 and 258, (xi) SEQ ID NOs: 259 and 260, (xii) SEQ ID NOs: 261 and 262, (xiii) SEQ ID NOs: 263 and 264, and (xiv) SEQ ID NOs: 265 and 266, as shown in FIGS. 18A-18C, as well as those disclosed herein. Additionally, any sequence included above from FIG. 18 can also be individually included or excluded. It should be noted that these ABDs can be included in any of the formats described herein.

An anti-human CEA antigen binding domain with sequences selected from the group including: a) a VH with the amino acid sequence shown in SEQ ID NO:195 and a VL with the amino acid sequence shown in SEQ ID NO:196; b) a VH with the amino acid sequence shown in SEQ ID NO:203 and a VL with the amino acid sequence shown in SEQ ID NO:204; and c) a VH with the amino acid sequence shown in SEQ ID NO:211 and a VL with the amino acid sequence shown in SEQ ID NO:212; wherein the sequences are as shown in Table 6 WO2018/157147, hereby expressly incorporated by reference for the sequences. Additionally, any sequence included above from Table 6 of WO2018/157147 can also be individually included or excluded.

In some embodiments, the NKEs of the invention include at least one anti-CEA antigen binding domain, as described herein and in the Figures. Described herein is a plurality of means for binding the ECD of human CEA.

12. CLDN18.2 Antigen Binding Domains

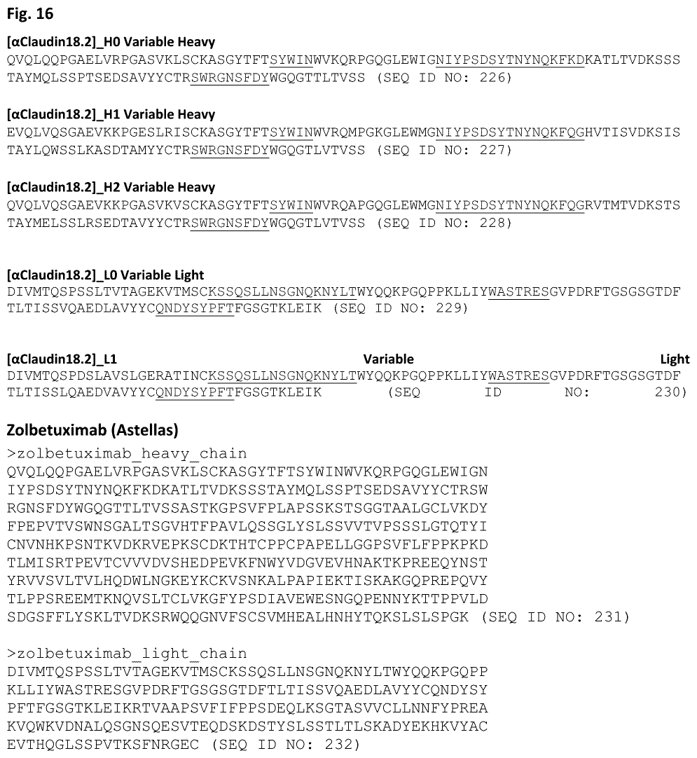

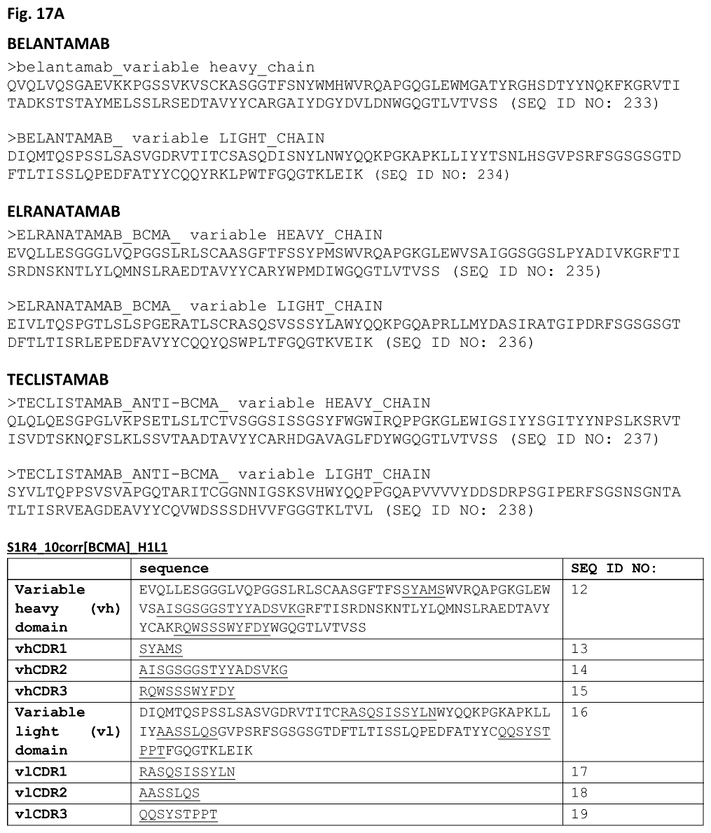

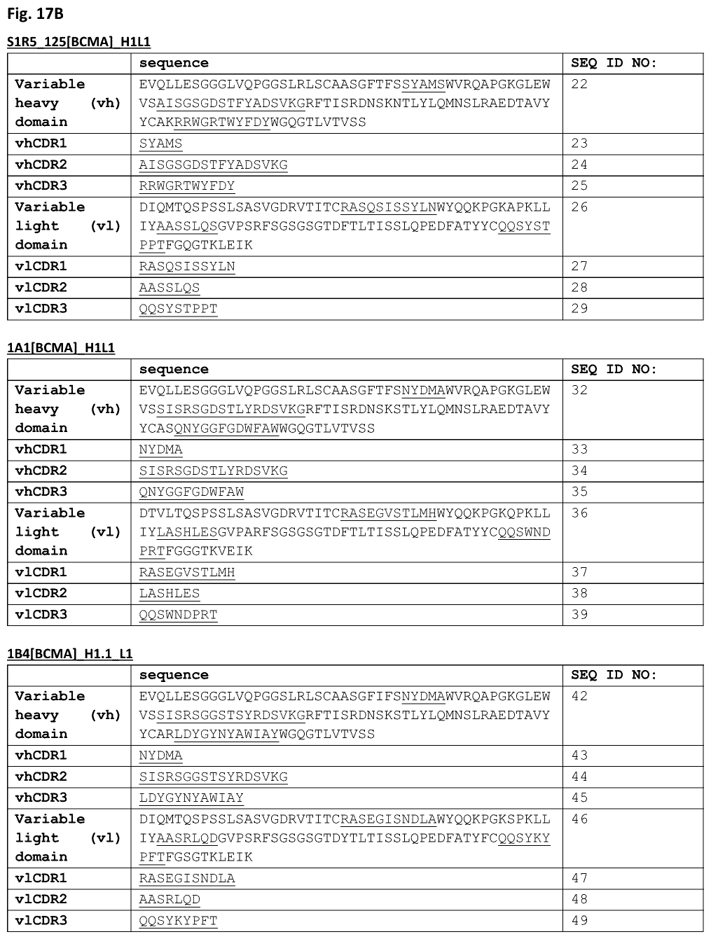

In some embodiments, the NKEs include an ABD that binds to the ECD of human CLDN18.2. Suitable variable heavy VH and variable light VL domains that bind to the ECD of human CLDN18.2 include, but are not limited to, VH/VL pairs selected from the group including: (i) SEQ ID NOs: 226 and 229, (ii) SEQ ID NOs: 226 and 230, (iii) SEQ ID NOs: 227 and 229, (iv) SEQ ID NOs: 227 and 230, (v) SEQ ID NOs: 228 and 229, (vi) SEQ ID NOs: 228 and 230, and (vii) SEQ ID NOs: 231 and 232, as shown in FIG. 16, as well as those disclosed herein. Additionally, any sequence included above from FIG. 16 can also be individually included or excluded. It should be noted that these ABDs can be included in any of the formats described herein.

An anti-human CLDN18.2 antigen binding domain comprising a variable heavy domain and a variable light domain selected from the group including: a) a V_{H} with the amino acid sequence depicted in SEQ ID NO:220 and a VL with the amino acid sequence depicted in SEQ ID NO:221; and b) a VL with the amino acid sequence depicted in SEQ ID NO:228 and a VL with the amino acid sequence depicted in SEQ ID NO:229, wherein the sequences are as depicted in WO2018/157147, hereby incorporated by reference for the sequences.

In some embodiments, the NKEs of the invention include at least one anti-CLDN18.2 antigen binding domain, as described herein and in the Figures. Described herein is a plurality of means for binding the ECD of human CLDN18.2.

13. BCMA Antigen Binding Domains

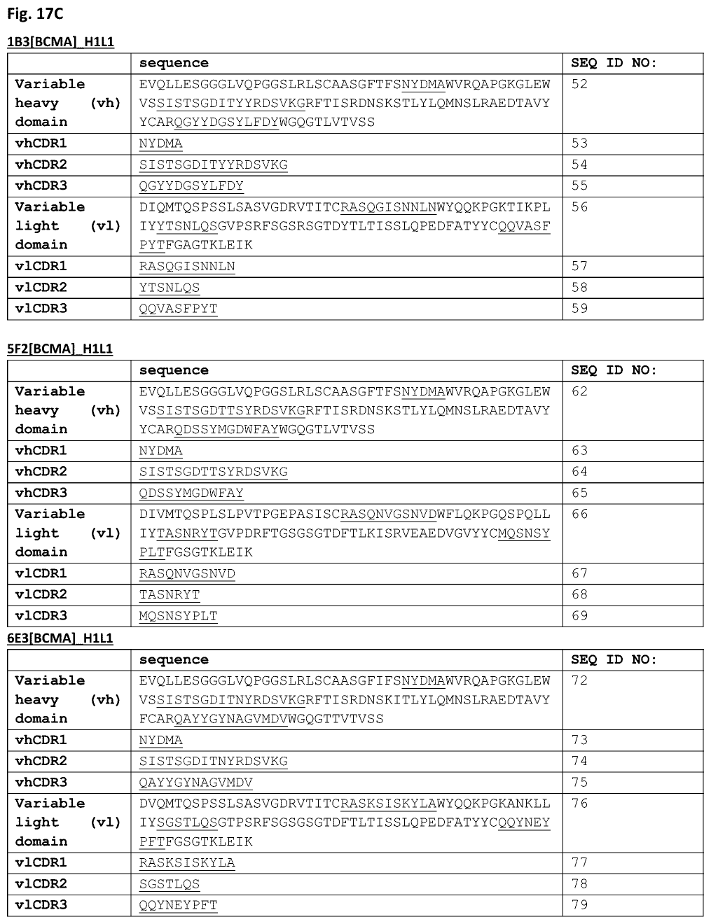

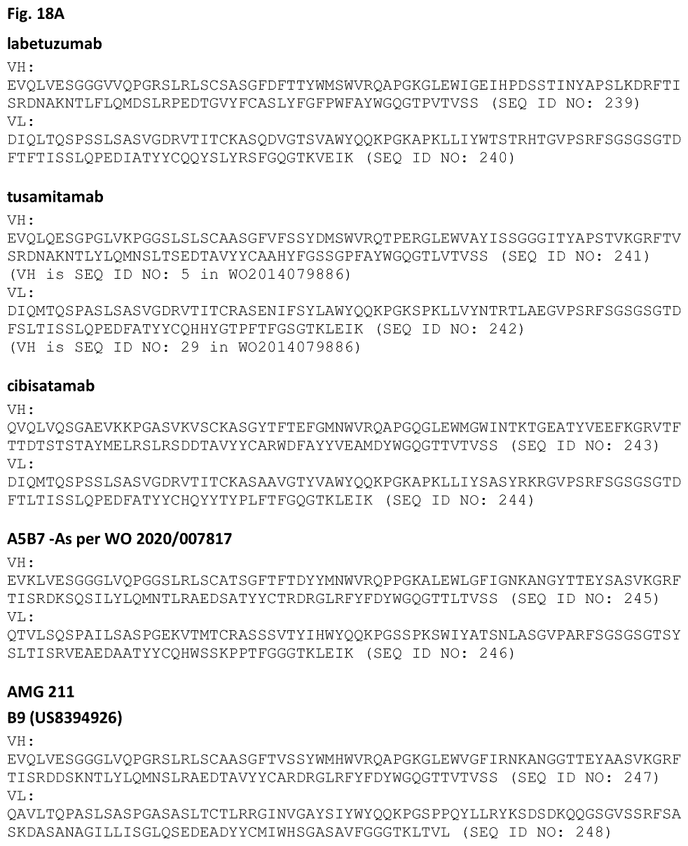

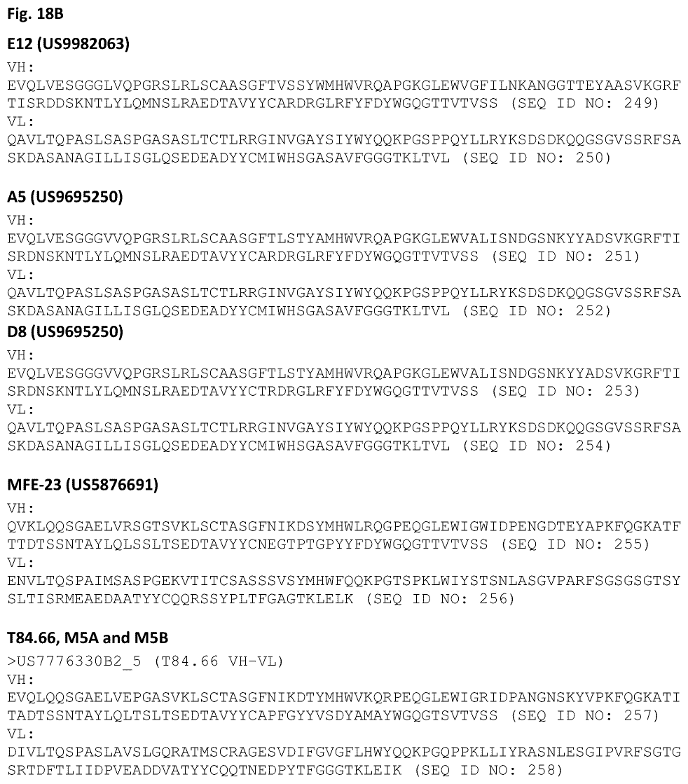

In some embodiments, the NKEs include an ABD that binds to the ECD of human BCMA. Suitable variable heavy VH and variable light VL domains that bind to the ECD of human BCMA include, but are not limited to, VH/VL pairs selected from the group including: (i) SEQ ID NOs: 233 and 234, (ii) SEQ ID NOs: 235 and 236, (iii) SEQ ID NOs: 237 and 238, (iv) SEQ ID NOs: 12 and 16, (v) SEQ ID NOs: 22 and 26, (vi) SEQ ID NOs: 32 and 36, (vii) SEQ ID NOs: 42 and 46, (viii) SEQ ID NOs: 52 and 56, (ix) SEQ ID NOs: 62 and 66, and (x) SEQ ID NOs: 72 and 76, as shown in FIGS. 17A-17C, as well as those disclosed herein. Additionally, any sequence included above from FIG. 17 can also be individually included or excluded. It should be noted that these ABDs can be included in any of the formats described herein.

Provided herein is an anti-human BCMA antigen binding domain having a VH with the amino acid sequence depicted in SEQ ID NO:65 and a VL with the amino acid sequence depicted in SEQ ID NO: 66, wherein the sequences as are depicted in WO2019/198501, hereby incorporated by reference for these sequences. Additionally, any sequence included above from WO2019/198501 can also be individually included or excluded.

Also provided herein, is an anti-BCMA antigen binding domain having a variable heavy and a variable light domain with amino acid sequences selected from the sets comprising: a) a VH with SEQ ID NO:1118 and a VL with SEQ ID NO:1119; b) a VH with SEQ ID NO:1123 and a VL with SEQ ID NO:1124; c) a VH with SEQ ID NO: 1127 and a VL with SEQ ID NO:1128; d) a VH with SEQ ID NO:1131 and a VL with SEQ ID NO:1132; e) a VH with SEQ ID NO:1153 and a VL with SEQ ID NO:1154; f) a VH with SEQ ID NO:79 and a VL with SEQ ID NO:94; g) a VH with SEQ ID NO:69 and a VL with SEQ ID NO:84; h) a VH with SEQ ID NO:70 and a VL with SEQ ID NO:85; i) a VH with SEQ ID NO:71 and a VL with SEQ ID NO:86; j) a VH with SEQ ID NO:72 and a VL with SEQ ID NO:87; k) a VH with SEQ ID NO:73 and a VL with SEQ ID NO:88; l) a VH with SEQ ID NO:74 and a VL with SEQ ID NO:89; m) a VH with SEQ ID NO:75 and a VL with SEQ ID NO:90; n) a VH with SEQ ID NO:76 and a VL with SEQ ID NO:91; o) a VH with SEQ ID N077: and a VL with SEQ ID NO:92; p) a VH with SEQ ID NO:78 and a VL with SEQ ID NO:93; q) a VH with SEQ ID NO:80 and a VL with SEQ ID NO:95; r) a VH with SEQ ID NO:81 and a VL with SEQ ID NO:96; s) a VH with SEQ ID NO:82 and a VL with SEQ ID NO:97; t) a VH with SEQ ID NO:83 and a VL with SEQ ID NO:98; u) a VH with SEQ ID NO:171 and a VL with SEQ ID NO:192; v) a VH with SEQ ID NO:172 and a VL with SEQ ID NO:193; w) a VH with SEQ ID NO: 173 and a VL with SEQ ID NO:194; x) a VH with SEQ ID NO:174 and a VL with SEQ ID NO:195; y) a VH with SEQ ID NO:175 and a VL with SEQ ID NO:196; z) a VH with SEQ ID NO:176 and a VL with SEQ ID NO:197; aa) a VH with SEQ ID NO:177 and a VL with SEQ ID NO:198; bb) a VH with SEQ ID NO:178 and a VL with SEQ ID NO:199; cc) a VH with SEQ ID NO:179 and a VL with SEQ ID NO:200; dd) a VH with SEQ ID NO:180 and a VL with SEQ ID NO:201; ee) a VH with SEQ ID NO:181 and a VL with SEQ ID NO:202; ff) a VH with SEQ ID NO:182 and a VL with SEQ ID NO:203; gg) a VH with SEQ ID NO:183 and a VL with SEQ ID NO:204; hh) a VH with SEQ ID NO:184 and a VL with SEQ ID NO:205; ii) a VH with SEQ ID NO:185 and a VL with SEQ ID NO:206; jj) a VH with SEQ ID NO:186 and a VL with SEQ ID NO:207; kk) a VH with SEQ ID NO:187 and a VL with SEQ ID NO:208; ll) a VH with SEQ ID NO:188 and a VL with SEQ ID NO:209; mm) a VH with SEQ ID NO:189 and a VL with SEQ ID NO:210; nn) a VH with SEQ ID NO:190 and a VL with SEQ ID NO:211; and oo) a VH with SEQ ID NO:191 and a VL with SEQ ID NO:212; wherein the sequences as are depicted in US2020/0179511, hereby incorporated by reference for these sequences. Additionally, any sequence included above from US2020/0179511 can also be individually included or excluded.

In some embodiments, the NKEs of the invention include at least one anti-BCMA antigen binding domain, as described herein and in the Figures. Described herein is a plurality of means for binding the ECD of human BCMA.

14. DLL3 Antigen Binding Domains

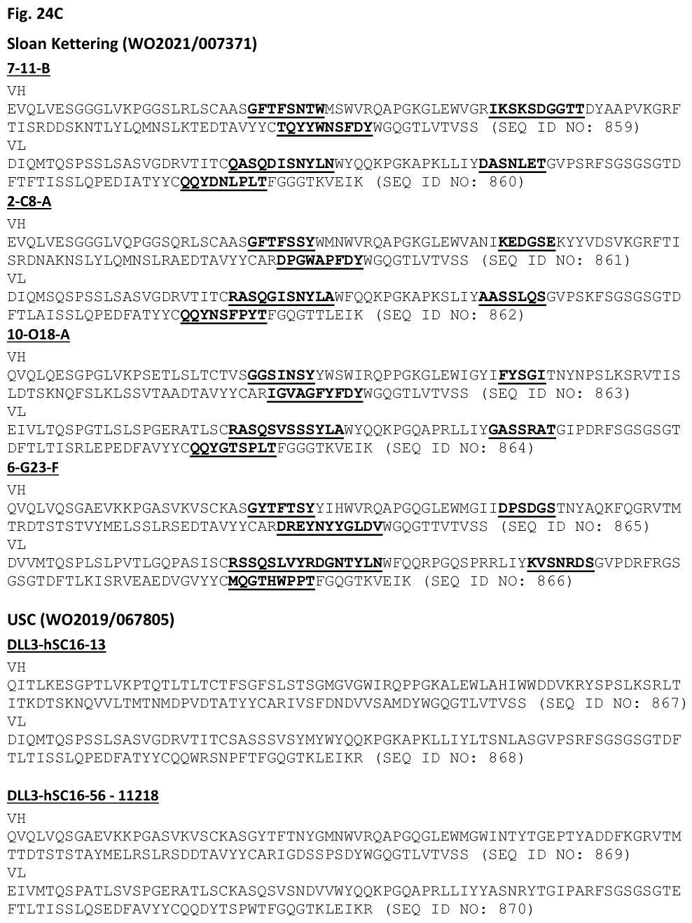

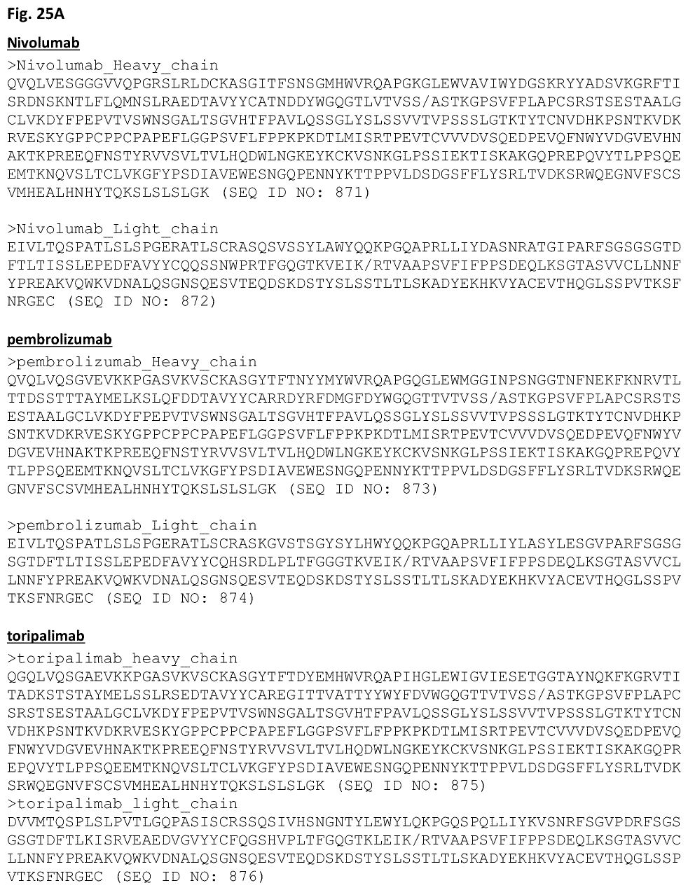

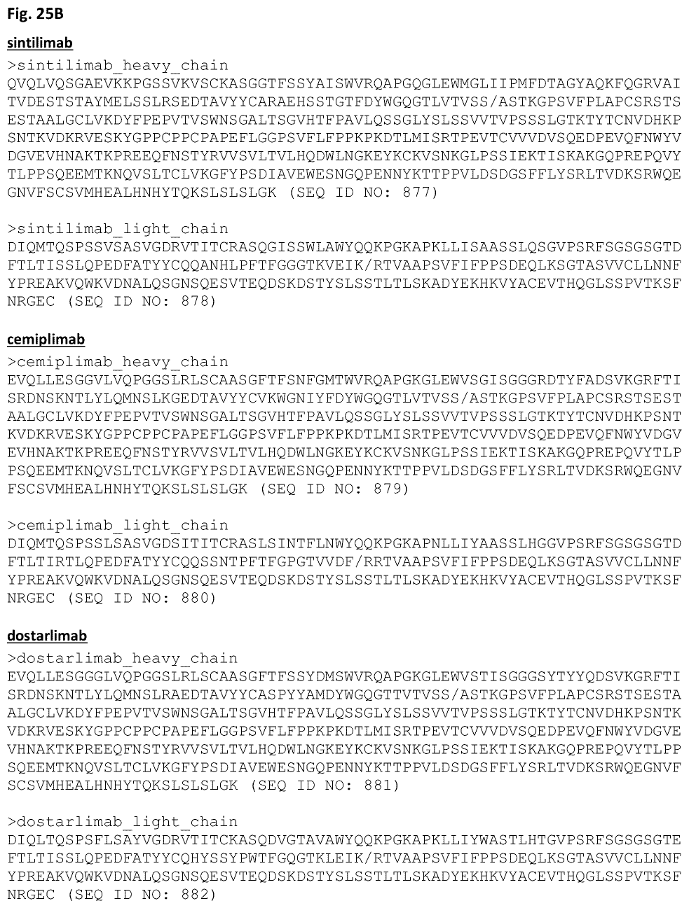

In some embodiments, the NKEs include an ABD that binds to the ECD of human DLL3. Suitable variable heavy VH and variable light VL domains that bind to the ECD of human DLL3 include, but are not limited to, VH/VL pairs selected from the group including: (i) SEQ ID NOs: 837 and 838, (ii) SEQ ID NOs: 839 and 840, (iii) SEQ ID NOs: 841 and 842, (iv) SEQ ID NOs: 843 and 844, (v) SEQ ID NOs: 845 and 846, (vi) SEQ ID NOs: 847 and 848, (vii) SEQ ID NOs: 849 and 850, (viii) SEQ ID NOs: 851 and 852, (ix) SEQ ID NOs: 853 and 854, (x) SEQ ID NOs: 855 and 856, (xi) SEQ ID NOs: 857 and 858, (xii) SEQ ID NOs: 859 and 860, (xiii) SEQ ID NOs: 861 and 862, (xiv) SEQ ID NOs: 863 and 864, (xv) SEQ ID NOs: 865 and 866, (xvi) SEQ ID NOs: 867 and 868, and (xvii) SEQ ID NOs: 869 and 870, as shown in FIGS. 24A-24C, as well as those disclosed herein. Additionally, any sequence included above from FIG. 24 can also be individually included or excluded. It should be noted that these ABDs can be included in any of the formats described herein.

Provided herein is an anti-human DLL3 antigen binding domain with sequences selected from the group including: (a) a VH with the amino acid sequence shown in SEQ ID NO:38 and a VL with the amino acid sequence shown in SEQ ID NO:37; (b) a VH with the amino acid sequence shown in SEQ ID NO:40 and a VL with the amino acid sequence shown in SEQ ID NO:39; (c) a VH with the amino acid sequence shown in SEQ ID NO:42 and a VL with the amino acid sequence shown in SEQ ID NO:41; (d) a VH with the amino acid sequence shown in SEQ ID NO:44 and a VL with the amino acid sequence shown in SEQ ID NO:43; (e) a VH with the amino acid sequence shown in SEQ ID NO:46 and a VL with the amino acid sequence shown in SEQ ID NO:45; (f) a VH with the amino acid sequence shown in SEQ ID NO:48 and a VL with the amino acid sequence shown in SEQ ID NO:47; (g) a VH with the amino acid sequence shown in SEQ ID NO:206 and a VL with the amino acid sequence shown in SEQ ID NO:205; (h) a VH with the amino acid sequence shown in SEQ ID NO:208 and a VL with the amino acid sequence shown in SEQ ID NO:207; (i) a VH with the amino acid sequence shown in SEQ ID N0210: and a VL with the amino acid sequence shown in SEQ ID NO:209; (j) a VH with the amino acid sequence shown in SEQ ID NO:212 and a VL with the amino acid sequence shown in SEQ ID NO:211; (k) a VH with the amino acid sequence shown in SEQ ID NO:214 and a VL with the amino acid sequence shown in SEQ ID NO:213; (1) a VH with the amino acid sequence shown in SEQ ID NO:216 and a VL with the amino acid sequence shown in SEQ ID NO:215; (m) a VH with the amino acid sequence shown in SEQ ID NO:218 and a VL with the amino acid sequence shown in SEQ ID NO:217; (n) a VH with the amino acid sequence shown in SEQ ID NO:220 and a VL with the amino acid sequence shown in SEQ ID NO:219; (o) a VH with the amino acid sequence shown in SEQ ID NO:222 and a VL with the amino acid sequence shown in SEQ ID NO:221; (p) a VH with the amino acid sequence shown in SEQ ID NO:224 and a VL with the amino acid sequence shown in SEQ ID NO:223; (q) a VH with the amino acid sequence shown in SEQ ID NO:226 and a VL with the amino acid sequence shown in SEQ ID NO:225; and (r) a VH with the amino acid sequence shown in SEQ ID NO:208 and a VL with the amino acid sequence shown in SEQ ID NO:227; wherein the sequences are as shown in WO2019/234220, hereby expressly incorporated by reference for the sequences. Additionally, any sequence included above from WO2019/234220 can also be individually included or excluded.

Provided herein is anti-human DLL3 antigen binding domain with sequences selected from the group including: (a) a VH with the amino acid sequence shown in SEQ ID NO:232 and a VL with the amino acid sequence shown in SEQ ID NO:236; (b) a VH with the amino acid sequence shown in SEQ ID NO:264 and a VL with the amino acid sequence shown in SEQ ID NO:265; (c) a VH with the amino acid sequence shown in SEQ ID NO:266 and a VL with the amino acid sequence shown in SEQ ID NO:267; and (d) a VH with the amino acid sequence shown in SEQ ID NO:268 and a VL with the amino acid sequence shown in SEQ ID NO:269; wherein the sequences are as shown in WO2021/200898, hereby expressly incorporated by reference for the sequences. Additionally, any sequence included above from WO2021/200898 can also be individually included or excluded.

In some embodiments, the NKEs of the invention include at least one anti-DLL3 antigen binding domain, as described herein and in the Figures. Described herein is a plurality of means for binding the ECD of human DLL3.

15. PD-1 Antigen Binding Domains

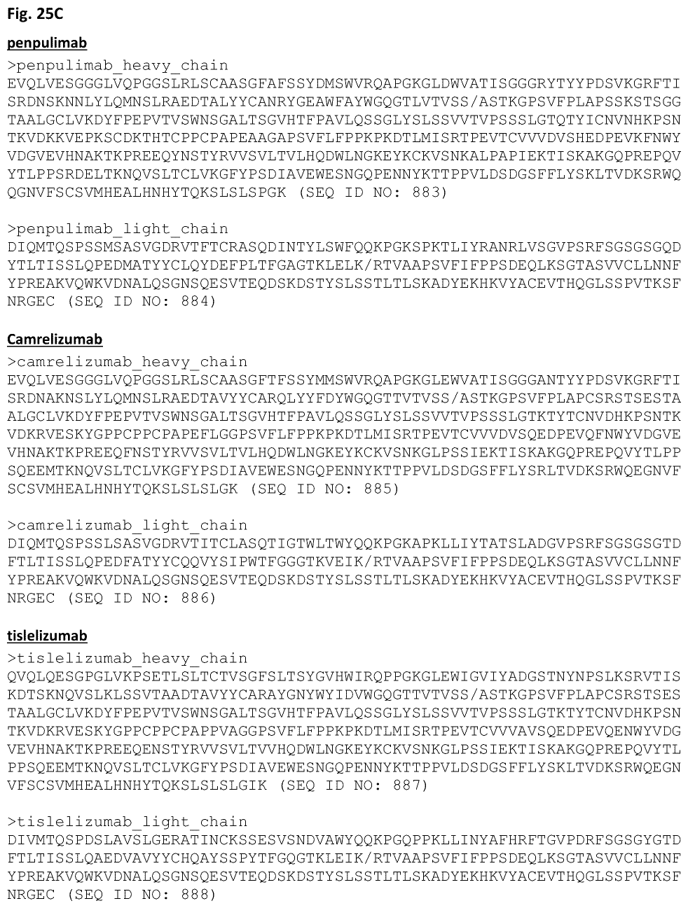

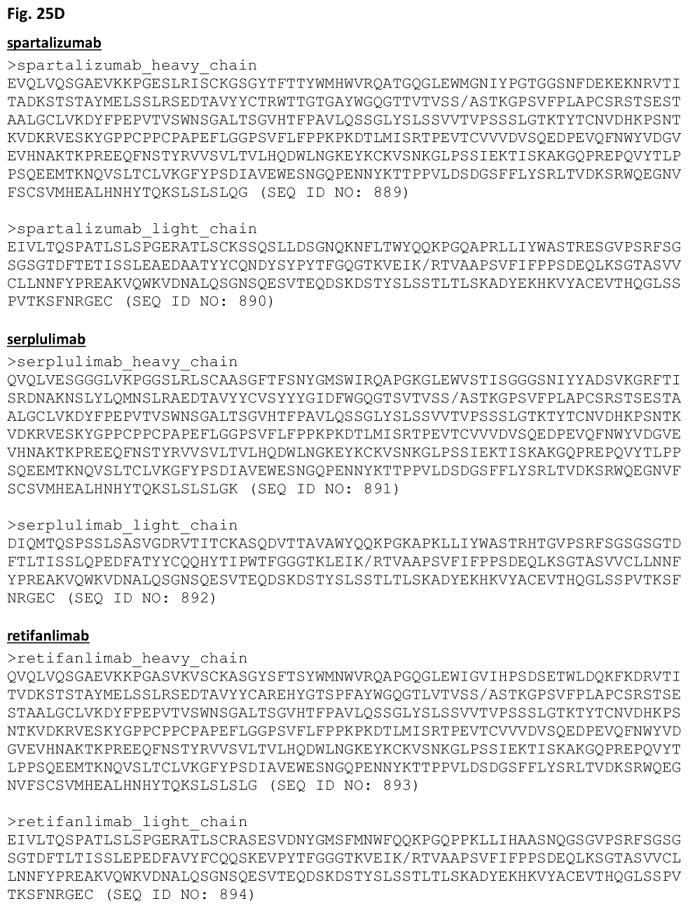

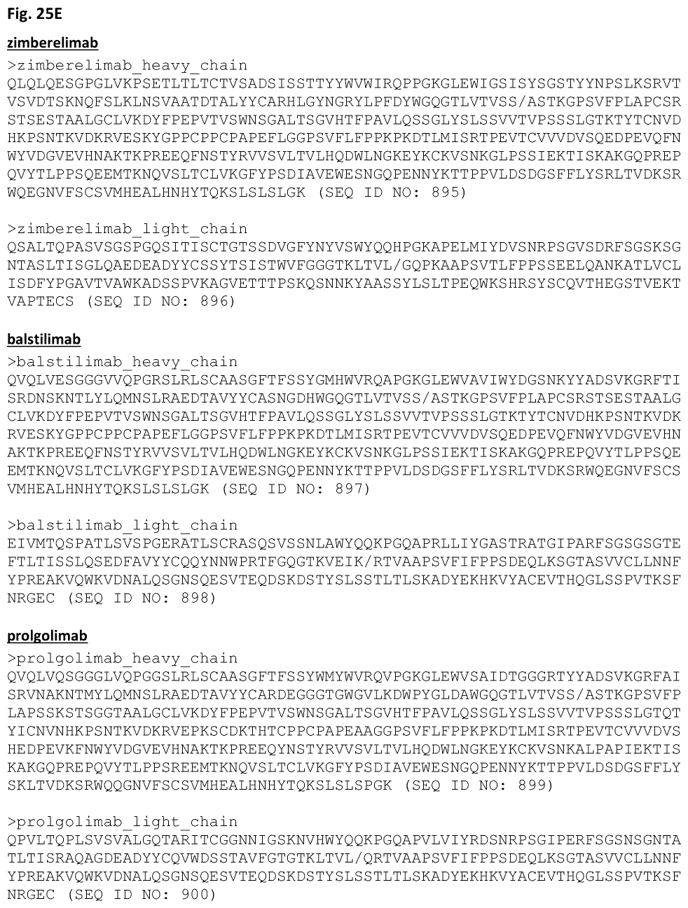

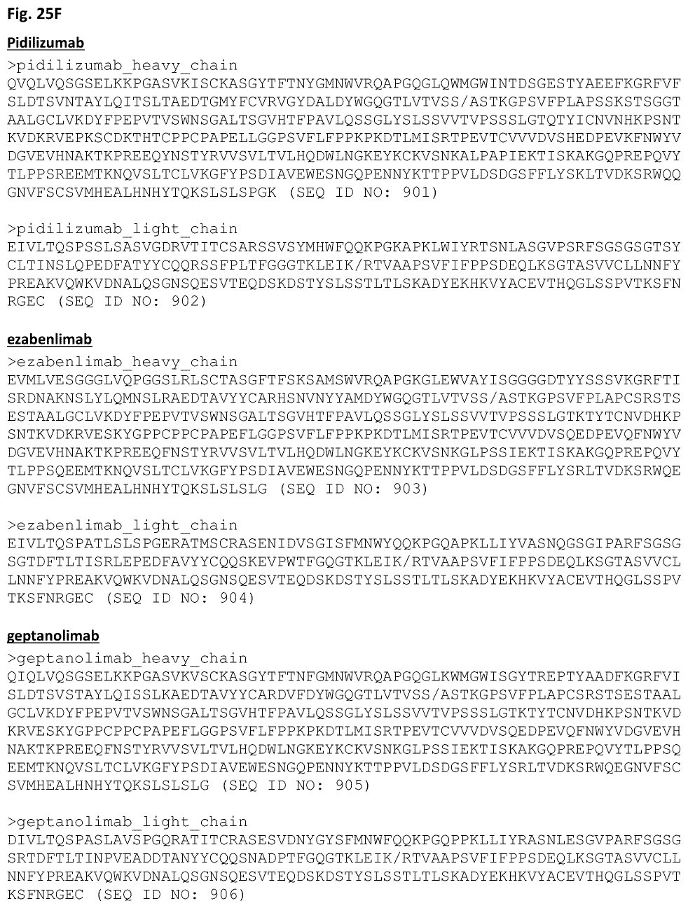

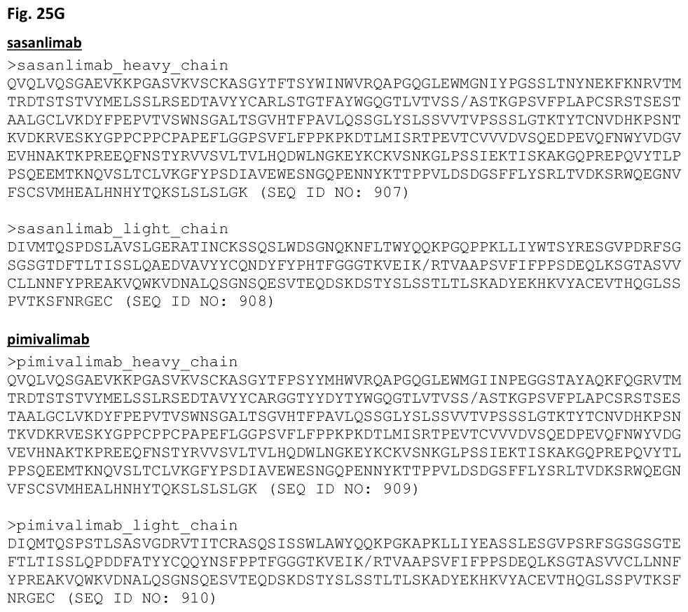

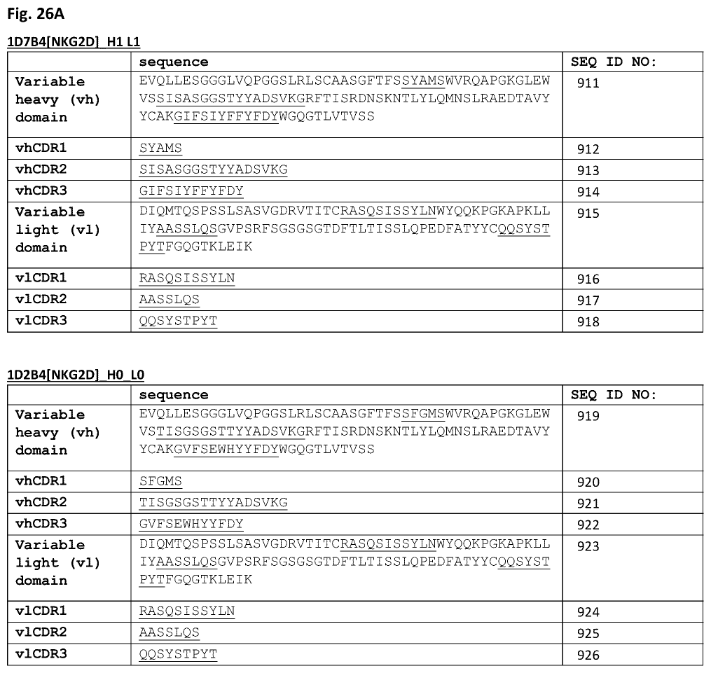

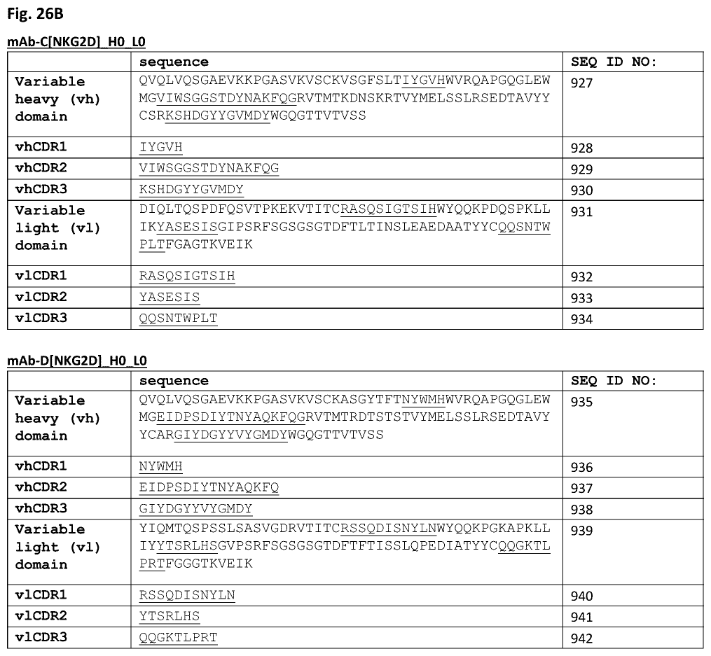

In some embodiments, the NKEs include an ABD that binds to the ECD of human PD-1. Suitable variable heavy VH and variable light VL domains that bind to the ECD of human PD-1 include, but are not limited to, VH/VL pairs selected from the group including: (i) SEQ ID NOs: 871 and 872, (ii) SEQ ID NOs: 873 and 874, (iii) SEQ ID NOs: 875 and 876, (iv) SEQ ID NOs: 877 and 878, (v) SEQ ID NOs: 879 and 880, (vi) SEQ ID NOs: 881 and 882, (vii) SEQ ID NOs: 883 and 884, (viii) SEQ ID NOs: 885 and 886, (ix) SEQ ID NOs: 887 and 888, (x) SEQ ID NOs: 889 and 890, (xi) SEQ ID NOs: 891 and 892, (xii) SEQ ID NOs: 893 and 894, (xiii) SEQ ID NOs: 895 and 896, (xiv) SEQ ID NOs: 897 and 898, (xv) SEQ ID NOs: 899 and 900, (xvi) SEQ ID NOs: 901 and 902, (xvii) SEQ ID NOs: 903 and 904, (xviii) SEQ ID NOs: 905 and 906, (xix) SEQ ID NOs: 907 and 908, and (xx) SEQ ID NOs: 909 and 910, as shown in FIGS. 25A-25G, as well as those disclosed herein. Additionally, any sequence included above from FIG. 25 can also be individually included or excluded. It should be noted that these ABDs can be included in any of the formats described herein.

In some embodiments, the NKEs of the invention include at least one anti-PD-1 antigen binding domain, as described herein and in the Figures. Described herein is a plurality of means for binding the ECD of human PD-1.

16. ANO1 Antigen Binding Domains

In some embodiments, the TTA binds to the ECD of human ANO1. In this case, the amino acid sequence of the variable heavy domain is shown in SEQ ID NO:97 of Table 3 of WO2018/157147 and the sequence of the variable light domain is shown in SEQ ID NO:98, the sequences of which are hereby incorporated by reference.

In some embodiments, the NKEs of the invention include at least one anti-ANO1 antigen binding domain, as described herein and in the Figures. Described herein is a means for binding the ECD of human ANO1 antigen.

17. CD22 Antigen Binding Domains

In some embodiments, the TTA binds to the ECD of human CD22. In some embodiments, the amino acid sequence of the variable heavy domain is shown in SEQ ID NO:30 and the variable light domain is shown in SEQ ID NO:28 of U.S. Pat. No. 7,355,011, the sequences of which are hereby incorporated by reference. In some embodiments, the CD22 ABD comprises the variable heavy and variable light domains of an antibody selected from the group including epratuzumab or moxetumomab.

In some embodiments, the NKEs of the invention include at least one anti-CD22 antigen binding domain, as described herein and in the Figures. Described herein is a means for binding the ECD of human CD22 antigen.

18. CD38 Antigen Binding Domains

In some embodiments, the TTA binds to the ECD of human CD38. In some embodiments, the CD22 ABD comprises the variable heavy and variable light domains of an antibody selected from the group including daratumumab, isatuximab, felzartamab, or mezagitamab.

In some embodiments, the NKEs of the invention include at least one anti-CD38 antigen binding domain, as described herein and in the Figures. Described herein is a means for binding the ECD of human CD38 antigen.

C. Linkers

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIGS. 5-7 (see, e.g., SEQ ID NOs: 1-11, 20, 21, 30, 31, 40, 41, 50, 51, 81-92, 94-120). In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n (SEQ ID NO: 120), (GSGGS)n (SEQ ID NO: 117), (GGGGS)n (SEQ ID NO: 118), and (GGGS)n (SEQ ID NO: 119), where n is an integer of at least one up to 10 (and generally from 3 to 4), glycine-alanine polymers, alanine-serine polymers, and other flexible linkers, some of which are shown in the Figures. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of C_{L}/CH1 domain but not all residues of C_{L}/CH1 domain; for example, the first 5-12 amino acid residues of the C_{L}/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker,” used to link any two domains as outlined herein together. For example, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker) (as are generally shown in the Figures). While any suitable linker can be used, many embodiments utilize a glycine-serine polymer as the domain linker, including for example (GS)n (SEQ ID NO: 120), (GSGGS)n (SEQ ID NO: 117), (GGGGS)n (SEQ ID NO: 118), and (GGGS)n (SEQ ID NO: 119), where n is an integer of at least one up to 10 (and generally from 3 to 4) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in FIGS. 5-7 (see, e.g., SEQ ID NOs: 1-11, 20, 21, 30, 31, 40, 41, 50, 51, 81-92, 94-120).

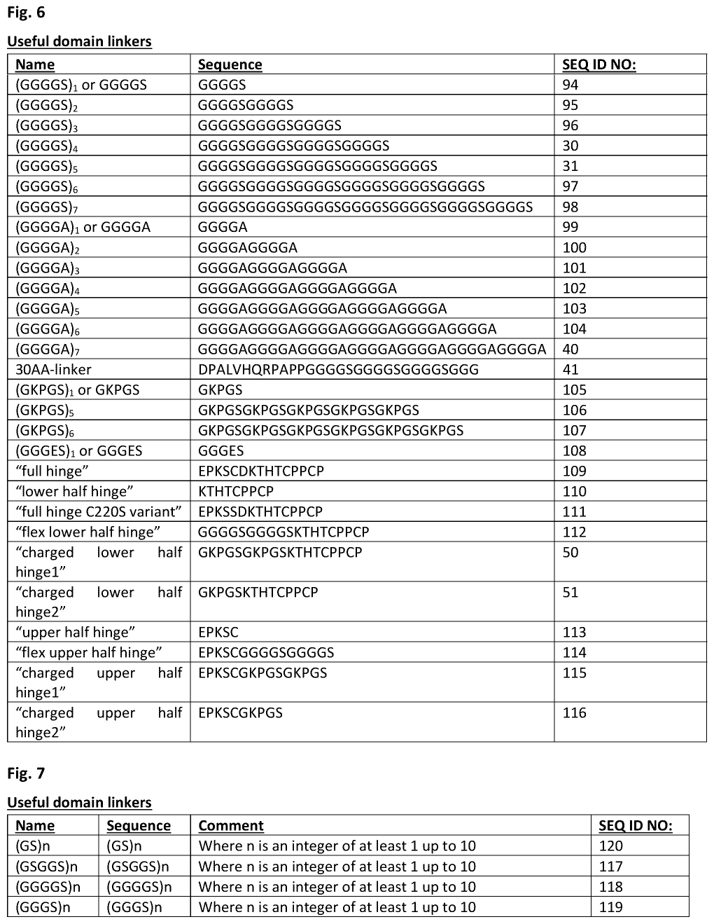

With particular reference to the domain linker used to attach the scFv domain to the Fc domain in the “2+1” format, there are several domain linkers that find particular use, including “full hinge C220S variant,” “flex half hinge,” “charged half hinge 1,” and “charged half hinge 2” as shown in FIG. 6 (see, e.g., SEQ ID NOs: 50, 51, and 111-116).

In some embodiments, the linker is a “scFv linker,” used to covalently attach the V_{H} and V_{L} domains as discussed herein. In many cases, the scFv linker is a charged scFv linker, a number of which are shown in FIG. 5 (see, e.g., SEQ ID NOs: 1-11, 20, 21, and 81-92). Accordingly, in some embodiments, the antibodies described herein further provide charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the antibodies described herein as well, and thus those included in the Figures can be used in any embodiment herein where a linker is utilized.

### VI. Antibodies

The antibodies provided herein include different antibody domains as is more fully described below. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains. It should be noted that the term “Fc domain” includes both the CH2-CH3 (and optionally the hinge, hinge-CH2-CH3) of a single monomer, as well as the dimer of two Fc domains that self-assemble. That is, the heavy chain of an antibody has an Fc domain that is a single polypeptide, while the assembled bispecific antibody has an Fc domain that contains two polypeptides. Various antibody domains included in the bispecific, heterodimeric antibodies are more fully described below.

In particular, the formats depicted in FIG. 8 are usually referred to as “heterodimeric antibodies,” meaning that the protein has at least two associated Fc sequences self-assembled into a heterodimeric Fc domain and at least two Fv regions, whether as Fabs or as scFvs.

A. Chimeric and Humanized Antibodies

In certain embodiments, the antibodies described herein comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI, and ablation variants herein; that is, the number of variants is generally low, prior to the introduction of the variants described herein). In certain cases, the humanized antibody may display no more than 5, or even no more than 4, 3, 2, or 1 amino acid difference from the amino acid sequence encoded by the germline immunoglobulin gene (again, prior to the introduction of any skew, pI, and ablation variants herein; that is, the number of variants is generally low, prior to the introduction of the variants described herein). In some embodiments, the amino acid differences are in one or more of the 6 CDRs. In some embodiments, the amino acid differences are in a V_{H} and/or V_{L} framework region.

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

B. Heterodimeric Antibodies as Natural Killer Cell Engagers

In one aspect, provided herein are Natural Killer cell Engager (NKE). Generally, NKEs are multifunctional molecules that target activating or inhibitory receptors (in particular the ECD of such receptors) expressed on the surface of NK cells, optionally bind to tumor target antigens (in particular the ECD of such antigens) and activate Fc gamma receptors expressed on effector cells of a subject's immune system. The different domains of an NKE including the NK cell antigen binding domain, the tumor target antigen binding domain and the Fc domains can modulate its activity and function.

Described below are useful variant Fc domains that include amino acid modifications (i.e., substitutions, insertions, or deletions) to enhance FcγR-mediated cytotoxicity, increase serum half-life, and facilitate the self-assembly and/or purification of the heterodimeric antibodies provided. Also, exemplary antibodies, including multispecific antibodies, that bind to NK cells can include such variant Fc domains described below and set forth in the figures.

1. Fc Domain Variants for Increasing Antibody-Dependent Cellular Cytotoxicity (ADCC)

There are a number of useful Fc substitutions that can be made to alter binding to one or more of the FcγR receptors. Substitutions that result in increased binding (or in some cases, decreased binding) can be useful. For example, it is known that increased binding to FcγRIIIa can result in increased ADCC (antibody dependent cell-mediated cytotoxicity). In some instances, decreased binding to FcγRIIb (an inhibitory receptor) can be beneficial as well. Amino acid substitutions that find use in the present invention include those listed in U.S. Ser. No. 11/124,620 (particularly FIG. 41), U.S. Ser. Nos. 11/174,287, 11/396,495, 11/538,406, all of which are expressly incorporated herein by reference in their entirety and specifically for the variants disclosed therein.

In some embodiments, provided herein are bispecific antibodies containing Fc variants that increase antibody-dependent cellular cytotoxicity (ADCC; the cell-mediated reaction wherein nonspecific cytotoxic cells that express FcγRs recognize bound antibody on a target cell and subsequently cause lysis of the target cell) activity of the antibodies. In other words, the heterodimeric antibodies encompassed by the disclosure herein include amino acid substitutions in each or both of the Fc domains of a parental sequence, usually IgG1, that can enhance ADCC.

In some embodiments, the Fc ADCC variants (e.g., ADCC-enhanced Fc variants) comprise amino acid substitution(s) selected from the group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, I332E/P247I/A339Q, S298A/E333A, S298A/E333A/K334A, V264I/I332E, S298A, S298A/I332E, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering. In some embodiments, the amino acid substitution(s) present in an Fc ADCC variants are selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, a first Fc domain and/or a second Fc domain of the bispecific antibody provided comprise an Fc ADCC variant selected from the group including: S239D, S239E, I332D, I332E, S239D/I332E, S239E/I332E, S239D/I332D, S239E/I332D, S239D/A330L/I332E, S239D/A330L, A330L/I332E, S239D/I332N, S239N/I332D, A330Y/I332E, A330L/I332E, L328V/I332E, L328T/I332E, S239Q/V264I/I332E, S239E/V264I/A330Y/I332E, L235D/S239D/A330Y/I332E, S239D/V240I/A330Y/I332E, S239D/V264T/A330Y/I332E, S239D/K326E/A330Y/I32E, S234D/K326T/A330Y/I332E, E274R, P227G, G236S, D221K, H224E, K246H, D249Y, R255Y, E258H, E258Y, T260H, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering.

In some embodiments, one or more of these variants can be included either in both of the Fe domains or in only one of the Fc domains of a heterodimeric antibody. In some embodiments, an anti-B7H3×anti-NKG2D bispecific antibody described includes ADCC-enhanced variants which includes one or more amino acid modifications in a first Fc domain and/or a second Fc domain, in other words, in the Fc domain of a first monomer, in the Fc domain of a second monomer, or in the Fc domains of both monomers. In some instances, a first Fc domain includes an Fc ADCC variant, and a second Fc domain does not include an Fc ADCC variant, resulting in an asymmetrical distribution of Fc ADCC variants. In other instances, a first Fc domain includes an Fc ADCC variant, and a second Fc domain includes an Fc ADCC variant. In one embodiment, the Fc ADCC variant of the first and second Fc domains can be the same amino acid substitution. Also, in one embodiment, the Fc ADCC variant of the first and second Fc domains can be different amino acid substitution.

In some embodiments, the Fc ADCC variants described bind with greater affinity to the FcγRIIIa (CD16A) human receptor. In some embodiments, the Fc variants have affinity for FcγRIIIa (CD16A) that is at least 1-fold, 5-fold, 10-fold, 100-fold, 200-fold, or 300-fold greater than that of the parental Fc domain.

In some embodiments, the Fc ADCC variants described can mediate effector function more effectively in the presence of effector cells. In some embodiments, the Fc variants mediate ADCC that is greater than that mediated by the parental Fc domain. In certain embodiments, the Fc variants mediate ADCC that is at least 5-fold, 10-fold, 20-fold, 30-fold, 40-fold, or 50-fold greater than that mediated by the parental Fc domain.

Additional detailed descriptions of Fc variants that may enhance ADCC are provided in WO2004/029207, which is expressly incorporated herein by reference in its entirety and specifically for the variants disclosed therein.

i. Fc v90 Variants

In some embodiments, an Fc domain with enhanced binding to human FcγRIIIa (CD16A) and thus increased ADCC activity (“an Fc ADCC variant”) utilizes the amino acid substitutions S239D/I332E (sometimes referred to as the “v90 variants”) in the CH2 domain of one or both of the monomeric Fc domains, according to EU numbering. In some embodiments, a bispecific antibody described herein comprises the Fc v90 variants (e.g., amino acid substitutions S239D/I332E) in both Fc domains. In some embodiments, a bispecific antibody described herein comprises the Fc v90 variants in only one of the monomeric Fc domains. In some embodiments, the antibody comprises the Fc v90 variants in one of the monomeric Fc domains and lacks the Fc v90 variants in another Fc domain. In some embodiments, the antibody comprises the Fc v90 variants in an Fc domain and an amino acid substitution S239D in the CH2 domain of another Fc domain, according to EU numbering. In certain embodiments, the antibody comprises the Fc v90 variants in an Fc domain and an amino acid substitution I332E in the CH2 domain of another Fc domain, according to EU numbering. In some embodiments, the antibody comprises the Fc v90 variants in an Fc domain and lacks an amino acid substitution selected from S239D, I332E and S239D/I332E in the CH2 domain of another Fc domain, according to EU numbering. In some embodiments, one monomeric Fc domain comprises the S239D variant and the other comprises the I332E variant. In some embodiments, one monomeric Fc domain comprises the S239D variant and the other comprises no Fc ADCC variant. In some embodiments, one monomeric Fc domain comprises the I332E variant and the other comprises no Fc ADCC variant.

As will be appreciated by those in the art, in the case of these asymmetrical Fc ADCC variants, which monomer receives which variant(s) can be based on the “strandedness” outlined herein; that is, it may be useful to calculate the pI of different combinations and utilize the Fc ADCC variants such that the pIs of the two monomers are different to facilitate purification.

In some embodiments, monomer 1 comprises a first Fc v90 variants, and monomer 2 comprises the amino acid substitution S239D or I332E. In some embodiments, monomer 1 comprises the Fc V90 variants, and monomer 2 does not comprise the amino acid substitution(s) S239D, I332E or S239D/I332E. In some embodiments, at least one of the Fc domains of the bispecific antibody comprises the Fc v90 variants. A first Fc domain may comprise the Fc v90 variants, or it may comprise a parental sequence relative to the Fc v90 variants (e.g., a wild-type Fc domain, a Fc domain with one or more amino acid modifications that improves ADCC but does not include S239D, I332E or S239D/I332E substitutions, and the like). In such instances where at least one of the Fc domains comprises a parental sequence, relative to the Fc v90 variants, for the purposes of this section, this Fc domain may be referred to as a “WT Fc domain” with respect to the S239 and 1332 positions of the Fc domain. In some embodiments, the antibody described herein comprises an Fc domain having an amino acid substitution of either S239D, I332E, or S239D/I332E, and another Fc domain having an amino acid substitution of either S239D, I332E, or S239D/I332E. In some embodiments, the antibody described herein comprises an Fc domain having an amino acid substitution of either S239D, I332E, or S239D/I332E, and another Fc domain without an amino acid substitution of either S239D, I332E, or S239D/I332E.

In some embodiments, the first Fc domain and the second Fc domain contain a set of ADCC-enhanced variant substitutions (first Fc domain variant: second Fc domain variant) selected from the group including: S239: I332E; S239D: S239D; S239D: WT; S239D: S239D/I332E; S239D/I332E: WT; S239D/I332E: S239D; S239D/I332E: I332E; S239D/I332E: S239D/I332E; I332E: WT; I332E: I332E; I332E: S239D; I332E: S239D/I332E; WT: S239D; WT: I332E; WT: S239D/I332E, according to EU numbering. In some embodiments, monomer 1 and monomer 2 contain a set of ADCC-enhanced variant substitutions (monomer 1 monomer 2) selected from the group including: S239: I332E; S239D: S239D; S239D: WT; S239D: S239D/I332E; S239D/I332E: WT; S239D/I332E: S239D; S239D/I332E: I332E; S239D/I332E: S239D/I332E; I332E: WT; I332E: I332E; I332E: S239D; I332E: S239D/I332E; WT: S239D; WT: I332E; WT: S239D/I332E, according to EU numbering.

In some embodiments, Fc domains with enhanced ADCC can further comprise one or more additional modifications at one or more of the following positions, including, but not limited to, 236, 243, 298, 299, or 330 in the CH2 domain, according to EU numbering. In some embodiments, the Fc variant domains comprise an amino acid substitution including, but not limited to: 236A, 243L, 298A, 299T, or 330L in the CH2 domain, according to EU numbering.

In some embodiments, an ADCC-enhanced Fc variant further includes, but is not limited, an amino acid substitution at one or more positions of the CH2 domain, according to EU numbering selected from the group including: position 236, 243, 298, 299, and 330. In some embodiments, an ADCC-enhanced Fc variant includes an amino acid substitution selected from the group including: 236A, 243L, 298A, 299T, 330L, 239D/332E, 236A/332E, 239D/332E/330L, 332E/330L, and any combination thereof in the CH2 domain, according to EU numbering. In some embodiments, the first Fc domain and/or the second Fc domain comprises an ADCC-enhanced Fc variant including, but not limited to, an amino acid substitution selected from the group including: 236A, 243L, 298A, 299T, 330L, 239D/332E, 236A/332E, 239D/332E/330L, 332E/330L, and any combination thereof in the CH2 domain, according to EU numbering, such that the Fc ADCC variant is the same in both Fc domain. Alternatively, the Fc ADCC variant is a different variant in each of the Fc domains.

Engineered antibodies comprising such ADCC-enhanced Fc variants can also have higher-affinity FcγRIIIa binding, thus resulting in stronger ADCC activity with NK cells. Bispecific antibodies having a variant Fc domain described herein can be useful and effective for NK cell-mediated killing of tumor cells.

ii. Fc Variants to Increase Binding to FcγRIIIa/CD16A

There are additional Fc substitutions that find use in enhancing FcγRIIIa binding. In some embodiments, the Fc domains of the bispecific antibodies provided include one or more Fe domains having increased binding to FcγRIIIa as compared to human IgG1 produced in standard research and production cell lines. In some embodiments, the Fc variants with improved binding affinity to at least FcγRIIIa have amino acid substitution(s) selected from the group including: V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E298R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E, according to EU numbering of the Fc domain. Additional Fc variants with enhanced binding affinity, specificity and/or avidity to FcγRIIIa are disclosed the specification and FIG. 41 of U.S. Pat. No. 8,188,231.

The described bispecific antibodies contain such Fc variants that provide enhanced effector function and substantial increases in affinity for FcγRIIIa. In some embodiments, the Fc variants improve binding to FcγRIIIa allotypes such as, for example, both V158 and F158 polymorphic forms of FcγRIIIa. The FcγR binding affinities of these Fc variants can be evaluated using assay recognized by those skilled in the art including, but not limited to, a Surface Plasmon Resonance (SPR) and/or a BLI binding assay (such as Biacore, Octet, or Caterra LSA).

2. Fc Variants for Increasing Binding to FcRn

Provided herein are additional Fc substitutions that find use in increased binding to the FcRn receptor and increased serum half-life, as specifically disclosed in U.S. Ser. No. 12/341,769, hereby incorporated by reference in its entirety, including, but not limited to, N434S, N434A, M428L, V308F, V259I, M428L/N434S, M428L/N434A, V259I/V308F, Y436I/M428L, Y436I/N434S, Y436V/N434S, Y436V/M428L, M252Y/S254T/T256E, and V259I/V308F/M428L. Such modification may be included in one or both Fc domains of the subject antibody.

In some embodiments, additional Fc variants can increase serum half-life of a bispecific antibody compared to a parental Fc domain. In some embodiments, the Fc variants have one or more amino acid modifications (i.e., substitutions, insertions or deletions) at one or more of the following amino acid residues or positions selected from the group including: 234, 235, 238, 250, 252, 254, 256, 265, 272, 286, 303, 305, 307, 311, 312, 317, 322, 340, 356, 360, 362, 376, 378, 380, 382, 413, 424, 428, and 434, according to EU numbering of the Fc region.

In some embodiments, the Fc variants have one or more amino acid substitutions selected from the group including: 234F, 235Q, 250E, 250Q, 252T, 252Y, 254T, 256E, 428L, 428F, 434S, 434A, 428L/434S, 428L/434A, 252Y/254T/256E, 234F/235Q/252T/254T/256E/322Q, 250E/428F, 250E/428L, 250Q/428F, and 250Q/428L, according to EU numbering.

In some embodiments, antibodies described can include M428L/N434S or M428L/N434A substitutions in one or both Fc domains, which can result in longer half-life in serum. In more embodiments, a first Fc domain or a second Fc domain include M428L/N434S substitutions. In more embodiments, a first Fc domain and a second Fc domain include M428L/N434S substitutions. In certain embodiments, a first Fc domain or a second Fc domain include M428L/N434A substitutions. In certain embodiments, a first Fc domain and a second Fc domain include M428L/N434A substitutions. In other embodiments, a first Fc domain and/or a second Fc domain include M252Y/S254T/T256E substitutions. Such substitutions can result in longer half-life in serum of molecules comprising such.

3. Fc Variants for Heterodimerization

In some embodiments, the antibodies, including multispecific antibodies provided herein are heterodimeric antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibodies away from homodimers are further discussed in detailed below.

There are a number of suitable pairs of sets of heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other; that is, these pairs of sets form an interface between the two monomers that encourages heterodimer formation and discourages homodimer formation, allowing the percentage of heterodimers that spontaneously form under biological conditions to be over 90%, rather than the expected 50% (25 homodimer A/A:50% heterodimer A/B:25% homodimer B/B).

i. Skew Variants

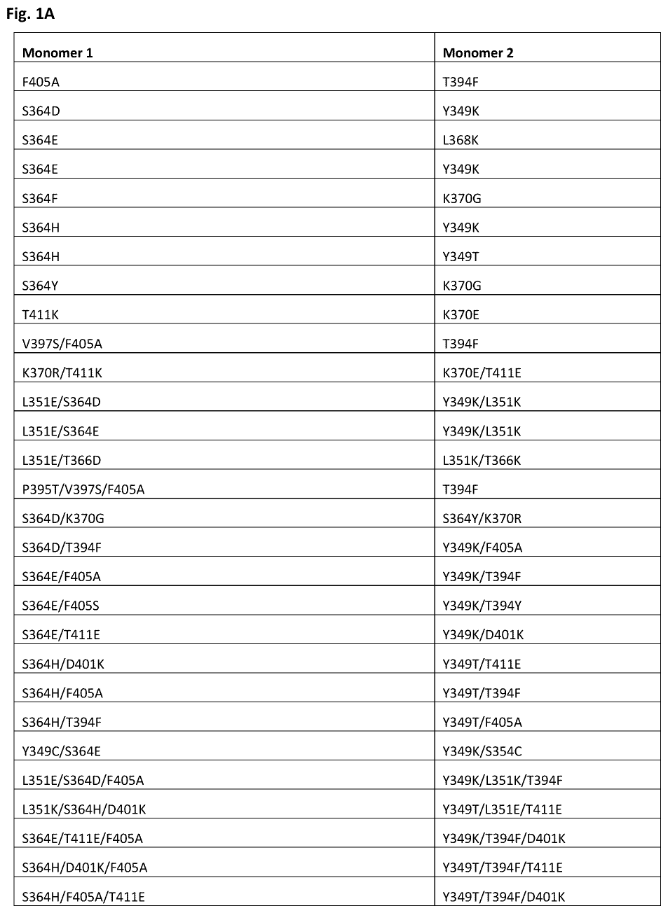

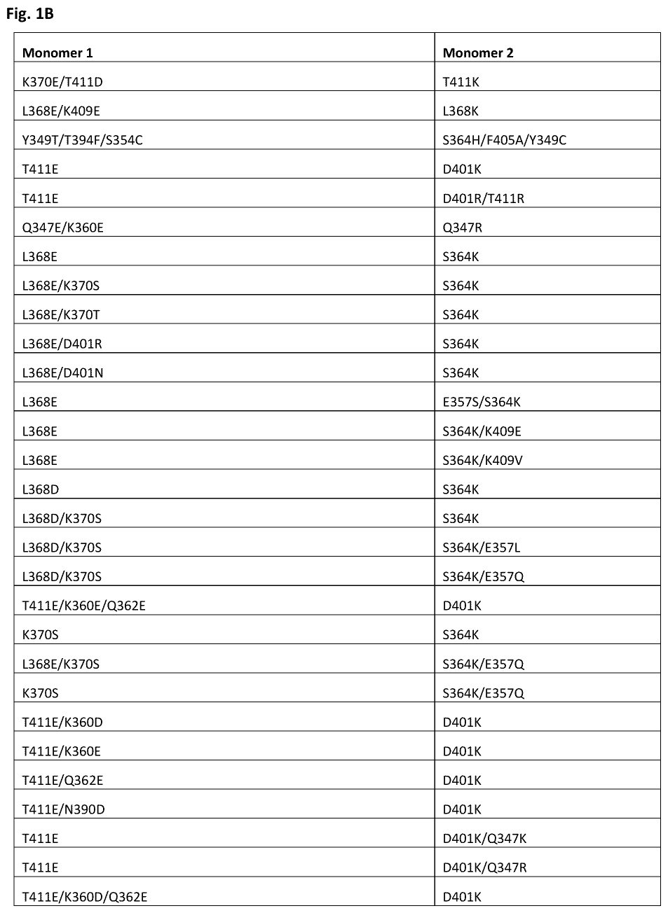

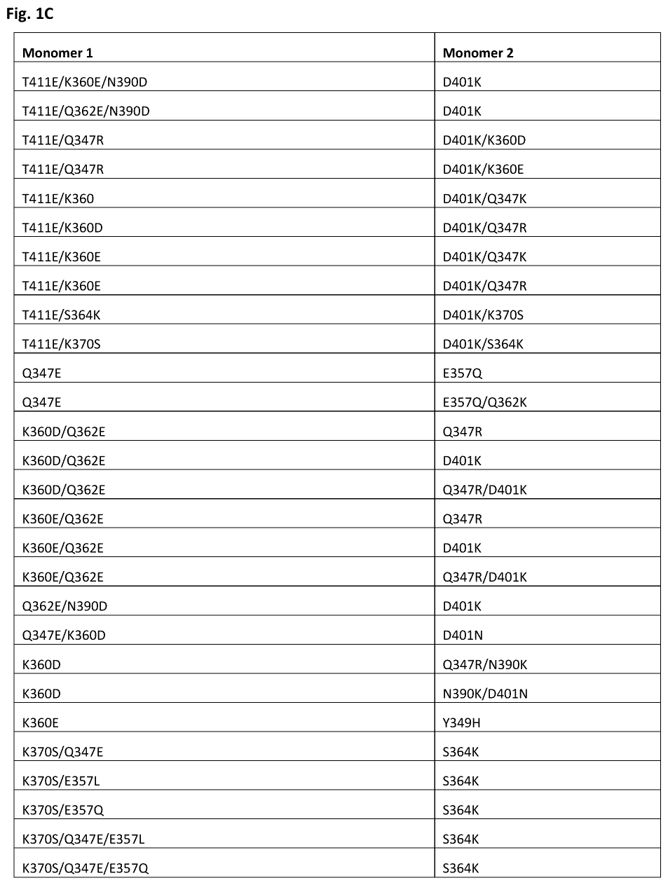

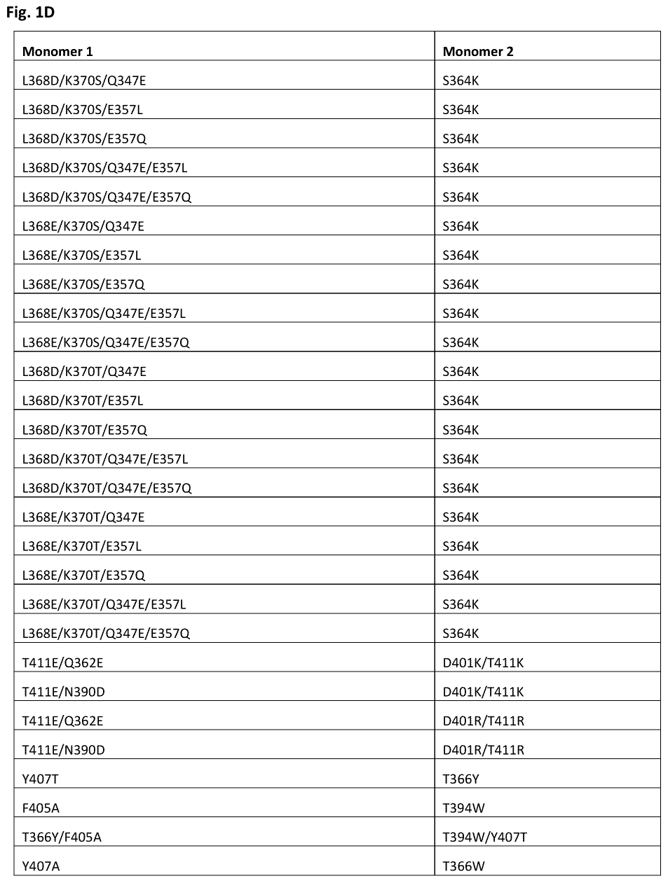

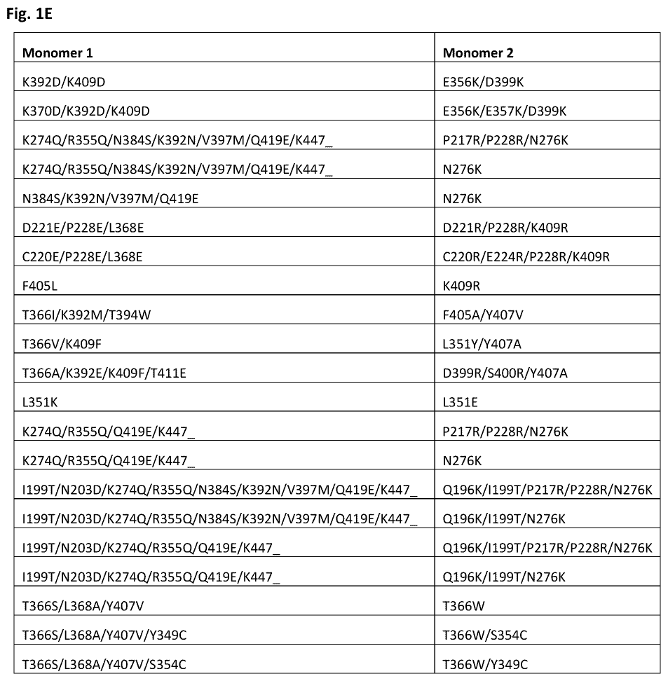

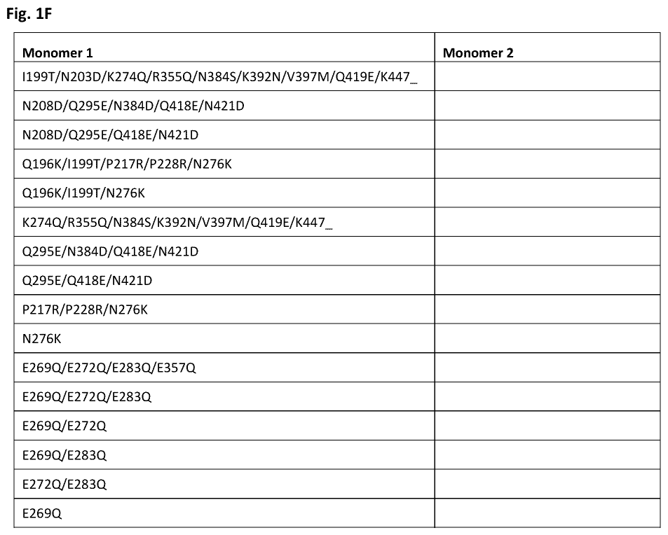

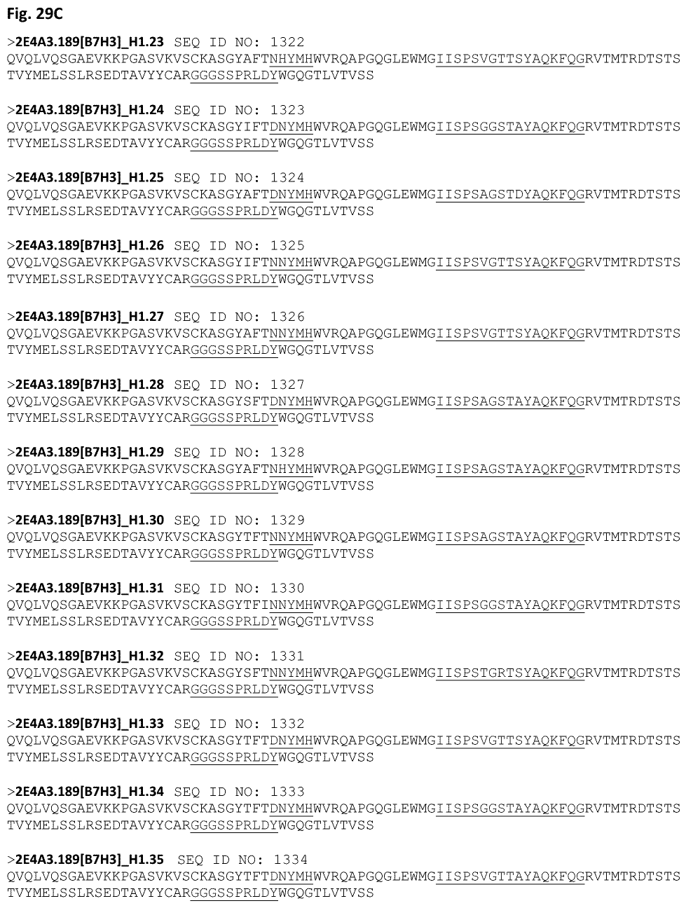

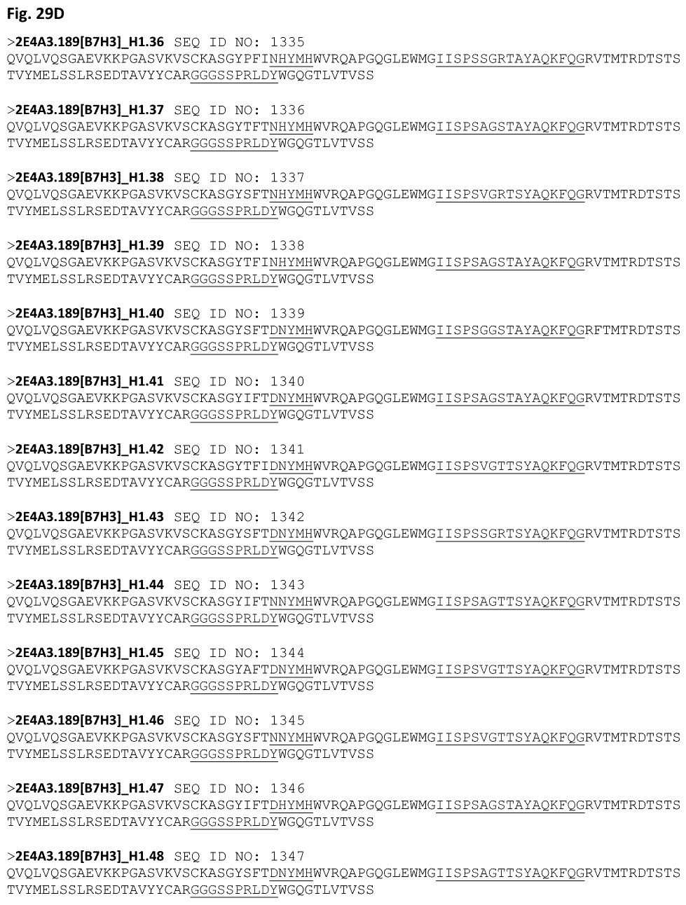

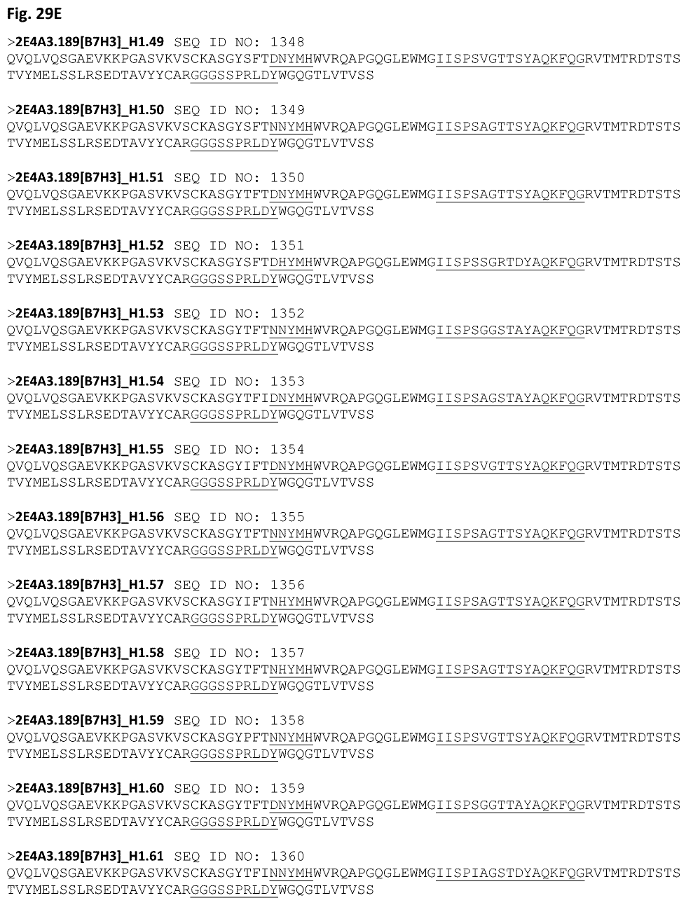

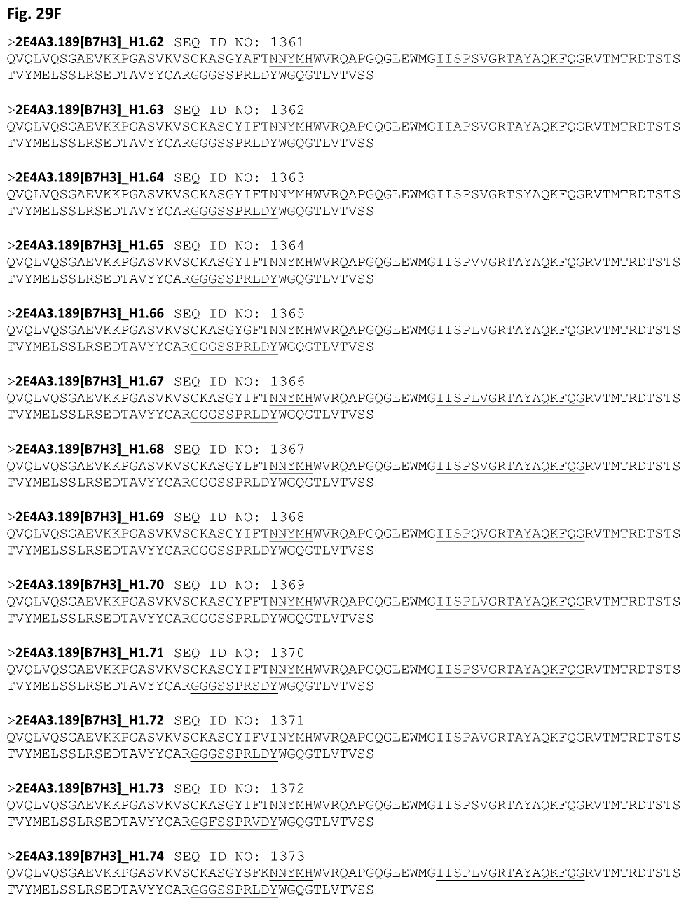

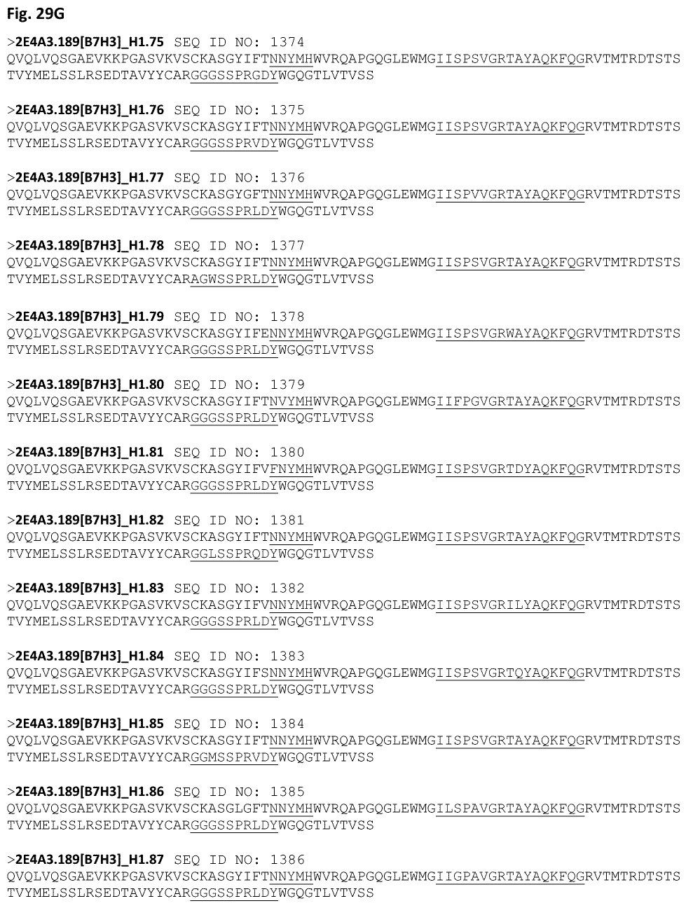

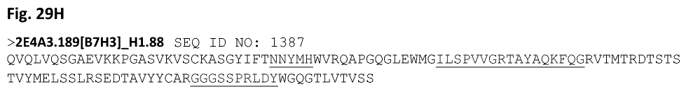

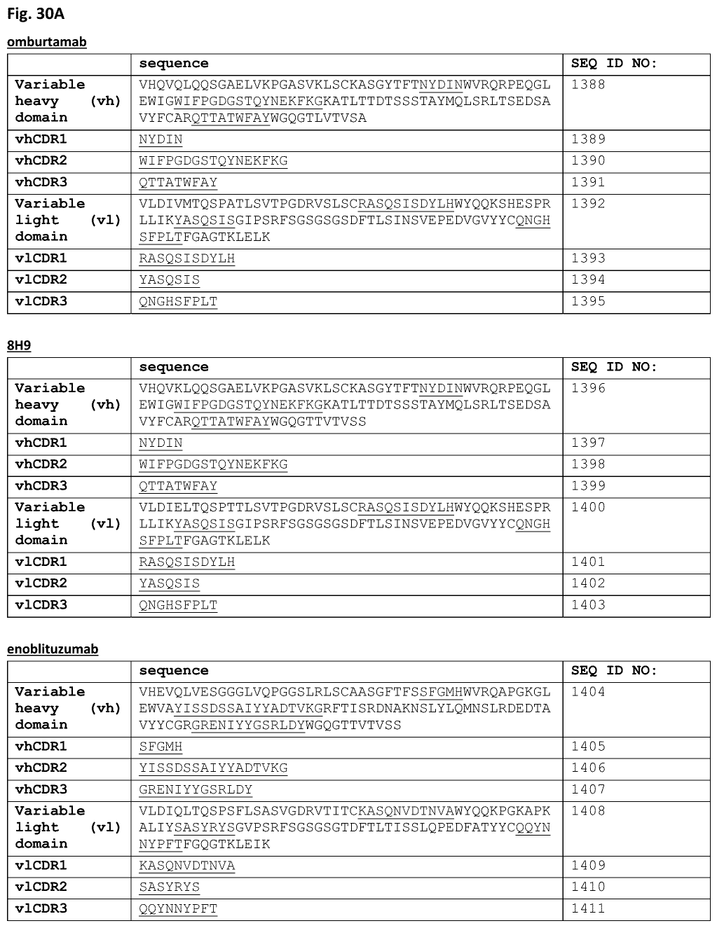

In some embodiments, the heterodimeric antibody includes skew (e.g., steric) variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in the figures, such as FIGS. 1A-1F.

Thus, suitable Fc heterodimerization variant pairs that will permit the formation of heterodimeric Fc regions are shown in FIGS. 1A-1F. Thus, a first Fc domain has first Fc heterodimerization variants and the second Fc domain has second Fc heterodimerization variants selected from the pairs in FIGS. 1A-1F.

One mechanism is generally referred to in the art as “knobs and holes,” referring to amino acid engineering that creates steric influences to favor heterodimeric formation and disfavor homodimeric formation can also optionally be used; this is sometimes referred to as “knobs and holes,” as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety. The Figures identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” as described in Gunasekaran et al., J. Biol. Chem. 285(25):19637 (2010), hereby incorporated by reference in its entirety. This mechanism is also sometimes referred to herein as “charge pairs”. In this embodiment, electrostatics are used to skew the formation towards heterodimerization. As those in the art will appreciate, these variants may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization skew variants are depicted in FIGS. 1A-1F. Such skew variants include, but are not limited to: S364K/E357Q:L368D/K370S; L368D/K370S:S364K; L368E/K370S:S364K; T411E/K360E/Q362E:D401K; L368D/K370S:S364K/E357L; K370S:S364K/E357Q (EU numbering). In terms of nomenclature, the pair “S364K/E357Q:L368D/K370S” means that one of the monomers has the double variant set S364K/E357Q and the other has the double variant set L368D/K370S.

In exemplary embodiments, the heterodimeric antibody includes Fc heterodimerization variants as sets: S364K/E357Q:L368D/K370S; L368D/K370S:S364K; L368E/K370S:S364K; T411E/K360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C or T366S/L368A/Y407V/S354C:T366W/Y349C) are all skew variant amino acid substitution sets of Fc heterodimerization variants. In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotypic variants, FcRn variants, ablation variants, etc. into one or both of the first and second Fc domains of the heterodimeric antibody. Further, individual modifications can also independently and optionally be included or excluded from the subject the heterodimeric antibody.

Additional monomer A and monomer B variants that can be combined with other variants, optionally and independently in any amount, such as pI variants outlined herein or other steric variants that are shown in FIG. 37 of US 2012/0149876, the figure and legend and SEQ ID NOs of which are incorporated expressly by reference herein.

In some embodiments, the steric variants outlined herein can be optionally and independently incorporated with any pI variant (or other variants such as, for example, Fc ADCC variants, FcRn variants, etc.) into one or both monomers, and can be independently and optionally included or excluded from the proteins of the antibodies described herein.

A subset of skew variants are “knobs in holes” (KIH) variants. Exemplary “knob-in-hole” variants are depicted in FIG. 7 of U.S. Pat. No. 8,216,805, which is incorporated herein by reference. Such “knob-in-hole” variants include, but are not limited to: an amino acid substitution at position 347, 349, 350, 351, 357, 366, 368, 370, 392, 394, 395, 397, 398, 399, 405, 407 and/or 409 of the CH3 constant domain of an IgG such as an IgG1, IgG2a, IgG2b, or IgG4 (Kabat numbering). In some embodiments, the “knob-in-hole” variants include, but are not limited to: an amino acid substitution at Y349, L351, E357, T366, L368, K370, N390, K392, T394, D399, S400, F405, Y407, K409, R409, T411, or any combination thereof of the CH3 domain of an IgG such as an IgG1, IgG2a, IgG2b, or IgG4 (EU numbering). In some embodiments, the “knob-in-hole” variants include, but are not limited to: one or more amino acid substitutions including Y349D/E, L351D/K/Y, E357K, T366A/K/Y, L368E, K370E, N390D/K/R, K392E/F/L/M/R, T394W, D399K/R/W/Y, S400D/E/K/R, F405A/I/M/S/T/V/W, Y407A/Y, K409E/D/F, R409E/D/F, and T411D/E/K/N/Q/R/W.

In some embodiments, such variants include one or more amino acid substitutions including, but not limited to: Y349C, E357K, S354C, T366S, T366W, T366Y, L368A, K370E, T394S T394W, D399K, F405A, F405W, Y407A, Y407T, Y407V, R409D, T366Y/F405A, T394W/Y407T, T366W/F405W, T394S/Y407A, F405W/Y407A, and T366W/T394S (EU numbering). In some embodiments, these variants include knob:hole paired substitutions including, but not limited to: T366W:Y407V; S354C/T366W:Y349C/T366S/Y407V; Y349C/T366W:S354C/T366S/L368A/Y407V; Y349C/T366W/R409D/K370E:S354C/T366S/L368A/Y407V/D399K/E357K; R409D/K370E:D399K/E357K; T366W:T366S/L368A/Y407V; T366W/R409D/K370E:T366S/L368A/Y407V/D399K/E357K; T366W:T366S/L368A/Y407V; T366W/Y366Y:T366S/L368A/T394W/F405A/Y407V; Y349C/T366W:S354C/T366S/L368A/Y407V; Y349C/T366W/R409D/K370E:S354C/T366S/L368A/Y407V/D399K/E357K paired substitutions, according to EU numbering.

Additional exemplary “knob-in-hole” variants as described by the amino acid substitutions of the CH3 domains can be found in, for example, Carter et al., J. Immunol. Methods, 248(1-2):7-15 (2001), Merchant et al. Nat. Biotechnol. 16(7):677-81 (1998), Ridgway et al. Protein Eng. 9(7):617-2 (1996), and U.S. Pat. Nos. 8,216,805 and 10,287,352, the disclosures of which are herein incorporated by reference in their entireties.

ii. pI (Isoelectric Point) Variants for Heterodimers

In some embodiments, the heterodimeric antibody includes purification variants that advantageously allow for the separation of heterodimeric antibody from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format and the “2+1 Fab₂-scFv-Fc” format, also allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc,” format can include charged scFv linkers (either positive or negative), that give a further pI boost for purification purposes. As will be appreciated by those in the art, some 1+1 Fab-scFv-Fc formats are useful with just charged scFv linkers and no additional pI adjustments, although the antibodies described herein do provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies for which pI is used as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B can be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (e.g., aspartic acid to lysine) or changing a charged residue to a neutral residue (e.g., loss of a charge; lysine to serine). A number of these variants are shown in the FIG. 2.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the antibodies described herein.

As will be appreciated by those in the art, the number of pI variants to be included on each or both monomer(s) to achieve good separation will depend in part on the starting pI of the components, for example in the 1+1 Fab-scFv-Fc and 2+1 Fab2-scFv-Fc formats, the starting pI of the scFv and Fab(s) of interest. That is, to determine which monomer to engineer or in which “direction” (e.g., more positive, or more negative), the Fv sequences of the two target antigens are calculated and a decision is made from there. As is known in the art, different Fvs will have different starting pIs which are exploited in the antibodies described herein. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively, or in addition to isotypic substitutions, the possibility of immunogenicity resulting from the pI variants is significantly reduced by utilizing isosteric substitutions (e.g., Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize, and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIG. 2. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)4 (SEQ ID NO: 10). However, as will be appreciated by those in the art, the first monomer includes a CH1 domain, including position 208. Accordingly, in constructs that do not include a CH1 domain (for example for fusion proteins that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 2 and the other monomer has a charged linker (either in the form of a charged scFv linker because that monomer comprises an scFv or a charged domain linker, as the format dictates, which can be selected from those depicted in FIG. 5 (see, e.g., SEQ ID NOs: 1-11, 20, 21, and 81-92)).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389, 392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In one embodiment, for example in the Figure formats, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)4 (SEQ ID NO: 93). However, as will be appreciated by those in the art, the first monomer includes a CH1 domain, including position 208. Accordingly, in constructs that do not include a CH1 domain (for example for antibodies that do not utilize a CH1 domain on one of the domains, for example in a dual scFv format or a “one-armed” format such as those depicted in FIG. 8), a preferred negative pI variant Fc set includes 295E/384D/418E/421D variants (Q295E/N384D/Q418E/N421D when relative to human IgG1).

Accordingly, in some embodiments, one monomer has a set of substitutions from the Figures and the other monomer has a charged linker (either in the form of a charged scFv linker because that monomer comprises an scFv or a charged domain linker, as the format dictates, which can be selected from those depicted in FIG. 5).

iii. Isotypic Variants

In addition, many embodiments of the antibodies described herein rely on the “importation” of pI amino acids at particular positions from one IgG isotype into another, thus reducing or eliminating the possibility of unwanted immunogenicity being introduced into the variants. A number of these are shown in FIG. 21 of U.S. Publ. App. No. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

iv. Calculating pI

The pI of each monomer can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of U.S. Publ. App. No. 2014/0370013. As discussed herein, which monomer to engineer is generally decided by the inherent pI of the Fv and scaffold regions. Alternatively, the pI of each monomer can be compared.

v. pI Variants that Also Confer Better FcRn In Vivo Binding

In the case where the pI variant decreases the pI of the monomer, they can have the added benefit of improving serum retention in vivo.

Although still under examination, Fc regions are believed to have longer half-lives in vivo, because binding to FcRn at pH 6 in an endosome sequesters the Fc (Ghetie and Ward, 1997, Immunol Today, 18(12): 592-598, entirely incorporated by reference). The endosomal compartment then recycles the Fc to the cell surface. Once the compartment opens to the extracellular space, the higher pH 7.4, induces the release of Fc back into the blood. In mice, Dall'Acqua et al. showed that Fc mutants with increased FcRn binding at pH 6 and pH 7.4 actually had reduced serum concentrations and the same half-life as wild-type Fc (Dall'Acqua et al., 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010, PEDS, 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

vi. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed herein.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

vii. Additional Heterodimerization Variants

In some embodiments, the first Fc domain comprises one or more amino acid substitutions selected from the group including: L351Y, D399R, D399K, S400D, S400E, S400R, S400K, F405A, F405I, F405M, F405T, F405S, F405V, F405W, Y407A, Y407I, Y407L, Y407V, and any combination thereof, and the second Fc domain comprises one or more amino acid substitutions selected from the group including: T350V, T366A, T366I, T366L, T366M, T366Y, T366S, T366C, T366V, T366W, N390D, N390E, N390R, K392L, K392M, K392I, K392D, K392E, T394W, K409F, K409W, T411N, T411R, T411Q, T411K, T411D, T411E, T411W, and any combination thereof.

In some embodiments, other heterodimerization pair variants include, but are not limited to, amino acid substitutions of L234A/L235A: wildtype; L234A/L235A: L234K/L235K; L234D/L235E: L234K/L235K; E233A/L234D/L235E: E233A/L234R/L235R; L234D/L235E: E233K/L234R/L235R; E233A/L234K/L235A: E233K/L234A/L235K; E269Q/D270N: E269K/D270R; and WT: L235K/A327K of the CH2 domain, according to the EU numbering.

In some embodiments, the first and/or second Fc domains comprise one or more amino acid substitutions selected from the group including: S239D, D265S, S267D, E269K, S298A, K326E, A330L and I332E. In certain instances, the Fc paired variants include, but are not limited to, S239D/D265S/I332E/E269K: S239D/D265S/S298A; S239D/K326E/A330L/I332E: S298A or S239D/K326E/A330L/I332E/E269K: S298A of the CH2 domain, according to EU numbering.

Additional descriptions of useful heterodimeric variants are disclosed in U.S. Pat. Nos. 9,732,155; 10,457,742 and 10,875,931 and U.S. Publ. App. Nos. 2021/0277150 and 2020/0087414, the disclosures of which, including the description of Fc domain variants are herein incorporated by reference in their entireties.

4. Ablation Variants

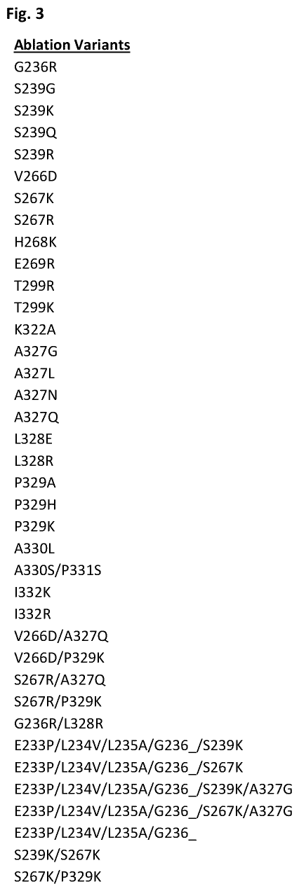

While in general NK engager multispecific antibodies retain at binding to CD16A (including “wild type” binding or increased binding to CD16A as outlined above), in some cases, surprisingly, NK engager activity can be seen even when binding to CD16A has been reduced or ablated. Accordingly, provided is another category of functional Fc variants to include are “FcγR ablation variants” or “Fc knock out (FcKO or KO)” variants. In these embodiments, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind a target antigen monovalently it is generally desirable to ablate FcγRIIIa binding to eliminate or significantly reduce ADCC activity. Wherein one of the Fc domains comprises one or more Fcγ receptor ablation variants. These ablation variants are depicted in FIG. 3, and each can be independently and optionally included or excluded, with preferred aspects utilizing ablation variants selected from the group including G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

5. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the Figures, other combinations can be generated, following the basic rule of altering the pI difference between two monomers to facilitate purification.

In addition, any of the heterodimerization variants, skew, and pI, are also independently and optionally combined with Fc ADCC variants, Fc variants, FcRn variants, or Fc ablation variants, as generally outlined herein.

Exemplary combination of variants that are included in some embodiments of the heterodimeric format antibodies are included in FIGS. 8A-8N. In certain embodiments, the heterodimeric format antibodies, including multispecific antibodies, bind to NK cells.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

6. Afucosylated Fc Domains

In some embodiments, the increased binding of a Fc domain to CD16A is the result of producing the NKE in a cell line that reduces or eliminates the incorporation of fucose into the glycosylation of the NKE. See, for example, Pereira et al., MAbs (2018) 10(5):693-711.

In some embodiments, antibodies comprising Fc domains described are produced in a host cell such that the Fc domains have reduced fucosylation or no fucosylation compared to a parental Fc domain. In some instances, antibodies described are produced in a genetically modified host cell, wherein the genetic modification to the host cell results in the overexpression of β(1,4)-N-acetylglucosaminyltransferase III (GnTIII), a glycosyltransferase catalyzing the formation of bisected oligosaccharides, which are generally also non-fucosylated. N-glycosylation of the Fc domain can play a role in binding to FcγR; and afucosylation of the N-glycan can increase the binding capacity of the Fc domain to FcγRIIIa. As discussed in further detail above, an increase in FcγRIIIa binding can enhance ADCC, which can be advantageous in certain antibody therapeutic applications in which cytotoxicity is desirable.

In some embodiments, an Fc domain is engineered such that it has reduced fucosylation or no fucosylation, compared to a parental Fc domain. In the context of an Fc domain, the terms “afucosylation,” “afucosylated,” “defucosylation,” and “defucosylated” are used interchangeably, and generally refer to the absence or removal of core-fucose from the N-glycan attached to the CH2 domain of an Fc domain. For instance, an afucosylated antibody lacks core fucosylation in the Fc domain. As used herein, the phrase “a low level of fucosylation” or “reduced fucosylation” generally refers to an overall fucosylation level in a specific Fc domain that is no more than about 10.0%, no more than 5.0%, no more than 2.5%, no more than 1.0%, no more than about 0.5%, no more than 0.25%, no more than about 0.1%, or no more than 0.01%, compared to the fucosylation level of parental Fc domain. The term “% fucosylation” generally refers to the level of fucosylation in a specific Fc domain compared to that of a parental Fc domain. The % fucosylation can be measured according to any suitable method known in the relevant art, such as, for example, by mass spectrometry (MS), HPLC-Chip Cube MS (Agilent), and reverse phase-HPLC.

In some embodiments, a particular level of fucosylation is desired. In some embodiments, a Fc variant is provided, wherein the Fc variant comprises a particular level of afucosylation. In some further embodiments, the fucosylation level of the Fc variant is no more than about 10.0%, no more than about 9.0%, no more than about 8.0%, no more than about 7.0%, no more than about 6.0%, no more than about 5.0%, no more than about 4.0%, no more than about 3.0%, no more than about 2.0%, no more than about 1.5%, no more than about 1.0%, no more than about 0.5%, no more than 0.25%, no more than about 0.1%, or no more than 0.01%, compared to that of a parental Fc domain.

In some embodiments, antibodies comprising afucosylated Fc domains can be enriched (to obtain a particular level of afucosylation) by affinity chromatography using resins conjugated with a fucose binding moiety, such as, for example, an antibody or lectin specific for fucose, with some embodiments finding particular utility when fucose is present in a 1-6 linkage (see, e.g., Kobayashi et al., 2012, J. Biol. Chem. 287:33973-82).

In some embodiments, the fucosylated species of the Fc domain can be separated from the afucosylated species of the Fc domain (to obtain a particular level of afucosylation) using an anti-fucose specific antibody in an affinity column. Alternatively, or in addition to, afucosylated species can be separated from fucosylated species based on the differential binding affinity to FcγRIIIa using affinity chromatography (again, to obtain a particular level of afucosylation).

C. Useful Formats of the Invention

As will be appreciated by those in the art, and discussed more fully below, the heterodimeric bispecific antibodies provided herein can take on a wide variety of configurations, as are generally depicted in FIGS. 8A-8N. Some figures depict “single ended” configurations, where there is one type of specificity on one “arm” of the molecule and a different specificity on the other “arm.” Other figures depict “dual ended” configurations, where there is at least one type of specificity at the “top” of the molecule and one or more different specificities at the “bottom” of the molecule. Thus, in some embodiments, the antibodies described herein are directed to novel immunoglobulin compositions that co-engage a first antigen and a second antigen that are different.

As will be appreciated by those in the art, the heterodimeric formats of the antibodies described herein can have different valences (e.g., bivalent, trivalent, etc.), as well as specificity (e.g., bispecific). That is, in some embodiments, heterodimeric antibodies of the antibodies described herein can be bivalent and bispecific, wherein one target antigen (e.g., a NK cell antigen) is bound by a first binding domain and the other target antigen (e.g., a TTA antigen) is bound by a second binding domain. In other embodiments, the heterodimeric antibodies can be trivalent and bispecific, wherein the first antigen is bound by two binding domains (i.e., a first binding domain and a second binding domain) and the second antigen is bound by the first binding domain or the second binding domain.

The antibodies described herein utilize anti-NK cell ABDs in combination with anti-TTA ABDs. As will be appreciated by those in the art, any collection of anti-NK cell CDRs, anti-NK cell antigen variable light and variable heavy domains, Fabs, and scFvs as described herein, and depicted in any of the Figures can be used, optionally and independently combined in any combination. Similarly, any of the anti-TTA ABDs can be used, whether CDRs, variable light and variable heavy domains, Fabs, and scFvs as described herein, and depicted in any of the Figures can be used, optionally and independently combined in any combination.

1. 1+1 Fab-scFv-Fc Format

One heterodimeric scaffold that finds particular use in the antibodies described herein is the “1+1 Fab-scFv-Fc” or “bottle-opener” or “1+1 Fab×scFv” format, as is shown in FIG. 8A. In this embodiment, one heavy chain monomer of the antibody contains a single chain Fv (“scFv,” as described below) and an Fc domain. The scFv includes a variable heavy domain (VH1) and a variable light domain (VL1), wherein the VH1 is attached to the VL1 using an scFv linker that can be charged (see, e.g., FIG. 5; SEQ ID NOs: 1-11, 20, 21, and 81-92). The scFv is attached to the heavy chain using a domain linker (see, e.g., FIGS. 6 and 7; SEQ ID NOs: 30, 31, 40, 41, 50, 51, and 94-119). The other heavy chain monomer is a “regular” heavy chain (V_{H}-CH1-hinge-CH2-CH3). The 1+1 Fab-scFv-Fc also include a light chain that interacts with the V_{H}-CH1 to form a Fab. This structure is sometimes referred to herein as the “bottle-opener” format due to a rough visual similarity to a bottle-opener. The two heavy chain monomers are brought together by the use of amino acid variants (e.g., heterodimerization variants, as discussed above) in the constant regions (e.g., the Fc domain, the CH1 domain, and/or the hinge region) that promote the formation of heterodimeric antibodies, as is described more fully below.

There are several distinct advantages to the present “1+1 Fab-scFv-Fc” format. As is known in the art, antibody analogs relying on two scFv constructs often have stability and aggregation problems, which can be alleviated in the antibodies described herein by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

Many of the embodiments outlined herein rely in general on the 1+1 Fab-scFv-Fc or “bottle opener” format antibody that comprises a first monomer comprising an scFv, comprising a variable heavy and a variable light domain, covalently attached using an scFv linker (charged, in many but not all instances), where the scFv is covalently attached to the N-terminus of a first Fc domain, usually through a domain linker. The domain linker can be either charged or uncharged, and exogenous or endogenous (e.g., all or part of the native hinge domain). Any suitable linker can be used to attach the scFv to the N-terminus of the first Fc domain. In some embodiments, the domain linker is chosen from the domain linkers in FIGS. 6 and 7. The second monomer of the 1+1 Fab-scFv-Fc format or “bottle opener” format is a heavy chain, and the composition further comprises a light chain.

In general, in many preferred embodiments, the scFv is the domain that binds to the NK cell antigen, and the Fab forms a TTA binding domain. However, in several preferred embodiments, the scFv is the domain that binds to the TTA, and the Fab forms a NK cell antigen binding domain. An exemplary anti-NK cell antigen x anti-TTA bispecific antibody of the 1+1 Fab-scFv-Fc format is depicted schematically in FIG. 8A.

In addition, the Fc domains of the antibodies described herein generally include one or more of: (a) Fc ADCC variants (see, e.g., FIG. 4), (b) skew variants (see, e.g., FIGS. 1A-1F; with particularly useful skew variants being selected from the group including: (i) S364K/E357Q:L368D/K370S, (ii) L368D/K370S:S364K, (iii) L368E/K370S:S364K, (iv) T411E/K360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), (c) optionally ablation variants (see, e.g., FIG. 3), (d) optionally charged scFv linkers (including those shown in FIG. 5; see, e.g., SEQ ID NOs: 1-11, 20, 21, and 81-92), and the heavy chain can comprise pI variants (including, but not limited to, those shown in FIGS. 1 and 2).

In certain embodiments the 1+1 Fab-scFv-Fc scaffold format includes a first monomer that includes a scFv-domain linker-CH2-CH3 monomer, a second monomer that includes a first variable heavy domain-CH1-hinge-CH2-CH3 monomer, and a third monomer that includes a first variable light domain. In some embodiments, the CH2-CH3 of the first monomer is a first variant Fc domain and the CH2-CH3 of the second monomer is a second variant Fc domain. In some embodiments, the scFv includes a scFv variable heavy domain and a scFv variable light domain that form a NK cell antigen binding moiety. In other embodiments, the scFv includes a scFv variable heavy domain and a scFv variable light domain that form a TTA binding moiety. In certain embodiments, the scFv variable heavy domain and scFv variable light domain are covalently attached using an scFv linker (charged, in many but not all instances; see, e.g., FIG. 5 and SEQ ID NOs: 1-11, 20, 21, and 81-92). In some embodiments, the first variable heavy domain and first variable light domain form a TTA binding domain. In other embodiments, the first variable heavy domain and first variable light domain form a NK cell antigen binding domain.

Any suitable NK cell ABD and/or TTA ABD can be included in the 1+1 Fab-scFv-Fc format antibody, including those provided herein. NK cell antigen binding domain sequences finding particular use in these embodiments include, but are not limited to, those shown in the Figures, or variants thereof. NK cell ABDs that are of particular use in these embodiments include, but are not limited to, V_{H} and V_{L} domains selected from V_{H}/V_{L} pairs selected from those shown in the Figures, or variants thereof. TTA binding domain sequences finding particular use in these embodiments include, but are not limited to those shown in the Figures, or variants thereof. TTA ABDs that are of particular use in these embodiments include, but are not limited to, V_{H} and V_{L} domains selected from V_{H}/V_{L} pairs selected from those shown in the Figures, or variants thereof.

In some embodiments, the 1+1 Fab-scFv-Fc format includes Fc ADCC variants (see, e.g., FIG. 4), skew variants (see, e.g., FIGS. 1A-1F), and/or pI variants (see, e.g., FIGS. 1 and 2). Accordingly, some embodiments include 1+1 Fab-scFv-Fc formats that comprise: (i) a first monomer (the “scFv monomer”) that comprises a charged scFv linker (with the “+H” sequence of FIG. 5 (i.e., SEQ ID NO: 10) being preferred in some embodiments, the Fc ADCC variants S239D/I332E, the skew variants S364K/E357Q, and an scFv that binds to a first target antigen as outlined herein, (ii) a second monomer (the “Fab monomer”) that comprises the skew variants L368D/K370S, the pI variants N208D/Q295E/N384D/Q418E/N421D, and a variable heavy domain (V_{H}), and (iii) a light chain that includes a variable light domain (V_{L}) and a constant light domain (C_{L}), wherein numbering is according to EU numbering. Further, bispecific antibodies in the 1+1 Fab-scFv-Fc format can also include one or more Fc domains with one or more ablation variants (see, e.g., FIG. 3). In some embodiments, the first target antigen is a NK cell antigen, and the first variable heavy domain and the first variable light domain make up a TTA binding moiety. In other embodiments, the first target antigen is a TTA, and the first variable heavy domain and the first variable light domain make up a NK cell antigen binding moiety. NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

In some embodiments, the 1+1 Fab-scFv-Fc format includes Fc ADCC variants (see, e.g., FIG. 4), skew variants (see, e.g., FIGS. 1A-1F), pI variants (see, e.g., FIGS. 1 and 2), and/or FcRn variants (e.g., M428L, N434S, N434A, M428L/N434S, M428L/N434A, M252Y/S254T/T256E, etc.). Accordingly, some embodiments include 1+1 Fab-scFv-Fc formats that comprise: (i) a first monomer (the “scFv monomer”) that comprises a charged scFv linker (with the “+H” sequence of FIG. 5 (i.e., SEQ ID NO: 10) being preferred in some embodiments), the Fc ADCC variants S239D/I332E, the skew variants S364K/E357Q, the FcRn variants M428L/N434S, and an scFv that binds to a first target antigen as outlined herein, (ii) a second monomer (the “Fab monomer”) that comprises the skew variants L368D/K370S, the pI variants N208D/Q295E/N384D/Q418E/N421D, the FcRn variants M428L/N434S or M428L/N434A, and a variable heavy domain, and (iii) a light chain that includes a variable light domain (V_{L}) and a constant light domain (C_{L}), wherein numbering is according to EU numbering. Further, bispecific antibodies in the 1+1 Fab-scFv-Fc format can also include one or more Fc domains with one or more ablation variants (see, e.g., FIG. 3). In some embodiments, the first target antigen is a NK cell antigen, and the first variable heavy domain and the first variable light domain make up a TTA binding moiety. In other embodiments, the first target antigen is a TTA, and the first variable heavy domain and the first variable light domain make up a NK cell antigen binding moiety. NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

FIGS. 32-35 show exemplary Fc domain sequences that are useful in the 1+1 Fab-scFv-Fc format antibodies. Generally, the “monomer 1” sequences (as depicted in the Figures) refers to the Fc domain of the “Fab-Fc heavy chain,” and the “monomer 2” sequences (as depicted in the Figures) refer to the Fc domains of the “scFv-Fc heavy chain.”

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of: (i) SEQ ID NOs: 1798 and 1799, (ii) SEQ ID NOs: 1800 and 1801, (iii) SEQ ID NOs: 1802 and 1803, (iv) SEQ ID NOs: 1804 and 1805, (v) SEQ ID NOs: 1806 and 1807, (vi) SEQ ID NOs: 1808 and 1809, (vii) SEQ ID NOs: 1810 and 1811, (viii) SEQ ID NOs: 1812 and 1813, (ix) SEQ ID NOs: 1814 and 1815, (x) SEQ ID NOs: 1816 and 1817, (xi) SEQ ID NOs: 1818 and 1819, as shown in FIGS. 31A-31C.

In some embodiments, one or both of the Fc domains comprising the heterodimeric Fc backbone pairs include amino acid substitutions such that one or both of the Fc domains have increased ADCC activity. In certain embodiments, the amino acid substitutions include: S239D, I332E, S239D/I332E, S239E, I332D, S239E/I332E, S239D/I332D, S239E/I332D, S239D/330L/I332E, S239D/A330L, A330L/I332E, F243L, F243L/R292P/Y300L/V305I/P396L, 332I/247I/339Q, S298A/E333A/K334A, V264I/I332E, S298A, S298A/I332E, S298A/E333A.K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, 3293R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E, S239D/K274E/A330L/I332E, and any of the Fc ADCC variants shown in FIG. 4. In many embodiments, the amino acid substitutions include: S239D, I332E, S239D/I332E, S239E, I332D, S239E/I332E, S239D/I332D, S239E/I332D. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) includes amino acid variants conferring an increase in ADCC activity. In some instances, monomer 1 and monomer 2 can contain identical amino acid substitutions (e.g., both monomers contain a S239D substitution, an I332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the I332E substitution); monomer 2 may contain the S239D/I332E substitutions, and monomer 1 contains the S239D substitution and is WT with respect to the 332 residue (or the reverse where monomer 1 is WT with respect to the 239 residue and contains the I332E substitution); etc.). In still other instances, monomer 1 and monomer 2 can contain amino acid substitutions that are unique to each monomer (e.g., S239D on monomer 1 and I332E on monomer 2, or the reverse). In some embodiments, the heterodimeric Fc backbone pairs have increased ADCC activity, including, but not limited to, those pairs of: (i) SEQ ID NOs: 1820 and 1821, (ii) SEQ ID NOs: 1822 and 1823, (iii) SEQ ID NOs: 1824 and 1825, (iv) SEQ ID NOs: 1826 and 1827, (v) SEQ ID NOs: 1828 and 1829, (vi) SEQ ID NOs: 1830 and 1831, (vii) SEQ ID NOs: 1832 and 1833, (viii) SEQ ID NOs: 1834 and 1835, (ix) SEQ ID NOs: 1836 and 1837, (x) SEQ ID NOs: 1838 and 1839, (xi) SEQ ID NOs: 1840 and 1841, (xii) SEQ ID NOs: 1842 and 1843, (xiii) SEQ ID NOs: 1844 and 1845, (xiv) SEQ ID NOs: 1846 and 1847, and (xv) SEQ ID NOs: 1848 and 1849, as shown in FIGS. 32A-32D (see, e.g., SEQ ID NOs: 1820-1849).

In some embodiments of the multispecific antibody, one or both of the Fc domains comprising a heterodimeric Fc backbone pair include amino acid substitutions which improve serum half-life of the antibody. In certain embodiments, the amino acid substitutions include one or more of: M252Y/S254T/T256E, M428L, N434S, N434A, M428L/N434S, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer). In some embodiments, the heterodimeric Fc backbone pairs have increased serum half-life, including, but not limited to, those pairs of Fc backbones shown in the Figures.

In some embodiments, one or both of the Fc domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, I332E, and S239D/I332E, or any of the Fc ADCC variants shown in FIG. 4, and (ii) one or more amino acid substitutions selected from the group including: M252Y/S254T/T256E, M428L, N434S, N434A, M428L/N434S, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of: (i) SEQ ID NOs: 1850 and 1851, (ii) SEQ ID NOs: 1852 and 1853, (iii) SEQ ID NOs: 1854 and 1855, (iv) SEQ ID NOs: 1856 and 1857, (v) SEQ ID NOs: 1858 and 1859, (vi) SEQ ID NOs: 1860 and 1861, (vii) SEQ ID NOs: 1862 and 1863, (viii) SEQ ID NOs: 1864 and 1865, (ix) SEQ ID NOs: 1866 and 1867, (x) SEQ ID NOs: 1868 and 1869, (xi) SEQ ID NOs: 1870 and 1871, (xii) SEQ ID NOs: 1872 and 1873, (xiii) SEQ ID NOs: 1874 and 1875, (xiv) SEQ ID NOs: 1876 and 1877, and (xv) SEQ ID NOs: 1878 and 1879, as shown in FIGS. 33A-33D.

In some embodiments, one or both of the Fc domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, only monomer 1 or monomer 2 of the heterodimeric Fc backbone pairs include amino acid substitutions that result in an ablated FcγR function (see, e.g., FIG. 3). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 3). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs have ablated FcγR function, including, but not limited to, those pairs of: (i) SEQ ID NOs: 1880 and 1881, (ii) SEQ ID NOs: 1882 and 1883, (iii) SEQ ID NOs: 1884 and 1885, (iv) SEQ ID NOs: 1886 and 1887, (v) SEQ ID NOs: 1888 and 1889, (vi) SEQ ID NOs: 1890 and 1891, (vii) SEQ ID NOs: 1892 and 1893, (viii) SEQ ID NOs: 1894 and 1895, (ix) SEQ ID NOs: 1896 and 1897, (x) SEQ ID NOs: 1898 and 1899, (xi) SEQ ID NOs: 1900 and 1901, (xii) SEQ ID NOs: 1902 and 1903, (xiii) SEQ ID NOs: 1904 and 1905, (xiv) SEQ ID NOs: 1906 and 1907, (xv) SEQ ID NOs: 1908 and 1909, (xvi) SEQ ID NOs: 1910 and 1911, (xvii) SEQ ID NOs: 1912 and 1913, (xviii) SEQ ID NOs: 1914 and 1915, (xix) SEQ ID NOs: 1916 and 1917, (xx) SEQ ID NOs: 1918 and 1919, (xxi) SEQ ID NOs: 1920 and 1921, (xxii) SEQ ID NOs: 1922 and 1923, (xxiii) SEQ ID NOs: 1924 and 1925, (xxiv) SEQ ID NOs: 1926 and 1927, (xxv) SEQ ID NOs: 1928 and 1929, (xxvi) SEQ ID NOs: 1930 and 1931, and (xxvii) SEQ ID NOs: 1932 and 1933, as shown in FIGS. 34 and 35.

Further, FIG. 41 provides useful C_{L} sequences that can be used with this format (see, e.g., SEQ ID NOs: 1969 and 1970).

In some embodiments, any of the V_{H} and V_{L} sequences depicted herein (including all V_{H} and V_{L} sequences depicted in the Figures and sequence listing, including those directed to NK cell antigens) can be added to the bottle opener backbone formats of the Figures as the “Fab side,” using any of the anti-NK cell antigen scFv sequences shown in the Figures and sequence listing. Alternatively, any of the V_{H} and V_{L} sequences depicted herein (including all V_{H} and V_{L} sequences depicted in the Figures and sequence listing, including those directed to TTAs) can be added to the bottle opener backbone formats of the Figures as the “Fab side,” using any of the anti-TTA scFv sequences shown in the Figures and sequence listing.

2. 2+1 Fab₂-scFv-Fc Format

One heterodimeric scaffold that finds particular use in the antibodies described herein is the “2+1 Fab₂-scFv-Fc” format (also referred to in previous related filings as the “central-scFv” or “2+1 Fab×Fab-scFv” format), as is shown in FIG. 8B. In this embodiment, the format relies on the use of an inserted scFv domain thus forming a third antigen binding domain, wherein the Fab portions of the two monomers bind TTAs and the “extra” scFv domain binds a NK cell antigen. The scFv domain is inserted between the Fc domain and the CH1-Fv region of one of the monomers, thus providing the third antigen binding domain. As described, αNK cell antigen×αTTA bispecific antibodies having the 2+1 Fab₂-scFv-Fc format may be potent in inducing redirected T cell cytotoxicity in cellular environments that express low levels of a TTA. Such antibodies may exhibit differences in selectivity for cells with different TTA expression, potencies for TTA expressing cells, ability to elicit cytokine release, and sensitivity to soluble the TTA. These TTA antibodies find use, for example, in the treatment of TTA-associated cancers. In some embodiments, the heterodimeric antibody of this format is bivalent for the NK cell antigen and monovalent for the TTA antigen.

In this embodiment, one monomer comprises a first heavy chain comprising a first variable heavy domain, a CH1 domain (and optional hinge) and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using optional domain linkers (VH1-CH1-[optional linker]-VH2-scFv linker-VL2-[optional linker]-CH2-CH3, or the opposite orientation for the scFv, VH1-CH1-[optional linker]-VL2-scFv linker-VH2-[optional linker including the hinge]-CH2-CH3). The optional linkers can be any suitable peptide linkers, including, for example, the domain linkers included in FIGS. 6 and 7 (see, e.g., SEQ ID NOs: 30, 31, 40, 41, 50, 51, and 94-119). In some embodiments, the optional linker is a hinge or a fragment thereof. The other monomer is a standard Fab side (i.e., VH1-CH1-hinge-CH2-CH3). This embodiment further utilizes a common light chain comprising a variable light domain and a constant light domain, that associates with the heavy chains to form two identical Fabs that bind TTAs. As for many of the embodiments herein, these constructs include skew variants, pI variants, ablation variants, additional Fc variants, etc. as desired and described herein (see, e.g., FIGS. 1-4).

In one embodiment, the 2+1 Fab₂-scFv-Fc format antibody includes an scFv with the V_{H} and V_{L} of a NK cell antigen binding domain sequence depicted in the Figures, or a variant thereof. In one embodiment, the 2+1 Fab₂-scFv-Fc format includes two Fabs having the V_{H} and V_{L} of a TTA binding domain as depicted in the Figures, or a variant thereof.

In exemplary embodiments, the TTA binding domain of the 2+1 Fab₂-scFv-Fc anti-NK cell antigen x anti-TTA bispecific antibody includes the V_{H} and V_{L} NK cell antigen binding domain sequences finding particular use in these embodiments include, but are not limited to, those shown in the Figures, or a variant thereof. Any suitable TTA ABD can be included in the 2+1 Fab₂-scFv-Fc format antibody, included those provided herein. TTA ABDs that are of particular use in these embodiments include, but are not limited to, V_{H} and V_{L} domains selected from the group including those shown in the Figures, or variants thereof.

In addition, the Fc domains of the antibodies described herein generally include one or more of: (a) Fc ADCC variants (see, e.g., FIG. 4), (b) skew variants (see, e.g., FIGS. 1A-1F; with particularly useful skew variants being selected from the group including: (i) S364K/E357Q:L368D/K370S, (ii) L368D/K370S:S364K, (iii) L368E/K370S:S364K, (iv) T411E/K360E/Q362E:D401K, (v) L368D/K370S:S364K/E357L, (vi) K370S:S364K/E357Q, (vii) T366S/L368A/Y407V:T366W, and (viii) T366S/L368A/Y407V/Y349C:T366W/S354C), (c) optionally ablation variants (see, e.g., FIG. 3), (d) optionally charged scFv linkers (including those shown in FIG. 5; see, e.g., SEQ ID NOs: 1-11, 20, 21, and 81-92), and the heavy chain can comprise pI variants (including, but not limited to, those shown in FIGS. 1 and 2).

In some embodiments, the 2+1 Fab₂-scFv-Fc format antibody includes Fc ADCC variants (see, e.g., FIG. 4), skew variants (see, e.g., FIGS. 1A-1F), and/or pI variants (see, e.g., FIGS. 1 and 2). Accordingly, some embodiments include 2+1 Fab₂-scFv-Fc formats that comprise: (i) a first monomer (the “Fab-scFv-Fc” monomer) that comprises the Fc ADCC variants S239D/I332E, the skew variants S364K/E357Q, and a variable heavy domain that, with the variable light domain of the common light chain, makes up an Fv that binds to a TTA as outlined herein, and an scFv domain that binds to a NK cell antigen, (ii) a second monomer (the “Fab-Fc” monomer) that comprises the skew variants L368D/K370S, the pI variants N208D/Q295E/N384D/Q418E/N421D, and a variable heavy domain that, with variable light domain of the common light chain, makes up an Fv that binds to a TTA as outlined herein, and (iii) a common light chain comprising the variable light domain and a constant light domain, where numbering is according to EU numbering. Further, bispecific antibodies in the 2+1 Fab₂-scFv-Fc format can also include one or more Fc domains with one or more ablation variants (see, e.g., FIG. 3). NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

In some embodiments, the 2+1 Fab₂-scFv-Fc format antibody includes Fc ADCC variants (see, e.g., FIG. 4), skew variants (see, e.g., FIGS. 1A-1F), pI variants (see, e.g., FIGS. 1 and 2), and/or FcRn variants (e.g., M428L, N434S, N434A, M428L/N434S, M428L/N434A, M252Y/S254T/T256E, etc.). Accordingly, some embodiments include 2+1 Fab₂-scFv-Fc formats that comprise: (i) a first monomer (the “Fab-scFv-Fc” monomer) that comprises the Fc ADCC variants S239D/I332E, the skew variants S364K/E357Q, the FcRn variants M428L/N434S, and a variable heavy domain that, with the variable light domain of the common light chain, makes up an Fv that binds to a TTA as outlined herein, and an scFv domain that binds to a NK cell antigen, (ii) a second monomer (the “Fab-Fc” monomer) that comprises the skew variants L368D/K370S, the pI variants N208D/Q295E/N384D/Q418E/N421D, the FcRn variants M428L/N434S, and a variable heavy domain that, with variable light domain of the common light chain, makes up an Fv that binds to a TTA as outlined herein, and (iii) a common light chain comprising the variable light domain and a constant light domain, where numbering is according to EU numbering. Further, bispecific antibodies in the 2+1 Fab₂-scFv-Fc format can also include one or more Fc domains with one or more ablation variants (see, e.g., FIG. 3). NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

FIGS. 36A-36C show some exemplary Fc domain sequences that are useful with the 2+1 Fab₂-scFv-Fc format. Generally, the “monomer 1” sequences (as depicted in the Figures) refer to the Fc domain of the “Fab-Fc heavy chain,” and the “monomer 2” sequences (as depicted in the Figures) refer to the Fc domain of the “Fab-scFv-Fc heavy chain.” In some embodiments, the heterodimeric Fc backbone pairs are those pairs of: (i) SEQ ID NOs: 1934 and 1935, (ii) SEQ ID NOs: 1936 and 1937, (iii) SEQ ID NOs: 1938 and 1939, (iv) SEQ ID NOs: 1940 and 1941, (v) SEQ ID NOs: 1942 and 1943, (vi) SEQ ID NOs: 1944 and 1945, (vii) SEQ ID NOs: 1946 and 1947, (viii) SEQ ID NOs: 1948 and 1949, and (ix) SEQ ID NOs: 1950 and 1951, as shown in FIGS. 36A-36C.

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains comprising the heterodimeric Fc backbone pairs include amino acid substitutions such that one or both of the Fc domains have increased ADCC activity. In certain embodiments, the amino acid substitutions include one or more of: S239D, I332E, or S239D/I332E, or any of the Fc ADCC variants shown in FIG. 4. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) includes amino acid variants conferring an increase in ADCC activity. In some instances, monomer 1 and monomer 2 can contain identical amino acid substitutions (e.g., both monomers contain a S239D substitution, an I332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the I332E substitution); monomer 2 may contain the S239D/I332E substitutions, and monomer 1 contains the S239D substitution and is WT with respect to the 332 residue (or the reverse where monomer 1 is WT with respect to the 239 residue and contains the I332E substitution); etc.). In still other instances, monomer 1 and monomer 2 can contain amino acid substitutions that are unique to each monomer (e.g., S239D on monomer 1 and I332E on monomer 2, or the reverse). In some embodiments, the heterodimeric Fc backbone pairs have increased ADCC activity, including, but not limited to, those pairs of Fc backbones shown in the Figures.

In some embodiments, one or both of the Fc domains comprising the heterodimeric Fc backbone pairs include amino acid substitutions such that one or both of the Fc domains have improved serum half-life. In certain embodiments, the amino acid substitutions include one or more of: M252Y/S254T/T256E, M428L, N434S, N434A, M428L/N434S, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer). In some embodiments, the heterodimeric Fc backbone pairs have increased serum half-life, including, but not limited to, those pairs of Fc backbones shown in the Figures.

In some embodiments, one or both of the Fc domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, I332E, and S239D/I332E, or any of the Fc ADCC variants shown in FIG. 4, and (ii) one or more amino acid substitutions selected from the group including: M252Y/S254T/T256E, M428L, N434S, N434A, M428L/N434S, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of Fc backbones shown in the Figures.

In some embodiments, one or both of the Fc domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, only monomer 1 or monomer 2 of the heterodimeric Fc backbone pairs include amino acid substitutions that result in an ablated FcγR function (see, e.g., FIG. 3). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 3). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs have ablated FcγR function, including, but not limited to, those pairs of Fc backbones shown in the Figures.

Further, FIG. 41 provides useful C_{L} sequences that can be used with this format (see, e.g., SEQ ID NOs: 1969 and 1970).

3. mAb-scFv Format

One heterodimeric scaffold that finds particular use in the antibodies described herein is the “mAb-scFv” (or “2+1 mAb-scFv”) format, as is shown in FIG. 8E. In this embodiment, the format relies on the use of a C-terminal attachment of a scFv to one of the monomers, thus forming a third ABD, wherein the Fab portions of the two monomers bind a TTA and the “extra” scFv domain binds a NK cell antigen. Thus, the first monomer comprises a first heavy chain (comprising a variable heavy domain and a constant domain), with a C-terminally covalently attached scFv comprising a scFv variable light domain, an scFv linker and a scFv variable heavy domain in either orientation (VH1-CH1-hinge-CH2-CH3-[optional linker]-VH2-scFv linker-VL2 or VH1-CH1-hinge-CH2-CH3-[optional linker]-VL2-scFv linker-VH2). This embodiment further utilizes a common light chain comprising a variable light domain and a constant light domain, that associates with the heavy chains to form two identical Fabs that bind a TTA. In some embodiments, an exemplary mAb-scFv format includes: (i) a first Fc comprising an N-terminal Fab arm that binds a TTA, (ii) a second Fc comprising an N-terminal Fab arm that binds a TTA, and (iii) a C-terminal scFv that binds a NK cell antigen. Such a format can include a first monomer comprising, from the N-terminus to the C-terminus, VH1-CH1-hinge-CH2-CH3, a second monomer comprising, from the N-terminus to the C-terminus, VH1-CH1-hinge-CH2-CH3-scFv, and a third monomer comprising, from the N-terminus to the C-terminus, V_{L}-C_{L}, wherein the first VH1-V_{L} pair bind a TTA, the second VH1-V_{L} pair bind a TTA, and the scFv binds a NK cell antigen. In another embodiment of the mAb-scFv, the first and second VH1-V_{L} pairs bind a NK cell antigen and the scFv binds a TTA. In some embodiments, the heterodimeric antibody of this format is bivalent for the NK cell antigen and monovalent for the TTA antigen. As for many of the embodiments herein, these constructs include Fc ADCC variants, skew variants, pI variants, FcRn variants, ablation variants, additional Fc variants, etc. as desired and described herein. In some embodiments, the heterodimeric antibody of this format is bivalent for the NK cell antigen and monovalent for the TTA antigen.

The antibodies described herein provide mAb-scFv formats, where the NK cell antigen domain sequences comprise variable heavy and variable light domains selected from the group including the variable heavy and variable light domains shown in the Figures, or a variant thereof; and the TTA binding domain sequences comprise variable heavy and variable light domains selected from the group including the variable heavy and variable light domains shown in the Figures, or a variant thereof. In particular embodiments, the αNK cell antigen V_{H} and V_{L} binding domain sequences are selected from the group including the V_{H} and V_{L} binding domain sequences shown in the Figures, or a variant thereof. In particular embodiments, the αTTA V_{H}/V_{L} pairs are selected from the group including the V_{H}/V_{L} pairs shown in the Figures, or a variant thereof.

In some embodiments, the mAb-scFv format includes one or more of: (a) Fc ADCC variants (see, e.g., FIG. 4), (b) skew variants (see, e.g., FIGS. 1A-1F), and/or pI variants (see, e.g., FIGS. 1 and 2). Accordingly, some embodiments include mAb-scFv formats that comprise: (i) a first monomer that comprises the Fc ADCC variants S239D/I332E, the skew variants S364K/E357Q, and a variable heavy domain that, with the variable light domain of the common light chain, makes up an Fv that binds to a TTA as outlined herein, and a scFv domain that binds to a NK cell antigen, (ii) a second monomer that comprises the skew variants L368D/K370S, the pI variants N208D/Q295E/N384D/Q418E/N421D, and a variable heavy domain that, with the variable light domain of the common light chain, makes up an Fv that binds to a TTA as outlined herein, and (iii) a common light chain comprising a variable light domain and a constant light domain. Further, bispecific antibodies in the mAb-scFv format can also include one or more Fc domains with one or more ablation variants (see, e.g., FIG. 3). NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

In some embodiments, the mAb-scFv format includes Fc ADCC variants (see, e.g., FIG. 4), skew variants (see, e.g., FIGS. 1A-1F), pI variants (see, e.g., FIGS. 1 and 2), and/or FcRn variants (e.g., M252Y/S254T/T256E, M428L, N434S, N434A, M428L/N434S, M428L/N434A, etc.). Accordingly, some embodiments include mAb-scFv formats that comprise: (i) a first monomer that comprises the Fc ADCC variants S239D/I332E, the skew variants S364K/E357Q, the FcRn variants M428L/N434S, and a variable heavy domain that, with the variable light domain of the common light chain, makes up an Fv that binds to B7H3 as outlined herein, and a scFv domain that binds to MICA/B, (ii) a second monomer that comprises the skew variants L368D/K370S, the pI variants N208D/Q295E/N384D/Q418E/N421D, the FcRn variants M428L/N434S, and a variable heavy domain that, with the variable light domain of the common light chain, makes up an Fv that binds to B7H3 as outlined herein, and (iii) a common light chain comprising a variable light domain and a constant light domain. Further, bispecific antibodies in the mAb-scFv format can also include one or more Fc domains with one or more ablation variants (see, e.g., FIG. 3). NK cell antigen binding domain sequences finding particular use in these embodiments include, but are not limited to, those shown in the Figures, or variants thereof. TTA binding domain sequences finding particular use in these embodiments include, but are not limited to, those shown in the Figures, or variants thereof.

FIGS. 43A-43G show some exemplary Fc domain sequences that are useful with the mAb-scFv format. Generally, the “monomer 1” sequences (as depicted in the Figures) refer to the Fc domain of the “Fab-Fc heavy chain,” and the “monomer 2” sequences (as depicted in the Figures) refer to the Fc domain of the “Fab-Fc-scFv heavy chain.”

In some embodiments, one or both of the Fc domains comprising the heterodimeric Fc backbone pairs are configured such that one or both of the Fc domains have wildtype FcγR effector function. In some embodiments, the heterodimeric Fc backbone pairs have wildtype FcγR effector function, including, but not limited to, those pairs of: SEQ ID NOs: 1798 and 1975, as shown in FIG. 43A.

In some embodiments, one or both of the Fc domains comprising the heterodimeric Fc backbone pairs include amino acid substitutions such that one or both of the Fc domains have increased ADCC activity. In certain embodiments, the amino acid substitutions include one or more of: S239D, I332E, or S239D/I332E, or any of the Fc ADCC variants shown in FIG. 4. In some instances, only monomer 1 or monomer 2 of the heterodimeric Fc backbone pairs include amino acid substitutions resulting in increased ADCC activity. In such an instance, the pair of heterodimeric Fc backbones may be referred to as an asymmetric, ADCC-enhanced heterodimeric Fc backbone pair. In other instances, both monomers (i.e., monomer 1 and monomer 2) of the heterodimeric Fc backbone pairs independently include one or more amino acid substitutions conferring an increased ADCC activity. In such an instance, the pair of heterodimeric Fc backbones may be referred to as a symmetric, ADCC-enhanced heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) includes amino acid variants conferring an increase in ADCC activity. In some instances, monomer 1 and monomer 2 can contain identical amino acid substitutions (e.g., both monomers contain a S239D substitution, an I332E substitution, or both substitutions are present on both monomers). In other instances, monomer 1 and monomer 2 can contain at least one amino acid substitution that is the same (e.g., monomer 1 may contain the S239D/I332E substitutions, and monomer 2 contains the S239D substitution and is WT with respect to the 332 residue (or the reverse where monomer 2 is WT with respect to the 239 residue and contains the I332E substitution); monomer 2 may contain the S239D/I332E substitutions, and monomer 1 contains the S239D substitution and is WT with respect to the 332 residue (or the reverse where monomer 1 is WT with respect to the 239 residue and contains the I332E substitution); etc.). In still other instances, monomer 1 and monomer 2 can contain amino acid substitutions that are unique to each monomer (e.g., S239D on monomer 1 and I332E on monomer 2, or the reverse). In some embodiments, the heterodimeric Fc backbone pairs have increased ADCC activity, including, but not limited to, those pairs of: (i) SEQ ID NOs: 1820 and 1976, (ii) SEQ ID NOs: 1822 and 1977, (iii) SEQ ID NOs: 1824 and 1978, (iv) SEQ ID NOs: 1826 and 1979, (v) SEQ ID NOs: 1828 and 1980, (vi) SEQ ID NOs: 1830 and 1981, (vii) SEQ ID NOs: 1832 and 1982, (viii) SEQ ID NOs: 1834 and 1983, (ix) SEQ ID NOs: 1836 and 1984, (x) SEQ ID NOs: 1838 and 1985, (xi) SEQ ID NOs: 1840 and 1986, (xii) SEQ ID NOs: 1842 and 1987, (xiii) SEQ ID NOs: 1844 and 1988, (xiv) SEQ ID NOs: 1846 and 1989, and (xv) SEQ ID NOs: 1848 and 1990, as shown in FIGS. 43A-43D.

In some embodiments, one or both of the Fc domains comprising the heterodimeric Fc backbone pairs include amino acid substitutions such that one or both of the Fc domains have improved serum half-life. In certain embodiments, the amino acid substitutions include one or more of: M252Y/S254T/T256E, M428L, N434S, N434A, M428L/N434S, or M428L/N434A. In some instances, only monomer 1 or monomer 2 of the heterodimeric Fc backbone pairs include amino acid substitutions resulting in improved serum half-life. In such an instance, the pair of heterodimeric Fc backbones may be referred to as an asymmetric, Xtend heterodimeric Fc backbone pair. In other instances, both monomers (i.e., monomer 1 and monomer 2) of the heterodimeric Fc backbone pairs independently include one or more amino acid substitutions conferring an increase serum half-life. In such an instance, the pair of heterodimeric Fc backbones may be referred to as a symmetric, Xtend heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increased serum half-life. In some instances, monomer 1 and monomer 2 can contain identical amino acid substitutions. In other instances, monomer 1 and monomer 2 can contain at least one amino acid substitution that is the same (e.g., both monomers contain the M428L substitution, one monomer further contains the N434S substitution, and the other monomer contains the N434A mutation). In some instances, monomer 1 and monomer 2 can contain amino acid substitutions that are unique to each monomer (e.g., N434S on one monomer, and N434A on the other monomer). In some embodiments, the heterodimeric Fc backbone pairs have increased serum half-life, including, but not limited to, those pairs of Fc backbones shown in the Figures.

In some embodiments, one or both of the Fc domains comprising the heterodimeric Fc backbone pairs include amino acid substitutions such that one or both of the Fc domains have increased ADCC activity and improved serum half-life. In certain embodiments, the amino acid substitutions include: (i) one or more of amino acid substitutions selected from the group including: S239D, I332E, and S239D/I332E, or any of the Fc ADCC variants shown in FIG. 4, and (ii) one or more amino acid substitutions selected from the group including: M252Y/S254T/T256E, M428L, N434S, N434A, M428L/N434S, and M428L/N434A. In some instances, only monomer 1 or monomer 2 of the heterodimeric Fc backbone pairs include amino acid substitutions resulting in increased ADCC and increased serum half-life. In such an instance, the pair of heterodimeric Fc backbones may be referred to as an asymmetric, ADCC-enhanced heterodimeric Fc backbone pair with Xtend. In other instances, both monomers (i.e., monomer 1 and monomer 2) of the heterodimeric Fc backbone pairs independently comprise amino acid substitutions resulting in increased ADCC and increased serum half-life. In such an instance, the pair of heterodimeric Fc backbones may be referred to as a symmetric, ADCC-enhanced heterodimeric Fc backbone pair with Xtend. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants conferring an increase in ADCC activity and improved serum half-life. In some instances, monomer 1 and monomer 2 can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity and increased serum-half-life, including, but not limited to, those pairs of: (i) SEQ ID NOs: 1850 and 1991, (ii) SEQ ID NOs: 1852 and 1992, (iii) SEQ ID NOs: 1854 and 1993, (iv) SEQ ID NOs: 1856 and 1994, (v) SEQ ID NOs: 1858 and 1995, (vi) SEQ ID NOs: 1860 and 1996, (vii) SEQ ID NOs: 1862 and 1997, (viii) SEQ ID NOs: 1864 and 1998, (ix) SEQ ID NOs: 1866 and 1999, (x) SEQ ID NOs: 1868 and 2000, (xi) SEQ ID NOs: 1870 and 2001, (xii) SEQ ID NOs: 1872 and 2002, (xiii) SEQ ID NOs: 1874 and 2003, (xiv) SEQ ID NOs: 1876 and 93, and (xv) SEQ ID NOs: 1878 and 80, as shown in FIGS. 43D-43G.

In some embodiments, one or both of the Fc domains comprising the heterodimeric Fc backbone pairs include amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, only monomer 1 or monomer 2 of the heterodimeric Fc backbone pairs include amino acid substitutions that result in an ablated FcγR function (see, e.g., FIG. 3). In such an instance, the pair of heterodimeric Fc backbones may be referred to as an asymmetric, FcKO heterodimeric Fc backbone pair. In other instances, both monomers (i.e., monomer 1 and monomer 2) of the heterodimeric Fc backbone pairs independently comprise one or more amino acid substitutions resulting in an ablated FcγR function (see, e.g., FIG. 3). In such an instance, the pair of heterodimeric Fc backbones may be referred to as a symmetric, FcKO heterodimeric Fc backbone pair. As used herein, the terms “asymmetric” and “symmetric” are not intended to mean that the heterodimeric Fc backbone pairs comprise identical mutations, but rather that only one Fc domain (in the case of asymmetric pairs) or both Fc domains (in the case of symmetric pairs) contains amino acid variants resulting in an ablated FcγR function. In some embodiments, the heterodimeric Fc backbone pairs have ablated FcγR function, including, but not limited to, those pairs of: SEQ ID NOs: 1973 and 1974, as shown in FIG. 43A.

4. 1+1 CLC Format

One heterodimeric scaffold that finds particular use in the antibodies described herein is the “1+1 common light chain” (or “1+1 CLC”) format, as is shown in FIG. 8C. The 1+1 CLC format antibody includes: a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer “common light chain” comprising V_{L}-C_{L}, wherein V_{L} is a common variable light domain and C_{L} is a constant light domain. In such embodiments, the V_{L} pairs with the VH1 to form a first binding domain with a first antigen binding specificity; and the V_{L} pairs with the VH2 to form a second binding domain with a second antigen binding specificity. In some embodiments, the 1+1 CLC format antibody is a bivalent antibody.

In some embodiments, the first and/or second Fc domains of the 1+1 CLC format are variant Fc domains. Generally, variant Fc domains can comprise one or more of: (a) one or more Fc ADCC variants (see, e.g., FIG. 4), (b) one or more skew variants (see, e.g., FIGS. 1A-1F), (c) one or more ablation variants (see, e.g., FIG. 3), (d) one or more FcRn variants (e.g., M428L, N434S, N434A, M428L/N434S, M428L/N434A, M252Y/S254T/T256E, etc.), (e) a scFv linker (including those shown in FIG. 5; see, e.g., SEQ ID NOs: 1-11, 20, 21, and 81-92), and one or both of the heavy chains can comprise one or more pI variants (see, e.g., FIGS. 1 and 2). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, I332E, or S239D/I332E, or any of the Fc ADCC variants shown in FIG. 4. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fe domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 1A-1F). Particularly useful heterodimerization skew variants include, but are not limited to: (i) S364K/E357Q: L368D/K370S, (ii) L368D/K370S: S364K, (iii) L368E/K370S: S364K, (iv) T411E/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fc domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are only included on one of the two Fc domains. In such instances, the heterodimerization skew variants are in an asymmetric configuration. In some other instances, the heterodimerization skew variants are included on both of the Fc domains. In such instances, the heterodimerization skew variants are in a symmetric configuration. As used herein, the terms “asymmetric configuration” and “symmetric configuration” are not intended to mean that the heterodimeric Fc backbone pairs comprise identical heterodimerization skew variants, but rather that only one Fc domain (in the case of asymmetric configurations) or both Fc domains (in the case of symmetric configurations) includes heterodimerization skew variants. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fc domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M252Y/S254T/T256E, M428L, N434S, N434A, M428L/N434S, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 1 and 2). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 1 and 2), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, one of the first or second antigen binding domains is a NK cell antigen binding domain. Any suitable NK cell antigen binding domain (as described herein and in the Figures, or a variant thereof) can be included in the subject 1+1 CLC format antibody.

In some embodiments, one of the first or second antigen binding domains is a TTA binding domain. Any suitable TTA binding domain (as described herein and in the Figures, or a variant thereof) can be included in the subject 1+1 CLC format antibody.

5. 2+1 CLC Format

One heterodimeric scaffold that finds particular use in the antibodies described herein is the “2+1 common light chain” (or (“2+1 CLC”) format, as is shown in FIG. 8D. The 2+1 CLC format includes: a first monomer that includes a VH1-CH1-linker-VH1-CH1-hinge-CH2-CH3, wherein the first and second VH1 are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” V_{L}-C_{L}, wherein V_{L} is a common variable light domain and C_{L} is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including, but not limited to, any one of the domain linkers described in FIGS. 6 and 7 (see, e.g., SEQ ID NOs: 30, 31, 40, 41, 50, 51, and 94-119). In some embodiments, the linker is EPKSCGKPGSGKPGS (SEQ ID NO: 115). In some embodiments, the 2+1 CLC format antibody is a trivalent antibody.

In some embodiments, the first and/or second Fc domains of the 2+1 CLC format are variant Fc domains. Generally, variant Fc domains can comprise one or more of: (a) one or more Fc ADCC variants (see, e.g., FIG. 4), (b) one or more skew variants (see, e.g., FIGS. 1A-1F), (c) one or more ablation variants (see, e.g., FIG. 3), (d) one or more FcRn variants (e.g., M252Y/S254T/T256E, M428L, N434S, N434A, M428L/N434S, M428L/N434A, etc.), (e) a scFv linker (including those shown in FIG. 5; see, e.g., SEQ ID NOs: 1-11, 20, 21, and 81-92), and one or both of the heavy chains can comprise one or more pI variants (see, e.g., FIGS. 1 and 2). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, I332E, or S239D/I332E, or any of the Fc ADCC variants shown in FIG. 4. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fc domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 1A-1F). Particularly useful heterodimerization skew variants include, but are not limited to: (i) S364K/E357Q: L368D/K370S, (ii) L368D/K370S: S364K, (iii) L368E/K370S: S364K, (iv) T411E/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fc domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are in an asymmetric configuration, as described above. In other embodiments, the heterodimerization skew variants are in a symmetric configuration, as described above. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fc domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M252Y/S254T/T256E, M428L, N434S, N434A, M428L/N434S, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 1 and 2). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 1 and 2), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, each of the first antigen binding domains or the second antigen binding domain is a NK cell antigen binding domain. Any suitable NK cell antigen binding domain (as described herein and in the Figures, or a variant thereof) can be included in the subject 2+1 CLC format antibody.

In some embodiments, each of the first antigen binding domains or the second antigen binding domain is a TTA binding domain. Any suitable TTA binding domain (as described herein and in the Figures, or a variant thereof) can be included in the subject 2+1 CLC format antibody.

6. mAb-Fv Format

One heterodimeric scaffold that finds particular use in the antibodies described herein is the “mAb-Fv” format, as is shown in FIG. 8K. In this embodiment, the format relies on the use of a C-terminal attachment of an “extra” variable heavy domain to one monomer and the C-terminal attachment of an “extra” variable light domain to the other monomer, thus forming a third ABD (i.e., an “extra” Fv domain), wherein the Fab portions of the two monomers bind a TTA and the “extra” scFv domain binds a NK cell antigen.

In this embodiment, the first monomer comprises: a first heavy chain, comprising a first variable heavy domain and a first constant heavy domain comprising a first Fc domain, with a first variable light domain covalently attached to the C-terminus of the first Fc domain using a domain linker (VH1-CH1-hinge-CH2-CH3-[optional linker]-VL2). The second monomer comprises: a second variable heavy domain, a second constant heavy domain comprising a second Fc domain, and a third variable heavy domain covalently attached to the C-terminus of the second Fc domain using a domain linker (VH1-CH1-hinge-CH2-CH3-[optional linker]-VH2). This embodiment further utilizes a common light chain comprising a variable light domain and a constant light domain, which associates with the heavy chains to form two identical Fabs that include two identical Fvs. The two C-terminally attached variable domains (VL2 and VH2) make up the “extra” third Fv. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject mAb-Fv format antibody.

Any suitable TTA binding domain (as described herein and in the Figures, or a variant thereof) can be included in the subject mAb-Fv format antibody.

7. Dual scFv Format

The antibodies described herein also provide dual scFv formats as are known in the art (see, e.g., FIG. 8F). In this embodiment, the anti-NK cell antigen x anti-TTA heterodimeric bispecific antibody is made up of two scFv-Fc monomers (both in either the V_{H}-scFv linker-V_{L}-[optional domain linker]-CH2-CH3 format, the V_{L}-scFv linker-V_{H}-[optional domain linker]-CH2-CH3 format, or with one monomer in one orientation and the other monomer in the other orientation).

In this case, all ABDs are in the scFv format. Any suitable NK cell ABD and TTA ABD can be included in the subject bispecific antibodies in the dual scFv format, including any of the NK cell ABDs and TTA ABDs described herein and in the Figures, as well as variants thereof.

In some embodiments, the first and/or second Fc domains of the dual scFv format are variant Fc domains. Generally, variant Fc domains can comprise one or more of: (a) one or more Fc ADCC variants (see, e.g., FIG. 4), (b) one or more skew variants (see, e.g., FIGS. 1A-1F), (c) one or more ablation variants (see, e.g., FIG. 3), (d) one or more FcRn variants (e.g., M428L, N434S, N434A, M428L/N434S, M428L/N434A, M252Y/S254T/T256E, etc.), (e) a scFv linker (including those shown in FIG. 5; see, e.g., SEQ ID NOs: 1-11, 20, 21, and 81-92), and one or both of the heavy chains can comprise one or more pI variants (see, e.g., FIGS. 1 and 2). The various types of variants can be used alone, or in combination with other variant types described herein.

In some embodiments, one or both of the Fc domains are configured such that one or both of the Fc domains have wildtype FcγR effector function. In some embodiments, the heterodimeric Fc backbone pairs have wildtype FcγR effector function, including, but not limited to, those pairs of Fc backbones shown in the Figures.

In some embodiments, one or both of the Fc domains include one or more amino acid substitutions such that one or both of the Fc domains have increased ADCC activity. In certain embodiments, the amino acid substitutions include one or more of: S239D, I332E, or S239D/I332E, or any of the Fc ADCC variants shown in FIG. 4. In some instances, the Fc backbone pair comprises an asymmetric, ADCC-enhanced heterodimeric Fc backbone pair (as described above). In other embodiments, the Fc backbone pair comprises a symmetric, ADCC-enhanced heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased ADCC activity, including, but not limited to, those pairs of Fc backbones shown in the Figures. In some embodiments, one or both of the Fc domains include one or more amino acid substitutions such that one or both of the Fc domains have increased ADCC activity, and optionally one or more of: (i) one or more skew variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include heterodimerization skew variants (e.g., a set of amino acid substitutions, including, but not limited to, those shown in FIGS. 1A-1F). Particularly useful heterodimerization skew variants include, but are not limited to: (i) S364K/E357Q: L368D/K370S, (ii) L368D/K370S: S364K, (iii) L368E/K370S: S364K, (iv) T411E/K360E/Q362E: D401K, (v) L368D/K370S: S364K/E357L, (vi) K370S: S364K/E357Q, (vii) T366S/L368A/Y407V: T366W, and (viii) T366S/L368A/Y407V/Y349C: T366W/S354C (according to EU numbering). In exemplary embodiments, one of the first or second variant Fe domains includes the heterodimerization skew variants L368D/K370S, and the other of the second or first variant Fc domain includes heterodimerization skew variants S364K/E357Q, wherein numbering is according to EU numbering. In some instances, the heterodimerization skew variants are in an asymmetric configuration, as described above. In other embodiments, the heterodimerization skew variants are in a symmetric configuration, as described above. In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, one or both of the Fc domains include heterodimerization skew variants, and optionally one or more of: (i) one or more Fc ADCC variants, (ii) one or more ablation variants, (iii) one or more variants that improve serum half-life, (iv) one or more pI variants in the constant domain of the first and/or second monomer, or (v) any combination thereof.

In some embodiments, one or both of the Fc domains include one or more amino acid substitutions such that one or both of the Fc domains have improved serum half-life. In certain embodiments, the amino acid substitutions include one or more of: M252Y/S254T/T256E, M428L, N434S, N434A, M428L/N434S, or M428L/N434A. In some instances, the Fc backbone pair comprises an asymmetric, Xtend heterodimeric Fc backbone pair (as described above). In other embodiments, the Fc backbone pair comprises a symmetric, Xtend heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have increased serum half-life, including, but not limited to, those pairs of Fc backbones shown in the Figures. In some embodiments, one or both of the Fc domains include one or more amino acid substitutions such that one or both of the Fc domains have improved serum half-life, and optionally one or more of: (i) one or more skew variants, (ii) one or more ablation variants, (iii) one or more Fc ADCC variants, (iv) one or more pI variants in the constant domain of the first and/or second monomer, or (v) any combination thereof.

In some embodiments, one or both of the Fc domains include one or more amino acid substitutions such that one or both of the Fc domains have an ablated FcγR function. Nonlimiting examples of amino acid substitutions (and combinations thereof) conferring an ablated FcγR function are shown in, for example, FIG. 3. In some instances, the Fc backbone pair comprises an asymmetric FcKO heterodimeric Fc backbone pair (as described above). In other embodiments, the Fc backbone pair comprises a symmetric, FcKO heterodimeric Fc backbone pair (as described above). In some instances, monomer 1 and monomer 2 (of the heterodimeric Fc backbone pair) can contain identical amino acid substitutions. In other instances, monomer 1 and monomer 2 can contain at least one amino acid substitution that is the same. In still other instances, monomer 1 and monomer 2 can contain amino acid substitutions that are unique to each monomer. In some embodiments, the heterodimeric Fc backbone pairs have ablated FcγR function, including, but not limited to, those pairs of Fc backbones shown in the Figures. In some embodiments, each of the first and second Fc domains include the ablation variants E233P/L234V/L235A/G236_/S267K, wherein numbering is according to EU numbering. In some embodiments, one or both of the Fc domains include one or more amino acid substitutions such that one or both of the Fc domains have an ablated FcγR function, and optionally one or more of: (i) one or more skew variants, (ii) one or more Fc ADCC variants, (iii) one or more variants that improve serum half-life, (iv) one or more pI variants in the constant domain of the first and/or second monomer, or (v) any combination thereof.

In some embodiments, the constant domain (CH1-hinge-CH2-CH3) of the first and/or second monomer includes pI variants (including, but not limited to, those shown in FIGS. 1 and 2). In exemplary embodiments, the constant domain (CH1-hinge-CH2-CH3) of the first or second monomer includes pI variants N208D/Q295E/N384D/Q418E/N421D, wherein numbering is according to EU numbering. In some embodiments, the constant domain of the first and/or second monomer includes pI variants (including, but not limited to, those shown in FIGS. 1 and 2), and the first and/or second Fc domain optionally contains one or more of: (i) one or more skew variants, (ii) one or more ablation variants, (iii) one or more variants that improve serum half-life, (iv) one or more Fc ADCC variants, or (v) any combination thereof.

In some embodiments, the first scFv or the second scFv is the domain that binds to the NK cell antigen. Any suitable NK cell antigen V_{H}/V_{L} pairs described herein (and in the Figures), or a variant thereof, can be included in the subject dual scFv format antibody.

In some embodiments, the first scFv or the second scFv is the domain that binds to the TTA. Any suitable TTA V_{H}/V_{L} pairs described herein (and in the Figures), or a variant thereof, can be included in the subject dual scFv format antibody.

8. One-Armed scFv-mAb Format

One heterodimeric scaffold that finds particular use in the antibodies described herein is the “one-armed scFv-mAb” format, as is shown in FIG. 8G. This format includes: 1) a first monomer that comprises an “empty” Fc domain; 2) a second monomer that includes a first variable heavy domain (V_{H}), a scFv domain (a second ABD), an Fc domain, where the scFv domain is attached to the N-terminus of the first variably heavy domain; and 3) a light chain that includes a first variable light domain and a constant light domain. The first variable heavy domain and the first variable light domain form a first antigen binding domain and the scFv is a second antigen binding domain. In this format, one of the first ABDs and the second ABDs binds a NK cell antigen, and the other ABD binds a TTA. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject one-armed scFv-mAb format antibody.

Any suitable TTA binding domain (as described herein and in the Figures, or a variant thereof) can be included in the subject one-armed scFv-mAb format antibody.

9. One-Armed Central-scFv Format

One heterodimeric scaffold that finds particular use in the antibodies described herein is the “one-armed central-scFv” format (also referred to herein as the “1+1 Empty×Fab-scFv” format), as is shown in FIG. 8J. In this embodiment, one monomer comprises just an Fc domain (i.e., a first Fc domain), while the other monomer includes a Fab domain (a first ABD), a scFv (a second ABD), and an Fc domain (i.e., a second Fc domain), where the scFv domain is inserted between the first Fc domain and the second Fc domain. In this format, the Fab portion binds one receptor target and the scFv binds another. In this format, either the Fab portion binds a TTA and the scFv binds a NK cell antigen, or vice versa.

In this embodiment, one monomer comprises a first heavy chain comprising a first variable heavy domain, a CH1 domain, and a Fc domain, with a scFv comprising a scFv variable light domain, an scFv linker, and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain that associates with the heavy chain to form a Fab. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject one-armed central-scFv format antibody.

Any suitable TTA binding domain (as described herein and in the Figures, or a variant thereof) can be included in the subject one-armed central-scFv format antibody.

10. Central-Fv Format

One heterodimeric scaffold that finds particular use in the antibodies described herein is the “central-Fv” format, as is shown in FIG. 8L. In this embodiment, the format relies on the use of an inserted Fv domain (i.e., the central Fv domain) thus forming an “extra” third ABD, wherein the Fab portions of the two monomers bind a TTA and the “extra” central Fv domain binds a NK cell antigen. The Fv domain is inserted between the Fc domain and the CH1-Fv region of the monomers, thus providing a third ABD, wherein each monomer contains a component of the Fv (e.g., one monomer comprises a variable heavy domain and the other comprises a variable light domain of the “extra” central Fv domain).

In this embodiment, one monomer comprises a first heavy chain comprising a first variable heavy domain, a CH1 domain, and Fc domain, and an additional variable light domain. The additional variable light domain is covalently attached between the C-terminus of the CH1 domain of the heavy constant domain and the N-terminus of the first Fc domain using domain linkers (VH1-CH1-[optional linker]-VL2-hinge-CH2-CH3). The other monomer comprises a first heavy chain comprising a first variable heavy domain, a CH1 domain and Fc domain, and an additional variable heavy domain (VH1-CH1-[optional linker]-VH2-hinge-CH2-CH3). The additional variable heavy domain is covalently attached between the C-terminus of the CH1 domain of the heavy constant domain and the N-terminus of the first Fc domain using domain linkers. This embodiment further utilizes a common light chain comprising a variable light domain and a constant light domain, that associates with the heavy chains to form two identical Fabs that each bind a TTA. The additional variable heavy domain and additional variable light domain form an “extra” central Fc that binds a NK cell antigen. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject central-Fv format antibody.

Any suitable TTA binding domain (as described herein and in the Figures, or a variant thereof) can be included in the subject central-Fv format antibody.

11. scFv-mAb Format

One heterodimeric scaffold that finds particular use in the antibodies described herein is the “scFv-mAb” format, as is shown in FIG. 8H. In this embodiment, the format relies on the use of a N-terminal attachment of a scFv to one of the monomers, thus forming a third ABD, wherein the Fab portions of the two monomers bind a TTA and the “extra” scFv domain binds a NK cell antigen.

In this embodiment, the first monomer comprises a first heavy chain (comprising a variable heavy domain and a constant domain), with a N-terminally covalently attached scFv comprising a scFv variable light domain, an scFv linker, and a scFv variable heavy domain in either orientation ((VH1-scFv linker-VL1-[optional domain linker]-VH2-CH1-hinge-CH2-CH3) or (with the scFv in the opposite orientation (VL1-scFv linker-VH1-[optional domain linker]-VH2-CH1-hinge-CH2-CH3))). This embodiment further utilizes a common light chain comprising a variable light domain and a constant light domain that associates with the heavy chains to form two identical Fabs that bind a TTA. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject scFv-mAb format antibody.

Any suitable TTA binding domain (as described herein and in the Figures, or a variant thereof) can be included in the subject scFv-mAb format antibody.

12. 2+1 Stack Fab₂-scFv-Fc Format

One heterodimeric scaffold that finds particular use in the antibodies described herein is the “2+1 stack Fab₂-scFv-Fc” (or “stack Fab₂-scFv-Fc” or “2+1 Fab2×scFv”) format, as is shown in FIG. 8N. In this embodiment, the format relies on the use of a “stacked” Fab portion that binds a first target antigen (e.g., a TTA), and an scFv domain that binds a second target antigen (e.g., a NK cell antigen). In this format, the first monomer comprises, from N-terminus to C-terminus, a first heavy chain (comprising a variable heavy domain and a constant domain), a domain linker, and a second heavy chain (comprising a variable heavy domain and a constant domain), and a first Fc domain; the second monomer comprises, from N-terminus to C-terminus, a scFv comprising a scFv variable light domain, an scFv linker, and a scFv variable heavy domain in either orientation ((VH1-CH1-hinge-CH2-CH3-[optional linker]-VH2-scFv linker-VL2) or (VH1-CH1-hinge-CH2-CH3-[optional linker]-VL2-scFv linker-VH2)), and a second Fc domain; and the third monomer comprises a common light chain including a variable light domain and a constant light domain, that associates with the heavy chains to form two identical Fabs that bind a TTA. As for many of the embodiments herein, these constructs can include one or more of: (i) Fc ADCC variants, (ii) pI variants, (iii) ablation variants, (iv) skew variants, (v) variants that improve serum half-life, as well as any combination thereof, as desired and described herein.

Any suitable NK cell antigen binding domain (as described herein and in the Figures, or a variant thereof) can be included in the subject 2+1 stack Fab₂-scFv-Fc format antibody.

Any suitable TTA binding domain (as described herein and in the Figures, or a variant thereof) can be included in the subject 2+1 stack Fab₂-scFv-Fc format antibody.

13. Non-Heterodimeric Bispecific Antibodies

As will be appreciated by those in the art, the anti-NK cell antigen x anti-TTA antibodies provided herein can also be included in non-heterodimeric bispecific formats (see, e.g., FIG. 8I).

In this format, the anti-NK cell antigen x anti-TTA antibody includes: (i) a first monomer comprising a VH1-CH1-hinge-CH2-CH3, (ii) a second monomer comprising a VH2-CH1-hinge-CH2-CH3, (iii) a first light chain comprising a VL1-C_{L}, and (iv) a second light chain comprising a VL2-C_{L}. In such embodiments, the VH1 and VL1 form a first ABD, and VH2 and VL2 form a second ABD. One of the first or second ABDS binds a TTA, and the other ABD binds a NK cell antigen.

Any suitable NK cell antigen binding domain (as described herein and in the Figures, or a variant thereof) can be included in the subject non-heterodimeric bispecific format antibody.

Any suitable TTA binding domain (as described herein and in the Figures, or a variant thereof) can be included in the subject non-heterodimeric bispecific format antibody. In some embodiments, the TTA binding domain binds to the extracellular domain of any human TTA selected from the group including EGFR, HER2, Trop2, B7H3, FLT3, DLL3, CD19, CD20, CD123, CEA, MSLN, BCMA, CAIX, CLDN18.2, PD-1 and ANOL.

14. Trident Format

In some embodiments, the bispecific antibodies described herein are in the “Trident” format as generally described in WO2015/184203, hereby expressly incorporated by reference in its entirety and in particular for the Figures, Legends, definitions, and sequences of “Heterodimer-Promoting Domains” or “HPDs”, including “K-coil” and “E-coil” sequences. Tridents rely on using two different HPDs that associate to form a heterodimeric structure as a component of the structure. In this embodiment, the Trident format includes a “traditional” heavy and light chain (e.g., VH1-CH1-hinge-CH2-CH3 and VL1-CL), a third chain comprising a first “diabody-type binding domain” or “DART®,” VH2-(linker)-VL3-HPD1, and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, the second and third ABDs bind the same antigen, in this instance generally a TTA, e.g., bivalently, with the first ABD binding a NK cell antigen monovalently.

Any suitable NK cell antigen binding domain (as described herein and in the Figures, or a variant thereof) can be included in the subject Trident format antibody.

Any suitable TTA binding domain (as described herein and in the Figures, or a variant thereof) can be included in the subject Trident format antibody. In some embodiments, the TTA binding domain binds to the extracellular domain of any human TTA selected from the group including EGFR, HER2, Trop2, B7H3, FLT3, DLL3, CD19, CD20, CD123, CEA, MSLN, BCMA, CAIX, CLDN18.2, PD-1 and ANO1.

D. Particular Embodiments of the Invention with Increased Binding to FcγRIIIa

As discussed herein, the disclosure provides NKE antibodies, including multispecific antibodies, that bind to NK antigens, CD16A, and a tumor target antigen (TTA), and have increased binding to CD16A as compared to human IgG1. As discussed herein, the ABDs that bind to either the NK antigen or the TTA can be monovalent or bivalent binding.

Preferred combinations of NK ABDs and TTAs include, but are not limited to, NKG2D and CD19, NKG2D and CAIX, NKG2D and 5T4, NKG2D and CD20, NKG2D and FLT3, NKG2D and EGFR, NKG2D and CXCR4, NKG2D and EpCAM, NKG2D and CLEC12A, NKG2D and CD33, NKG2D and HER2, NKG2D and BCMA, NKG2D and FAP, NKG2D and CD22, NKG2D and CD38, NKG2D and DLL3, NKG2D and P-Cadherin, NKG2D and cMET, and NKG2D and B7H3, wherein the ABDs for each are selected from the sequences depicted herein.

Preferred combinations of NK ABDs and TTAs include, but are not limited to, NKp46 and CD19, NKp46 and CAIX, NKp46 and 5T4, NKp46 and CD20, NKp46 and FLT3, NKp46 and EGFR, NKp46 and CXCR4, NKp46 and EpCAM, NKp46 and CLEC12A, NKp46 and CD33, NKp46 and HER2, NKp46 and BCMA, NKp46 and FAP, NKp46 and CD22, NKp46 and CD38, NKp46 and DLL3, NKp46 and P-Cadherin, NKp46 and cMET, and NKp46 and B7H3, wherein the ABDs for each are selected from the sequences depicted herein.

Preferred combinations of NK ABDs and TTAs include, but are not limited to, NKp30 and CD19, NKp30 and CAIX, NKp30 and 5T4, NKp30 and CD20, NKp30 and FLT3, NKp30 and EGFR, NKp30 and CXCR4, NKp30 and EpCAM, NKp30 and CLEC12A, NKp30 and CD33, NKp30 and HER2, NKp30 and BCMA, NKp30 and FAP, NKp30 and CD22, NKp30 and CD38, NKp30 and DLL3, NKp30 and P-Cadherin, NKp30 and cMET, and NKp30 and B7H3, wherein the ABDs for each are selected from the sequences depicted herein.

In some embodiments, the NKE multispecific antibodies are as generally described in WO2019/101695, hereby incorporated by reference in its entirety, with the addition of Fc variants that increase the binding of the Fc domain to CD16A.

Embodiment (A): In some embodiments, the NKE antibody comprises: a) means for binding the extracellular domain of human NKG2D; and b) a variant IgG1 Fc dimeric domain comprising an amino acid substitution as compared to human IgG1, wherein the variant has increased in vitro binding to the human FcγRIIIa receptor as compared to human IgG1. Suitable variable heavy VH and variable light VL domains that bind to the ECD of human NKG2D include, but are not limited to, VH/VL pairs selected from the group including: (i) SEQ ID NOs: 911 and 915, (ii) SEQ ID NOs: 919 and 923, (iii) SEQ ID NOs: 927 and 931, (iv) SEQ ID NOs: 935 and 939, (v) SEQ ID NOs: 943 and 944, (vi) SEQ ID NOs: 945 and 946, (vii) SEQ ID NOs: 947 and 948, (viii) SEQ ID NOs: 949 and 950, (ix) SEQ ID NOs: 951 and 952, (x) SEQ ID NOs: 953 and 954, (xi) SEQ ID NOs: 955 and 956, (xii) SEQ ID NOs: 957 and 958, (xiii) SEQ ID NOs: 959 and 960, (xiv) SEQ ID NOs: 961 and 962, (xv) SEQ ID NOs: 963 and 964, (xvi) SEQ ID NOs: 965 and 966, (xvii) SEQ ID NOs: 967 and 968, (xviii) SEQ ID NOs: 969 and 970, (xix) SEQ ID NOs: 971 and 972, (xx) SEQ ID NOs: 973 and 2004, (xxi) SEQ ID NOs: 974 and 975, (xxii) SEQ ID NOs: 976 and 977, (xxiii) SEQ ID NOs: 978 and 979, (xxiv) SEQ ID NOs: 980 and 981, (xxv) SEQ ID NOs: 982 and 983, (xxvi) SEQ ID NOs: 984 and 985, (xxvii) SEQ ID NOs: 986 and 987, (xxviii) SEQ ID NOs: 988 and 989, (xxix) SEQ ID NOs: 990 and 991, (xxx) SEQ ID NOs: 992 and 993, (xxxi) SEQ ID NOs: 994 and 995, (xxxii) SEQ ID NOs: 996 and 997, (xxxiii) SEQ ID NOs: 998 and 999, (xxxiv) SEQ ID NOs: 1000 and 1001, (xxxv) SEQ ID NOs: 1002 and 1003, (xxxvi) SEQ ID NOs: 1004 and 1005, (xxxvii) SEQ ID NOs: 1006 and 1007, (xxxviii) SEQ ID NOs: 1008 and 1009, (xxxix) SEQ ID NOs: 1010 and 1011, (xl) SEQ ID NOs: 1012 and 1013, (xli) SEQ ID NOs: 1014 and 1015, (xlii) SEQ ID NOs: 1016 and 1017, (xliii) SEQ ID NOs: 1018 and 1019, (xliv) SEQ ID NOs: 1020 and 1021, (xlv) SEQ ID NOs: 1024 and 1025, (xlvi) SEQ ID NOs: 1026 and 1027, (xlvii) SEQ ID NOs: 1028 and 1029, (xlviii) SEQ ID NOs: 1030 and 1031, (xlix) SEQ ID NOs: 1032 and 1033, (1) SEQ ID NOs: 1034 and 1035, (li) SEQ ID NOs: 1036 and 1037, (lii) SEQ ID NOs: 1038 and 1039, (liii) SEQ ID NOs: 1040 and 1041, (liv) SEQ ID NOs: 1042 and 1043, (lv) SEQ ID NOs: 1042 and 1045, (lvi) SEQ ID NOs: 1044 and 1043, (lvii) SEQ ID NOs: 1044 and 1045, (lviii) SEQ ID NOs: 1046 and 1049, (lix) SEQ ID NOs: 1046 and 1050, (lx) SEQ ID NOs: 1046 and 1051, (lxi) SEQ ID NOs: 1047 and 1049, (lxii) SEQ ID NOs: 1047 and 1050, (lxiii) SEQ ID NOs: 1047 and 1051, (lxiv) SEQ ID NOs: 1048 and 1049, (lxv) SEQ ID NOs: 1048 and 1050, (lxvi) SEQ ID NOs: 1048 and 1051, (lxvii) SEQ ID NOs: 1052 and 1054, (lxviii) SEQ ID NOs: 1052 and 1055, (lxix) SEQ ID NOs: 1053 and 1054, (lxx) SEQ ID NOs: 1053 and 1055, (lxxi) SEQ ID NOs: 1056 and 1057, (lxxii) SEQ ID NOs: 1058 and 1059, (lxxiii) SEQ ID NOs: 1060 and 1061, (lxxiv) SEQ ID NOs: 1062 and 1063, (lxxv) SEQ ID NOs: 1064 and 1065, (lxxvi) SEQ ID NOs: 1066 and 1067, (lxxvii) SEQ ID NOs: 1068 and 1069, (lxxviii) SEQ ID NOs: 1070 and 915, (lxxix) SEQ ID NOs: 1074 and 915, (lxxx) SEQ ID NOs: 1078 and 915, (lxxxi) SEQ ID NOs: 1082 and 915, (lxxxii) SEQ ID NOs: 1086 and 915, (lxxxiii) SEQ ID NOs: 1090 and 915, (lxxxix) SEQ ID NOs: 1094 and 915, (lxxxv) SEQ ID NOs: 1098 and 915, (lxxxvi) SEQ ID NOs: 1102 and 915, (lxxxvii) SEQ ID NOs: 1106 and 915, (lxxxviii) SEQ ID NOs: 1110 and 915, (lxxxix) SEQ ID NOs: 1114 and 915, (xc) SEQ ID NOs: 1118 and 915, (xci) SEQ ID NOs: 1122 and 915, (xcii) SEQ ID NOs: 1126 and 915, (xciii) SEQ ID NOs: 1130 and 915, (xciv) SEQ ID NOs: 1134 and 915, (xcv) SEQ ID NOs: 1138 and 915, (xcvi) SEQ ID NOs: 1142 and 915, (xcvii) SEQ ID NOs: 1146 and 915, (xcviii) SEQ ID NOs: 1150 and 915, (xcix) SEQ ID NOs: 1154 and 915, (c) SEQ ID NOs: 1158 and 915, (ci) SEQ ID NOs: 1162 and 915, (cii) SEQ ID NOs: 1166 and 915, (ciii) SEQ ID NOs: 1170 and 915, (civ) SEQ ID NOs: 1174 and 915, (cv) SEQ ID NOs: 1178 and 915, (cvi) SEQ ID NOs: 1182 and 915, (cvii) SEQ ID NOs: 1186 and 915, (cviii) SEQ ID NOs: 1189 and 915, (cix) SEQ ID NOs: 1192 and 915, (cx) SEQ ID NOs: 1196 and 915, (cxi) SEQ ID NOs: 1200 and 915, (cxii) SEQ ID NOs: 1204 and 915, (cxiii) SEQ ID NOs: 1208 and 915, (cxiv) SEQ ID NOs: 1212 and 915, (cxv) SEQ ID NOs: 1216 and 915, (cxvi) SEQ ID NOs: 1220 and 915, (cxvii) SEQ ID NOs: 1224 and 915, (cxviii) SEQ ID NOs: 1228 and 915, (cxix) SEQ ID NOs: 1232 and 915, (cxx) SEQ ID NOs: 1236 and 915, (cxxi) SEQ ID NOs: 1240 and 915, (cxxii) SEQ ID NOs: 1244 and 915, (cxxiii) SEQ ID NOs: 1248 and 915, (cxxiv) SEQ ID NOs: 1252 and 915, and (cxxv) SEQ ID NOs: 1256 and 915, as shown in FIGS. 26A-26Z. In some further embodiments, the NKE antibody (such as the NKG2D NKE antibody described herein) further comprises means for binding the ECD of a human TTA selected from a group including: (1) B7H3, (2) EGFR, (3) HER2, (4) CD19, (5) CD20, (6) CD123, (7) CAIX, (8) FLT3, (9) MSLN, (10) Trop2, (11) CEA, (12) CLDN18.2, (13) BCMA, (14) DLL3, (15) PD-1, (16) ANO1, (17) CD22, and (18) CD38, as described in further detail below. In still some further embodiments, the variant IgG1 Fc domain comprises an amino acid substitution selected from a group including: S239D, S239E, S239D/I332E, I332E, and I332D. In still some other further embodiments, the variant IgG1 Fc domain comprises an amino acid substitution selected from a group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

Embodiment (A)(1): In some further embodiments of an NKG2D NKE antibody comprising means for binding the ECD of a human TTA, the human TTA comprises human B7H3. Suitable variable heavy VH and variable light VL domains that bind to the ECD of human B7H3 include, but are not limited to, VH/VL pairs selected from the group including: (i) SEQ ID NOs: 1260 and 1264, (ii) SEQ ID NOs: 1268 and 1272, (iii) SEQ ID NOs: 1276 and 1280, (iv) SEQ ID NOs: 1284 and 1288, (v) SEQ ID NOs: 1292 and 1296, (vi) SEQ ID NOs: 1300 and 1296, (vii) SEQ ID NOs: 1301 and 1296, (viii) SEQ ID NOs: 1302 and 1296, (ix) SEQ ID NOs: 1303 and 1296, (x) SEQ ID NOs: 1304 and 1296, (xi) SEQ ID NOs: 1305 and 1296, (xii) SEQ ID NOs: 1306 and 1296, (xiii) SEQ ID NOs: 1307 and 1296, (xiv) SEQ ID NOs: 1308 and 1296, (xv) SEQ ID NOs: 1309 and 1296, (xvi) SEQ ID NOs: 1310 and 1296, (xvii) SEQ ID NOs: 1311 and 1296, (xviii) SEQ ID NOs: 1312 and 1296, (xix) SEQ ID NOs: 1313 and 1296, (xx) SEQ ID NOs: 1314 and 1296, (xxi) SEQ ID NOs: 1315 and 1296, (xxii) SEQ ID NOs: 1316 and 1296, (xxiii) SEQ ID NOs: 1317 and 1296, (xxiv) SEQ ID NOs: 1318 and 1296, (xxv) SEQ ID NOs: 1319 and 1296, (xxvi) SEQ ID NOs: 1320 and 1296, (xxvii) SEQ ID NOs: 1321 and 1296, (xxviii) SEQ ID NOs: 1322 and 1296, (xxix) SEQ ID NOs: 1323 and 1296, (xxx) SEQ ID NOs: 1324 and 1296, (xxxi) SEQ ID NOs: 1325 and 1296, (xxxii) SEQ ID NOs: 1326 and 1296, (xxxiii) SEQ ID NOs: 1327 and 1296, (xxxiv) SEQ ID NOs: 1328 and 1296, (xxxv) SEQ ID NOs: 1329 and 1296, (xxxvi) SEQ ID NOs: 1330 and 1296, (xxxvii) SEQ ID NOs: 1331 and 1296, (xxxviii) SEQ ID NOs: 1332 and 1296, (xxxix) SEQ ID NOs: 1333 and 1296, (xl) SEQ ID NOs: 1334 and 1296, (xli) SEQ ID NOs: 1335 and 1296, (xlii) SEQ ID NOs: 1336 and 1296, (xliii) SEQ ID NOs: 1337 and 1296, (xliv) SEQ ID NOs: 1338 and 1296, (xlv) SEQ ID NOs: 1339 and 1296, (xlvi) SEQ ID NOs: 1340 and 1296, (xlvii) SEQ ID NOs: 1341 and 1296, (xlviii) SEQ ID NOs: 1342 and 1296, (xlix) SEQ ID NOs: 1343 and 1296, (1) SEQ ID NOs: 1344 and 1296, (li) SEQ ID NOs: 1345 and 1296, (lii) SEQ ID NOs: 1346 and 1296, (liii) SEQ ID NOs: 1347 and 1296, (liv) SEQ ID NOs: 1348 and 1296, (lv) SEQ ID NOs: 1349 and 1296, (lvi) SEQ ID NOs: 1350 and 1296, (lvii) SEQ ID NOs: 1351 and 1296, (lviii) SEQ ID NOs: 1352 and 1296, (lix) SEQ ID NOs: 1353 and 1296, (lx) SEQ ID NOs: 1354 and 1296, (lxi) SEQ ID NOs: 1355 and 1296, (lxii) SEQ ID NOs: 1356 and 1296, (lxiii) SEQ ID NOs: 1357 and 1296, (lxiv) SEQ ID NOs: 1358 and 1296, (lxv) SEQ ID NOs: 1359 and 1296, (lxvi) SEQ ID NOs: 1360 and 1296, (lxvii) SEQ ID NOs: 1361 and 1296, (lxviii) SEQ ID NOs: 1362 and 1296, (lxix) SEQ ID NOs: 1363 and 1296, (lxx) SEQ ID NOs: 1364 and 1296, (lxxi) SEQ ID NOs: 1365 and 1296, (lxxii) SEQ ID NOs: 1366 and 1296, (lxxiii) SEQ ID NOs: 1367 and 1296, (lxxiv) SEQ ID NOs: 1368 and 1296, (lxxv) SEQ ID NOs: 1369 and 1296, (lxxvi) SEQ ID NOs: 1370 and 1296, (lxxvii) SEQ ID NOs: 1371 and 1296, (lxxviii) SEQ ID NOs: 1372 and 1296, (lxxix) SEQ ID NOs: 1373 and 1296, (lxxx) SEQ ID NOs: 1374 and 1296, (lxxxi) SEQ ID NOs: 1375 and 1296, (lxxxii) SEQ ID NOs: 1376 and 1296, (lxxxiii) SEQ ID NOs: 1377 and 1296, (lxxxix) SEQ ID NOs: 1378 and 1296, (lxxxv) SEQ ID NOs: 1379 and 1296, (lxxxvi) SEQ ID NOs: 1380 and 1296, (lxxxvii) SEQ ID NOs: 1381 and 1296, (lxxxviii) SEQ ID NOs: 1382 and 1296, (lxxxix) SEQ ID NOs: 1383 and 1296, (xc) SEQ ID NOs: 1384 and 1296, (xci) SEQ ID NOs: 1385 and 1296, (xcii) SEQ ID NOs: 1386 and 1296, and (xciii) SEQ ID NOs: 1387 and 1296, as shown in FIGS. 27, 28, 29A-29H, and 30A-30R, as well as those disclosed herein (see, e.g., Section (V)(B)(1) above).

Embodiment (A)(2): In some other further embodiments of an NKG2D NKE antibody comprising means for binding the ECD of a human TTA, the human TTA comprises human EGFR. Suitable variable heavy VH and variable light VL domains that bind to the ECD of human EGFR include, but are not limited to, VH/VL pairs selected from the group including: (i) SEQ ID NOs: 164 and 165, (ii) SEQ ID NOs: 166 and 167, (iii) SEQ ID NOs: 168 and 169, (iv) SEQ ID NOs: 170 and 171, (v) SEQ ID NOs: 172 and 173, (vi) SEQ ID NOs: 174 and 175, (vii) SEQ ID NOs: 176 and 177, (viii) SEQ ID NOs: 178 and 179, (ix) SEQ ID NOs: 180 and 181, (x) SEQ ID NOs: 182 and 183, (xi) SEQ ID NOs: 184 and 185, and (xii) SEQ ID NOs: 186 and 187, as shown in FIGS. 13A-13C, as well as those disclosed herein (see, e.g., Section (V)(B)(2) above).

Embodiment (A)(3): In some other further embodiments of an NKG2D NKE antibody comprising means for binding the ECD of a human TTA, the human TTA comprises human HER2. Suitable variable heavy VH and variable light VL domains that bind to the ECD of human HER2 include, but are not limited to, VH/VL pairs selected from the group including: (i) SEQ ID NOs: 275 and 276, (ii) SEQ ID NOs: 277 and 278, (iii) SEQ ID NOs: 279 and 280, (iv) SEQ ID NOs: 281 and 282, (v) SEQ ID NOs: 283 and 284, (vi) SEQ ID NOs: 285 and 286, and (vii) SEQ ID NOs: 287 and 288, as shown in FIGS. 20A-20B.

Embodiment (A)(4): In some other further embodiments of an NKG2D NKE antibody comprising means for binding the ECD of a human TTA, the human TTA comprises human CD19. Suitable variable heavy VH and variable light VL domains that bind to the ECD of human CD19 include, but are not limited to, VH/VL pairs selected from the group including: (i) SEQ ID NOs: 188 and 189, (ii) SEQ ID NOs: 190 and 191, (iii) SEQ ID NOs: 192 and 193, and (iv) SEQ ID NOs: 194 and 195, as shown in FIG. 14.

Embodiment (A)(5): In some other further embodiments of an NKG2D NKE antibody comprising means for binding the ECD of a human TTA, the human TTA comprises human CD20. Suitable variable heavy VH and variable light VL domains that bind to the ECD of human CD20 include, but are not limited to, VH/VL pairs selected from the group including: (i) SEQ ID NOs: 196 and 197, (ii) SEQ ID NOs: 198 and 199, (iii) SEQ ID NOs: 200 and 201, (iv) SEQ ID NOs: 202 and 203, (v) SEQ ID NOs: 204 and 205, (vi) SEQ ID NOs: 206 and 207, (vii) SEQ ID NOs: 208 and 209, (viii) SEQ ID NOs: 210 and 211, (ix) SEQ ID NOs: 212 and 213, (x) SEQ ID NOs: 214 and 215, (xi) SEQ ID NOs: 216 and 217, (xii) SEQ ID NOs: 218 and 219, (xiii) SEQ ID NOs: 220 and 221, (xiv) SEQ ID NOs: 222 and 223, and (xv) SEQ ID NOs: 224 and 225, as shown in FIGS. 15A-15C.

Embodiment (A)(6): In some other further embodiments of an NKG2D NKE antibody comprising means for binding the ECD of a human TTA, the human TTA comprises human CD123. Suitable variable heavy VH and variable light VL domains that bind to the ECD of human CD123 include, but are not limited to, VH/VL pairs selected from the group including: (i) SEQ ID NOs: 833 and 834, and (ii) SEQ ID NOs: 835 and 836, as shown in FIG. 23.

Embodiment (A)(7): In some other further embodiments of an NKG2D NKE antibody comprising means for binding the ECD of a human TTA, the human TTA comprises human CAIX. Suitable variable heavy VH and variable light VL domains that bind to the ECD of human CAIX include, but are not limited to, the pair of SEQ ID NOs: 831 and 832, as shown in FIG. 23, as well as those disclosed herein (see, e.g., Section (V)(B)(7) above).

Embodiment (A)(8): In some other further embodiments of an NKG2D NKE antibody comprising means for binding the ECD of a human TTA, the human TTA comprises human FLT3. Suitable variable heavy VH and variable light VL domains that bind to the ECD of human FLT3 include, but are not limited to, VH/VL pairs selected from the group including: (i) SEQ ID NOs: 267 and 268, (ii) SEQ ID NOs: 269 and 270, (iii) SEQ ID NOs: 271 and 272, (iv) SEQ ID NOs: 273 and 274, (v) SEQ ID NOs: 275 and 276, (vi) SEQ ID NOs: 277 and 278, (vii) SEQ ID NOs: 279 and 280, and (viii) SEQ ID NOs: 281 and 282, as shown in FIG. 19, as well as those disclosed herein (see, e.g., Section (V)(B)(8) above).

Embodiment (A)(9): In some other further embodiments of an NKG2D NKE antibody comprising means for binding the ECD of a human TTA, the human TTA comprises human MSLN. Suitable variable heavy VH and variable light VL domains that bind to the ECD of human MSLN include, but are not limited to, VH/VL pairs selected from the group including: (i) SEQ ID NOs: 289 and 293, (ii) SEQ ID NOs: 297 and 301, (iii) SEQ ID NOs: 305 and 309, (iv) SEQ ID NOs: 313 and 314, (v) SEQ ID NOs: 315 and 316, (vi) SEQ ID NOs: 317 and 318, (vii) SEQ ID NOs: 319 and 320, (viii) SEQ ID NOs: 321 and 322, (ix) SEQ ID NOs: 323 and 324, (x) SEQ ID NOs: 325 and 326, (xi) SEQ ID NOs: 327 and 328, (xii) SEQ ID NOs: 329 and 330, (xiii) SEQ ID NOs: 331 and 332, (xiv) SEQ ID NOs: 333 and 334, (xv) SEQ ID NOs: 335 and 336, (xvi) SEQ ID NOs: 337 and 338, (xvii) SEQ ID NOs: 339 and 340, (xviii) SEQ ID NOs: 341 and 342, (xix) SEQ ID NOs: 343 and 344, (xx) SEQ ID NOs: 345 and 346, (xxi) SEQ ID NOs: 347 and 348, (xxii) SEQ ID NOs: 349 and 350, (xxiii) SEQ ID NOs: 351 and 352, (xxiv) SEQ ID NOs: 353 and 354, (xxv) SEQ ID NOs: 355 and 356, (xxvi) SEQ ID NOs: 357 and 358, (xxvii) SEQ ID NOs: 359 and 360, (xxviii) SEQ ID NOs: 361 and 362, (xxix) SEQ ID NOs: 363 and 364, (xxx) SEQ ID NOs: 365 and 366, (xxxi) SEQ ID NOs: 367 and 368, (xxxii) SEQ ID NOs: 369 and 370, (xxxiii) SEQ ID NOs: 371 and 372, (xxxiv) SEQ ID NOs: 373 and 374, (xxxv) SEQ ID NOs: 375 and 376, (xxxvi) SEQ ID NOs: 377 and 378, (xxxvii) SEQ ID NOs: 379 and 380, (xxxviii) SEQ ID NOs: 381 and 382, (xxxix) SEQ ID NOs: 383 and 384, (xl) SEQ ID NOs: 385 and 386, (xli) SEQ ID NOs: 387 and 388, (xlii) SEQ ID NOs: 389 and 390, (xliii) SEQ ID NOs: 391 and 392, (xliv) SEQ ID NOs: 393 and 394, (xlv) SEQ ID NOs: 395 and 396, (xlvi) SEQ ID NOs: 397 and 398, (xlvii) SEQ ID NOs: 399 and 400, (xlviii) SEQ ID NOs: 401 and 402, (xlix) SEQ ID NOs: 403 and 404, (1) SEQ ID NOs: 405 and 406, (li) SEQ ID NOs: 405 and 408, (lii) SEQ ID NOs: 407 and 406, (liii) SEQ ID NOs: 407 and 408, (liv) SEQ ID NOs: 409 and 410, (lv) SEQ ID NOs: 411 and 412, (lvi) SEQ ID NOs: 413 and 414, (lvii) SEQ ID NOs: 415 and 416, (lviii) SEQ ID NOs: 417 and 418, (lix) SEQ ID NOs: 419 and 420, (lx) SEQ ID NOs: 421 and 422, (lxi) SEQ ID NOs: 423 and 424, (lxii) SEQ ID NOs: 425 and 426, (lxiii) SEQ ID NOs: 427 and 428, (lxiv) SEQ ID NOs: 429 and 430, (lxv) SEQ ID NOs: 431 and 432, (lxvi) SEQ ID NOs: 433 and 434, (lxvii) SEQ ID NOs: 435 and 436, (lxviii) SEQ ID NOs: 437 and 438, (lxix) SEQ ID NOs: 439 and 440, (lxx) SEQ ID NOs: 441 and 442, (lxxi) SEQ ID NOs: 443 and 444, (lxxii) SEQ ID NOs: 445 and 446, (lxxiii) SEQ ID NOs: 447 and 448, (lxxiv) SEQ ID NOs: 449 and 450, (lxxv) SEQ ID NOs: 451 and 452, (lxxvi) SEQ ID NOs: 453 and 454, (lxxvii) SEQ ID NOs: 455 and 456, (lxxviii) SEQ ID NOs: 457 and 458, (lxxix) SEQ ID NOs: 459 and 460, (lxxx) SEQ ID NOs: 461 and 462, (lxxxi) SEQ ID NOs: 463 and 464, (lxxxii) SEQ ID NOs: 465 and 466, (lxxxiii) SEQ ID NOs: 467 and 468, (lxxxix) SEQ ID NOs: 469 and 470, (lxxxv) SEQ ID NOs: 471 and 472, (lxxxvi) SEQ ID NOs: 473 and 474, (lxxxvii) SEQ ID NOs: 475 and 476, (lxxxviii) SEQ ID NOs: 477 and 478, (lxxxix) SEQ ID NOs: 479 and 480, (xc) SEQ ID NOs: 481 and 482, (xci) SEQ ID NOs: 483 and 484, (xcii) SEQ ID NOs: 485 and 486, (xciii) SEQ ID NOs: 487 and 488, (xciv) SEQ ID NOs: 489 and 490, (xcv) SEQ ID NOs: 491 and 492, (xcvi) SEQ ID NOs: 493 and 494, (xcvii) SEQ ID NOs: 495 and 496, (xcviii) SEQ ID NOs: 497 and 498, (xcix) SEQ ID NOs: 499 and 500, (c) SEQ ID NOs: 501 and 502, (ci) SEQ ID NOs: 503 and 504, (cii) SEQ ID NOs: 505 and 506, (ciii) SEQ ID NOs: 507 and 508, (civ) SEQ ID NOs: 509 and 510, (cv) SEQ ID NOs: 511 and 512, (cvi) SEQ ID NOs: 513 and 514, (cvii) SEQ ID NOs: 515 and 516, (cviii) SEQ ID NOs: 517 and 518, (cix) SEQ ID NOs: 519 and 520, (cx) SEQ ID NOs: 521 and 522, (cxi) SEQ ID NOs: 523 and 524, (cxii) SEQ ID NOs: 525 and 526, (cxiii) SEQ ID NOs: 527 and 528, (cxiv) SEQ ID NOs: 529 and 530, (cxv) SEQ ID NOs: 531 and 532, (cxvi) SEQ ID NOs: 533 and 534, (cxvii) SEQ ID NOs: 535 and 536, (cxviii) SEQ ID NOs: 537 and 538, (cxix) SEQ ID NOs: 539 and 540, (cxx) SEQ ID NOs: 541 and 542, (cxxi) SEQ ID NOs: 543 and 544, (cxxii) SEQ ID NOs: 545 and 546, (cxxiii) SEQ ID NOs: 547 and 548, (cxxiv) SEQ ID NOs: 549 and 550, (cxxv) SEQ ID NOs: 551 and 552, (cxxvi) SEQ ID NOs: 553 and 554, (cxxvii) SEQ ID NOs: 555 and 556, (cxxviii) SEQ ID NOs: 557 and 558, (cxxix) SEQ ID NOs: 559 and 560, (cxxx) SEQ ID NOs: 561 and 562, (cxxxi) SEQ ID NOs: 563 and 564, (cxxxii) SEQ ID NOs: 565 and 566, (cxxxiii) SEQ ID NOs: 567 and 568, (cxxxiv) SEQ ID NOs: 569 and 570, (cxxxv) SEQ ID NOs: 571 and 572, (cxxxvi) SEQ ID NOs: 573 and 574, (cxxxvii) SEQ ID NOs: 575 and 576, (cxxxviii) SEQ ID NOs: 577 and 578, (cxl) SEQ ID NOs: 579 and 580, (cxli) SEQ ID NOs: 581 and 582, (cxlii) SEQ ID NOs: 583 and 584, (cxliii) SEQ ID NOs: 585 and 586, (cxliv) SEQ ID NOs: 587 and 588, (cxlv) SEQ ID NOs: 589 and 590, (cxlvi) SEQ ID NOs: 591 and 592, (cxlvii) SEQ ID NOs: 593 and 594, (cxlviii) SEQ ID NOs: 595 and 596, (cxlix) SEQ ID NOs: 597 and 598, (cl) SEQ ID NOs: 599 and 600, (cli) SEQ ID NOs: 601 and 602, (clii) SEQ ID NOs: 603 and 604, (cliii) SEQ ID NOs: 605 and 606, (cliv) SEQ ID NOs: 607 and 608, (clv) SEQ ID NOs: 609 and 610, (clvi) SEQ ID NOs: 611 and 612, (clvii) SEQ ID NOs: 613 and 614, (clviii) SEQ ID NOs: 615 and 616, (clviii) SEQ ID NOs: 617 and 618, (clix) SEQ ID NOs: 619 and 620, (clx) SEQ ID NOs: 621 and 622, (clxi) SEQ ID NOs: 623 and 624, (clxii) SEQ ID NOs: 625 and 626, (clxiii) SEQ ID NOs: 627 and 628, (clxiv) SEQ ID NOs: 629 and 630, (clxv) SEQ ID NOs: 631 and 632, (clxvi) SEQ ID NOs: 633 and 634, (clxvii) SEQ ID NOs: 635 and 636, (clxviii) SEQ ID NOs: 637 and 638, and (clxix) any one of SEQ ID NOs: 639-649 and any one of SEQ ID NOs: 650-669, as shown in FIGS. 21A-21FF, as well as those disclosed herein (see, e.g., Section (V)(B)(9) above).

Embodiment (A)(10): In some other further embodiments of an NKG2D NKE antibody comprising means for binding the ECD of a human TTA, the human TTA comprises human Trop2. Suitable variable heavy VH and variable light VL domains that bind to the ECD of human Trop2 include, but are not limited to, VH/VL pairs selected from the group including: (i) SEQ ID NOs: 670 and 671, (ii) SEQ ID NOs: 672 and 673, (iii) SEQ ID NOs: 674 and 675, (iv) SEQ ID NOs: 676 and 677, (v) SEQ ID NOs: 678 and 679, (vi) SEQ ID NOs: 680 and 681, (vii) SEQ ID NOs: 682 and 683, (viii) SEQ ID NOs: 684 and 681, (ix) SEQ ID NOs: 685 and 686, (x) SEQ ID NOs: 687 and 688, (xi) SEQ ID NOs: 689 and 690, (xii) SEQ ID NOs: 691 and 692, (xiii) SEQ ID NOs: 693 and 694, (xiv) SEQ ID NOs: 695 and 696, (xv) SEQ ID NOs: 697 and 698, (xvi) SEQ ID NOs: 699 and 700, (xvii) SEQ ID NOs: 701 and 702, (xviii) SEQ ID NOs: 703 and 704, (xix) SEQ ID NOs: 705 and 706, (xx) SEQ ID NOs: 707 and 708, (xxi) SEQ ID NOs: 709 and 710, (xxii) SEQ ID NOs: 711 and 712, (xxiii) SEQ ID NOs: 713 and 714, (xxiv) SEQ ID NOs: 715 and 716, (xxv) SEQ ID NOs: 717 and 718, (xxvi) SEQ ID NOs: 719 and 720, (xxvii) SEQ ID NOs: 721 and 722, (xxviii) SEQ ID NOs: 723 and 724, (xxix) SEQ ID NOs: 725 and 726, (xxx) SEQ ID NOs: 727 and 728, (xxxi) SEQ ID NOs: 729 and 730, (xxxii) SEQ ID NOs: 731 and 732, (xxxiii) SEQ ID NOs: 733 and 734, (xxxiv) SEQ ID NOs: 735 and 736, (xxxv) SEQ ID NOs: 737 and 738, (xxxvi) SEQ ID NOs: 739 and 740, (xxxvii) SEQ ID NOs: 741 and 742, (xxxviii) SEQ ID NOs: 743 and 744, (xxxix) SEQ ID NOs: 745 and 746, (xl) SEQ ID NOs: 747 and 748, (xli) SEQ ID NOs: 749 and 750, (xlii) SEQ ID NOs: 751 and 752, (xliii) SEQ ID NOs: 753 and 754, (xliv) SEQ ID NOs: 755 and 756, (xlv) SEQ ID NOs: 757 and 758, (xlvi) SEQ ID NOs: 759 and 760, (xlvii) SEQ ID NOs: 761 and 762, (xlviii) SEQ ID NOs: 763 and 764, (xlix) SEQ ID NOs: 765 and 766, (1) SEQ ID NOs: 767 and 768, (li) SEQ ID NOs: 769 and 770, (lii) SEQ ID NOs: 771 and 772, (liii) SEQ ID NOs: 773 and 774, (liv) SEQ ID NOs: 775 and 776, (lv) SEQ ID NOs: 777 and 778, (lvi) SEQ ID NOs: 779 and 780, (lvii) SEQ ID NOs: 781 and 782, (lviii) SEQ ID NOs: 783 and 784, (lix) SEQ ID NOs: 785 and 786, (lx) SEQ ID NOs: 787 and 788, (lxi) SEQ ID NOs: 789 and 790, (lxii) SEQ ID NOs: 791 and 792, (lxiii) SEQ ID NOs: 793 and 794, (lxiv) SEQ ID NOs: 795 and 800, (lxv) SEQ ID NOs: 795 and 801, (lxvi) SEQ ID NOs: 795 and 802, (lxvii) SEQ ID NOs: 796 and 800, (lxviii) SEQ ID NOs: 796 and 801, (lxix) SEQ ID NOs: 796 and 802, (lxx) SEQ ID NOs: 797 and 800, (lxxi) SEQ ID NOs: 797 and 801, (lxxii) SEQ ID NOs: 797 and 802, (lxxiii) SEQ ID NOs: 798 and 800, (lxxiv) SEQ ID NOs: 798 and 801, (lxxv) SEQ ID NOs: 798 and 802, (lxxvi) SEQ ID NOs: 799 and 800, (lxxvii) SEQ ID NOs: 799 and 801, (lxxviii) SEQ ID NOs: 799 and 802, (lxxix) SEQ ID NOs: 803 and 806, (lxxx) SEQ ID NOs: 803 and 807, (lxxxi) SEQ ID NOs: 803 and 808, (lxxxii) SEQ ID NOs: 804 and 806, (lxxxiii) SEQ ID NOs: 804 and 807, (lxxxix) SEQ ID NOs: 804 and 808, (lxxxv) SEQ ID NOs: 805 and 806, (lxxxvi) SEQ ID NOs: 805 and 807, (lxxxvii) SEQ ID NOs: 805 and 808, (lxxxviii) SEQ ID NOs: 809 and 812, (lxxxix) SEQ ID NOs: 809 and 813, (xc) SEQ ID NOs: 810 and 812, (xci) SEQ ID NOs: 810 and 813, (xcii) SEQ ID NOs: 811 and 812, (xciii) SEQ ID NOs: 811 and 813, (xciv) SEQ ID NOs: 814 and 816, (xcv) SEQ ID NOs: 814 and 817, (xcvi) SEQ ID NOs: 814 and 818, (xcvii) SEQ ID NOs: 815 and 816, (xcviii) SEQ ID NOs: 815 and 817, (xcix) SEQ ID NOs: 815 and 818, (c) SEQ ID NOs: 819 and 824, (ci) SEQ ID NOs: 819 and 825, (cii) SEQ ID NOs: 819 and 826, (ciii) SEQ ID NOs: 819 and 827, (civ) SEQ ID NOs: 819 and 828, (cv) SEQ ID NOs: 820 and 824, (cvi) SEQ ID NOs: 820 and 825, (cvii) SEQ ID NOs: 820 and 826, (cviii) SEQ ID NOs: 820 and 827, (cix) SEQ ID NOs: 820 and 828, (cx) SEQ ID NOs: 821 and 824, (cxi) SEQ ID NOs: 821 and 825, (cxii) SEQ ID NOs: 821 and 826, (cxiii) SEQ ID NOs: 821 and 827, (cxiv) SEQ ID NOs: 821 and 828, (cxv) SEQ ID NOs: 822 and 824, (cxvi) SEQ ID NOs: 822 and 825, (cxvii) SEQ ID NOs: 822 and 826, (cxviii) SEQ ID NOs: 822 and 827, (cxix) SEQ ID NOs: 822 and 828, (cxx) SEQ ID NOs: 823 and 824, (cxxi) SEQ ID NOs: 823 and 825, (cxxii) SEQ ID NOs: 823 and 826, (cxxiii) SEQ ID NOs: 823 and 827, (cxxiv) SEQ ID NOs: 823 and 828, and (cxxv) SEQ ID NOs: 829 and 830, as shown in FIGS. 22A-22R, as well as those disclosed herein (see, e.g., Section (V)(B)(10) above).

Embodiment (A)(11): In some other further embodiments of an NKG2D NKE antibody comprising means for binding the ECD of a human TTA, the human TTA comprises human CEA. Suitable variable heavy VH and variable light VL domains that bind to the ECD of human CEA include, but are not limited to, VH/VL pairs selected from the group including: (i) SEQ ID NOs: 239 and 240, (ii) SEQ ID NOs: 241 and 242, (iii) SEQ ID NOs: 243 and 244, (iv) SEQ ID NOs: 245 and 246, (v) SEQ ID NOs: 247 and 248, (vi) SEQ ID NOs: 249 and 250, (vii) SEQ ID NOs: 251 and 252, (viii) SEQ ID NOs: 253 and 254, (ix) SEQ ID NOs: 255 and 256, (x) SEQ ID NOs: 257 and 258, (xi) SEQ ID NOs: 259 and 260, (xii) SEQ ID NOs: 261 and 262, (xiii) SEQ ID NOs: 263 and 264, and (xiv) SEQ ID NOs: 265 and 266, as shown in FIGS. 18A-18C, as well as those disclosed herein (see, e.g., Section (V)(B)(11) above).

Embodiment (A)(12): In some other further embodiments of an NKG2D NKE antibody comprising means for binding the ECD of a human TTA, the human TTA comprises human CLDN18.2. Suitable variable heavy VH and variable light VL domains that bind to the ECD of human CLDN18.2 include, but are not limited to, VH/VL pairs selected from the group including: (i) SEQ ID NOs: 226 and 229, (ii) SEQ ID NOs: 226 and 230, (iii) SEQ ID NOs: 227 and 229, (iv) SEQ ID NOs: 227 and 230, (v) SEQ ID NOs: 228 and 229, (vi) SEQ ID NOs: 228 and 230, and (vii) SEQ ID NOs: 231 and 232, as shown in FIG. 16, as well as those disclosed herein (see, e.g., Section (V)(B)(12) above).

Embodiment (A)(13): In some other further embodiments of an NKG2D NKE antibody comprising means for binding the ECD of a human TTA, the human TTA comprises human BCMA. Suitable variable heavy VH and variable light VL domains that bind to the ECD of human BCMA include, but are not limited to, VH/VL pairs selected from the group including: (i) SEQ ID NOs: 233 and 234, (ii) SEQ ID NOs: 235 and 236, (iii) SEQ ID NOs: 237 and 238, (iv) SEQ ID NOs: 12 and 16, (v) SEQ ID NOs: 22 and 26, (vi) SEQ ID NOs: 32 and 36, (vii) SEQ ID NOs: 42 and 46, (viii) SEQ ID NOs: 52 and 56, (ix) SEQ ID NOs: 62 and 66, and (x) SEQ ID NOs: 72 and 76, as shown in FIGS. 17A-17C, as well as those disclosed herein (see, e.g., Section (V)(B)(13) above).

Embodiment (A)(14): In some other further embodiments of an NKG2D NKE antibody comprising means for binding the ECD of a human TTA, the human TTA comprises human DLL3. Suitable variable heavy VH and variable light VL domains that bind to the ECD of human DLL3 include, but are not limited to, VH/VL pairs selected from the group including: (i) SEQ ID NOs: 837 and 838, (ii) SEQ ID NOs: 839 and 840, (iii) SEQ ID NOs: 841 and 842, (iv) SEQ ID NOs: 843 and 844, (v) SEQ ID NOs: 845 and 846, (vi) SEQ ID NOs: 847 and 848, (vii) SEQ ID NOs: 849 and 850, (viii) SEQ ID NOs: 851 and 852, (ix) SEQ ID NOs: 853 and 854, (x) SEQ ID NOs: 855 and 856, (xi) SEQ ID NOs: 857 and 858, (xii) SEQ ID NOs: 859 and 860, (xiii) SEQ ID NOs: 861 and 862, (xiv) SEQ ID NOs: 863 and 864, (xv) SEQ ID NOs: 865 and 866, (xvi) SEQ ID NOs: 867 and 868, and (xvii) SEQ ID NOs: 869 and 870, as shown in FIGS. 24A-24C, as well as those disclosed herein (see, e.g., Section (V)(B)(14) above).

Embodiment (A)(15): In some other further embodiments of an NKG2D NKE antibody comprising means for binding the ECD of a human TTA, the human TTA comprises human PD-1. Suitable variable heavy VH and variable light VL domains that bind to the ECD of human PD-1 include, but are not limited to, VH/VL pairs selected from the group including: (i) SEQ ID NOs: 871 and 872, (ii) SEQ ID NOs: 873 and 874, (iii) SEQ ID NOs: 875 and 876, (iv) SEQ ID NOs: 877 and 878, (v) SEQ ID NOs: 879 and 880, (vi) SEQ ID NOs: 881 and 882, (vii) SEQ ID NOs: 883 and 884, (viii) SEQ ID NOs: 885 and 886, (ix) SEQ ID NOs: 887 and 888, (x) SEQ ID NOs: 889 and 890, (xi) SEQ ID NOs: 891 and 892, (xii) SEQ ID NOs: 893 and 894, (xiii) SEQ ID NOs: 895 and 896, (xiv) SEQ ID NOs: 897 and 898, (xv) SEQ ID NOs: 899 and 900, (xvi) SEQ ID NOs: 901 and 902, (xvii) SEQ ID NOs: 903 and 904, (xviii) SEQ ID NOs: 905 and 906, (xix) SEQ ID NOs: 907 and 908, and (xx) SEQ ID NOs: 909 and 910, as shown in FIGS. 25A-25G, as well as those disclosed herein (see, e.g., Section (V)(B)(15) above).

Embodiment (A)(16): In some other further embodiments of an NKG2D NKE antibody comprising means for binding the ECD of a human TTA, the human TTA comprises human ANOL. Suitable variable heavy VH and variable light VL domains that bind to the ECD of human ANO1 include, but are not limited to, the sequences disclosed herein (see, e.g., Section (V)(B)(16) above).

Embodiment (A)(17): In some other further embodiments of an NKG2D NKE antibody comprising means for binding the ECD of a human TTA, the human TTA comprises human CD22. Suitable variable heavy VH and variable light VL domains that bind to the ECD of human CD22 include, but are not limited to, the sequences disclosed herein (see, e.g., Section (V)(B)(17) above).

Embodiment (A)(18): In some other further embodiments of an NKG2D NKE antibody comprising means for binding the ECD of a human TTA, the human TTA comprises human CD38. Suitable variable heavy VH and variable light VL domains that bind to the ECD of human CD38 include, but are not limited to, the sequences disclosed herein (see, e.g., Section (V)(B)(18) above).

Embodiment (B): In some embodiments, the NKE antibody comprises: a) means for binding the extracellular domain of human NKp46; and b) a variant IgG1 Fc dimeric domain comprising an amino acid substitution as compared to human IgG1, wherein the variant has increased in vitro binding to the human FcγRIIIa receptor as compared to human IgG1. Suitable variable heavy VH and variable light VL domains that bind to the ECD of human NKp46 include, but are not limited to, VH/VL pairs selected from the group including: (i) SEQ ID NOs: 133 and 134, (ii) SEQ ID NOs: 135 and 136, (iii) SEQ ID NOs: 137 and 138, (iv) SEQ ID NOs: 139 and 140, (v) SEQ ID NOs: 141 and 142, (vi) SEQ ID NOs: 143 and 144, (vii) SEQ ID NOs: 146 and 147, (viii) SEQ ID NOs: 148 and 149, (ix) SEQ ID NOs: 150 and 151, (x) SEQ ID NOs: 152 and 153, (xi) SEQ ID NOs: 154 and 155, and (xii) SEQ ID NOs: 156 and 157, as shown in FIGS. 11A-11C. In some further embodiments, the NKE antibody (such as the NKp46 NKE antibody described herein) further comprises means for binding the ECD of a human TTA selected from a group including: (1) B7H3, (2) EGFR, (3) HER2, (4) CD19, (5) CD20, (6) CD123, (7) CAIX, (8) FLT3, (9) MSLN, (10) Trop2, (11) CEA, (12) CLDN18.2, (13) BCMA, (14) DLL3, (15) PD-1, (16) ANO1, (17) CD22, and (18) CD38, as described in further detail below. In still some further embodiments, the variant IgG1 Fc domain comprises an amino acid substitution selected from a group including: S239D, S239E, S239D/I332E, I332E, and 1332D. In still some other further embodiments, the variant IgG1 Fc domain comprises an amino acid substitution selected from a group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

Embodiment (B)(1): In some further embodiments of the NKE antibody comprising means for binding the ECD of human NKp46 and means for binding the ECD of a human TTA, the human TTA comprises human B7H3. Suitable variable heavy VH and variable light VL domains that bind to the ECD of human B7H3 include, but are not limited to, VH/VL pairs selected from the group including: (i) SEQ ID NOs: 1260 and 1264, (ii) SEQ ID NOs: 1268 and 1272, (iii) SEQ ID NOs: 1276 and 1280, (iv) SEQ ID NOs: 1284 and 1288, (v) SEQ ID NOs: 1292 and 1296, (vi) SEQ ID NOs: 1300 and 1296, (vii) SEQ ID NOs: 1301 and 1296, (viii) SEQ ID NOs: 1302 and 1296, (ix) SEQ ID NOs: 1303 and 1296, (x) SEQ ID NOs: 1304 and 1296, (xi) SEQ ID NOs: 1305 and 1296, (xii) SEQ ID NOs: 1306 and 1296, (xiii) SEQ ID NOs: 1307 and 1296, (xiv) SEQ ID NOs: 1308 and 1296, (xv) SEQ ID NOs: 1309 and 1296, (xvi) SEQ ID NOs: 1310 and 1296, (xvii) SEQ ID NOs: 1311 and 1296, (xviii) SEQ ID NOs: 1312 and 1296, (xix) SEQ ID NOs: 1313 and 1296, (xx) SEQ ID NOs: 1314 and 1296, (xxi) SEQ ID NOs: 1315 and 1296, (xxii) SEQ ID NOs: 1316 and 1296, (xxiii) SEQ ID NOs: 1317 and 1296, (xxiv) SEQ ID NOs: 1318 and 1296, (xxv) SEQ ID NOs: 1319 and 1296, (xxvi) SEQ ID NOs: 1320 and 1296, (xxvii) SEQ ID NOs: 1321 and 1296, (xxviii) SEQ ID NOs: 1322 and 1296, (xxix) SEQ ID NOs: 1323 and 1296, (xxx) SEQ ID NOs: 1324 and 1296, (xxxi) SEQ ID NOs: 1325 and 1296, (xxxii) SEQ ID NOs: 1326 and 1296, (xxxiii) SEQ ID NOs: 1327 and 1296, (xxxiv) SEQ ID NOs: 1328 and 1296, (xxxv) SEQ ID NOs: 1329 and 1296, (xxxvi) SEQ ID NOs: 1330 and 1296, (xxxvii) SEQ ID NOs: 1331 and 1296, (xxxviii) SEQ ID NOs: 1332 and 1296, (xxxix) SEQ ID NOs: 1333 and 1296, (xl) SEQ ID NOs: 1334 and 1296, (xli) SEQ ID NOs: 1335 and 1296, (xlii) SEQ ID NOs: 1336 and 1296, (xliii) SEQ ID NOs: 1337 and 1296, (xliv) SEQ ID NOs: 1338 and 1296, (xlv) SEQ ID NOs: 1339 and 1296, (xlvi) SEQ ID NOs: 1340 and 1296, (xlvii) SEQ ID NOs: 1341 and 1296, (xlviii) SEQ ID NOs: 1342 and 1296, (xlix) SEQ ID NOs: 1343 and 1296, (1) SEQ ID NOs: 1344 and 1296, (li) SEQ ID NOs: 1345 and 1296, (lii) SEQ ID NOs: 1346 and 1296, (liii) SEQ ID NOs: 1347 and 1296, (liv) SEQ ID NOs: 1348 and 1296, (lv) SEQ ID NOs: 1349 and 1296, (lvi) SEQ ID NOs: 1350 and 1296, (lvii) SEQ ID NOs: 1351 and 1296, (lviii) SEQ ID NOs: 1352 and 1296, (lix) SEQ ID NOs: 1353 and 1296, (lx) SEQ ID NOs: 1354 and 1296, (lxi) SEQ ID NOs: 1355 and 1296, (lxii) SEQ ID NOs: 1356 and 1296, (lxiii) SEQ ID NOs: 1357 and 1296, (lxiv) SEQ ID NOs: 1358 and 1296, (lxv) SEQ ID NOs: 1359 and 1296, (lxvi) SEQ ID NOs: 1360 and 1296, (lxvii) SEQ ID NOs: 1361 and 1296, (lxviii) SEQ ID NOs: 1362 and 1296, (lxix) SEQ ID NOs: 1363 and 1296, (lxx) SEQ ID NOs: 1364 and 1296, (lxxi) SEQ ID NOs: 1365 and 1296, (lxxii) SEQ ID NOs: 1366 and 1296, (lxxiii) SEQ ID NOs: 1367 and 1296, (lxxiv) SEQ ID NOs: 1368 and 1296, (lxxv) SEQ ID NOs: 1369 and 1296, (lxxvi) SEQ ID NOs: 1370 and 1296, (lxxvii) SEQ ID NOs: 1371 and 1296, (lxxviii) SEQ ID NOs: 1372 and 1296, (lxxix) SEQ ID NOs: 1373 and 1296, (lxxx) SEQ ID NOs: 1374 and 1296, (lxxxi) SEQ ID NOs: 1375 and 1296, (lxxxii) SEQ ID NOs: 1376 and 1296, (lxxxiii) SEQ ID NOs: 1377 and 1296, (lxxxix) SEQ ID NOs: 1378 and 1296, (lxxxv) SEQ ID NOs: 1379 and 1296, (lxxxvi) SEQ ID NOs: 1380 and 1296, (lxxxvii) SEQ ID NOs: 1381 and 1296, (lxxxviii) SEQ ID NOs: 1382 and 1296, (lxxxix) SEQ ID NOs: 1383 and 1296, (xc) SEQ ID NOs: 1384 and 1296, (xci) SEQ ID NOs: 1385 and 1296, (xcii) SEQ ID NOs: 1386 and 1296, and (xciii) SEQ ID NOs: 1387 and 1296, as shown in FIGS. 27, 28, 29A-29H, and 30A-30R, as well as those disclosed herein (see, e.g., Section (V)(B)(1) above).

Embodiment (B)(2): In some other further embodiments of an NKp46 NKE antibody comprising means for binding the ECD of a human TTA, the human TTA comprises human EGFR. Suitable variable heavy VH and variable light VL domains that bind to the ECD of human EGFR include, but are not limited to, VH/VL pairs selected from the group including: (i) SEQ ID NOs: 164 and 165, (ii) SEQ ID NOs: 166 and 167, (iii) SEQ ID NOs: 168 and 169, (iv) SEQ ID NOs: 170 and 171, (v) SEQ ID NOs: 172 and 173, (vi) SEQ ID NOs: 174 and 175, (vii) SEQ ID NOs: 176 and 177, (viii) SEQ ID NOs: 178 and 179, (ix) SEQ ID NOs: 180 and 181, (x) SEQ ID NOs: 182 and 183, (xi) SEQ ID NOs: 184 and 185, and (xii) SEQ ID NOs: 186 and 187, as shown in FIGS. 13A-13C, as well as those disclosed herein (see, e.g., Section (V)(B)(2) above).

Embodiment (B)(3): In some other further embodiments of an NKp46 NKE antibody comprising means for binding the ECD of a human TTA, the human TTA comprises human HER2. Suitable variable heavy VH and variable light VL domains that bind to the ECD of human HER2 include, but are not limited to, VH/VL pairs selected from the group including: (i) SEQ ID NOs: 275 and 276, (ii) SEQ ID NOs: 277 and 278, (iii) SEQ ID NOs: 279 and 280, (iv) SEQ ID NOs: 281 and 282, (v) SEQ ID NOs: 283 and 284, (vi) SEQ ID NOs: 285 and 286, and (vii) SEQ ID NOs: 287 and 288, as shown in FIGS. 20A-20B.

Embodiment (B)(4): In some other further embodiment of an NKp46 NKE antibody comprising means for binding the ECD of a human TTA s, the human TTA comprises human CD19. Suitable variable heavy VH and variable light VL domains that bind to the ECD of human CD19 include, but are not limited to, VH/VL pairs selected from the group including: (i) SEQ ID NOs: 188 and 189, (ii) SEQ ID NOs: 190 and 191, (iii) SEQ ID NOs: 192 and 193, and (iv) SEQ ID NOs: 194 and 195, as shown in FIG. 14.

Embodiment (B)(5): In some other further embodiments of an NKp46 NKE antibody comprising means for binding the ECD of a human TTA, the human TTA comprises human CD20. Suitable variable heavy VH and variable light VL domains that bind to the ECD of human CD20 include, but are not limited to, VH/VL pairs selected from the group including: (i) SEQ ID NOs: 196 and 197, (ii) SEQ ID NOs: 198 and 199, (iii) SEQ ID NOs: 200 and 201, (iv) SEQ ID NOs: 202 and 203, (v) SEQ ID NOs: 204 and 205, (vi) SEQ ID NOs: 206 and 207, (vii) SEQ ID NOs: 208 and 209, (viii) SEQ ID NOs: 210 and 211, (ix) SEQ ID NOs: 212 and 213, (x) SEQ ID NOs: 214 and 215, (xi) SEQ ID NOs: 216 and 217, (xii) SEQ ID NOs: 218 and 219, (xiii) SEQ ID NOs: 220 and 221, (xiv) SEQ ID NOs: 222 and 223, and (xv) SEQ ID NOs: 224 and 225, as shown in FIGS. 15A-15C.

Embodiment (B)(6): In some other further embodiments of an NKp46 NKE antibody comprising means for binding the ECD of a human TTA, the human TTA comprises human CD123. Suitable variable heavy VH and variable light VL domains that bind to the ECD of human CD123 include, but are not limited to, VH/VL pairs selected from the group including: (i) SEQ ID NOs: 833 and 834, and (ii) SEQ ID NOs: 835 and 836, as shown in FIG. 23.

Embodiment (B)(7): In some other further embodiments of an NKp46 NKE antibody comprising means for binding the ECD of a human TTA, the human TTA comprises human CAIX. Suitable variable heavy VH and variable light VL domains that bind to the ECD of human CAIX include, but are not limited to, the pair of SEQ ID NOs: 831 and 832, as shown in FIG. 23, as well as those disclosed herein (see, e.g., Section (V)(B)(7) above).

Embodiment (B)(8): In some other further embodiments of an NKp46 NKE antibody comprising means for binding the ECD of a human TTA, the human TTA comprises human FLT3. Suitable variable heavy VH and variable light VL domains that bind to the ECD of human FLT3 include, but are not limited to, VH/VL pairs selected from the group including: (i) SEQ ID NOs: 267 and 268, (ii) SEQ ID NOs: 269 and 270, (iii) SEQ ID NOs: 271 and 272, (iv) SEQ ID NOs: 273 and 274, (v) SEQ ID NOs: 275 and 276, (vi) SEQ ID NOs: 277 and 278, (vii) SEQ ID NOs: 279 and 280, and (viii) SEQ ID NOs: 281 and 282, as shown in FIG. 19, as well as those disclosed herein (see, e.g., Section (V)(B)(8) above).

Embodiment (B)(9): In some other further embodiments of an NKp46 NKE antibody comprising means for binding the ECD of a human TTA, the human TTA comprises human MSLN. Suitable variable heavy VH and variable light VL domains that bind to the ECD of human MSLN include, but are not limited to, VH/VL pairs selected from the group including: (i) SEQ ID NOs: 289 and 293, (ii) SEQ ID NOs: 297 and 301, (iii) SEQ ID NOs: 305 and 309, (iv) SEQ ID NOs: 313 and 314, (v) SEQ ID NOs: 315 and 316, (vi) SEQ ID NOs: 317 and 318, (vii) SEQ ID NOs: 319 and 320, (viii) SEQ ID NOs: 321 and 322, (ix) SEQ ID NOs: 323 and 324, (x) SEQ ID NOs: 325 and 326, (xi) SEQ ID NOs: 327 and 328, (xii) SEQ ID NOs: 329 and 330, (xiii) SEQ ID NOs: 331 and 332, (xiv) SEQ ID NOs: 333 and 334, (xv) SEQ ID NOs: 335 and 336, (xvi) SEQ ID NOs: 337 and 338, (xvii) SEQ ID NOs: 339 and 340, (xviii) SEQ ID NOs: 341 and 342, (xix) SEQ ID NOs: 343 and 344, (xx) SEQ ID NOs: 345 and 346, (xxi) SEQ ID NOs: 347 and 348, (xxii) SEQ ID NOs: 349 and 350, (xxiii) SEQ ID NOs: 351 and 352, (xxiv) SEQ ID NOs: 353 and 354, (xxv) SEQ ID NOs: 355 and 356, (xxvi) SEQ ID NOs: 357 and 358, (xxvii) SEQ ID NOs: 359 and 360, (xxviii) SEQ ID NOs: 361 and 362, (xxix) SEQ ID NOs: 363 and 364, (xxx) SEQ ID NOs: 365 and 366, (xxxi) SEQ ID NOs: 367 and 368, (xxxii) SEQ ID NOs: 369 and 370, (xxxiii) SEQ ID NOs: 371 and 372, (xxxiv) SEQ ID NOs: 373 and 374, (xxxv) SEQ ID NOs: 375 and 376, (xxxvi) SEQ ID NOs: 377 and 378, (xxxvii) SEQ ID NOs: 379 and 380, (xxxviii) SEQ ID NOs: 381 and 382, (xxxix) SEQ ID NOs: 383 and 384, (xl) SEQ ID NOs: 385 and 386, (xli) SEQ ID NOs: 387 and 388, (xlii) SEQ ID NOs: 389 and 390, (xliii) SEQ ID NOs: 391 and 392, (xliv) SEQ ID NOs: 393 and 394, (xlv) SEQ ID NOs: 395 and 396, (xlvi) SEQ ID NOs: 397 and 398, (xlvii) SEQ ID NOs: 399 and 400, (xlviii) SEQ ID NOs: 401 and 402, (xlix) SEQ ID NOs: 403 and 404, (1) SEQ ID NOs: 405 and 406, (li) SEQ ID NOs: 405 and 408, (lii) SEQ ID NOs: 407 and 406, (liii) SEQ ID NOs: 407 and 408, (liv) SEQ ID NOs: 409 and 410, (lv) SEQ ID NOs: 411 and 412, (lvi) SEQ ID NOs: 413 and 414, (lvii) SEQ ID NOs: 415 and 416, (lviii) SEQ ID NOs: 417 and 418, (lix) SEQ ID NOs: 419 and 420, (lx) SEQ ID NOs: 421 and 422, (lxi) SEQ ID NOs: 423 and 424, (lxii) SEQ ID NOs: 425 and 426, (lxiii) SEQ ID NOs: 427 and 428, (lxiv) SEQ ID NOs: 429 and 430, (lxv) SEQ ID NOs: 431 and 432, (lxvi) SEQ ID NOs: 433 and 434, (lxvii) SEQ ID NOs: 435 and 436, (lxviii) SEQ ID NOs: 437 and 438, (lxix) SEQ ID NOs: 439 and 440, (lxx) SEQ ID NOs: 441 and 442, (lxxi) SEQ ID NOs: 443 and 444, (lxxii) SEQ ID NOs: 445 and 446, (lxxiii) SEQ ID NOs: 447 and 448, (lxxiv) SEQ ID NOs: 449 and 450, (lxxv) SEQ ID NOs: 451 and 452, (lxxvi) SEQ ID NOs: 453 and 454, (lxxvii) SEQ ID NOs: 455 and 456, (lxxviii) SEQ ID NOs: 457 and 458, (lxxix) SEQ ID NOs: 459 and 460, (lxxx) SEQ ID NOs: 461 and 462, (lxxxi) SEQ ID NOs: 463 and 464, (lxxxii) SEQ ID NOs: 465 and 466, (lxxxiii) SEQ ID NOs: 467 and 468, (lxxxix) SEQ ID NOs: 469 and 470, (lxxxv) SEQ ID NOs: 471 and 472, (lxxxvi) SEQ ID NOs: 473 and 474, (lxxxvii) SEQ ID NOs: 475 and 476, (lxxxviii) SEQ ID NOs: 477 and 478, (lxxxix) SEQ ID NOs: 479 and 480, (xc) SEQ ID NOs: 481 and 482, (xci) SEQ ID NOs: 483 and 484, (xcii) SEQ ID NOs: 485 and 486, (xciii) SEQ ID NOs: 487 and 488, (xciv) SEQ ID NOs: 489 and 490, (xcv) SEQ ID NOs: 491 and 492, (xcvi) SEQ ID NOs: 493 and 494, (xcvii) SEQ ID NOs: 495 and 496, (xcviii) SEQ ID NOs: 497 and 498, (xcix) SEQ ID NOs: 499 and 500, (c) SEQ ID NOs: 501 and 502, (ci) SEQ ID NOs: 503 and 504, (cii) SEQ ID NOs: 505 and 506, (ciii) SEQ ID NOs: 507 and 508, (civ) SEQ ID NOs: 509 and 510, (cv) SEQ ID NOs: 511 and 512, (cvi) SEQ ID NOs: 513 and 514, (cvii) SEQ ID NOs: 515 and 516, (cviii) SEQ ID NOs: 517 and 518, (cix) SEQ ID NOs: 519 and 520, (cx) SEQ ID NOs: 521 and 522, (cxi) SEQ ID NOs: 523 and 524, (cxii) SEQ ID NOs: 525 and 526, (cxiii) SEQ ID NOs: 527 and 528, (cxiv) SEQ ID NOs: 529 and 530, (cxv) SEQ ID NOs: 531 and 532, (cxvi) SEQ ID NOs: 533 and 534, (cxvii) SEQ ID NOs: 535 and 536, (cxviii) SEQ ID NOs: 537 and 538, (cxix) SEQ ID NOs: 539 and 540, (cxx) SEQ ID NOs: 541 and 542, (cxxi) SEQ ID NOs: 543 and 544, (cxxii) SEQ ID NOs: 545 and 546, (cxxiii) SEQ ID NOs: 547 and 548, (cxxiv) SEQ ID NOs: 549 and 550, (cxxv) SEQ ID NOs: 551 and 552, (cxxvi) SEQ ID NOs: 553 and 554, (cxxvii) SEQ ID NOs: 555 and 556, (cxxviii) SEQ ID NOs: 557 and 558, (cxxix) SEQ ID NOs: 559 and 560, (cxxx) SEQ ID NOs: 561 and 562, (cxxxi) SEQ ID NOs: 563 and 564, (cxxxii) SEQ ID NOs: 565 and 566, (cxxxiii) SEQ ID NOs: 567 and 568, (cxxxiv) SEQ ID NOs: 569 and 570, (cxxxv) SEQ ID NOs: 571 and 572, (cxxxvi) SEQ ID NOs: 573 and 574, (cxxxvii) SEQ ID NOs: 575 and 576, (cxxxviii) SEQ ID NOs: 577 and 578, (cxl) SEQ ID NOs: 579 and 580, (cxli) SEQ ID NOs: 581 and 582, (cxlii) SEQ ID NOs: 583 and 584, (cxliii) SEQ ID NOs: 585 and 586, (cxliv) SEQ ID NOs: 587 and 588, (cxlv) SEQ ID NOs: 589 and 590, (cxlvi) SEQ ID NOs: 591 and 592, (cxlvii) SEQ ID NOs: 593 and 594, (cxlviii) SEQ ID NOs: 595 and 596, (cxlix) SEQ ID NOs: 597 and 598, (cl) SEQ ID NOs: 599 and 600, (cli) SEQ ID NOs: 601 and 602, (clii) SEQ ID NOs: 603 and 604, (cliii) SEQ ID NOs: 605 and 606, (cliv) SEQ ID NOs: 607 and 608, (clv) SEQ ID NOs: 609 and 610, (clvi) SEQ ID NOs: 611 and 612, (clvii) SEQ ID NOs: 613 and 614, (clviii) SEQ ID NOs: 615 and 616, (clviii) SEQ ID NOs: 617 and 618, (clix) SEQ ID NOs: 619 and 620, (clx) SEQ ID NOs: 621 and 622, (clxi) SEQ ID NOs: 623 and 624, (clxii) SEQ ID NOs: 625 and 626, (clxiii) SEQ ID NOs: 627 and 628, (clxiv) SEQ ID NOs: 629 and 630, (clxv) SEQ ID NOs: 631 and 632, (clxvi) SEQ ID NOs: 633 and 634, (clxvii) SEQ ID NOs: 635 and 636, (clxviii) SEQ ID NOs: 637 and 638, and (clxix) any one of SEQ ID NOs: 639-649 and any one of SEQ ID NOs: 650-669, as shown in FIGS. 21A-21FF, as well as those disclosed herein (see, e.g., Section (V)(B)(9) above).

Embodiment (B)(10): In some other further embodiments of an NKp46 NKE antibody comprising means for binding the ECD of a human TTA, the human TTA comprises human Trop2. Suitable variable heavy VH and variable light VL domains that bind to the ECD of human Trop2 include, but are not limited to, VH/VL pairs selected from the group including: (i) SEQ ID NOs: 670 and 671, (ii) SEQ ID NOs: 672 and 673, (iii) SEQ ID NOs: 674 and 675, (iv) SEQ ID NOs: 676 and 677, (v) SEQ ID NOs: 678 and 679, (vi) SEQ ID NOs: 680 and 681, (vii) SEQ ID NOs: 682 and 683, (viii) SEQ ID NOs: 684 and 681, (ix) SEQ ID NOs: 685 and 686, (x) SEQ ID NOs: 687 and 688, (xi) SEQ ID NOs: 689 and 690, (xii) SEQ ID NOs: 691 and 692, (xiii) SEQ ID NOs: 693 and 694, (xiv) SEQ ID NOs: 695 and 696, (xv) SEQ ID NOs: 697 and 698, (xvi) SEQ ID NOs: 699 and 700, (xvii) SEQ ID NOs: 701 and 702, (xviii) SEQ ID NOs: 703 and 704, (xix) SEQ ID NOs: 705 and 706, (xx) SEQ ID NOs: 707 and 708, (xxi) SEQ ID NOs: 709 and 710, (xxii) SEQ ID NOs: 711 and 712, (xxiii) SEQ ID NOs: 713 and 714, (xxiv) SEQ ID NOs: 715 and 716, (xxv) SEQ ID NOs: 717 and 718, (xxvi) SEQ ID NOs: 719 and 720, (xxvii) SEQ ID NOs: 721 and 722, (xxviii) SEQ ID NOs: 723 and 724, (xxix) SEQ ID NOs: 725 and 726, (xxx) SEQ ID NOs: 727 and 728, (xxxi) SEQ ID NOs: 729 and 730, (xxxii) SEQ ID NOs: 731 and 732, (xxxiii) SEQ ID NOs: 733 and 734, (xxxiv) SEQ ID NOs: 735 and 736, (xxxv) SEQ ID NOs: 737 and 738, (xxxvi) SEQ ID NOs: 739 and 740, (xxxvii) SEQ ID NOs: 741 and 742, (xxxviii) SEQ ID NOs: 743 and 744, (xxxix) SEQ ID NOs: 745 and 746, (xl) SEQ ID NOs: 747 and 748, (xli) SEQ ID NOs: 749 and 750, (xlii) SEQ ID NOs: 751 and 752, (xliii) SEQ ID NOs: 753 and 754, (xliv) SEQ ID NOs: 755 and 756, (xlv) SEQ ID NOs: 757 and 758, (xlvi) SEQ ID NOs: 759 and 760, (xlvii) SEQ ID NOs: 761 and 762, (xlviii) SEQ ID NOs: 763 and 764, (xlix) SEQ ID NOs: 765 and 766, (1) SEQ ID NOs: 767 and 768, (li) SEQ ID NOs: 769 and 770, (lii) SEQ ID NOs: 771 and 772, (liii) SEQ ID NOs: 773 and 774, (liv) SEQ ID NOs: 775 and 776, (lv) SEQ ID NOs: 777 and 778, (lvi) SEQ ID NOs: 779 and 780, (lvii) SEQ ID NOs: 781 and 782, (lviii) SEQ ID NOs: 783 and 784, (lix) SEQ ID NOs: 785 and 786, (lx) SEQ ID NOs: 787 and 788, (lxi) SEQ ID NOs: 789 and 790, (lxii) SEQ ID NOs: 791 and 792, (lxiii) SEQ ID NOs: 793 and 794, (lxiv) SEQ ID NOs: 795 and 800, (lxv) SEQ ID NOs: 795 and 801, (lxvi) SEQ ID NOs: 795 and 802, (lxvii) SEQ ID NOs: 796 and 800, (lxviii) SEQ ID NOs: 796 and 801, (lxix) SEQ ID NOs: 796 and 802, (lxx) SEQ ID NOs: 797 and 800, (lxxi) SEQ ID NOs: 797 and 801, (lxxii) SEQ ID NOs: 797 and 802, (lxxiii) SEQ ID NOs: 798 and 800, (lxxiv) SEQ ID NOs: 798 and 801, (lxxv) SEQ ID NOs: 798 and 802, (lxxvi) SEQ ID NOs: 799 and 800, (lxxvii) SEQ ID NOs: 799 and 801, (lxxviii) SEQ ID NOs: 799 and 802, (lxxix) SEQ ID NOs: 803 and 806, (lxxx) SEQ ID NOs: 803 and 807, (lxxxi) SEQ ID NOs: 803 and 808, (lxxxii) SEQ ID NOs: 804 and 806, (lxxxiii) SEQ ID NOs: 804 and 807, (lxxxix) SEQ ID NOs: 804 and 808, (lxxxv) SEQ ID NOs: 805 and 806, (lxxxvi) SEQ ID NOs: 805 and 807, (lxxxvii) SEQ ID NOs: 805 and 808, (lxxxviii) SEQ ID NOs: 809 and 812, (lxxxix) SEQ ID NOs: 809 and 813, (xc) SEQ ID NOs: 810 and 812, (xci) SEQ ID NOs: 810 and 813, (xcii) SEQ ID NOs: 811 and 812, (xciii) SEQ ID NOs: 811 and 813, (xciv) SEQ ID NOs: 814 and 816, (xcv) SEQ ID NOs: 814 and 817, (xcvi) SEQ ID NOs: 814 and 818, (xcvii) SEQ ID NOs: 815 and 816, (xcviii) SEQ ID NOs: 815 and 817, (xcix) SEQ ID NOs: 815 and 818, (c) SEQ ID NOs: 819 and 824, (ci) SEQ ID NOs: 819 and 825, (cii) SEQ ID NOs: 819 and 826, (ciii) SEQ ID NOs: 819 and 827, (civ) SEQ ID NOs: 819 and 828, (cv) SEQ ID NOs: 820 and 824, (cvi) SEQ ID NOs: 820 and 825, (cvii) SEQ ID NOs: 820 and 826, (cviii) SEQ ID NOs: 820 and 827, (cix) SEQ ID NOs: 820 and 828, (cx) SEQ ID NOs: 821 and 824, (cxi) SEQ ID NOs: 821 and 825, (cxii) SEQ ID NOs: 821 and 826, (cxiii) SEQ ID NOs: 821 and 827, (cxiv) SEQ ID NOs: 821 and 828, (cxv) SEQ ID NOs: 822 and 824, (cxvi) SEQ ID NOs: 822 and 825, (cxvii) SEQ ID NOs: 822 and 826, (cxviii) SEQ ID NOs: 822 and 827, (cxix) SEQ ID NOs: 822 and 828, (cxx) SEQ ID NOs: 823 and 824, (cxxi) SEQ ID NOs: 823 and 825, (cxxii) SEQ ID NOs: 823 and 826, (cxxiii) SEQ ID NOs: 823 and 827, (cxxiv) SEQ ID NOs: 823 and 828, and (cxxv) SEQ ID NOs: 829 and 830, as shown in FIGS. 22A-22R, as well as those disclosed herein (see, e.g., Section (V)(B)(10) above).

Embodiment (B)(11): In some other further embodiments, the human TTA comprises human CEA. Suitable variable heavy VH and variable light VL domains that bind to the ECD of human CEA include, but are not limited to, VH/VL pairs selected from the group including: (i) SEQ ID NOs: 239 and 240, (ii) SEQ ID NOs: 241 and 242, (iii) SEQ ID NOs: 243 and 244, (iv) SEQ ID NOs: 245 and 246, (v) SEQ ID NOs: 247 and 248, (vi) SEQ ID NOs: 249 and 250, (vii) SEQ ID NOs: 251 and 252, (viii) SEQ ID NOs: 253 and 254, (ix) SEQ ID NOs: 255 and 256, (x) SEQ ID NOs: 257 and 258, (xi) SEQ ID NOs: 259 and 260, (xii) SEQ ID NOs: 261 and 262, (xiii) SEQ ID NOs: 263 and 264, and (xiv) SEQ ID NOs: 265 and 266, as shown in FIGS. 18A-18C, as well as those disclosed herein (see, e.g., Section (V)(B)(11) above).

Embodiment (B)(12): In some other further embodiments of an NKp46 NKE antibody comprising means for binding the ECD of a human TTA, the human TTA comprises human CLDN18.2. Suitable variable heavy VH and variable light VL domains that bind to the ECD of human CLDN18.2 include, but are not limited to, VH/VL pairs selected from the group including: (i) SEQ ID NOs: 226 and 229, (ii) SEQ ID NOs: 226 and 230, (iii) SEQ ID NOs: 227 and 229, (iv) SEQ ID NOs: 227 and 230, (v) SEQ ID NOs: 228 and 229, (vi) SEQ ID NOs: 228 and 230, and (vii) SEQ ID NOs: 231 and 232, as shown in FIG. 16, as well as those disclosed herein (see, e.g., Section (V)(B)(12) above).

Embodiment (B)(13): In some other further embodiments of an NKp46 NKE antibody comprising means for binding the ECD of a human TTA, the human TTA comprises human BCMA. Suitable variable heavy VH and variable light VL domains that bind to the ECD of human BCMA include, but are not limited to, VH/VL pairs selected from the group including: (i) SEQ ID NOs: 233 and 234, (ii) SEQ ID NOs: 235 and 236, (iii) SEQ ID NOs: 237 and 238, (iv) SEQ ID NOs: 12 and 16, (v) SEQ ID NOs: 22 and 26, (vi) SEQ ID NOs: 32 and 36, (vii) SEQ ID NOs: 42 and 46, (viii) SEQ ID NOs: 52 and 56, (ix) SEQ ID NOs: 62 and 66, and (x) SEQ ID NOs: 72 and 76, as shown in FIGS. 17A-17C, as well as those disclosed herein (see, e.g., Section (V)(B)(13) above).

Embodiment (B)(14): In some other further embodiments of an NKp46 NKE antibody comprising means for binding the ECD of a human TTA, the human TTA comprises human DLL3. Suitable variable heavy VH and variable light VL domains that bind to the ECD of human DLL3 include, but are not limited to, VH/VL pairs selected from the group including: (i) SEQ ID NOs: 837 and 838, (ii) SEQ ID NOs: 839 and 840, (iii) SEQ ID NOs: 841 and 842, (iv) SEQ ID NOs: 843 and 844, (v) SEQ ID NOs: 845 and 846, (vi) SEQ ID NOs: 847 and 848, (vii) SEQ ID NOs: 849 and 850, (viii) SEQ ID NOs: 851 and 852, (ix) SEQ ID NOs: 853 and 854, (x) SEQ ID NOs: 855 and 856, (xi) SEQ ID NOs: 857 and 858, (xii) SEQ ID NOs: 859 and 860, (xiii) SEQ ID NOs: 861 and 862, (xiv) SEQ ID NOs: 863 and 864, (xv) SEQ ID NOs: 865 and 866, (xvi) SEQ ID NOs: 867 and 868, and (xvii) SEQ ID NOs: 869 and 870, as shown in FIGS. 24A-24C, as well as those disclosed herein (see, e.g., Section (V)(B)(14) above).

Embodiment (B)(15): In some other further embodiments of an NKp46 NKE antibody comprising means for binding the ECD of a human TTA, the human TTA comprises human PD-1. Suitable variable heavy VH and variable light VL domains that bind to the ECD of human PD-1 include, but are not limited to, VH/VL pairs selected from the group including: (i) SEQ ID NOs: 871 and 872, (ii) SEQ ID NOs: 873 and 874, (iii) SEQ ID NOs: 875 and 876, (iv) SEQ ID NOs: 877 and 878, (v) SEQ ID NOs: 879 and 880, (vi) SEQ ID NOs: 881 and 882, (vii) SEQ ID NOs: 883 and 884, (viii) SEQ ID NOs: 885 and 886, (ix) SEQ ID NOs: 887 and 888, (x) SEQ ID NOs: 889 and 890, (xi) SEQ ID NOs: 891 and 892, (xii) SEQ ID NOs: 893 and 894, (xiii) SEQ ID NOs: 895 and 896, (xiv) SEQ ID NOs: 897 and 898, (xv) SEQ ID NOs: 899 and 900, (xvi) SEQ ID NOs: 901 and 902, (xvii) SEQ ID NOs: 903 and 904, (xviii) SEQ ID NOs: 905 and 906, (xix) SEQ ID NOs: 907 and 908, and (xx) SEQ ID NOs: 909 and 910, as shown in FIGS. 25A-25G, as well as those disclosed herein (see, e.g., Section (V)(B)(15) above).

Embodiment (B)(16): In some other further embodiments of an NKp46 NKE antibody comprising means for binding the ECD of a human TTA, the human TTA comprises human ANOL. Suitable variable heavy VH and variable light VL domains that bind to the ECD of human ANO1 include, but are not limited to, the sequences disclosed herein (see, e.g., Section (V)(B)(16) above).

Embodiment (B)(17): In some other further embodiments of an NKp46 NKE antibody comprising means for binding the ECD of a human TTA, the human TTA comprises human CD22. Suitable variable heavy VH and variable light VL domains that bind to the ECD of human CD22 include, but are not limited to, the sequences disclosed herein (see, e.g., Section (V)(B)(17) above).

Embodiment (B)(18): In some other further embodiments of an NKp46 NKE antibody comprising means for binding the ECD of a human TTA, the human TTA comprises human CD38. Suitable variable heavy VH and variable light VL domains that bind to the ECD of human CD38 include, but are not limited to, the sequences disclosed herein (see, e.g., Section (V)(B)(18) above).

Embodiment (C): In some embodiments, the NKE antibody comprises: a) means for binding the extracellular domain of human NKp30; and b) a variant IgG1 Fc dimeric domain comprising an amino acid substitution as compared to human IgG1, wherein the variant has increased in vitro binding to the human FcγRIIIa receptor as compared to human IgG1. Suitable variable heavy VH and variable light VL domains that bind to the ECD of human NKp30 include, but are not limited to, VH/VL pairs selected from the group including: (i) SEQ ID NOs: 158 and 159, (ii) SEQ ID NOs: 160 and 161, and (iii) SEQ ID NOs: 162 and 163, as shown in FIGS. 12, as well as those disclosed herein. In some further embodiments, the NKE antibody (such as the NKp30 NKE antibody described herein) further comprises means for binding the ECD of a human TTA selected from a group including: (1) B7H3, (2) EGFR, (3) HER2, (4) CD19, (5) CD20, (6) CD123, (7) CAIX, (8) FLT3, (9) MSLN, (10) Trop2, (11) CEA, (12) CLDN18.2, (13) BCMA, (14) DLL3, (15) PD-1, (16) ANO1, (17) CD22, and (18) CD38, as described in further detail below. In still some further embodiments, the variant IgG1 Fc domain comprises an amino acid substitution selected from a group including: S239D, S239E, S239D/I332E, I332E, and I332D. In still some other further embodiments, the variant IgG1 Fc domain comprises an amino acid substitution selected from a group including: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

Embodiment (C)(1): In some further embodiments of an NKp30 NKE antibody comprising means for binding the ECD of a human TTA, the human TTA comprises human B7H3. Suitable variable heavy VH and variable light VL domains that bind to the ECD of human B7H3 include, but are not limited to, VH/VL pairs selected from the group including: (i) SEQ ID NOs: 1260 and 1264, (ii) SEQ ID NOs: 1268 and 1272, (iii) SEQ ID NOs: 1276 and 1280, (iv) SEQ ID NOs: 1284 and 1288, (v) SEQ ID NOs: 1292 and 1296, (vi) SEQ ID NOs: 1300 and 1296, (vii) SEQ ID NOs: 1301 and 1296, (viii) SEQ ID NOs: 1302 and 1296, (ix) SEQ ID NOs: 1303 and 1296, (x) SEQ ID NOs: 1304 and 1296, (xi) SEQ ID NOs: 1305 and 1296, (xii) SEQ ID NOs: 1306 and 1296, (xiii) SEQ ID NOs: 1307 and 1296, (xiv) SEQ ID NOs: 1308 and 1296, (xv) SEQ ID NOs: 1309 and 1296, (xvi) SEQ ID NOs: 1310 and 1296, (xvii) SEQ ID NOs: 1311 and 1296, (xviii) SEQ ID NOs: 1312 and 1296, (xix) SEQ ID NOs: 1313 and 1296, (xx) SEQ ID NOs: 1314 and 1296, (xxi) SEQ ID NOs: 1315 and 1296, (xxii) SEQ ID NOs: 1316 and 1296, (xxiii) SEQ ID NOs: 1317 and 1296, (xxiv) SEQ ID NOs: 1318 and 1296, (xxv) SEQ ID NOs: 1319 and 1296, (xxvi) SEQ ID NOs: 1320 and 1296, (xxvii) SEQ ID NOs: 1321 and 1296, (xxviii) SEQ ID NOs: 1322 and 1296, (xxix) SEQ ID NOs: 1323 and 1296, (xxx) SEQ ID NOs: 1324 and 1296, (xxxi) SEQ ID NOs: 1325 and 1296, (xxxii) SEQ ID NOs: 1326 and 1296, (xxxiii) SEQ ID NOs: 1327 and 1296, (xxxiv) SEQ ID NOs: 1328 and 1296, (xxxv) SEQ ID NOs: 1329 and 1296, (xxxvi) SEQ ID NOs: 1330 and 1296, (xxxvii) SEQ ID NOs: 1331 and 1296, (xxxviii) SEQ ID NOs: 1332 and 1296, (xxxix) SEQ ID NOs: 1333 and 1296, (xl) SEQ ID NOs: 1334 and 1296, (xli) SEQ ID NOs: 1335 and 1296, (xlii) SEQ ID NOs: 1336 and 1296, (xliii) SEQ ID NOs: 1337 and 1296, (xliv) SEQ ID NOs: 1338 and 1296, (xlv) SEQ ID NOs: 1339 and 1296, (xlvi) SEQ ID NOs: 1340 and 1296, (xlvii) SEQ ID NOs: 1341 and 1296, (xlviii) SEQ ID NOs: 1342 and 1296, (xlix) SEQ ID NOs: 1343 and 1296, (1) SEQ ID NOs: 1344 and 1296, (li) SEQ ID NOs: 1345 and 1296, (lii) SEQ ID NOs: 1346 and 1296, (liii) SEQ ID NOs: 1347 and 1296, (liv) SEQ ID NOs: 1348 and 1296, (lv) SEQ ID NOs: 1349 and 1296, (lvi) SEQ ID NOs: 1350 and 1296, (lvii) SEQ ID NOs: 1351 and 1296, (lviii) SEQ ID NOs: 1352 and 1296, (lix) SEQ ID NOs: 1353 and 1296, (lx) SEQ ID NOs: 1354 and 1296, (lxi) SEQ ID NOs: 1355 and 1296, (lxii) SEQ ID NOs: 1356 and 1296, (lxiii) SEQ ID NOs: 1357 and 1296, (lxiv) SEQ ID NOs: 1358 and 1296, (lxv) SEQ ID NOs: 1359 and 1296, (lxvi) SEQ ID NOs: 1360 and 1296, (lxvii) SEQ ID NOs: 1361 and 1296, (lxviii) SEQ ID NOs: 1362 and 1296, (lxix) SEQ ID NOs: 1363 and 1296, (lxx) SEQ ID NOs: 1364 and 1296, (lxxi) SEQ ID NOs: 1365 and 1296, (lxxii) SEQ ID NOs: 1366 and 1296, (lxxiii) SEQ ID NOs: 1367 and 1296, (lxxiv) SEQ ID NOs: 1368 and 1296, (lxxv) SEQ ID NOs: 1369 and 1296, (lxxvi) SEQ ID NOs: 1370 and 1296, (lxxvii) SEQ ID NOs: 1371 and 1296, (lxxviii) SEQ ID NOs: 1372 and 1296, (lxxix) SEQ ID NOs: 1373 and 1296, (lxxx) SEQ ID NOs: 1374 and 1296, (lxxxi) SEQ ID NOs: 1375 and 1296, (lxxxii) SEQ ID NOs: 1376 and 1296, (lxxxiii) SEQ ID NOs: 1377 and 1296, (lxxxix) SEQ ID NOs: 1378 and 1296, (lxxxv) SEQ ID NOs: 1379 and 1296, (lxxxvi) SEQ ID NOs: 1380 and 1296, (lxxxvii) SEQ ID NOs: 1381 and 1296, (lxxxviii) SEQ ID NOs: 1382 and 1296, (lxxxix) SEQ ID NOs: 1383 and 1296, (xc) SEQ ID NOs: 1384 and 1296, (xci) SEQ ID NOs: 1385 and 1296, (xcii) SEQ ID NOs: 1386 and 1296, and (xciii) SEQ ID NOs: 1387 and 1296, as shown in FIGS. 27, 28, 29A-29H, and 30A-30R, as well as those disclosed herein (see, e.g., Section (V)(B)(1) above).

Embodiment (C)(2): In some other further embodiments of an NKp30 NKE antibody comprising means for binding the ECD of a human TTA, the human TTA comprises human EGFR. Suitable variable heavy VH and variable light VL domains that bind to the ECD of human EGFR include, but are not limited to, VH/VL pairs selected from the group including: (i) SEQ ID NOs: 164 and 165, (ii) SEQ ID NOs: 166 and 167, (iii) SEQ ID NOs: 168 and 169, (iv) SEQ ID NOs: 170 and 171, (v) SEQ ID NOs: 172 and 173, (vi) SEQ ID NOs: 174 and 175, (vii) SEQ ID NOs: 176 and 177, (viii) SEQ ID NOs: 178 and 179, (ix) SEQ ID NOs: 180 and 181, (x) SEQ ID NOs: 182 and 183, (xi) SEQ ID NOs: 184 and 185, and (xii) SEQ ID NOs: 186 and 187, as shown in FIGS. 13A-13C, as well as those disclosed herein (see, e.g., Section (V)(B)(2) above).

Embodiment (C)(3): In some other further embodiments of an NKp30 NKE antibody comprising means for binding the ECD of a human TTA, the human TTA comprises human HER2. Suitable variable heavy VH and variable light VL domains that bind to the ECD of human HER2 include, but are not limited to, VH/VL pairs selected from the group including: (i) SEQ ID NOs: 275 and 276, (ii) SEQ ID NOs: 277 and 278, (iii) SEQ ID NOs: 279 and 280, (iv) SEQ ID NOs: 281 and 282, (v) SEQ ID NOs: 283 and 284, (vi) SEQ ID NOs: 285 and 286, and (vii) SEQ ID NOs: 287 and 288, as shown in FIGS. 20A-20B.

Embodiment (C)(4): In some other further embodiments of an NKp30 NKE antibody comprising means for binding the ECD of a human TTA, the human TTA comprises human CD19. Suitable variable heavy VH and variable light VL domains that bind to the ECD of human CD19 include, but are not limited to, VH/VL pairs selected from the group including: (i) SEQ ID NOs: 188 and 189, (ii) SEQ ID NOs: 190 and 191, (iii) SEQ ID NOs: 192 and 193, and (iv) SEQ ID NOs: 194 and 195, as shown in FIG. 14.

Embodiment (C)(5): In some other further embodiments of an NKp30 NKE antibody comprising means for binding the ECD of a human TTA, the human TTA comprises human CD20. Suitable variable heavy VH and variable light VL domains that bind to the ECD of human CD20 include, but are not limited to, VH/VL pairs selected from the group including: (i) SEQ ID NOs: 196 and 197, (ii) SEQ ID NOs: 198 and 199, (iii) SEQ ID NOs: 200 and 201, (iv) SEQ ID NOs: 202 and 203, (v) SEQ ID NOs: 204 and 205, (vi) SEQ ID NOs: 206 and 207, (vii) SEQ ID NOs: 208 and 209, (viii) SEQ ID NOs: 210 and 211, (ix) SEQ ID NOs: 212 and 213, (x) SEQ ID NOs: 214 and 215, (xi) SEQ ID NOs: 216 and 217, (xii) SEQ ID NOs: 218 and 219, (xiii) SEQ ID NOs: 220 and 221, (xiv) SEQ ID NOs: 222 and 223, and (xv) SEQ ID NOs: 224 and 225, as shown in FIGS. 15A-15C.

Embodiment (C)(6): In some other further embodiments of an NKp30 NKE antibody comprising means for binding the ECD of a human TTA, the human TTA comprises human CD123. Suitable variable heavy VH and variable light VL domains that bind to the ECD of human CD123 include, but are not limited to, VH/VL pairs selected from the group including: (i) SEQ ID NOs: 833 and 834, and (ii) SEQ ID NOs: 835 and 836, as shown in FIG. 23.

Embodiment (C)(7): In some other further embodiments of an NKp30 NKE antibody comprising means for binding the ECD of a human TTA, the human TTA comprises human CAIX. Suitable variable heavy VH and variable light VL domains that bind to the ECD of human CAIX include, but are not limited to, the pair of SEQ ID NOs: 831 and 832, as shown in FIG. 23, as well as those disclosed herein (see, e.g., Section (V)(B)(7) above).

Embodiment (C)(8): In some other further embodiments of an NKp30 NKE antibody comprising means for binding the ECD of a human TTA, the human TTA comprises human FLT3. Suitable variable heavy VH and variable light VL domains that bind to the ECD of human FLT3 include, but are not limited to, VH/VL pairs selected from the group including: (i) SEQ ID NOs: 267 and 268, (ii) SEQ ID NOs: 269 and 270, (iii) SEQ ID NOs: 271 and 272, (iv) SEQ ID NOs: 273 and 274, (v) SEQ ID NOs: 275 and 276, (vi) SEQ ID NOs: 277 and 278, (vii) SEQ ID NOs: 279 and 280, and (viii) SEQ ID NOs: 281 and 282, as shown in FIG. 19, as well as those disclosed herein (see, e.g., Section (V)(B)(8) above).

Embodiment (C)(9): In some other further embodiments of an NKp30 NKE antibody comprising means for binding the ECD of a human TTA, the human TTA comprises human MSLN. Suitable variable heavy VH and variable light VL domains that bind to the ECD of human MSLN include, but are not limited to, VH/VL pairs selected from the group including: (i) SEQ ID NOs: 289 and 293, (ii) SEQ ID NOs: 297 and 301, (iii) SEQ ID NOs: 305 and 309, (iv) SEQ ID NOs: 313 and 314, (v) SEQ ID NOs: 315 and 316, (vi) SEQ ID NOs: 317 and 318, (vii) SEQ ID NOs: 319 and 320, (viii) SEQ ID NOs: 321 and 322, (ix) SEQ ID NOs: 323 and 324, (x) SEQ ID NOs: 325 and 326, (xi) SEQ ID NOs: 327 and 328, (xii) SEQ ID NOs: 329 and 330, (xiii) SEQ ID NOs: 331 and 332, (xiv) SEQ ID NOs: 333 and 334, (xv) SEQ ID NOs: 335 and 336, (xvi) SEQ ID NOs: 337 and 338, (xvii) SEQ ID NOs: 339 and 340, (xviii) SEQ ID NOs: 341 and 342, (xix) SEQ ID NOs: 343 and 344, (xx) SEQ ID NOs: 345 and 346, (xxi) SEQ ID NOs: 347 and 348, (xxii) SEQ ID NOs: 349 and 350, (xxiii) SEQ ID NOs: 351 and 352, (xxiv) SEQ ID NOs: 353 and 354, (xxv) SEQ ID NOs: 355 and 356, (xxvi) SEQ ID NOs: 357 and 358, (xxvii) SEQ ID NOs: 359 and 360, (xxviii) SEQ ID NOs: 361 and 362, (xxix) SEQ ID NOs: 363 and 364, (xxx) SEQ ID NOs: 365 and 366, (xxxi) SEQ ID NOs: 367 and 368, (xxxii) SEQ ID NOs: 369 and 370, (xxxiii) SEQ ID NOs: 371 and 372, (xxxiv) SEQ ID NOs: 373 and 374, (xxxv) SEQ ID NOs: 375 and 376, (xxxvi) SEQ ID NOs: 377 and 378, (xxxvii) SEQ ID NOs: 379 and 380, (xxxviii) SEQ ID NOs: 381 and 382, (xxxix) SEQ ID NOs: 383 and 384, (xl) SEQ ID NOs: 385 and 386, (xli) SEQ ID NOs: 387 and 388, (xlii) SEQ ID NOs: 389 and 390, (xliii) SEQ ID NOs: 391 and 392, (xliv) SEQ ID NOs: 393 and 394, (xlv) SEQ ID NOs: 395 and 396, (xlvi) SEQ ID NOs: 397 and 398, (xlvii) SEQ ID NOs: 399 and 400, (xlviii) SEQ ID NOs: 401 and 402, (xlix) SEQ ID NOs: 403 and 404, (1) SEQ ID NOs: 405 and 406, (li) SEQ ID NOs: 405 and 408, (lii) SEQ ID NOs: 407 and 406, (liii) SEQ ID NOs: 407 and 408, (liv) SEQ ID NOs: 409 and 410, (lv) SEQ ID NOs: 411 and 412, (lvi) SEQ ID NOs: 413 and 414, (lvii) SEQ ID NOs: 415 and 416, (lviii) SEQ ID NOs: 417 and 418, (lix) SEQ ID NOs: 419 and 420, (lx) SEQ ID NOs: 421 and 422, (lxi) SEQ ID NOs: 423 and 424, (lxii) SEQ ID NOs: 425 and 426, (lxiii) SEQ ID NOs: 427 and 428, (lxiv) SEQ ID NOs: 429 and 430, (lxv) SEQ ID NOs: 431 and 432, (lxvi) SEQ ID NOs: 433 and 434, (lxvii) SEQ ID NOs: 435 and 436, (lxviii) SEQ ID NOs: 437 and 438, (lxix) SEQ ID NOs: 439 and 440, (lxx) SEQ ID NOs: 441 and 442, (lxxi) SEQ ID NOs: 443 and 444, (lxxii) SEQ ID NOs: 445 and 446, (lxxiii) SEQ ID NOs: 447 and 448, (lxxiv) SEQ ID NOs: 449 and 450, (lxxv) SEQ ID NOs: 451 and 452, (lxxvi) SEQ ID NOs: 453 and 454, (lxxvii) SEQ ID NOs: 455 and 456, (lxxviii) SEQ ID NOs: 457 and 458, (lxxix) SEQ ID NOs: 459 and 460, (lxxx) SEQ ID NOs: 461 and 462, (lxxxi) SEQ ID NOs: 463 and 464, (lxxxii) SEQ ID NOs: 465 and 466, (lxxxiii) SEQ ID NOs: 467 and 468, (lxxxix) SEQ ID NOs: 469 and 470, (lxxxv) SEQ ID NOs: 471 and 472, (lxxxvi) SEQ ID NOs: 473 and 474, (lxxxvii) SEQ ID NOs: 475 and 476, (lxxxviii) SEQ ID NOs: 477 and 478, (lxxxix) SEQ ID NOs: 479 and 480, (xc) SEQ ID NOs: 481 and 482, (xci) SEQ ID NOs: 483 and 484, (xcii) SEQ ID NOs: 485 and 486, (xciii) SEQ ID NOs: 487 and 488, (xciv) SEQ ID NOs: 489 and 490, (xcv) SEQ ID NOs: 491 and 492, (xcvi) SEQ ID NOs: 493 and 494, (xcvii) SEQ ID NOs: 495 and 496, (xcviii) SEQ ID NOs: 497 and 498, (xcix) SEQ ID NOs: 499 and 500, (c) SEQ ID NOs: 501 and 502, (ci) SEQ ID NOs: 503 and 504, (cii) SEQ ID NOs: 505 and 506, (ciii) SEQ ID NOs: 507 and 508, (civ) SEQ ID NOs: 509 and 510, (cv) SEQ ID NOs: 511 and 512, (cvi) SEQ ID NOs: 513 and 514, (cvii) SEQ ID NOs: 515 and 516, (cviii) SEQ ID NOs: 517 and 518, (cix) SEQ ID NOs: 519 and 520, (cx) SEQ ID NOs: 521 and 522, (cxi) SEQ ID NOs: 523 and 524, (cxii) SEQ ID NOs: 525 and 526, (cxiii) SEQ ID NOs: 527 and 528, (cxiv) SEQ ID NOs: 529 and 530, (cxv) SEQ ID NOs: 531 and 532, (cxvi) SEQ ID NOs: 533 and 534, (cxvii) SEQ ID NOs: 535 and 536, (cxviii) SEQ ID NOs: 537 and 538, (cxix) SEQ ID NOs: 539 and 540, (cxx) SEQ ID NOs: 541 and 542, (cxxi) SEQ ID NOs: 543 and 544, (cxxii) SEQ ID NOs: 545 and 546, (cxxiii) SEQ ID NOs: 547 and 548, (cxxiv) SEQ ID NOs: 549 and 550, (cxxv) SEQ ID NOs: 551 and 552, (cxxvi) SEQ ID NOs: 553 and 554, (cxxvii) SEQ ID NOs: 555 and 556, (cxxviii) SEQ ID NOs: 557 and 558, (cxxix) SEQ ID NOs: 559 and 560, (cxxx) SEQ ID NOs: 561 and 562, (cxxxi) SEQ ID NOs: 563 and 564, (cxxxii) SEQ ID NOs: 565 and 566, (cxxxiii) SEQ ID NOs: 567 and 568, (cxxxiv) SEQ ID NOs: 569 and 570, (cxxxv) SEQ ID NOs: 571 and 572, (cxxxvi) SEQ ID NOs: 573 and 574, (cxxxvii) SEQ ID NOs: 575 and 576, (cxxxviii) SEQ ID NOs: 577 and 578, (cxl) SEQ ID NOs: 579 and 580, (cxli) SEQ ID NOs: 581 and 582, (cxlii) SEQ ID NOs: 583 and 584, (cxliii) SEQ ID NOs: 585 and 586, (cxliv) SEQ ID NOs: 587 and 588, (cxlv) SEQ ID NOs: 589 and 590, (cxlvi) SEQ ID NOs: 591 and 592, (cxlvii) SEQ ID NOs: 593 and 594, (cxlviii) SEQ ID NOs: 595 and 596, (cxlix) SEQ ID NOs: 597 and 598, (cl) SEQ ID NOs: 599 and 600, (cli) SEQ ID NOs: 601 and 602, (clii) SEQ ID NOs: 603 and 604, (cliii) SEQ ID NOs: 605 and 606, (cliv) SEQ ID NOs: 607 and 608, (clv) SEQ ID NOs: 609 and 610, (clvi) SEQ ID NOs: 611 and 612, (clvii) SEQ ID NOs: 613 and 614, (clviii) SEQ ID NOs: 615 and 616, (clviii) SEQ ID NOs: 617 and 618, (clix) SEQ ID NOs: 619 and 620, (clx) SEQ ID NOs: 621 and 622, (clxi) SEQ ID NOs: 623 and 624, (clxii) SEQ ID NOs: 625 and 626, (clxiii) SEQ ID NOs: 627 and 628, (clxiv) SEQ ID NOs: 629 and 630, (clxv) SEQ ID NOs: 631 and 632, (clxvi) SEQ ID NOs: 633 and 634, (clxvii) SEQ ID NOs: 635 and 636, (clxviii) SEQ ID NOs: 637 and 638, and (clxix) any one of SEQ ID NOs: 639-649 and any one of SEQ ID NOs: 650-669, as shown in FIGS. 21A-21FF, as well as those disclosed herein (see, e.g., Section (V)(B)(9) above).

Embodiment (C)(10): In some other further embodiments of an NKp30 NKE antibody comprising means for binding the ECD of a human TTA, the human TTA comprises human Trop2. Suitable variable heavy VH and variable light VL domains that bind to the ECD of human Trop2 include, but are not limited to, VH/VL pairs selected from the group including: (i) SEQ ID NOs: 670 and 671, (ii) SEQ ID NOs: 672 and 673, (iii) SEQ ID NOs: 674 and 675, (iv) SEQ ID NOs: 676 and 677, (v) SEQ ID NOs: 678 and 679, (vi) SEQ ID NOs: 680 and 681, (vii) SEQ ID NOs: 682 and 683, (viii) SEQ ID NOs: 684 and 681, (ix) SEQ ID NOs: 685 and 686, (x) SEQ ID NOs: 687 and 688, (xi) SEQ ID NOs: 689 and 690, (xii) SEQ ID NOs: 691 and 692, (xiii) SEQ ID NOs: 693 and 694, (xiv) SEQ ID NOs: 695 and 696, (xv) SEQ ID NOs: 697 and 698, (xvi) SEQ ID NOs: 699 and 700, (xvii) SEQ ID NOs: 701 and 702, (xviii) SEQ ID NOs: 703 and 704, (xix) SEQ ID NOs: 705 and 706, (xx) SEQ ID NOs: 707 and 708, (xxi) SEQ ID NOs: 709 and 710, (xxii) SEQ ID NOs: 711 and 712, (xxiii) SEQ ID NOs: 713 and 714, (xxiv) SEQ ID NOs: 715 and 716, (xxv) SEQ ID NOs: 717 and 718, (xxvi) SEQ ID NOs: 719 and 720, (xxvii) SEQ ID NOs: 721 and 722, (xxviii) SEQ ID NOs: 723 and 724, (xxix) SEQ ID NOs: 725 and 726, (xxx) SEQ ID NOs: 727 and 728, (xxxi) SEQ ID NOs: 729 and 730, (xxxii) SEQ ID NOs: 731 and 732, (xxxiii) SEQ ID NOs: 733 and 734, (xxxiv) SEQ ID NOs: 735 and 736, (xxxv) SEQ ID NOs: 737 and 738, (xxxvi) SEQ ID NOs: 739 and 740, (xxxvii) SEQ ID NOs: 741 and 742, (xxxviii) SEQ ID NOs: 743 and 744, (xxxix) SEQ ID NOs: 745 and 746, (xl) SEQ ID NOs: 747 and 748, (xli) SEQ ID NOs: 749 and 750, (xlii) SEQ ID NOs: 751 and 752, (xliii) SEQ ID NOs: 753 and 754, (xliv) SEQ ID NOs: 755 and 756, (xlv) SEQ ID NOs: 757 and 758, (xlvi) SEQ ID NOs: 759 and 760, (xlvii) SEQ ID NOs: 761 and 762, (xlviii) SEQ ID NOs: 763 and 764, (xlix) SEQ ID NOs: 765 and 766, (1) SEQ ID NOs: 767 and 768, (li) SEQ ID NOs: 769 and 770, (lii) SEQ ID NOs: 771 and 772, (liii) SEQ ID NOs: 773 and 774, (liv) SEQ ID NOs: 775 and 776, (lv) SEQ ID NOs: 777 and 778, (lvi) SEQ ID NOs: 779 and 780, (lvii) SEQ ID NOs: 781 and 782, (lviii) SEQ ID NOs: 783 and 784, (lix) SEQ ID NOs: 785 and 786, (lx) SEQ ID NOs: 787 and 788, (lxi) SEQ ID NOs: 789 and 790, (lxii) SEQ ID NOs: 791 and 792, (lxiii) SEQ ID NOs: 793 and 794, (lxiv) SEQ ID NOs: 795 and 800, (lxv) SEQ ID NOs: 795 and 801, (lxvi) SEQ ID NOs: 795 and 802, (lxvii) SEQ ID NOs: 796 and 800, (lxviii) SEQ ID NOs: 796 and 801, (lxix) SEQ ID NOs: 796 and 802, (lxx) SEQ ID NOs: 797 and 800, (lxxi) SEQ ID NOs: 797 and 801, (lxxii) SEQ ID NOs: 797 and 802, (lxxiii) SEQ ID NOs: 798 and 800, (lxxiv) SEQ ID NOs: 798 and 801, (lxxv) SEQ ID NOs: 798 and 802, (lxxvi) SEQ ID NOs: 799 and 800, (lxxvii) SEQ ID NOs: 799 and 801, (lxxviii) SEQ ID NOs: 799 and 802, (lxxix) SEQ ID NOs: 803 and 806, (lxxx) SEQ ID NOs: 803 and 807, (lxxxi) SEQ ID NOs: 803 and 808, (lxxxii) SEQ ID NOs: 804 and 806, (lxxxiii) SEQ ID NOs: 804 and 807, (lxxxix) SEQ ID NOs: 804 and 808, (lxxxv) SEQ ID NOs: 805 and 806, (lxxxvi) SEQ ID NOs: 805 and 807, (lxxxvii) SEQ ID NOs: 805 and 808, (lxxxviii) SEQ ID NOs: 809 and 812, (lxxxix) SEQ ID NOs: 809 and 813, (xc) SEQ ID NOs: 810 and 812, (xci) SEQ ID NOs: 810 and 813, (xcii) SEQ ID NOs: 811 and 812, (xciii) SEQ ID NOs: 811 and 813, (xciv) SEQ ID NOs: 814 and 816, (xcv) SEQ ID NOs: 814 and 817, (xcvi) SEQ ID NOs: 814 and 818, (xcvii) SEQ ID NOs: 815 and 816, (xcviii) SEQ ID NOs: 815 and 817, (xcix) SEQ ID NOs: 815 and 818, (c) SEQ ID NOs: 819 and 824, (ci) SEQ ID NOs: 819 and 825, (cii) SEQ ID NOs: 819 and 826, (ciii) SEQ ID NOs: 819 and 827, (civ) SEQ ID NOs: 819 and 828, (cv) SEQ ID NOs: 820 and 824, (cvi) SEQ ID NOs: 820 and 825, (cvii) SEQ ID NOs: 820 and 826, (cviii) SEQ ID NOs: 820 and 827, (cix) SEQ ID NOs: 820 and 828, (cx) SEQ ID NOs: 821 and 824, (cxi) SEQ ID NOs: 821 and 825, (cxii) SEQ ID NOs: 821 and 826, (cxiii) SEQ ID NOs: 821 and 827, (cxiv) SEQ ID NOs: 821 and 828, (cxv) SEQ ID NOs: 822 and 824, (cxvi) SEQ ID NOs: 822 and 825, (cxvii) SEQ ID NOs: 822 and 826, (cxviii) SEQ ID NOs: 822 and 827, (cxix) SEQ ID NOs: 822 and 828, (cxx) SEQ ID NOs: 823 and 824, (cxxi) SEQ ID NOs: 823 and 825, (cxxii) SEQ ID NOs: 823 and 826, (cxxiii) SEQ ID NOs: 823 and 827, (cxxiv) SEQ ID NOs: 823 and 828, and (cxxv) SEQ ID NOs: 829 and 830, as shown in FIGS. 22A-22R, as well as those disclosed herein (see, e.g., Section (V)(B)(10) above).

Embodiment (C)(11): In some other further embodiments of an NKp30 NKE antibody comprising means for binding the ECD of a human TTA, the human TTA comprises human CEA. Suitable variable heavy VH and variable light VL domains that bind to the ECD of human CEA include, but are not limited to, VH/VL pairs selected from the group including: (i) SEQ ID NOs: 239 and 240, (ii) SEQ ID NOs: 241 and 242, (iii) SEQ ID NOs: 243 and 244, (iv) SEQ ID NOs: 245 and 246, (v) SEQ ID NOs: 247 and 248, (vi) SEQ ID NOs: 249 and 250, (vii) SEQ ID NOs: 251 and 252, (viii) SEQ ID NOs: 253 and 254, (ix) SEQ ID NOs: 255 and 256, (x) SEQ ID NOs: 257 and 258, (xi) SEQ ID NOs: 259 and 260, (xii) SEQ ID NOs: 261 and 262, (xiii) SEQ ID NOs: 263 and 264, and (xiv) SEQ ID NOs: 265 and 266, as shown in FIGS. 18A-18C, as well as those disclosed herein (see, e.g., Section (V)(B)(11) above).

Embodiment (C)(12): In some other further embodiments of an NKp30 NKE antibody comprising means for binding the ECD of a human TTA, the human TTA comprises human CLDN18.2. Suitable variable heavy VH and variable light VL domains that bind to the ECD of human CLDN18.2 include, but are not limited to, VH/VL pairs selected from the group including: (i) SEQ ID NOs: 226 and 229, (ii) SEQ ID NOs: 226 and 230, (iii) SEQ ID NOs: 227 and 229, (iv) SEQ ID NOs: 227 and 230, (v) SEQ ID NOs: 228 and 229, (vi) SEQ ID NOs: 228 and 230, and (vii) SEQ ID NOs: 231 and 232, as shown in FIG. 16, as well as those disclosed herein (see, e.g., Section (V)(B)(12) above).

Embodiment (C)(13): In some other further embodiments of an NKp30 NKE antibody comprising means for binding the ECD of a human TTA, the human TTA comprises human BCMA. Suitable variable heavy VH and variable light VL domains that bind to the ECD of human BCMA include, but are not limited to, VH/VL pairs selected from the group including: (i) SEQ ID NOs: 233 and 234, (ii) SEQ ID NOs: 235 and 236, (iii) SEQ ID NOs: 237 and 238, (iv) SEQ ID NOs: 12 and 16, (v) SEQ ID NOs: 22 and 26, (vi) SEQ ID NOs: 32 and 36, (vii) SEQ ID NOs: 42 and 46, (viii) SEQ ID NOs: 52 and 56, (ix) SEQ ID NOs: 62 and 66, and (x) SEQ ID NOs: 72 and 76, as shown in FIGS. 17A-17C, as well as those disclosed herein (see, e.g., Section (V)(B)(13) above).

Embodiment (C)(14): In some other further embodiments of an NKp30 NKE antibody comprising means for binding the ECD of a human TTA, the human TTA comprises human DLL3. Suitable variable heavy VH and variable light VL domains that bind to the ECD of human DLL3 include, but are not limited to, VH/VL pairs selected from the group including: (i) SEQ ID NOs: 837 and 838, (ii) SEQ ID NOs: 839 and 840, (iii) SEQ ID NOs: 841 and 842, (iv) SEQ ID NOs: 843 and 844, (v) SEQ ID NOs: 845 and 846, (vi) SEQ ID NOs: 847 and 848, (vii) SEQ ID NOs: 849 and 850, (viii) SEQ ID NOs: 851 and 852, (ix) SEQ ID NOs: 853 and 854, (x) SEQ ID NOs: 855 and 856, (xi) SEQ ID NOs: 857 and 858, (xii) SEQ ID NOs: 859 and 860, (xiii) SEQ ID NOs: 861 and 862, (xiv) SEQ ID NOs: 863 and 864, (xv) SEQ ID NOs: 865 and 866, (xvi) SEQ ID NOs: 867 and 868, and (xvii) SEQ ID NOs: 869 and 870, as shown in FIGS. 24A-24C, as well as those disclosed herein (see, e.g., Section (V)(B)(14) above).

Embodiment (C)(15): In some other further embodiments of an NKp30 NKE antibody comprising means for binding the ECD of a human TTA, the human TTA comprises human PD-1. Suitable variable heavy VH and variable light VL domains that bind to the ECD of human PD-1 include, but are not limited to, VH/VL pairs selected from the group including: (i) SEQ ID NOs: 871 and 872, (ii) SEQ ID NOs: 873 and 874, (iii) SEQ ID NOs: 875 and 876, (iv) SEQ ID NOs: 877 and 878, (v) SEQ ID NOs: 879 and 880, (vi) SEQ ID NOs: 881 and 882, (vii) SEQ ID NOs: 883 and 884, (viii) SEQ ID NOs: 885 and 886, (ix) SEQ ID NOs: 887 and 888, (x) SEQ ID NOs: 889 and 890, (xi) SEQ ID NOs: 891 and 892, (xii) SEQ ID NOs: 893 and 894, (xiii) SEQ ID NOs: 895 and 896, (xiv) SEQ ID NOs: 897 and 898, (xv) SEQ ID NOs: 899 and 900, (xvi) SEQ ID NOs: 901 and 902, (xvii) SEQ ID NOs: 903 and 904, (xviii) SEQ ID NOs: 905 and 906, (xix) SEQ ID NOs: 907 and 908, and (xx) SEQ ID NOs: 909 and 910, as shown in FIGS. 25A-25G, as well as those disclosed herein (see, e.g., Section (V)(B)(15) above).

Embodiment (C)(16): In some other further embodiments of an NKp30 NKE antibody comprising means for binding the ECD of a human TTA, the human TTA comprises human ANOL. Suitable variable heavy VH and variable light VL domains that bind to the ECD of human ANO1 include, but are not limited to, the sequences disclosed herein (see, e.g., Section (V)(B)(16) above).

Embodiment (C)(17): In some other further embodiments of an NKp30 NKE antibody comprising means for binding the ECD of a human TTA, the human TTA comprises human CD22. Suitable variable heavy VH and variable light VL domains that bind to the ECD of human CD22 include, but are not limited to, the sequences disclosed herein (see, e.g., Section (V)(B)(17) above).

Embodiment (C)(18): In some other further embodiments of an NKp30 NKE antibody comprising means for binding the ECD of a human TTA, the human TTA comprises human CD38. Suitable variable heavy VH and variable light VL domains that bind to the ECD of human CD38 include, but are not limited to, the sequences disclosed herein (see, e.g., Section (V)(B)(18) above).

### VII. Nucleic Acids

The disclosure further provides nucleic acid compositions encoding the NK cell ABD-containing antibodies provided herein, including, but not limited to, multispecific antibodies and monospecific antibodies.

As will be appreciated by those in the art, the nucleic acid compositions will depend on the format and scaffold of the heterodimeric protein. Thus, for example, when the format requires three amino acid sequences, such as for the 1+1 Fab-scFv-Fc format (e.g., a first amino acid monomer comprising an Fc domain and a scFv, a second amino acid monomer comprising a heavy chain and a light chain), three nucleic acid sequences can be incorporated into one or more expression vectors for expression. Similarly, some formats (e.g., dual scFv formats such as disclosed in FIG. 8F) only two nucleic acids are needed; again, they can be put into one or two expression vectors.

As is known in the art, the nucleic acids encoding the components of the antibodies described herein can be incorporated into expression vectors as is known in the art, and depending on the host cells used to produce the heterodimeric antibodies described herein.

Generally, the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the antibodies described herein are then transformed into any number of different types of host cells as is well known in the art, including mammalian, bacterial, yeast, insect and/or fungal cells, with mammalian cells (e.g., CHO cells), finding use in many embodiments.

In some embodiments, nucleic acids encoding each monomer and the optional nucleic acid encoding a light chain, as applicable depending on the format, are each contained within a single expression vector, generally under different or the same promoter controls. In embodiments of particular use in the antibodies described herein, each of these two or three nucleic acids are contained on a different expression vector. As shown herein and in U.S. Pat. No. 9,822,186, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The heterodimeric antibodies described herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI, and the heterodimer also has a distinct pI, thus facilitating isoelectric purification of the “1+1 Fab-scFv-Fc” and “2+1” heterodimers (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

Additionally, as is known in the art, N- and/or C-terminal clipping can occur during protein synthesis, whereby the heavy chains depicted herein may have the C-terminal lysine (K447) removed, as well as the penultimate glycine (G446), and optionally additional amino acids residues (e.g. from 1, 2, 3, 4, 5, 6, 7, 8, 9, 10 or more C-terminal amino acids can be removed). Alternatively, the nucleic acids encoding the heavy chains of the bispecific antibodies can be engineered such that these terminal residues are eliminated entirely to facilitate additional homogeneity.

### VIII. Biological and Biochemical Functionality of the Heterodimeric Bispecific Antibodies

Generally, the antibodies described herein are administered to patients with cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### IX. Treatments

Once made, the compositions of the invention find use in a number of oncology applications, by treating cancer, generally by enhancing immune responses, including, activating NK cells, enhancing NK cell mediated lysis of tumor cells and providing co-stimulation to T cells in the tumor environment. Such compositions can be combined with proinflammatory cytokines for increased cytotoxicity against tumor cells.

A. Antibody Compositions for In Vivo Administration

Formulations of the antibodies used in accordance with the antibodies described herein are prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions.

B. Administration Modalities

The antibodies provided herein administered to a subject, in accord with known methods, such as intravenous administration as a bolus or by continuous infusion over a period of time.

C. Treatment Modalities

In the methods described herein, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MM) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the disclosure includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for cancer therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

All cited references are herein expressly incorporated by reference in their entirety.

Whereas particular embodiments of the disclosure have been described above for purposes of illustration, it will be appreciated by those skilled in the art that numerous variations of the details may be made without departing from the invention as described in the appended claims.

1. An antibody comprising:
a) means for binding the extracellular domain of human NKG2D; and
b) a variant IgG1 Fc dimeric domain comprising an amino acid substitution as compared to human IgG1, wherein the variant that has increased in vitro binding to the human FcγRIIIa receptor as compared to human IgG1.

2. An antibody comprising:
a) means for binding the extracellular domain of human NKp46; and
b) a variant IgG1 Fc domain comprising an amino acid substitution as compared to human IgG1, wherein the variant that has increased in vitro binding to the human FcγRIIIa receptor as compared to human IgG1.

3. An antibody comprising:
a) means for binding the extracellular domain of human NKp30; and
b) a variant IgG1 Fc domain comprising an amino acid substitution as compared to human IgG1, wherein the variant that has increased in vitro binding to the human FcγRIIIa receptor as compared to human IgG1.

4. The antibody according to claim 1, wherein the variant IgG1 Fc domain comprises an amino acid substitution selected from the group consisting of: S239D, S239E, I332D, I332E, S239D/I332E, S239D/I332D, S239E/I332D and S239E/I332E.

5. (canceled)

6. (canceled)

7. The antibody according to claim 1, wherein the variant IgG1 Fc domain comprises an amino acid substitution selected from the group consisting of: S239D, S239E, I332D, I332E, S239D/I332E, S239E/I332E, S239D/I332D, S239E/I332D, S239D/A330L/I332E, S239D/A330L, A330L/I332E, F243L, F243L/R292P/Y300L/V305I/P396L, 332I/P247I/A339Q, S298A/E333A/K334A, V264I/I332E, S298A, S298A/I332E, S298A/E333A/K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, E293R, S324G, E272R, P227G, G236S, D221K, H224E, K246H, D249Y, R255Y, E258H, T260H, G281D, E283H, E283L, V284E, S239D/E272I/I332E, S239D/E272Y/A330L/I332E, S239D/E272I/A330L/I332E, S239D/K274E/I332E, S239D/K326T/I332E, S239D/K326E/I332E and S239D/K274E/A330L/I332E.

8. The antibody according to claim 1, wherein the antibody further comprises means for binding a human tumor antigen selected from the group consisting of: EGFR, Trop2, CD20, B7H3, FLT3, DLL3, CD19, CD22, CD38, CEA, MSLN, BCMA, CAIX, ANO1, CD19, CD123, HER2 and CLDN18.2.

9. A heterodimeric antibody comprising:
a) means for binding the extracellular domain of human NKG2D;
b) means for binding the extracellular domain of a tumor antigen selected from the group consisting of: EGFR, Trop2, CD20, B7H3, FLT3, DLL3, CD19, CEA, MSLN, BCMA, CAIX, ANO1, CD19, CD123, CD22, CD38, HER2 and CLDN18.2; and
c) a variant IgG1 dimeric Fc domain comprising an amino acid substitution as compared to human IgG1 wherein the variant that has increased in vitro binding to the human FcγRIIIa receptor as compared to human IgG1.

10. The heterodimeric antibody according to claim 9, wherein the variant IgG1 dimeric Fc domain comprises two monomeric Fc domains, wherein one of the monomeric Fc domains comprises an amino acid substitution(s) selected from the group consisting of: S239D, S239E, I332D, I332E, S239D/I332E, S239E/I332E, S239D/I332D, S239E/I332D, S239D/330L/I332E, 239D/330L, 330L/332E, 243L, F243L/R292P/Y300L/V305I/P396L, 332I/247I/339Q, S298A/E333A/K334A, V264I/I332E, S298A, S298A/I332E, S298A/E333A.K334A, S239Q/I332E, D265G, Y296Q, S298T, L328I/I332E, V264T, V266I, S239D/I332N, S239E/I332N, S239E/I332Q, S239N/I332E, S239Q/I332D, K326E, A330Y/I332E, V264I/A330Y/I332E, A330L/I332E, V264I/A330L/I332E, L234D, L234E, L234I, L235D, L235T, A330F, L328V/I332E, S239Q/V264I/I332E, S239E/V264I/A330Y/I332E, K274R, N276Y, S324T, K334I, K334F, L234I/L235D, L235D/S239D/A330Y/I332E, S239D/V240I/A330Y/I332E, S239D/V264T/A330Y/I332E, S239D/K326E/A330Y/I332E, S239D/K326T/A330Y/I332E, 3293R, S324G, E272R, P227G, G236S, D221K, H224E, K246H, D249Y, R255Y, E258H, T260H, G281D, E283H, E283L, V284E, S239D/3272I/I332E, S239D/E272Y/A330L/I332E, S239D/E272I/A330L/I332E, S239D/K274E/I332E, S239D/K326T/I332E, S239D/K326E/I332E AND S239D/K274E/A330L/I332E.

11. The heterodimeric antibody according to claim 10, wherein both of the monomeric Fc domains comprises the amino acid substitution(s).

12. The heterodimeric antibody according to claim 10, wherein one of the monomeric Fc domains comprises an amino acid substitution selected from the group consisting of: S239D, S239E, S239D/I332E, I332E, and 1332D.

13. A heterodimeric antibody comprising:
a) means for binding the extracellular domain of human NKp46;
b) means for binding the extracellular domain of a tumor antigen selected from the group consisting of: EGFR, Trop2, CD20, B7H3, FLT3, DLL3, CD19, CEA, MSLN, BCMA, CAIX, ANO1, CD19, CD123, CD22, CD38, HER2 and CLDN18.2; and
c) a variant IgG1 dimeric Fc domain comprising an amino acid substitution as compared to human IgG1 wherein the variant that has increased in vitro binding to the human FcγRIIIa receptor as compared to human IgG1.

14. (canceled)

15. (canceled)

16. (canceled)

17. A heterodimeric antibody comprising:
a) means for binding the extracellular domain of human NKp30;
b) means for binding the extracellular domain of a tumor antigen selected from the group consisting of: EGFR, Trop2, CD20, B7H3, FLT3, DLL3, CD19, CEA, MSLN, BCMA, CAIX, ANO1, CD19, CD123, CD22, CD38, HER2 and CLDN18.2; and
c) a variant IgG1 dimeric Fc domain comprising an amino acid substitution as compared to human IgG1 wherein the variant that has increased in vitro binding to the human FcγRIIIa receptor as compared to human IgG1.

18. (canceled)

19. (canceled)

20. (canceled)

21. An antibody comprising:
a) an antigen binding domain (ABD) that binds the extracellular domain of human NKG2D; and
b) a variant IgG1 Fc dimeric domain comprising an amino acid substitution as compared to human IgG1, wherein the variant that has increased in vitro binding to the human FcγRIIIa receptor as compared to human IgG1.

22. An antibody comprising:
a) an ABD that binds the extracellular domain of human NKp46; and
b) a variant IgG1 Fc dimeric domain comprising an amino acid substitution as compared to human IgG1, wherein the variant that has increased in vitro binding to the human FcγRIIIa receptor as compared to human IgG1.

23. An antibody comprising:
a) an ABD that binds the extracellular domain of human NKp30; and
b) a variant IgG1 Fc dimeric domain comprising an amino acid substitution as compared to human IgG1, wherein the variant that has increased in vitro binding to the human FcγRIIIa receptor as compared to human IgG1.
